(19) United States
(12) Patent Application Publication
(10) Pub. No.: US 2008/0241110 A1
(43) Pub. Date: Oct. 2, 2008

(54) Targeting of sall4 for the treatment and diagnosis of proliferative disorders associated with myelodysplastic syndrome (MDS)

(75) Inventor: Yupo Ma, Las Vegas, NV (US)

Correspondence Address: DLA PIPER US LLP, 4365 EXECUTIVE DRIVE, SUITE 1100, SAN DIEGO, CA, 92121-2133, US

(73) Assignee: Nevada Cancer Institute, Las Vegas, NV (US)

(21) Appl. No.: 11/809,871

(22) Filed: Jun. 1, 2007

Related U.S. Application Data
(63) Continuation-in-part of application No. 11/606,619, filed on Nov. 29, 2006.
(60) Provisional application No. 60/741,015, filed on Nov. 29, 2005.

(51) Int. Cl. A61K 35/12; C12Q 1/68; C12Q 1/02; A61K 31/7052; A61K 38/07; A61K 31/7068; C07H 21/04

(52) U.S. Cl. 424/93.7; 435/6; 435/29; 514/44; 514/18; 514/49; 536/23.1

(57) ABSTRACT

The present invention discloses nucleic acids, proteins, and antibodies for SALL4 (including isoforms SALL4A, SALL4B, and SALL4C), a zinc finger transcriptional factor. Further, methods are disclosed which demonstrate that constitutive expression of SALL4 increases leukemogenic potential in cells of model animal systems. Moreover, constitutive expression of select isoforms (e.g., SALL4B) in transgenic mice demonstrate that these animals develop myelodysplastic syndrome (MDS)-like signs and symptoms, including subsequent acute myeloid leukemia (AML), which is transplantable. The disclosure also provides methods for identifying and purifying embryonic stem cells, adult stem cells, cancer stem cells, including leukemia stem cells, methods for identifying substances which bind to and/or modulate SALL4, methods for diagnosing MDS in a subject, and methods of treating a subject presenting MDS, AML and other forms of leukemia.

### RELATED APPLICATIONS

This application is a Continuation-in-Part which claims priority under 35 U.S.C. § 120 as a continuation-in-part of U.S. application Ser. No. 11/606,619, filed Nov. 29, 2006, which claims the benefit under 35 U.S.C. §119(e) to U.S. Application Ser. No. 60/741,015, filed Nov. 29, 2005. The disclosure of the prior applications is considered part of and is incorporated by reference in the disclosure of this application.

### STATEMENT OF GOVERNMENT SUPPORT

This invention was made in part with government support under Grant No. K08 CA097185 awarded by the National Institutes of Health (NIH). The government has certain rights in this invention.

### BACKGROUND OF THE INVENTION

1. Field of the Invention

The invention relates generally to factors associated with the Wnt/β-catenin signaling pathway and, more specifically, to interaction between transcription components of the pathway, including the SALL protein family and OCT4 and nanog, which are involved in the regulation of embryonic and cancer stem cells, including methods for the diagnosis and treatment of proliferative disorders by targeting such interaction. Further, SALL4 shutdown induces cancer stem cells to undergo apoptosis and cell-cycle arrest, which cells can be rescued by SALL4 downstream targets, including Bmi-1.

2. Background Information

ES cells derived from the inner cell mass (ICM) of the blastocyst are able to undergo self-renewing cell division and maintain their pluripotency over an indefinite period of time. ES cells can also differentiate into a variety of different cell types when cultured in vitro. The Wnt/β-catenin signaling pathway has been associated with the self-renewal of normal human stem cells (HSCs) and the granulocyte-macrophage progenitors (GMPs) of chronic myeloid leukemia (CML). Further, the transcriptional factor, OCT4, has been identified as a key regulator for the formation of ICM during preimplantation development. Moreover, OCT4 protein seems to plays a central role in maintaining the pluripotency of embryonic stem (ES) cells by regulating a wide range of genes.

The role of stem cells has been considered in the etiology of cancer. There has been increasing evidence that tumors might contain such cancer stems cells, i.e., rare cells that account for the growth of tumors. These rare cells with indefinite proliferative potential may account for the resistance observed for cancer cells in response to conventional therapeutic modalities. It is known that stem cells can be identified in adult tissues, where such cells arise from a specific tissue; e.g., hematopoietic cells. As the self renewal property of stem cells is tightly controlled in normal organogenesis, the de-regulation of self-renewal might result in carcinogenesis.

Myelodysplastic syndrome (MDS), for example, is a hematological disease marked by the accumulation of genomic abnormalities at the hematopoietic stem cell (HSC) level leading to pancytopenia, multilineage differentiation impairment, and bone marrow apoptosis.

Mortality in this disease results from pancytopenia or transformation to acute myeloid leukemia (AML). AML is a hematological cancer characterized by the accumulation of immature myeloid precursors in the bone marrow and peripheral blood.

From the analysis of genetic translocation in bone marrow samples from AML patients, it is clear that transcription factors critical for hematopoiesis play an important role in leukemogenesis. The pathogenesis of AML is considered to involve multistep genetic alternations. Because only HSCs are considered to have the ability to self-renew, they are the best candidates for the accumulation of multistep, preleukemic genetic changes and transforming them into so-called “leukemia stem cells” (LSCs).

Alternatively, downstream progenitors can acquire self-renewal capacity and give rise to leukemia. LSCs are not targeted specifically under current chemotherapy regimens yet such cells have been found to account for drug resistance and leukemia relapse.

The SALL gene family, SALL1, SALL2, SALL3, and SALL4, were originally cloned on the basis of their DNA sequence homology to Drosophila spalt (sal). In Drosophila, spalt is a homeotic gene essential for development of posterior head and anterior tail segments. It plays an important role in tracheal development, terminal differentiation of photoreceptors, and wing vein placement. In humans, the SALL gene family is associated with normal development, as well as tumorigenesis. SALL proteins belong to a group of C₂H₂ zinc finger transcription factors characterized by multiple finger domains distributed over the entire protein. During the tracheal development of Drosophila, spalt is an activated downstream target of Wingless, a Wnt ortholog. It has been demonstrated that SALL1 interacts with β-catenin by functioning as a coactivator, suggesting that the interaction between SALL and the Wnt/β-catenin pathway is bidirectional.

### SUMMARY OF THE INVENTION

The present invention relates to SALL4, a human homolog to Drosophila spalt, which is a zinc finger transcriptional factor essential for development. SALL4 and its isoforms (SALL4A, SALL4B, and SALL4C) were cloned and sequenced. The present disclosure demonstrates that SALL4 failed to be turned off in human primary AML. Further, the leukemogenic potential of constitutive expression of SALL4 in a murine model is demonstrated. Moreover, SALL4B-transgenic mice which develop myelodysplastic syndrome (MDS)-like signs and symptoms and subsequent transplantable AML are described.

Increased apoptosis associated with dysmyelopoiesis is evident in transgenic mouse marrow and colony-formation (CFU) assays. Both isoforms are able to bind to β-catenin and synergistically enhance the Wnt/β-catenin signaling pathway. This demonstrates that the constitutive expression of SALL4 causes MDS/AML, and that such expression impinges on the Wnt/β-catenin pathway. In a related aspect, the murine model disclosed provides a platform to study human MDS/AML transformation, and the Wnt/β-catenin pathway's role in the pathogenesis of leukemia stem cells.

In one embodiment, an antibody or antibody fragment is disclosed which binds to a polypeptide that includes an amino acid sequence as set forth in SEQ ID NO: 13.

In another embodiment, a method of treating myelodysplastic syndrome (MDS) in a subject is disclosed, including administering a therapeutically effective amount of an antibody which binds to a polypeptide that includes an amino acid sequence as set forth in SEQ ID NO: 13 to the subject.

In another embodiment, a method of treating myelodysplastic syndrome (MDS) in a subject is provided, including administering to the subject a composition of a polynucleotide having a sequence as set forth in SEQ ID NO: 1, SEQ ID NO: 3, SEQ ID NO: 5, a complement of SEQ ID NO: 1, a complement of SEQ ID NO: 3, a complement of SEQ ID NO: 5, and fragments thereof including at least 15 consecutive nucleotides of a polynucleotide encoding the amino acid sequence as set forth in SEQ ID NO: 2, SEQ ID NO: 4, or SEQ ID NO:6.

In one embodiment, a method of treating myelodysplastic syndrome (MDS) in a subject is disclosed, including administering to the subject a polypeptide composition having a sequence as set forth in SEQ ID NO: 2, SEQ ID NO: 4 and/or SEQ ID NO: 6.

In a related aspect, the MDS is acute myeloid leukemia (AML).

In one embodiment, a method of diagnosing myelodysplastic syndrome (MDS) in a subject is disclosed, including, providing a biological sample from the subject, contacting the biological sample with a probe comprising a fragment of at least 15 consecutive nucleotides of a polynucleotide having a sequence set forth in SEQ ID NO: 1, SEQ ID NO: 3, SEQ ID NO: 5, a complement of SEQ ID NO: 1, a complement of SEQ ID NO: 3, or a complement of SEQ ID NO: 5 under hybridization conditions, and detecting the hybridization between the probe and the biological sample, where detecting of hybridization correlates with MDS.

In another embodiment, a method of diagnosing a myelodysplastic syndrome (MDS) in a subject is disclosed, including providing a biological sample from the subject, contacting the biological sample with an antibody which binds to a polypeptide comprising an amino acid sequence as set forth in SEQ ID NO: 2, SEQ ID NO: 4, or SEQ ID NO: 6, and detecting the binding of the antibody to the sample, where detecting binding correlates with MDS.

In one embodiment, a method for isolating leukemia stem cells is provided, including obtaining a sample of cells from a subject, sorting cells that express a polypeptide comprising an amino acid sequence as set forth in SEQ ID NO: 13 from cells that do not express the amino acid sequence, and selecting, by a myeloid surface marker, leukemia stem cells from the sample of cells that express the polypeptide comprising the amino acid sequence as set forth in SEQ ID NO: 13.

In another embodiment, a transgenic animal having a human SALL4 gene is provided, where the animal is modified to expresses a sequence of a human SALL4 gene comprising nucleotides encoding an amino acid as set forth in SEQ ID NO: 2, SEQ ID NO: 4, or SEQ ID NO: 6. In a related aspect, the animal constitutively expresses the inserted SALL4 gene.

In one embodiment, a method of preparing a transgenic animal comprising a human SALL4 gene is disclosed, where the animal is modified to constitutively express a sequence of a human SALL4 gene comprising nucleotides encoding an amino acid as set forth in SEQ ID NO: 2, SEQ ID NO: 4, or SEQ ID NO: 6, including introducing into embryonic cells a nucleic acid molecule a comprising a construct of human SALL4 gene comprising nucleotides encoding an amino acid as set forth in SEQ ID NO: 2, SEQ ID NO: 4, or SEQ ID NO: 6, generating a transgenic animal from the cells resulting from step the introduction of the construct, breeding the transgenic animal to obtain a transgenic animal homozygous for the human SALL4 gene, and detecting human SALL4 transcripts from tissue from the transgenic animal.

In one embodiment, a method of modulating the cellular expression of a polynucleotide encoding an amino acid sequence as set forth in SEQ ID NO: 2, SEQ ID NO: 4, or SEQ ID NO: 6 is disclosed, including introducing a double stranded RNA (dsRNA) which hybridizes to the polynucleotide, or an antisense RNA which hybridizes to the polynucleotide, or a fragment thereof, into a cell.

In one embodiment, a method of identifying a cell possessing pluripotent potential is disclosed including contacting a cell isolated from an inner cell mass (ICM), a neoplastic tissue, or a tumor with an agent that detects the expression of a SALL family member protein, and determining whether a SALL family member protein is expressed in the cell, where determining the expression of the SALL family member protein positively correlates with induction of self-renewal in the cell, whereby such expression is indicative of pluripotency.

In one aspect, the SALL family member includes SALL1, SALL3, and SALL4. In a related aspect, SALL4 is SALL4A or SALL4B.

In another aspect, the agent is an antibody directed against the SALL family member protein or a nucleic acid which is complementary to a mRNA encoding the SALL family member protein. In a related aspect, the SALL family member protein sequence includes SEQ ID NO: 2, SEQ ID NO:4, SEQ ID NO:6, SEQ ID NO:22, and SEQ ID NO:24. In another related aspect, the nucleic acid is complementary to a sense strand of a nucleic acid sequence including SEQ ID NO:1, SEQ ID NO:3, SEQ ID NO:5; SEQ ID NO:21, and SEQ ID NO:23.

In one aspect, the cell is an embryonic stem (ES) cell, an embryonic carcinoma (EC) cell, an adult stem cell, or a cancer stem cell. In a related aspect, the tissues is plasma or a biopsy sample from a subject. In a further related aspect, the subject is a human.

In one embodiment, a method of identifying an agent which modulates the effect of a SALL family member protein on OCT4 expression is disclosed including co-transfecting a cell with a vector comprising a promoter-reporter construct, where the construct comprises an operatively linked OCT4 promoter and a nucleic acid encoding gene expression reporter protein, and a vector comprising a nucleic acid encoding a SALL family member protein, contacting the cell with an agent, and determining the activity of the promoter-reporter construct in the presence and absence of the agent, where determining the activity of the promoter-reporter construct correlates with the effect of the agent on SALL family member protein/OCT4 interaction.

In a related aspect, the promoter region comprises nucleic acid sequence as set forth in SEQ ID NO:26 and the expression reporter protein is luciferase.

In another embodiment, a method of diagnosing a neoplastic or proliferative disorder is disclosed including contacting a cell of a subject with an agent that detects the expression of a SALL family member protein and determining whether a SALL family member protein is expressed in the cell, where determining the expression of the SALL family member protein positively correlates with induction of self-renewal in the cell, whereby such expression is indicative of neoplasia or proliferation.

In one aspect, the agent is labeled and the determining step includes detection of the agent by exposing the subject to a device which images the location of the agent. In a related aspect, the images are generated by magnetic resonance, X-rays, or radionuclide emission.

In one embodiment, a method of treating a neoplastic or proliferative disorder, where cells of a subject exhibit de-regulation of self-renewal, is disclosed including administering to the subject a pharmaceutical composition containing an agent which inhibits the expression of SALL4.

In another embodiment, a kit for identifying a cell possessing pluripotent potential is disclosed including an agent for detecting one or more SALL family member proteins, reagents and buffers to provide conditions sufficient for agent-cell interaction and labeling of the agent, instructions for labeling the detection reagent and for contacting the agent with the cell, and a container comprising the components.

A method of detecting cells associated with progression of a proliferative disease or neoplastic cell formation is disclosed including contacting the cells with an antibody directed against SALL4, applying cells bound to the antibody to a surface delimited cavity comprising at least two apertures for ingress and egress of fluids and cells, and allowing cells and fluids to pass through the cavity, where antibody bound cells in a fluid mixture are detected by optical detectors, and where voltage is applied to the fluid whereby the voltage assorts the bound cells in one or more collectors.

In one embodiment, a method of diagnosing disorders of primordial cell origin in a subject is disclosed including determining the expression of SALL4 in a tissue sample from the subject. In one aspect, the disorder is associated with a germ cell tumor (GCT). Further, the GCT includes classic seminoma, spermatocytic seminoma, embryonal carcinoma, yolk sac tumor, or immature teratoma.

In another aspect, the tissue sample comprises cells of testicular origin, including that substantially all mature testicular cell types present in the sample do not express SALL4. Further, the tissue sample may be obtained from a site which comprises cells that have metastasized from a GCT.

In another embodiment, a method of monitoring engraftment of transplanted stem cells in a subject is disclosed including determining the level of expression of SALL4 in stem cells prior to transplantation into a subject, grafting the cells into the subject, and determining the level of expression of SALL4 in the grafted stem cells at time intervals post-transplantation, where a decrease in SALL4 expression over the time intervals correlates with differentiation of the stem cells, and where such differentiation is indicative of positive engraftment of cells in the subject.

In one aspect, an increase in SALL4 expression over the time intervals correlates with repression of differentiation, and where such repression is indicative of negative engraftment of cells in the subject.

In another aspect, the transplanted cell is transformed by a vector encoding an exogenous or endogenous gene product.

In one embodiment, a method for isolating stem cells from cord blood disclosed including obtaining umbilical cord cells (UBC) from a subject, sorting cells that express SALL4 from cells that do not express SALL4, where UBCs expressing SALL4 are indicative of isolated stem cells. Further, the method may include, optionally, selecting cells from the sorted cells that express SALL4 using one or more additional markers.

In one aspect, the one or more markers are selected from the group consisting of SSEA-1, SSEA-2, SSEA-4, TRA-1-60, TRA-1-81, CD34⁺, CD59⁺, Thy1/CD90⁺, CD38^{lo/-}, C-kit^{-/lo}, lin−, SH2, vimentin, periodic acid Schiff activity (PAS), FLK1, BAP, and acid phosphatase.

In another embodiment, a method of treating a cancer of stem cell or progenitor cell origin is disclosed including administrating to a subject in need thereof a composition containing an agent which reduces the expression level of SALL4.

In one aspect, the agent is an oligonucleotide sequence selected from SEQ ID NO:30,

SEQ ID NO:31; or SEQ ID NO:32. In another aspect, the composition comprises a methylation inhibitor, including but not limited to, 5′ azacytidine, 5′ aza-2-deoxycytidine, 1-B-D-arabinofuranosyl-5-azacytosine, or dihydroxy-5-azacytidine. In a related aspect, the composition further comprises a proteasome inhibitor, including but not limited to,

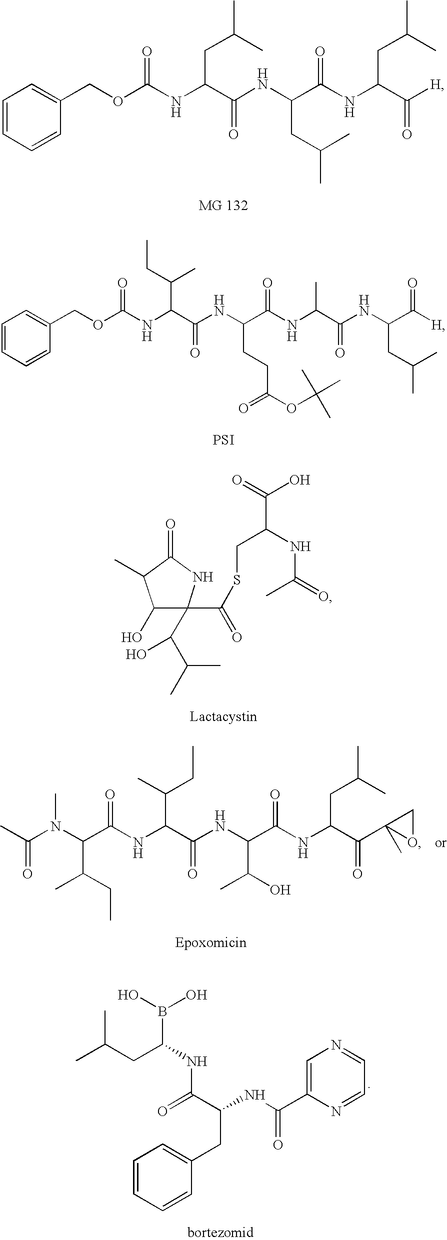

In another embodiment, an isolated oligonucleotide is disclosed, which is selected from SEQ ID NO:30, SEQ ID NO:31; SEQ ID NO:32; SEQ ID NO:33; or SEQ ID NO:34.

Exemplary methods and compositions according to this invention are described in greater detail below.

### BRIEF DESCRIPTION OF THE DRAWINGS

FIGS. 1(a-c) illustrate properties of the three SALL4 isoforms (SALL4A, SEQ ID NO: I[GenBank Acc. No.: AY172738]; SALL4B, SEQ ID NO: 3 [GenBank Acc. No. AY170621]); and SALL4C, SEQ ID NO: 5 [GenBank Acc. No. AY170622]. Alternative splicing generates two variant forms of SALL4 mRNA. FIG. 1(a) SALL4A and SALL4B vary in protein length and in the presence of different numbers of characteristic sal-like zinc finger domains. SALL4A (encoding 1,067 amino acids) contains eight zinc finger domains, while SALL4B (encoding 623 amino acids) has three zinc finger domains. SALL4C contains 276 amino acids and lacks the region corresponding to amino acids 43 to 820 of the full length SALL4A. Both variants have exons 1, 3, and 4, and SALL4A contains all exons from 1 to 4. However, SALL4B uses an alternative splice acceptor that results in deletion of the large 3′ portion of exon 2. FIG. 1(b) shows the RT-PCR analysis of SALL4 variants in different tissues. Four exons of SALL4 and their potential coding structures are illustrated, with arrows indicating the primers used for PCR amplification of the SALL4 transcripts (A). Tissue-dependent expression of SALL4 transcripts by RT-PCR (B). A 315-bp expected product that was specific for SALL4A with primers A1 (exon 2) and B1 (exon 4) was amplified with cDNAs of various tissues. Primers D1 (exon 4) and C1 (exon 1) were used to amplify the 1,851-bp expected product of SALL4B. Comparable amounts of cDNA were determined by GAPDH. FIG. 1(c) shows SALL4 protein products, SALL4A, and SALL4B identified by a SALL4 peptide antibody. Lysates from Cos-7 cells transiently expressing His-SALL4B (lane 1), His-SALL4A (lane 2), or control vector (lane 8), or lysates from different human tissues were resolved by 10% SDS-PAGE gel, transferred onto a nitrocellulose membrane, and probed with the N-terminal SALL4 peptide antibody.

FIG. 2 demonstrates the expression of SALL4 in human primary AML and myeloid leukemia cell lines. Real-time PCR quantification of SALL4A and SALL4B normalized to GAPDH showed that both SALL4A and SALL4B were expressed in purified CD34+ cells, but SALL4A was rapidly downregulated and SALL4B turned off in normal bone marrow (N=3) and normal peripheral blood (N=3) cells. In contrast, in 15 primary AML samples and three myeloid leukemia cell lines (Kasumi-1, THP-1, and KG.1), the expression of SALL4A or SALL4B, or both, failed to be down-regulated. The results were calibrated against the expression of SALL4A or SALL4B in purified CD34+ cells.

FIGS. 3(a-e) show that SALL4B transgenic mice have an MDS-like/AML phenotype. FIG. 3(a) illustrates the generation of SALL4B transgenic mice: CMV/SALL4B transgenic construct and PCR analysis of transgenic line 507. (A) Schematic diagram of transgenic construct. The approximately 1.8-kb cDNA of SALL4B was subcloned into a pCEP4 vector, and the CMV/SALL4 construct was excited by digestion with SalI. (B) Tissue distribution of SALL4B in transgenic mice. The location of primers used for RT-PCR amplification is indicated by arrows in part A. A primer specific for human SALL4B at the C-terminus was used as a 5′ primer, in combination with SV40-noncoding sequence-specific primers for RT-PCR of various tissues. FIG. 3(b) shows the flow cytometric analysis of AML in SALL4B transgenic mice. AML cells were positive for CD45, c-kit, Gr-1, and Mac-1; negative for B220, CD3, and Ter119. FIG. 3(c) illustrates the comparison between bone marrow of SALL4B transgenic and control mice. SALL4B transgenic mouse bone marrow showed increased cellularity, myeloid population (Gr-1/Mac-1 double positive), immature population (c-kit positive), and apoptosis (Annexin V positive, PI negative), compared with control WT mice. FIG. 3(d) shows that there are an increased number of immature cells and apoptosis in CFUs from SALL4B transgenic mice. On day 7 of culture, a greater number of immature cells (B, C, and D, red arrows) and apoptotic cells (B, C, and D, double red arrows) were observed in transgenic mouse CFUs than in control CFUs (A). Consistent with this morphologic observation, there was increased apoptosis (Annexin V positive, PI negative, E) and more CD34+ immature cells (F). FIG. 3(e) illustrates the comparison between bone marrow CFUs of SALL4B transgenic and control mice. Percentage of different types of colonies found in CFU assays of SALL4B transgenic and control mice (A). CFUs from SALL4B transgenic mice compared with control mice showed a statistically significant increase in CFU-GM (B) (transgenic: 53.6±10.3, N=13 vs. WT: 38.1±3.1, N=8; P=0.002) and decrease in BFU-E (transgenic: 7.8±3.8, N=13 vs. WT: 14.1±2.7, N=8; P=0.001).

FIGS. 4(a-c) demonstrate the interaction between SALL4 and the Wnt/β-catenin signaling pathway. FIG. 4(a) shows that both SALL4A and SALL4B can interact with β-catenin. Nuclear extracts (lysates) prepared from Cos-7 cells were transiently transfected with HA-SALL4A or HA-SALL4B. (A) Anti-HA antibody recognized both SALL4A (165 kDa) and SALL4B (95 kDa). (B) P-Catenin was detected in the lysates. (C) Immunoprecipitation was performed with the use of an HA affinity resin and detected with an anti-β-catenin antibody. β-Catenin was readily detected in both HA-SALL4A and HA-SALL4B pull-downs. FIG. 4(b) shows the activation of the Wnt/β-catenin signaling pathway by both SALL4A and SALL4B. NIH3T3 cells were transfected with 1.0 μg of either SALL4A or SALL4B plasmid and TOPflash reporter plasmid (Upstate USA, Chicago, Ill.). After 24-h stimulation with Wnt1 or the mock, luciferase activity was measured. FIG. 4(c) illustrates a working hypothesis. SALL4 is expressed in human stem cells/progenitors but is absent in mature hematopoietic cells during normal hematopoiesis. Constitutive expression of SALL4 isoforms (failure to turn off SALL4) results in blocked differentiation and constitutive renewal with aberrant expansion of the stem cell pool that lead to leukemic transformation (+, presence of SALL4 expression; −, absence of SALL4 expression).

FIG. 5 illustrates dose-dependent effect of SALL4B on the OCT4 promoter. 0.3 μg of OCT4-Luc construct (PMOct4) was cotransfected with 0.1 μg of renilla plasmid and increasing amounts (0-1.0 μg) of SALL4B or pcDNA3 vector control.

FIG. 6 demonstrates the effect of OCT4 on SALL gene family member promoters. Each (0.3 μg) SALL-Luc promoter construct (i.e., pSALL1, pSALL3, and pSALL4) was co-transfected with 0.9 μg of OCT4 or pcDNA3 vector control in HEK-293 cells. After 24 hr post-transfection, luciferase activity was evaluated for each group.

FIG. 7 shows the effect of SALL4 isoforms A and B on SALL4 promoter activity. 0.3 μg of SALL4-Luc was cotransfected with 0.1 μg of either SALL4A or SALL4B expressing plasmid in different cell lines (HEK-293 or COS-7); pcDNA3 vector was used as the control. Luciferase activity was normalized for renilla reporter activity. The values represent the mean±s.e. of three experiments.

FIG. 8 demonstrates the dose dependent effect of SALL4A on SALL4 promoter activity. In HEK-293 cells, 0.3 μg of the SALL4-Luc was co-transfected with 0.1 μg of renilla plasmid and increasing ratios of the SALL4A construct and the control pcDNA3 vector. The Luciferase activity is normalized for the Renilla reporter activity.

FIG. 9 shows the effect of SALL4 on SALL1 and SALL3 promoter activity. Each (0.3 μg) SALL-Luc promoter construct was transiently co-transfected with 0.9 μg of SALL4A plasmid or pcDNA3 vector (control) in HEK-293 cells.

FIG. 10 shows the effect of OCT4 on the SALL4 promoter in the presence of excess SALL4A. 0.25 μg of SALL4-Luc construct (pSALL4) was transiently co-transfected with equal amounts (0.5 μg) of SALL4A and OCT4 plasmid in the HEK-293 cells. pcDNA3 was used as a control.

FIG. 11 shows the effect of OCT4 on other SALL member promoters in the presence of SALL4. HEK-293 cells seeded in a 24 well plate were transiently co-transfected with a different SALL member promoter reporter (pSALL1 or pSALL3) and OCT4 plasmid and/or SALL4A construct. pcDNA3 was used as a control.

FIG. 12 shows the effect of self promoter interaction on promoter activity for other SALL protein family members. HEK-293 cells were seeded on a 24 well plate and transiently transfected or co-transfected with 0.3 μg SALL1-Luc reporter construct with various amounts of SALL1 plasmid (0.45 and 0.9 μg) SIX1, previously found to activate SALL1 promoter, was used as a positive control. Luciferase activity was normalized for renilla reporter activity.

FIG. 13 shows that SALL4 binds genes to Oct4 and Nanog as well as their networks. (A) Comparison with published data shows that SALL4 binds genes common to Oct4 and Nanog binding locations. (B) and (C) Western blots for SALL4, Oct4 and Nanog. These suggests that these three proteins work together to maintain pluripotency in ES cells.

FIG. 14 shows that SALL4 functions to maintain pluripotency. (A) Genes identified as pluripotency markers for each of the four cell lineages bound in the ChIP-chip. (B) Using real-time PCR we analyzed mRNA levels for various markers for pluripotency after SALL4 shutdown. Levels of mRNA increased for endoderm, ectoderm and trophectoderm markers, indicating that SALL4 represses differentiation into these cell lineages.

FIG. 15 shows that SALL4 binds to downstream targets of PRC1 and PRC2. (A) To better illustrate the regulatory mechanisms of PRC1 and PRC2 we compared the transcription factors bound by SALL4, Rnf2 and Suz12. For example, Suz12 only has two unique transcription factors and shares others with Rnf2, SALL4, or both SALL4 and Rnf2. (B) Representation of developmentally important genes bound by SALL4. Included are multiple members of the HOX (homeobox protein), PAX (paired box), DLX (distal-less homeobox), SIX (sine oculis homeobox homologue), RBX (reproductive homeobox), H6 (H6 homeobox), OBX (oocyte specific homeobox), LHX (LIM homeobox), FBX (F-box), FOX (forkhead box), and TBX (T-box) families along with various other developmental genes.

FIG. 16 shows that SALL4 regulates methylation events associated with H3K4 and H3K27.

FIG. 17 shows that SALL4 binds to signaling pathways vital to cell fate decisions. (A) SALL4 binds gene promoters belonging to various pathways and we suggest that it plays a regulatory role in these pathways. (B) Quantitative representation of pathways bound by SALL4. The values reflect genes bound directly in the pathway or as downstream targets of the pathway. (C) Using the Wnt/B-catenin signaling pathway, we show the effects of SALL4 shutdown on the canonical pathway (green is down-regulation, red is up-regulation of expression).

FIG. 18 demonstrates that expansion of HSC and HPC were correlated with disease progression in SALL4B transgenic mice. Increased c-kit positive HSCs/HPCs in SALL4B transgenic mice are contrasted with WT control mice where c-kit positive cells are approximately 6.5+2.5% of the total bone marrow cells. This population was increased in pre-leukemic (MDS) SALL4B transgenic mice and became even more prominent in leukemic SALL4B bone marrow.

FIG. 19 shows LSCs in SALL4B transgenic mice. Whole bone marrows from SALL4B transgenic mice were sorted to HSCs, CMPs (common myeloid progenitors), GMPs (granulocyte/macrophage progenitors), and MEPs (megakryocyte/erythroid progenitors) and then transplanted into the primary NOD-SCID recipients. After the primary recipients developed leukemia, their bone marrow cells were sorted into HSCs, CMPs, GMPs, and MEPs and transplanted into secondary NOD-SCID recipients. Representative FACS-staining profiles of HSCs and HPCs from bone marrows of WT NOD-SCID mice, primary leukemic NOD-SCID recipients, and secondary leukemic NOD-SCID mice showed that GMP cells were substantially increased during leukemic transplantation. The increase of HSCs in leukemic SALL4B transgenic mice and leukemic NOD-SCID recipients were variable.

FIG. 20 shows caspase-3 activity, cell cycle and cellular DNA synthesis in SALL4 suppressed-NB4. A and D, NB4 transduced with control retrovirus. B and E, NB4 cells transduced with SALL4 siRNA retroviruses; C and F, restoration of Bmi-1 by ectopically expressing Bmi-1. Evidence showing that siRNA shutdown of SALL4 induces apoptosis in NB4 cells (A and B). SALL4 shutdown NB4 cells can be rescued from apoptosis (C). Monitor cell-cycle changes and cellular DNA synthesis in NB4 and SALL4 shutdown NB4 cells by both BrdU incorporation assay and FACS (3% background debris are excluded). SALL4 knockdown induces cell cycle arrest and increased DNA synthesis (D and E). By ectopically expressing Bmi-1, SALL4 shutdown cells can be rescued from cell cycle arrested and DNA synthesis (F). Two siRNA retroviral constructs that target different regions of the SALL4 are made, and their ability to reduce SALL4 mRNA in NB4 cells are confirmed by Q-RT-PCR. In both SALL4 siRNA constructs, down-regulation of SALL4 also significantly reduced Bmi-1 levels.

FIG. 21 demonstrates that treatment with 5-azacytidine (5AC) significantly suppresses SALL4 and its downstream target, Bmi-1, but increases expression of the tumor suppressor gene, p16INK4a. After 48 hours of 5AC treatment, marked knockdown of Bmi-1 and SALL4 expression were observed in a dose-dependent manner of about 50-95% and 64-98%, respectively. Conversely, p16^{INK4A} mRNA expression significantly increased by 5-6 folds compared to the untreated control.

FIG. 22 shows dose-dependent activation of Bmi-1 promoter by SALL4 in HEK-293 cells. 0.25 μg of the Bmi-1-Luc construct was co-transfected with 0.04 μg of Renilla Luciferase plasmid and increasing ratios of either the SALL4A or SALL4B expressing construct; pcDNA3 was used as the control. Data represent the mean of three individual experiments. HEK-293 cells, rather than 32D or HL60 cells, were used in these transfection experiments as these hematopoietic cells exhibit low transfection efficiency.

FIG. 23 shows the mapping of the SALL4 functional site within the Bmi-1 promoter region by a luciferase reporter gene assay. In HEK-293 cells, 0.3 μg of different length Bmi-1-Luc constructs were co-transfected with 0.04 μg of Renilla Luciferase plasmid and 0.9 μg of either SALL4A or SALL4B plasmid. The ^{Δ}P1254 and ^{Δ}P683 refer to Bmi-1 mutant promoter constructs, −1254 or −683, in which the −270 to −168 sequence was deleted. (A) Deletion constructs of the Bmi-1 promoter and their corresponding promoter activity stimulated by either SALL4A or SALL4B. (B) SALL4A and SALL4B stimulation of −1254 and −683 or ΔP1254 and ΔP683 Bmi-1 promoter constructs.

FIG. 24 shows that SALL4 specifically binds to the endogenous mouse Bmi-1 promoter (−450 to 1+) using ChIP assays. (A) Schematic representation of the primer sets specific for Bmi-1 promoter. (B) Chip assays were performed by using an antibody against HA (lane +) or preimmune sera (lane −); enriched chromatin was analyzed by PCR with primers as shown in A. (C) Relative enrichment of Bmi-1 promoter regions in 32D cells that were transfected with SALL4 isoforms tagged with HA or the control, pcDNA3. Chip assays were performed using HA antibody. Amplicons were quantitated by Q-PCR. Endogenous SALL4 also bound to the human Bmi-1 promoter at the same position as seen in the human HEK-293 cells, leukemia cell lines, and NB4 using SALL4 antibodies.

FIG. 25 shows the effects of endogenous Bmi-1 expression levels. (A) siRNA mediated SALL4 suppression in leukemia cells: Three siRNA oligonucleotides, targeting the SALL4 gene at position 890, 1682, and 1705, respectively, were cloned into a pSUPER retrovirus vector; PT67 packaging cells were transfected and HL-60 cells were infected with the virus collected after 48 hr of infection. Stable infected cells were collected under G418 selection. Total RNA was extracted by Trizol, RT PCR was performed, and the relative amount of target gene mRNA was analyzed. The SALL4/GAPDH ratio in noninfected cells was set at 1; values are the mean of duplicate reactions. Bars indicate SD. (B) SALL4+/− heterozygous bone marrow cells showed decreased levels of Bmi-1 expression. Bone marrow cells from SALL4+/− and SALL4+/+ mice were isolated. QRT PCR was performed to analyze expression levels of SALL4 and Bmi-1. Values are the mean of duplicate reactions. (C) Up-regulation of Bmi-1 in SALL4B transgenic mice associated with disease progression. RT-PCR analysis was performed on (1) total bone marrow cells from two WT control mice (lanes 1, 2) and two pre-leukemic transgenic mice (lanes 3, 4) and (2) leukemic bone marrow cells from two leukemic transgenic SALL4B mice (lanes 5, 6).

FIG. 26 demonstrates that mRNA expression of Bmi-1 and SALL4 in human AML blast samples showed a strong correlation between Bmi-1 and SALL4 expression. Twelve randomly selected blastic AML samples were analyzed using RT PCR to enhanced expression quantify relative mRNA expression of Bmi-1 and SALL4 genes. Ten out of 12 AML samples showed significant Bmi-1 gene amplification ranging from 1.10- to 22.32-fold increase relative to the averaged normal controls (Normal). Interestingly, the same 10 of 12 AML samples also showed elevated SALL4 gene expression amplification, ranging from a 3.93- to 653.03-fold increase relative to the averaged normal controls. The Log10 scale represents the relative quantification of genes of interest. Using data for the 12 AML samples, we preformed a statistical analysis and determined the correlation coefficient to be 0.703 with a p-value of 0.0159.

FIG. 27 shows that SALL4 specifically binds to the endogenous mouse Bmi-1 promoter (−450 to 1+) resulting in histone 3 lysine 4 and lysine 79 methylation using chromatin immunoprecipitation (ChIP) assays. Enriched chromatin was analyzed by PCR with the primers shown in FIG. 3A. FIGS. 6A and 6B are distributions of the histone 3 trimethylation levels of H3-K4 and H3-K79 on the Bmi-1 promoter regions, respectively, in 32D cells that were transfected with SALL4A tagged with HA or control DNA, pcDNA3. ChIP assays were performed using histone H3-K4 trimethylation antibody (A) and histone H3-K79 methylation antibody (B). Amplicons were quantitated by Q-PCR. Experiments were repeated three times with similar results.

FIG. 28 shows that SALL4 expression is decreased during NTERA2 cell differentiation. A) Differentiation was induced in an embryonal carcinoma cell line using retinoic acid. To determine the differentiation status of these cells, Q-RT-PCR was performed to analyze markers that represent lineage-specific cell differentiation. Retinoic acid induction (5 μM) of NTERA2 cells resulted in an up-regulation of a panel of ectoderm markers. In addition, some endodermal, mesodermal, and trophectodermal genes were also up-regulated. B) Following retinoic-acid-induced differentiation, SALL4 expression is significantly reduced in NTRA2 cells treated with different concentrations of retinoic acid when compared with untreated NTERA2 cells.

FIG. 29 shows the effects of endogenous Bmi-1 expression levels and cell differentiation by SALL4 knockdown are shown. A) Relative endogenous Bmi-1 and SALL4 expression levels after SALL4 knockdown are shown. Two siRNA oligonucleotides (#7410, #7412) targeting different regions of the SALL4 gene are transfected in PT67 packaging cells. NTERA2 cells are infected with the virus collected 48 hours post-transfection. Total RNA is extracted and Q-RT-PCR is performed to analyze the relative amount of target gene mRNA. The SALL4/GAPDH ratio in noninfected cells is set at one. Values are the mean of duplicates, and bars indicate standard deviation. B, Effect of SALL4 knockdown on NTERA2 cell differentiation. Quantitative PCR analysis of stem-cell marker genes in NTERA2 cells after SALL4 siRNA (GCCGACCTATGTCAAGGTTGAAGTTCCTG (SEQ ID NO:33) and GATGCCTTGAAACAAGCCAAGCTACCTCA (SEQ ID NO:34) virus infection shows that no primitive germ-layer markers were detected.

FIG. 30 shows representative FACS data of caspase-3 activity in NTERA2 and SALL4-deleted NTERA2 cells. Evidence showing that siRNA shutdown of SALL4 induces apoptosis in NTERA2 cells (A and B). By overexpressing Bmi-1, SALL4 shutdown cells can be rescued from apoptosis (C). However, overexpression of Bmi-1 has little effect on caspase-3 activity in WT NTERA2 cells (D).

FIG. 31 shows Monitored cell-cycle changes and cellular DNA synthesis in SALL4-depleted NTERA2 and NTERA2 cells by both BrdU incorporation assay and FACS. SALL4 knockdown induces cell cycle arrest and increased DNA synthesis (A and B). By ectopically expressing Bmi-1, SALL4 shutdown cells can be rescued from cell cycle arrested and DNA synthesis (C) but a control vector does not (data not shown). overexpression of Bmi-1 has little effect on cell cycle arrest and increased DNA synthesis in wild type NTERA2 cells (D).

### DETAILED DESCRIPTION OF THE INVENTION

Before the present composition, methods, and culturing methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “a nucleic acid” includes one or more nucleic acids, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure. All publications mentioned herein are incorporated herein by reference in their entirety.

SALL4 is a member of a family of C₂H₂ zinc-finger transcription factors. SALL4 was originally cloned based on its homology to Drosophila splat. In Drosophila, sal is a homeotic gene and essential in the development of posterior head and anterior tail segments. In humans, an autosomal-dominant mutation is associated with Okihiro syndrome (also called Duane-radial ray syndrome), which causes defects in multiple organ systems. Mutations in the SALL4 gene severely hinder development in many animal models.

SALL4 seems to regulate embryonic stem cell (ESC) pluripotency through interaction with major regulatory proteins including Oct4 and Bmi-1.

Bmi-1 is a member of the polycomb group (PcG) of proteins initially identified in Drosophila as a repressor of homeotic genes. In humans, the polycomb gene Bmi-1 plays an essential role in regulating adult, self-renewing, hematopoietic stem cells (HSCs) and leukemia stem cells (LSCs). Bmi-1 is expressed highly in purified HSCs, and its expression declines with differentiation. Knockout of the Bmi-1 gene in mice results in a progressive loss of all hematopoietic lineages. This loss results from the inability of the Bmi-1 (−/−) stem cells to self-renew. In addition, Bmi-1 (−/−) cells display altered expression of the cell cycle inhibitor genes p16INK4a and p19ARF. The expression of Bmi-1 appears to be important in accumulation of leukemic cells. Interestingly, inhibiting self-renewal in tumor stem cells after deleting Bmi-1 can prevent leukemic recurrence. Recently, Bmi-1 expression has been used as an important marker for predicting the development of MDS and disease progression to AML.

Knockdown of SALL4 expression using small interfering RNAs causes ESCs to differentiate into the trophoblast lineage, demonstrating that SALL4 must be expressed to maintain pluripotency. Further, it seems that SALL4 is necessary for the inner cell mass to differentiate into the epiblast and primitive endoderm during early embryogenesis. Expression of SALL4 protein can be correlated with stem and progenitor cell populations in various organ systems including bone marrow. The human Okihiro syndrome may result from premature depletion of different stem cell or progenitor cell pools depending on the genetic background.

Embryonic stem cells have become the focus of scientific research due to their regenerative capacity and potential uses in disease therapies. Stem cells have been shown to give rise to all three germ layers (ectoderm, mesoderm, and endoderm) during embryogenesis emphasizing their pluripotent potential. Cellular machinery that governs ES cells is vital to their function because it regulates the differentiation signals and pluripotency maintenance signals necessary for proper development.

ES cells are derived from the inner cell mass (ICM) of the developing embryo. During this critical time, ES cell pluripotency is regulated in part by Oct4, Sox2, and Nanog as well as through the two Polycomb Repressive Complexes (PRCs): PRC1 and PRC2. SALL4 may play a vital role in governing ES cells proliferation and pluripotency. For example, embryonic endoderm ES cells cannot be established from SALL4 deficient blastocyts. SALL4 is expressed by cells of the early embryo and germ cells, exhibiting a similar expression pattern to that of both Oct4 and Sox2. This suggests that SALL4 may be a regulator of a network of genes implicated in maintaining ES cell pluripotency.

Homeobox and homeotic genes play important roles in normal development. Some homeobox genes, such as Hox and Pax, also function as oncogenes or as tumor suppressors in tumorigenesis or leukemogenesis. The important role of SALL4, a homeotic gene and a transcriptional factor, in human development was recognized because heterozygous SALL4 mutations lead to Duane Radial Ray syndrome. In a related aspect, SALL4's oncogenic role in leukemogenesis is described herein.

In one embodiment, the present disclosure identifies two SALL4 isoforms, SALL4A and SALL4B. In a related aspect, the disclosure provides an analysis of SALL4 nucleic acids and proteins as tools for diagnosing and treating patients having proliferation disorders such as hematologic malignancies and other tumors involving constitutive expression of SALL4 nucleic acid and protein. In a related aspect, SALL4 serves as a malignant stem cell marker for diagnosis and treatment of cancers.

For example, during normal hematopoiesis, SALL4 isoforms are expressed in the CD34+ HSC/HPC population and rapidly turned off (SALL4B) or down-regulated (SALL4A) in normal human bone marrow and peripheral blood. In contrast, SALL4 is constitutively expressed in all AML samples (N=81) that were examined, and failed to turn off in human primary AML and myeloid leukemia cell lines. In a related aspect, the leukemogenic potential of constitutive expression of SALL4 in vivo was directly tested via generation of SALL4B transgenic mice. Such transgenic mice exhibit dysregulated hematopoiesis, much like that of human MDS, and exhibited AML that was transplantable. The MDS-like features in these SALL4B transgenic mice do not require cooperating mutations and are observed as early as 2 months of age. The ineffective hematopoiesis observed in these mice is characterized, as it is in human MDS, by hypercellular bone marrow and paradoxical peripheral blood cytopenias (neutropenia and anemia) and dysplasia, which are probably secondary to the increased apoptosis noted in the bone marrow. While not being bound to theory, a reason for the late onset of leukemia development in these transgenic mice may be the accumulation of additional genetic damage during the ≧8 months of replicative stress. Late onset of disease may also be a consequence of SALL4-induced genomic instability.

Further, specific, recurrent chromosomal translocations characterize many leukemias, which can result from a breakdown in the normal process of immunoglobulin or T-cell receptor gene rearrangement, causing inter-chromosomal translocations rather than normal intra-chromosomal rearrangement. The flow of genetic information from genes at chromosomal translocation breakpoints to proteins has several points which therapeutic reagents could intervene. Sequence specific binding elements that exploit zinc-finger binding protein domains can be used to create de novo sequence specific binding elements that could act as gene switches which can target chromosomal fusion junctions to turn off expression of aberrant gene fusion products.

In one embodiment, SALL4 can be used as a component of a fusion protein which targets chromosomal fusion junctions as a gene switch to modulate the expression of gene fusion products. Production of recombinant fusion protein is well known in the art (see, e.g., Sambrook et al., Molecular Cloning, A Laboratory Manual, 2nd Ed.; Cold Spring Harbor Laboratory Press, Cold Spring Harbor, N.Y. (1989).

In one embodiment, SALL4 proteins and/or nucleic acids are detected for diagnosing subpopulations of lymphomas and leukemias or other types of cancers. In another embodiment, the detection of the SALL4 proteins and nucleic acids can be used to identify a subject, including, but not limited to, a human subject, at risk for developing/acquiring a proliferative disease.

In a further embodiment, methods for identifying compounds which alter SALL4 protein and nucleic acid levels are disclosed. In a related aspect, SALL4 can serve as a therapeutic target, where blocking SALL4 function can inhibit tumor development and progression.

In another aspect, investigation of the potential mechanism of SALL4 involvement in leukemogenesis demonstrates that both SALL4A and SALL4B interacted with β-catenin, an essential component of the Wnt signaling pathway involving self-renewal of HSCs. In addition, both are able to activate the Wnt/β-catenin pathway in a reporter gene assay, consistent with SALL family function in Drosophila and humans. Furthermore, similar to the situation with β-catenin, SALL4 expression in CML varied at different phases of the disease: SALL4 expression being absent in the chronic phase, became detectable in the accelerated phase only in immature blasts, and is strongly positive in the blast phase.

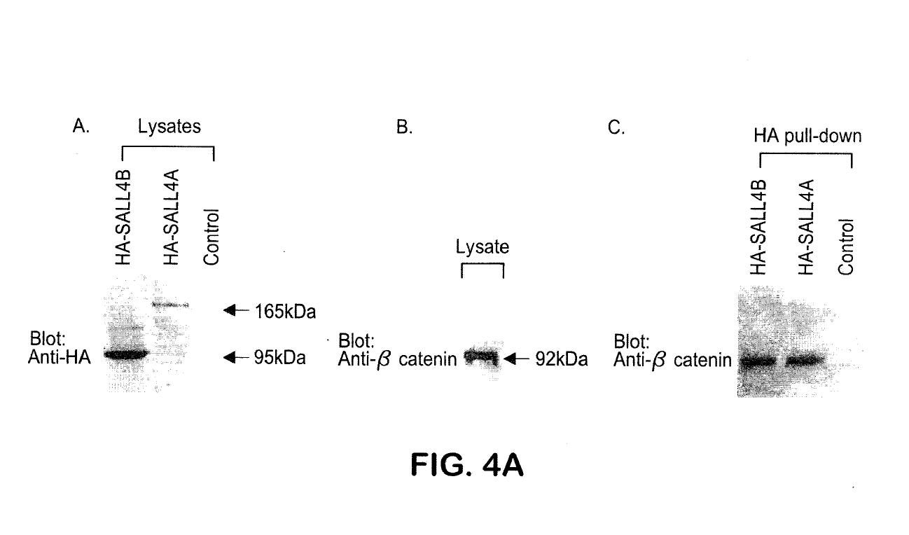

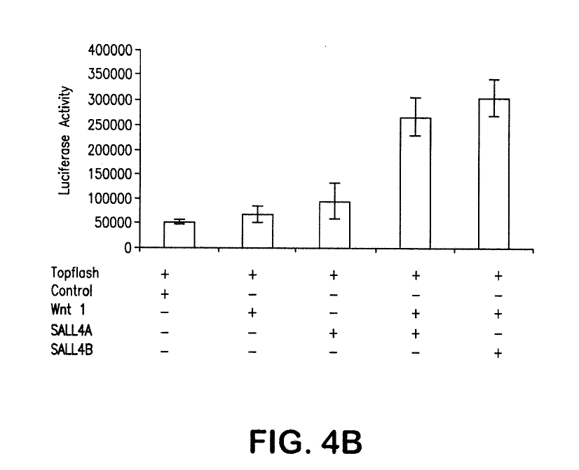

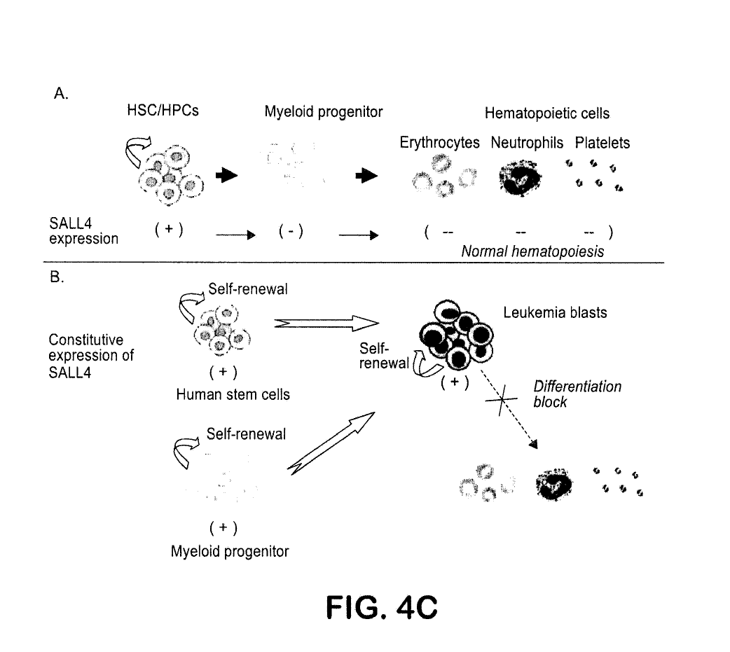

On the basis of these studies, a working hypothesis is disclosed (e.g., see FIG. 4d). While not being bound to theory, constitutive expression of SALL4 in AML may enable leukemic blasts to gain stem cell properties, such as self-renewal and/or dedifferentiation, and thus become LSCs. This hypothetical model would parallel what is seen in the case of β-catenin. For example, in normal myelopoiesis, β-catenin is only activated in HSCs bearing a self-renewal property. In the blast phase of CML, β-catenin gains function by becoming activated in the GMPs, resulting in leukemic transformation.

In another aspect, the oncogene SALL4 plays an important role in normal hematopoiesis and leukemogenesis. SALL4B transgenic mice exhibit MDS-like phenotype with subsequently AML transformation that is transplantable. Few animal models are currently available for the study of human MDS. The SALL4B transgenic mice that were generated by the methods described herein provide a suitable animal model for understanding and treating human MDS and its subsequent transformation to AML. The interaction between SALL4 and the Wnt/β-catenin signaling pathway not only provides a plausible mechanism for SALL4 involvement in leukemogenesis but also advances the understanding of the activation of the Wnt/β-catenin signaling pathway in CML blastic transformation.

As disclosed herein, the identification of SALL4 isoforms and their constitutive expression in all human AML were examined. The direct impact of SALL4 expression in AML was tested in vivo. The disclosure demonstrates that constitutive expression of SALL4 in mice is sufficient to induce MDS-like symptoms and transformation to AML that is transplantable. The disclosure also demonstrates that SALL4 is able to bind β-catenin and activate the Wnt/β-catenin signaling pathway. SALL4 and β-catenin share similar expression patterns at different phases of CML.

In one embodiment, an isolated polynucleotide comprising a sequence encoding an amino acid sequence as set forth in SEQ ID NO: 2 (GenBank Acc. No. AAO44950), SEQ ID NO: 4 (GenBank Acc. No. AAO16566), or SEQ ID NO: 6 (GenBank Acc. No. AAO16567) is provided. In a related aspect, such sequences comprise a nucleic acid sequence as set forth in SEQ ID NO: 1 (GenBank Acc. No. AY172738), SEQ ID NO: 3 (GenBank Acc. No. AY170621), SEQ ID NO: 5 (GenBank Acc. No. AY170622), or complements thereof. In another related aspect, a vector comprising such polynucleotides are also disclosed, including, but not limited to, expression vectors which are operably linked to a regulatory sequence which directs the expression of the polynucleotide in a host cell.

In another embodiment, an isolated polypeptide comprising an amino acid sequence as set forth in SEQ ID NO: 2, SEQ ID NO: 4, or SEQ ID NO: 6 is disclosed. In one aspect, a method of treating a myelodysplastic syndrome (MDS) in an individual including administering such a polypeptide is provided. In another aspect, antibodies or binding fragments thereof which bind to such a polypeptide are also disclosed.

Antibodies that are used in the methods disclosed include antibodies that specifically bind polypeptides comprising SALL4, or their isoforms as set forth in SEQ ID NO: 2, SEQ ID NO: 4, or SEQ ID NO: 6. In one aspect, a fragment of SEQ ID NO: 2, SEQ ID NO: 4, or SEQ ID NO: 6 is used to generate such antibodies. In a related aspect, such a fragment consists essentially of SEQ ID NO: 13.

In one embodiment, a method of identifying a cell possessing pluripotent potential is disclosed including contacting a cell isolated from an inner cell mass (ICM), a neoplastic tissue, or a tumor with an agent that detects the expression of a SALL family member protein, and determining whether a SALL family member protein is expressed in the cell, where determining the expression of the SALL family member protein positively correlates with induction of self-renewal in the cell, whereby such expression is indicative of pluripotency.

In one aspect, the SALL family member includes SALL1, SALL3, and SALL4. In a related aspect, SALL4 is SALL4A or SALL4B.

In another aspect, the agent is an antibody directed against the SALL family member protein or a nucleic acid which is complementary to a mRNA encoding the SALL family member protein. In a related aspect, the SALL family member protein sequence includes SEQ ID NO: 2, SEQ ID NO:4, SEQ ID NO:6, SEQ ID NO:22, and SEQ ID NO:24. In another related aspect, the nucleic acid is complementary to a sense strand of a nucleic acid sequence including SEQ ID NO:1, SEQ ID NO:3, SEQ ID NO:5; SEQ ID NO:21, and SEQ ID NO:23.

In one aspect, the cell is an embryonic stem (ES) cell, an embryonic carcinoma (EC) cell, an adult stem cell, or a cancer stem cell. In a related aspect, the tissues is plasma or a biopsy sample from a subject. In a further related aspect, the subject is a human.

As used herein, “primordial cell” means an originally or earliest formed cell in the growth of an individual or organ.

As used herein, “progenitor cell” means a parent cell that gives rise to a distinct cell lineage by a series of cell divisions.

As used herein, “pluripotent potential” means the ability of a cell to renew itself by mitosis.

As used herein “positively correlates” means affirmatively associated with the phenomenon observed. For example, induction of SALL4A or SALL4B is associated with increased cell renewal ability.

As used herein, “neoplasm,” including grammatical variations thereof, means new and abnormal growth of tissue, which may be benign or cancerous.

As used herein “consisting essentially of” includes a specific molecular entity (e.g., but not limited to, a specific sequence identifier) and other molecular entities that do not materially affect the properties associated with the specific molecular entity. For example, a fusion protein comprising SEQ ID NO: 13 and an adjuvant, for generating an immunogenic response against SEQ ID NO: 2, SEQ ID NO: 4, and/or SEQ ID NO: 6, would consist essentially of SEQ ID NO: 13.

Antibodies are well-known in the art and discussed, for example, in U.S. Pat. No. 6,391,589. Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA, and IgY), class (e.g., IgG1, IgG2, IgG3, IgG4, IgA1, and IgA2) or subclass of immunoglobulin molecule.

Antibodies of the invention include antibody fragments that include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. In one aspect, the antibodies are human, murine (e.g., mouse and rat), donkey, sheep, rabbit, goat, guinea pig, camel, horse, or chicken. Further, such antibodies may be humanized versions of animal antibodies (see, e.g., U.S. Pat. No. 6,949,245). The antibodies of the invention may be monospecific, bispecific, trispecific or of greater multispecificity.

The antibodies of the invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art. Further, antibodies and antibody-like binding proteins may be made by phage display (see, e.g., Smith and Petrenko, Chem Rev (1997) 97(2):391-410).

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example; in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

In one embodiment, a method for isolating leukemia stem cells using such antibodies is provided, including obtaining a sample of cells from a subject, sorting cells that express an amino acid sequence as set forth in SEQ ID NO: 13 from cells that do not express the amino acid sequence, and selecting, by a myeloid surface marker, leukemia stem cells from the sample of cells that express the amino acid sequence as set forth in SEQ ID NO: 13. In a related aspect, the step of sorting includes sorting by fluorescent activated cell sorting and/or magnetic bead sorting.

In one aspect, the marker is CD34, c-kit, Gr-1, Mac-1, MPO, and/or nonspecific esterase. In another aspect, the marker is SSEA-1, SSEA-2, SSEA-4, TRA-1-60, TRA-1-81, CD34⁺, CD59⁺, Thy1/CD90⁺, CD38^{lo/-}, C-kit^{-/lo}, lin⁻, SH2, vimentin, periodic acid Schiff activity (PAS), FLK1, BAP, or acid phosphatase. In a further related aspect, wherein the leukemia stem cells are negative for B-cell (B220 and CD19), T-cell (CD4, CD8, CD3, and CD5), megakaryocytic (CD41), and erythroid (Ter119) markers. Alternatively, markers can include those as set forth in Table 1.

TABLE 1
Markers Commonly Used to Identify Stem Cells and to Characterize Differentiated Cell Types
Blood Vessel

| | | |
|---|---|---|
| Fetal liver kinase-1 | Endothelial | Cell-surface receptor protein that identifies endothelial cell |
| (Flk1) | | progenitor; marker of cell-cell contacts |
| Smooth muscle cell- | Smooth muscle | Identifies smooth muscle cells in the wall of blood vessels |
| specific myosin | | |
| heavy chain | | |
| Vascular endothelial | Smooth muscle | Identifies smooth muscle cells in the wall of blood vessels |
| cell cadherin | | |

Bone

| | | |
|---|---|---|
| Bone-specific | Osteoblast | Enzyme expressed in osteoblast; activity indicates bone |
| alkaline | | formation |
| phosphatase (BAP) | | |
| Hydroxyapatite | Osteoblast | Minerlized bone matrix that provides structural integrity; |
| | | marker of bone formation |
| Osteocalcin (OC) | Osteoblast | Mineral-binding protein uniquely synthesized by osteoblast; |
| | | marker of bone formation |

Bone Marrow and Blood

| | | |
|---|---|---|
| Bone | Mesenchymal stem and | Important for the differentiation of committed |
| morphogenetic | progenitor cells | mesenchymal cell types from mesenchymal stem and |
| protein receptor | | progenitor cells; BMPR identifies early mesenchymal |
| (BMPR) | | lineages (stem and progenitor cells) |
| CD4 and CD8 | White blood cell (WBC) | Cell-surface protein markers specific for mature T |
| | | lymphocyte (WBC subtype) |
| CD34 | Hematopoietic stem | Cell-surface protein on bone marrow cell, indicative of a |
| | cell (HSC), satellite, | HSC and endothelial progenitor; CD34 also identifies |
| | endothelial progenitor | muscle satellite, a muscle stem cell |
| CD34⁺Sca1⁺ Lin⁻ | Mesencyhmal stem cell | Identifies MSCs, which can differentiate into adipocyte, |
| profile | (MSC) | osteocyte, chondrocyte, and myocyte |
| CD38 | Absent on HSC | Cell-surface molecule that identifies WBC lineages. |
| | Present on WBC | Selection of CD34⁺/CD38⁻ cells allows for purification of |
| | lineages | HSC populations |
| CD44 | Mesenchymal | A type of cell-adhesion molecule used to identify specific |
| | | types of mesenchymal cells |
| c-Kit | HSC, MSC | Cell-surface receptor on BM cell types that identifies HSC |
| | | and MSC; binding by fetal calf serum (FCS) enhances |
| | | proliferation of ES cells, HSCs, MSCs, and hematopoietic |
| | | progenitor cells |
| Colony-forming unit | HSC, MSC progenitor | CFU assay detects the ability of a single stem cell or |
| (CFU) | | progenitor cell to give rise to one or more cell lineages, |
| | | such as red blood cell (RBC) and/or white blood cell (WBC) |
| | | lineages |
| Fibroblast colony- | Bone marrow fibroblast | An individual bone marrow cell that has given rise to a |
| forming unit (CFU- | | colony of multipotent fibroblastic cells; such identified cells |
| F) | | are precursors of differentiated mesenchymal lineages |
| Hoechst dye | Absent on HSC | Fluorescent dye that binds DNA; HSC extrudes the dye and |
| | | stains lightly compared with other cell types |
| Leukocyte common | WBC | Cell-surface protein on WBC progenitor |
| antigen (CD45) | | |
| Lineage surface | HSC, MSC | Thirteen to 14 different cell-surface proteins that are |
| antigen (Lin) | Differentiated RBC and | markers of mature blood cell lineages; detection of Lin- |
| | WBC lineages | negative cells assists in the purification of HSC and |
| | | hematopoietic progenitor populations |
| Mac-1 | WBC | Cell-surface protein specific for mature granulocyte and |
| | | macrophage (WBC subtypes) |
| Muc-18 (CD146) | Bone marrow | Cell-surface protein (immunoglobulin superfamily) found on |
| | fibroblasts, endothelial | bone marrow fibroblasts, which may be important in |
| | | hematopoiesis; a subpopulation of Muc-18+ cells are |
| | | mesenchymal precursors |
| Stem cell antigen | HSC, MSC | Cell-surface protein on bone marrow (BM) cell, indicative of |
| (Sca-1) | | HSC and MSC Bone Marrow and Blood cont. |
| Stro-1 antigen | Stromal | Cell-surface glycoprotein on subsets of bone marrow |
| | (mesenchymal) | stromal (mesenchymal) cells; selection of Stro-1+ cells |
| | precursor cells, | assists in isolating mesenchymal precursor cells, which are |
| | hematopoietic cells | multipotent cells that give rise to adipocytes, osteocytes, |
| | | smooth myocytes, fibroblasts, chondrocytes, and blood |
| | | cells |
| Thy-1 | HSC, MSC | Cell-surface protein; negative or low detection is suggestive |
| | | of HSC |

Cartilage

| | | |
|---|---|---|
| Collagen types II | Chondrocyte | Structural proteins produced specifically by chondrocyte |
| and IV | | |
| Keratin | Keratinocyte | Principal protein of skin; identifies differentiated |
| | | keratinocyte |
| Sulfated | Chondrocyte | Molecule found in connective tissues; synthesized by |
| proteoglycan | | chondrocyte |

Fat

| | | |
|---|---|---|
| Adipocyte lipid- | Adipocyte | Lipid-binding protein located specifically in adipocyte |
| binding protein | | |
| (ALBP) | | |
| Fatty acid | Adipocyte | Transport molecule located specifically in adipocyte |
| transporter (FAT) | | |
| Adipocyte lipid- | Adipocyte | Lipid-binding protein located specifically in adipocyte |
| binding protein | | |
| (ALBP) | | |

General

| | | |
|---|---|---|
| Y chromosome | Male cells | Male-specific chromosome used in labeling and detecting |
| | | donor cells in female transplant recipients |
| Karyotype | Most cell types | Analysis of chromosome structure and number in a cell |

Liver

| | | |
|---|---|---|
| Albumin | Hepatocyte | Principal protein produced by the liver; indicates |
| | | functioning of maturing and fully differentiated hepatocytes |
| B-1 integrin | Hepatocyte | Cell-adhesion molecule important in cell-cell interactions; |
| | | marker expressed during development of liver |

Nervous System

| | | |
|---|---|---|
| CD133 | Neural stem cell, HSC | Cell-surface protein that identifies neural stem cells, which |
| | | give rise to neurons and glial cells |
| Glial fibrillary acidic | Astrocyte | Protein specifically produced by astrocyte |
| protein (GFAP) | | |
| Microtubule- | Neuron | Dendrite-specific MAP; protein found specifically in dendritic |
| associated protein-2 | | branching of neuron |
| (MAP-2) | | |
| Myelin basic protein | Oligodendrocyte | Protein produced by mature oligodendrocytes; located in |
| (MPB) | | the myelin sheath surrounding neuronal structures |
| Nestin | Neural progenitor | Intermediate filament structural protein expressed in |
| | | primitive neural tissue |
| Neural tubulin | Neuron | Important structural protein for neuron; identifies |
| | | differentiated neuron |
| Neurofilament (NF) | Neuron | Important structural protein for neuron; identifies |
| | | differentiated neuron |
| Neurosphere | Embryoid body (EB), | Cluster of primitive neural cells in culture of differentiating |
| | ES | ES cells; indicates presence of early neurons and glia |
| Noggin | Neuron | A neuron-specific gene expressed during the development |
| | | of neurons |
| O4 | Oligodendrocyte | Cell-surface marker on immature, developing |
| | | oligodendrocyte |
| O1 | Oligodendrocyte | Cell-surface marker that characterizes mature |
| | | oligodendrocyte |
| Synaptophysin | Neuron | Neuronal protein located in synapses; indicates connections |
| | | between neurons |
| Tau | Neuron | Type of MAP; helps maintain structure of the axon |

Pancreas

| | | |
|---|---|---|
| Cytokeratin 19 | Pancreatic epithelium | CK19 identifies specific pancreatic epithelial cells that are |
| (CK19) | | progenitors for islet cells and ductal cells |
| Glucagon | Pancreatic islet | Expressed by alpha-islet cell of pancreas |
| Insulin | Pancreatic islet | Expressed by beta-islet cell of pancreas |
| Insulin-promoting | Pancreatic islet | Transcription factor expressed by beta-islet cell of pancreas |
| factor-1 (PDX-1) | | |
| Nestin | Pancreatic progenitor | Structural filament protein indicative of progenitor cell lines |
| | | including pancreatic |
| Pancreatic | Pancreatic islet | Expressed by gamma-islet cell of pancreas |
| polypeptide | | |
| Somatostatin | Pancreatic islet | Expressed by delta-islet cell of pancreas |

Pluripotent Stem Cells

| | | |
|---|---|---|
| Alkaline | Embryonic stem (Es), | Elevated expression of this enzyme is associated with |
| phosphatase | embryonal carcinoma | undifferentiated pluripotent stem cell (PSC) |
| | (EC) | |
| Alpha-fetoprotein | Endoderm | Protein expressed during development of primitive |
| (AFP) | | endoderm; reflects endodermal differentiation Pluripotent |
| | | Stem Cells |
| Bone | Mesoderm | Growth and differentiation factor expressed during early |
| morphogenetic | | mesoderm formation and differentiation |
| protein-4 | | |
| Brachyury | Mesoderm | Transcription factor important in the earliest phases of |
| | | mesoderm formation and differentiation; used as the |
| | | earliest indicator of mesoderm formation |
| Cluster designation | ES, EC | Surface receptor molecule found specifically on PSC |
| 30 (CD30) | | |
| Cripto (TDGF-1) | ES, cardiomyocyte | Gene for growth factor expressed by ES cells, primitive |
| | | ectoderm, and developing cardiomyocyte |
| GATA-4 gene | Endoderm | Expression increases as ES differentiates into endoderm |
| GCTM-2 | ES, EC | Antibody to a specific extracellular-matrix molecule that is |
| | | synthesized by undifferentiated PSCs |
| Genesis | ES, EC | Transcription factor uniquely expressed by ES cells either in |
| | | or during the undifferentiated state of PSCs |
| Germ cell nuclear | ES, EC | Transcription factor expressed by PSCs |
| factor | | |
| Hepatocyte nuclear | Endoderm | Transcription factor expressed early in endoderm formation |
| factor-4 (HNF-4) | | |
| Nestin | Ectoderm, neural and | Intermediate filaments within cells; characteristic of |
| | pancreatic progenitor | primitive neuroectoderm formation |
| Neuronal cell- | Ectoderm | Cell-surface molecule that promotes cell-cell interaction; |
| adhesion molecule | | indicates primitive neuroectoderm formation |
| (N-CAM) | | |
| Oct-4 | ES, EC | Transcription factor unique to PSCs; essential for |
| | | establishment and maintenance of undifferentiated PSCs |
| Pax6 | Ectoderm | Transcription factor expressed as ES cell differentiates into |
| | | neuroepithelium |
| Stage-specific | ES, EC | Glycoprotein specifically expressed in early embryonic |
| embryonic antigen- | | development and by undifferentiated PSCs |
| 3 (SSEA-3) | | |
| Stage-specific | ES, EC | Glycoprotein specifically expressed in early embryonic |
| embryonic antigen- | | development and by undifferentiated PSCs |
| 4 (SSEA-4) | | |
| Stem cell factor | ES, EC, HSC, MSC | Membrane protein that enhances proliferation of ES and EC |
| (SCF or c-Kit | | cells, hematopoietic stem cell (HSCs), and mesenchymal |
| ligand) | | stem cells (MSCs); binds the receptor c-Kit |
| Telomerase | ES, EC | An enzyme uniquely associated with immortal cell lines; |
| | | useful for identifying undifferentiated PSCs |
| TRA-1-60 | ES, EC | Antibody to a specific extracellular matrix molecule is |
| | | synthesized by undifferentiated PSCs |
| TRA-1-81 | ES, EC | Antibody to a specific extracellular matrix molecule |
| | | normally synthesized by undifferentiated PSCs |
| Vimentin | Ectoderm, neural and | Intermediate filaments within cells; characteristic of |
| | pancreatic, progenitor | primitive neuroectoderm formation |

Skeletal Muscle/Cardiac/Smooth Muscle

| | | |
|---|---|---|
| MyoD and Pax7 | Myoblast, myocyte | Transcription factors that direct differentiation of myoblasts |
| | | into mature myocytes |
| Myogenin and MR4 | Skeletal myocyte | Secondary transcription factors required for differentiation |
| | | of myoblasts from muscle stem cells |
| Myosin heavy chain | Cardiomyocyte | A component of structural and contractile protein found in |
| | | cardiomyocyte |
| Myosin light chain | Skeletal myocyte | A component of structural and contractile protein found in |
| | | skeletal myocyte |

In one embodiment, a kit for identifying a cell possessing pluripotent potential is disclosed including an agent for detecting on or more SALL family member protein markers, reagents and buffers to provide conditions sufficient for agent-cell interaction and labeling of the agent, instructions for labeling the detection reagent and for contacting the agent with the cell, and a container comprising the components.

One identifies stem cells according to the method of the disclosure by first sorting, from a population of cells, cells that are positive for expression of a marker comprising SEQ ID NO: 13 from cells that are not. One then selects from the positive marker cells the stem cell of interest; this is performed by sorting cells by their expression of a known cell marker. Any marker that is known to be associated with the stem cells of interest may be used (see, e.g., Table 1).

Any population of cells where stem cells are suspected of being found may be sorted according to the methods disclosed. In one aspect, cells are obtained from the bone marrow of a non-fetal animal, including, but not limited to, human cells. Fetal cells may also be used.

Cell sorting may be by any method known in the art to sort cells, including sorting by fluorescent activated cell sorting (FACS) (see, e.g., Baumgarth and Roederer, J Immunol Methods (2000) 243:77-97) and Magnetic bead cell sorting (MACS). The conventional MACS procedure is described by Miltenyi et al., “High Gradient Magnetic Cell Separation with MACS,” Cytometry 11:231-238 (1990). To sort cells by MACS, one labels cells with magnetic beads and passes the cells through a paramagnetic separation column. The separation column is placed in a strong permanent magnet, thereby creating a magnetic field within the column. Cells that are magnetically labeled are trapped in the column; cells that are not pass through. One then elutes the trapped cells from the column. In one embodiment, an antibody directed against SALL4 is used in cell sorting to isolate embryonic stem cells, adult stem cells and/or cancer stem cells. In another embodiment, an antibody directed against SALL4 is used in flow: cytometry analysis to detect cells expressing SALL4, where such cells are associated with proliferative disease progression or neoplastic cell formation. In a related aspect, SALL4 is SALL4A or SALL4B.

Myelodysplastic Syndrome (MDS) remains an incurable hematopoietic stem cell (HSC) malignancy that occurs most frequently among the elderly, with about 14,000 new cases each year in the USA. About 30-40 percent of MDS cases progress to Acute Myeloid Leukemia (AML). The incidence of MDS continues to increase as our population ages. Even though MDS and AML have been studied intensely, to date no satisfactory treatments have been developed, and the precise cellular or molecular events that induce progression of MDS to AML still remain poorly understood. Until very recently, no suitable cell line or animal model has been available for studying MDS and its progression to AML. Consequently, little progress has been made in understanding the molecular basis of this disease thus the development of potential therapeutic treatments has been extremely slow and discouraging. An innovative approach is urgently needed if the research community is going to succeed in unraveling MDS and AML biology and creating a breakthrough in the development of new therapies for a persistent disease that has claimed many lives.

Up to now, therapies for MDS and AML have focused on the leukemic blast cells because they are very abundant and clearly represent the most immediate problem for patients. However, an important fact centers on leukemic stem cells (LSCs) being quite different from most other leukemia cells (“blast” cells), and these LSCs constitute a rare subpopulation. While killing blast cells can provide short-term relief for MDS patients, LSCs, if not destroyed, will always re-grow causing the patient to relapse. It is imperative that the LSCs are destroyed in order to achieve durable cures for MDS disease. Unfortunately, standard drug regimens are not effective against the LSCs of either MDS or AML. To address this deficiency, a critical element in our proposed studies focuses on the development of new therapies that can specifically target LSCs. To this end, we have discovered that a reduction in the expression level of the SALL4 stem cell gene leads to apoptosis in LSCs and, importantly, spares normal stem cells.

As disclosed herein, SALL4 is a critical stem gene that modulates stem cell pluripotency. For example, SALL4 knockdown results in massive apoptosis associated with reduction of Bmi-1. The SALL4-induced apoptosis can be fully rescued by restoring Bmi-1 to a normal level. While not being bound by theory, it seems that SALL4-induced apoptosis involves through regulation of Bmi-1.

Further, the present invention demonstrates that overexpression of SALL4 in mice transforms HSCs/HPCs into LSCs with up-regulation of Bmi-1. Moreover, SALL4 is able to bind to the Bmi-1 promoter. In one embodiment, a method of modulating apoptosis and cell-cycle arrest is disclosed, where neoplastic cells are contacted with an agent that modulates expression of SALL4 and/or modulates the expression of Bmi-1. In one aspect, such sells are AML cells. In another aspect, the modulation reduces expression levels of SALL4 and/or Bmi-1 to induce cell cycle arrest and/or apoptosis. In a related aspect, such cells can be rescued by restoring Bmi-1 levels to substantially normal.

In one aspect, apoptosis and cell cycle arrest may be achieved by targeting SALL4 or Bmi-1, or by targeting the combination. In another aspect, the induction of apoptosis and/or cell cycle arrest may be accomplished by targeting SALL4 downstream targets. In one embodiment, a method of modulation of Bmi-1 via SALL4 targeting is disclosed, where such modulation results in apoptosis/cell cycle arrest in cancer stem cells and/or leukemic stem cells, thereby treating cancer in a subject in need thereof.

As disclosed herein, SALL4 is an important survival and proliferative factor for NTERA2 cells. Given the observation that SALL4 is also present in other cancer stem cells, SALL4 may be an attractive target for the induction of cancer stem cells to undergo apoptosis.

In one embodiment, SALL4B transgenic mice that exhibit MDS/AML associated with expansion of LSCs are disclosed. In one aspect, 5′ azacytidine (5AC) or a combination with bortezomib, a proteasome inhibitors, is administered to SALL4B transgenic mice and changes are monitored in HSC and HPC subpopulations. In a related aspect, SALL4B transgenic mice will be treated with a variety of doses. Further, the data will be used to identify an optimal dose that maximizes inhibition of LSC expansion associated with therapeutic responses in SALL4B transgenic mice.

In another embodiment, 5AC is administered alone or a combination with bortezomib to evaluate their effects on the long-term self-renewal ability of LSCs in vitro using serial replating assays. In one aspect, the effects of apoptosis on LSCs are also examined by, for example, but not limited to, TUNEL assay and measurement of caspase-3 activity. In another aspect, the method determines changes in the expression levels of SALL4B; its downstream target, Bmi-1; and its pathways associated with cell growth and/or cell death in HSCs, such as p16 and p19 in transgenic mice, during treatment of 5AC or bortezomib alone or together, by for example, Q-RT-PCR and western blotting. Peripheral blood samples may be obtained from SALL4B transgenic mice treated with 5AC or a bortezomib combination with age-matched, untreated control mice. Complete blood cell counts with automated differentials may be determined weekly. The differentials may be confirmed on smears. Further, latency of AML transformation may be compared between SALL4B mice treated with 5AC or a combination with bortezomib and untreated SALL4B mice. The onset of AML may be monitored by analysis of peripheral blood smears and bone marrow biopsies.

In one embodiment, a method of treating a cancer of stem cell or progenitor cell origin is disclosed including administrating to a subject in need thereof a composition containing an agent which reduces the expression level of SALL4.

In one aspect, the agent is an oligonucleotide sequence selected from SEQ ID NO:30, SEQ ID NO:31; or SEQ ID NO:32. In another aspect, the composition comprises a methylation inhibitor, including but not limited to, 5′ azacytidine, 5′ aza-2-deoxycytidine, 1-B-D-arabinofuranosyl-5-azacytosine, or dihydroxy-5-azacytidine. In a related aspect, the composition further comprises a proteasome inhibitor, including but not limited to, MG 132, PSI, lactacystin, epoxomicin, or bortezomib.

Germ cell tumors (GCTs) are a diverse group of neoplasms that often present a challenge in clinical diagnoses and are most often diagnosed solely based on the histological presentation of the specimen. However, this can be difficult in many cases. Often a biopsy specimen is so small that accurate diagnosis of mixed GCTs is insufficient.

Immunohistochemistry staining with SALL4 antibodies produces a specific and sensitive signal, the nuclear staining is consistent with the role of SALL4 as a transcription factor, and its lack of background staining provided distinct evidence of its expression in the positively stained cells. Our data show that SALL4 is expressed solely in cells with a pluripotent potential. Seminoma and embryonal carcinoma are clearly primitive cells with the potential to differentiate into many other cell lines. Immature teratomas and yolk sac tumors are called tissue stem cells because they have a pluripotent potential but can only differentiate further into cells of a specific tissue. The mature teratomas do not express SALL4, which is consistent with the fact that they do not have the ability to differentiate any further.

As disclosed, the staining of SALL4 in spermatogenesis shows that SALL4 is strongly expressed in germ cells but not in any other cells of the seminiferous tubules. Similarly, SALL4 is expressed in an undifferentiated embryonal carcinoma cell line, but after induced differentiation, its expression is down-regulated. SALL4 is also not expressed in a significant number of cells derived from normal of cancerous epithelial tissues. For example, the tissue types represented in a tissue array may contain less than 2% of cells that stain positive for SALL4. Thus, cells that stain positive for SALL4 in the arrays are indicative of tissue stem cells.

Further, the staining of the seminiferous tubules with the SALL4 antibody is unique in that only the germ cells of the tubule stained positive for SALL4. Moreover, both germ cells of seminiferous tubules and those of various primitive malignant GCTs stain positive for SALL4.

In one embodiment, a method of diagnosing disorders of primordial cell origin in a subject is disclosed including determining the expression of SALL4 in a tissue sample from the subject. In one aspect, the disorder is associated with a germ cell tumor (GCT). Further, the GCT includes classic seminoma, spermatocytic seminoma, embryonal carcinoma, yolk sac tumor, or immature teratoma.

In another aspect, the tissue sample comprises cells of testicular origin, including that substantially all mature testicular cell types present in the sample do not express SALL4. Further, the tissue sample may be obtained from a site which comprises cells that have metastasized from a GCT.

In another embodiment, a method of monitoring engraftment of transplanted stem cells in a subject is disclosed including determining the level of expression of SALL4 in stems cells prior to transplantation into a subject, grafting the cells into the subject, and determining the level of expression of SALL4 in the grafted stem cells at time intervals post-transplantation, where a decrease in SALL4 expression over the time interval correlates with differentiation of the stem cells, and wherein such differentiation is indicative of positive engraftment of cells in the subject.

In one aspect, an increase in SALL4 expression over the time interval correlates with repression of differentiation, and wherein such repression is indicative of negative engraftment of cells in the subject.

Such intervals may be from about 1 to 4 hour, about 4 to 12 hours, about 12 to 24 hours, about 24 to 48 hours, about 48 to 72 hours, about 3 to 7 days, about 7 days to 2 weeks, about 2 weeks to 1 month, about 1 to 6 months, and/or about 6 months to a year.

In another aspect, the cell is transformed by a vector encoding an exogenous or endogenous gene product.

In one embodiment, a method for isolating stem cells from cord blood disclosed including obtaining umbilical cord cells (UBC) from a subject, sorting cells that express SALL4 from cells that do not express SALL4, where UBCs expressing SALL4 are indicative of isolated stem cells. Further, the method may include, optionally, selecting by one or more markers, cells from the sorted cells that express SALL4.

In one aspect, the one or more markers are selected from the group consisting of SSEA-1, SSEA-2, SSEA-4, TRA-1-60, TRA-1-81, CD34+, CD59+, Thy1/CD90+, CD38^{lo/-}, C-kit−/lo, lin−, SH2, vimentin, periodic acid Schiff activity (PAS), FLK1, BAP, and acid phosphatase.

In one embodiment, a method for detecting the presence or absence of the polynucleotide comprising a nucleic acid sequence encoding SEQ ID NO: 2, SEQ ID NO: 4, or SEQ ID NO: 6 in a biological sample is disclosed including, but not limited to, contacting the biological sample under hybridizing conditions with a probe comprising a fragment of at least 15 consecutive nucleotides of a polynucleotide having a sequence set forth in SEQ ID NO: 1, SEQ ID NO: 3, or SEQ ID NO: 5, or a complement of SEQ ID NO: 1, SEQ ID NO: 3 or SEQ ID NO: 5, and detecting hybridization between the probe and the sample, where hybridization is indicative of the presence of the polynucleotide.

In another embodiment, a method for detecting a polypeptide comprising an amino acid sequence as set forth in SEQ ID NO: 2, SEQ ID NO: 4, or SEQ ID NO: 6 present in a biological sample is disclosed including, but not limited to, providing an antibody that binds to the polypeptide, contacting the biological sample with the antibody, and determining the binding between the antibody to the biological sample, where binding is indicative of the presence of the polypeptide.

In one embodiment, a method of treating myelodysplastic syndrome (MDS) in a subject is described, including administering to the subject a polynucleotide having a nucleic acid sequence as set forth in SEQ ID NO: 1, SEQ ID NO: 3, SEQ ID NO: 5, a complement of SEQ ID NO: 1, a complement of SEQ ID NO: 3, a complement of SEQ ID NO: 5, or fragments thereof comprising at least 15 consecutive nucleotides of a polynucleotide encoding the amino acid sequence as set forth in SEQ ID NO: 2, SEQ ID NO: 4, or SEQ ID NO: 6. In a related aspect, the method includes administering a polynucleotide as set forth in SEQ ID NO: 1, SEQ ID NO: 3, or SEQ ID NO: 5. In one aspect, the MDS is acute myeloid leukemia (AML).

In one embodiment, a method of identifying an agent which modulates the effect of a SALL family member protein on OCT4 expression is disclosed including co-transfecting a cell with a vector comprising a promoter-reporter construct, wherein the construct comprises an operatively linked OCT4 promoter and a nucleic acid encoding gene expression reporter protein, and a vector comprising a nucleic acid encoding a SALL family member protein, contacting the cell with an agent, and determining the activity of the promoter-reporter construct in the presence and absence of the agent, where determining the activity of the promoter-reporter construct correlates with the effect of the agent on SALL family member protein/OCT4 interaction.

In a related aspect, the promoter region comprises nucleic acid sequence including but not limited to, SEQ ID NO:26, and the expression reporter protein is luciferase.

In another embodiment, a method of treating a neoplastic or proliferative disorder, where cells of a subject exhibit de-regulation of self-renewal, is disclosed including administering to the subject a pharmaceutical composition containing an agent which inhibits the expression of SALL4.

In another embodiment, a method of identifying a substance which binds to a polypeptide including an amino acid sequence as set forth in SEQ ID NO: 2, SEQ ID NO: 4, or SEQ ID NO: 6 is provided, where the method comprises contacting the polypeptide with a candidate substance and detecting the binding of the substance to the polypeptide.

In one embodiment, a method of identifying a substance which modulates the function of a polypeptide including an amino acid sequence as set forth in SEQ ID NO: 2, SEQ ID NO: 4, or SEQ ID NO: 6 is disclosed, where the method includes contacting the polypeptide with a candidate substance and determining the activity of the polypeptide, and where a change in the activity in the presence of the candidate substance is indicative of the substance modulating the function of the polypeptide.

In another embodiment, a method of diagnosing myelodysplastic syndrome (MDS) in a subject is described including, but not limited to, providing a biological sample from the subject, contacting the biological sample with a probe having a fragment of at least 15 consecutive nucleotides of a polynucleotide sequence set forth in SEQ ID NO: 1, SEQ ID NO: 3, SEQ ID NO: 5, a complement of SEQ ID NO: 1, a complement of SEQ ID NO: 3, or a complement of SEQ ID NO: 5 under hybridization conditions, and detecting the hybridization between the probe and the biological sample, where detecting of hybridization correlates with MDS. In one aspect, the MDS is acute myeloid leukemia (AML).

In another embodiment, a method of diagnosing a myelodysplastic syndrome (MDS) in a subject is described, including, but not limited to, providing a biological sample from the subject, contacting the biological sample with an antibody which binds to a polypeptide comprising an amino acid as set forth in SEQ ID NO: 2, SEQ ID NO: 4, or SEQ ID NO: 6, and detecting the binding of the antibody to the sample, where detecting binding correlates with MDS. In one aspect, the MDS is acute myeloid leukemia (AML).

In one embodiment, a method of diagnosing a neoplastic or proliferative disorder is disclosed including contacting a cell of a subject with an agent that detects the expression of a SALL family member protein and determining whether a SALL family member protein is expressed in the cell, where determining the expression of the SALL family member protein positively correlates with induction of self-renewal in the cell, whereby such expression is indicative of neoplasia or proliferation.

In one aspect, the agent is labeled and the determining step includes detection of the agent by exposing the subject to a device which images the location of the agent. In a related aspect, the images are generated by magnetic resonance, X-rays, or radionuclide emission.

In one embodiment, a method of modulating the cellular expression of a polynucleotide encoding a zinc finger transcriptional factor which is constitutively expressed in primary acute myeloid leukemia cells, including introducing a double stranded RNA (dsRNA) which hybridizes to the polynucleotide, or an antisense RNA which hybridizes to the polynucleotide, or a fragment thereof, into a cell. In a related aspect, the modulating is down-regulating.

Infantile hemangeomas are very common in newborn and young children. Almost 10% of the Caucasian population have hemangiomas. Sixty percent of the hemangiomas occur on the head and neck and most of the hemangiomas go through a proliferative phase of growth, expanding rapidly after birth and involuting as the child gets older. Some of these hemangiomas may become large enough that they destroy head and neck structures. Many are severely disfiguring and can cause children to have psychosocial stigmata that can prevent normal maturation.

In one embodiment, antibody directed against human SALL4 is used to characterize subsets of stem cells in hemangiomas, where such antibodies bind to SALL4 expressing cells, which cells are putative pluripotent stem cells. In a related aspect, 5 to 10% of the cells comprising hemangiomas bind to such SALL4 directed antibodies. Further, diagnosis and monitoring of hemangioma involution can be determined by as decrease in SALL4 binding by such antibodies. In one aspect, the monitoring may include, but is not limited to, flow cytometry and/or examination of tissue sections of cells immunohistochemically stained with anti-SALL4.

In another embodiment, non-surgical treatment for infantile hemangiomas is disclosed, where an agent which reduces SALL4 expression is administered to a subject in need thereof in an amount sufficient to cause induction of involution of the hemangiomas in the subject.

In another embodiment, a transgenic animal is disclosed. In a general aspect, a transgenic animal is produced by the introduction of a foreign gene in a manner that permits the expression of the transgene. Methods for producing transgenic animals are generally described by Wagner and Hoppe (U.S. Pat. No. 4,873,191; which is incorporated herein by reference), Brinster et al. (1985); which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press (1994); which is incorporated herein by reference in its entirety).

Typically, a gene is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish.

DNA clones for microinjection can be prepared by any means known in the art. For example, DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris, pH 7.4, and 1 mM EDTA) and purified on an Elutip-D™ column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer.

The present invention also provides pharmaceutical compositions comprising at least one compound capable of treating a disorder in an amount effective therefore, and a pharmaceutically acceptable vehicle or diluent. The compositions of the present invention may contain other therapeutic agents as described, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

Pharmaceutical compositions employed as a component of invention articles of manufacture can be used in the form of a solid, a solution, an emulsion, a dispersion, a micelle, a liposome, and the like, where the resulting composition contains one or more of the compounds described above as an active ingredient, in admixture with an organic or inorganic carrier or excipient suitable for enteral or parenteral applications. Compounds employed for use as a component of invention articles of manufacture may be combined, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used.

Invention pharmaceutical compositions may be administered by any suitable means, for example, orally, such as in the form of tablets, capsules, granules or powders; sublingually; buccally; parenterally, such as by subcutaneous, intravenous, intramuscular, or intracisternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The present compounds may also be administered liposomally.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

The subjects treated in the above methods, in which cells targeted for modulation is desired, are mammals, including, but not limited to, cows, sheep, goats, horses, dogs, cats, guinea pigs, rats or other bovine, ovine, equine, canine, feline, rodent or murine species, and preferably a human being, male or female.

The term “therapeutically effective amount” means the amount of the subject compound that will elicit the biological or medical response of a tissue, system, animal or human that is being sought by the researcher, veterinarian, medical doctor or other clinician.

The term “composition,” as used herein, is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for control release.

Formulations for oral use may also be presented as hard gelatin capsules where the active ingredient is mixed with an inert solid diluent, for example, calcium carbonate, calcium phosphate or kaolin, or as soft gelatin capsules where the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles).

Nucleic acid according to the present disclosure, encoding a polypeptide or peptide able to interfere with SALL4 may be used in methods of gene therapy, for instance in treatment of individuals with the aim of preventing or curing (wholly or partially) a tumor e.g., in cancer, or other disorder involving loss of proper regulation of the cell-cycle and/or cell growth, or other disorder in which specific cell death is desirable.

Vectors such as viral vectors have been used in the art to introduce nucleic acid into a wide variety of different target cells. Typically the vectors are exposed to the target cells so that transfection can take place in a sufficient proportion of the cells to provide a useful therapeutic or prophylactic effect from the expression of the desired polypeptide. The transfected nucleic acid may be permanently incorporated into the genome of each of the targeted tumour cells, providing long lasting effect, or alternatively the treatment may have to be repeated periodically.

A variety of vectors, both viral vectors and plasmid vectors, are known in the art, see U.S. Pat. No. 5,252,479 and WO 93/07282. In particular, a number of viruses have been used as gene transfer vectors, including papovaviruses, such as SV40, vaccinia virus, herpesviruses, including HSV and EBV, and retroviruses. Many gene therapy protocols in the art have used disabled murine retroviruses.

As an alternative to the use of viral vectors other known methods of introducing nucleic acid into cells includes electroporation, calcium phosphate co-precipitation, mechanical techniques such as microinjection, ballistic methods, transfer mediated by liposomes, and direct DNA uptake and receptor-mediated DNA transfer.

Receptor-mediated gene transfer, in which the nucleic acid is linked to a protein ligand via polylysine, with the ligand being specific for a receptor present on the surface of the target cells, is an example of a technique for specifically targeting nucleic acid to particular cells.

In the treatment of a subject where cells are targeted for modulation, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The following examples are intended to illustrate but not limit the invention.

### EXAMPLES

### Methods

### Molecular Cloning

Plasmid construction and DNA sequencing were performed in accordance with standard procedures. For cloning of SALL4 isoforms, PCR primers were designed, based on the genomic clone RP5-1112F19 (SEQ ID NO: 25) (GenBank accession no. AL034420). SALL4 isoforms were cloned with the use of the Marathon-Ready cDNA library derived from human fetal kidney (BD Biosciences Clontech, Palo Alto, Calif.), according to the supplier's protocol. The amplified PCR products were cloned into a TA Cloning vector (Invitrogen Corp., Carlsbad, Calif.), and the nucleotide sequences were determined by DNA sequencing. The GAL4-SALL4B construct was generated by PCR with the use of a 5′ primer and a 3′ primer with a restriction enzyme site, BamHI, at each end:

5′ primer:

(SEQ ID NO: 7)

5′-TTATCAGGATCCTGGTCGAGGCGCAAGCAGGCGAAACCC-3′;
and
3′ primer:

(SEQ ID NO: 8)

5′-CCAGGATCCTTAGCTGACCGCCAATCTTGTTTC-3′.

The GAL4-SALL4B construct was expected to encode 93 amino acids of minimal GAL4 DNA-binding domain and the full length of SALL4B, except for the first amino acid, methionine.

### Determination of Alternative Splicing Patterns in Different Tissues

Reverse transcription (RT)-PCR was used to evaluate mRNA expression patterns of SALL4 in adult tissues. A panel of eight normalized first-strand cDNA preparations, derived from different adult tissues, was purchased from BD Biosciences Clontech. PCR amplification was performed in a 50-μl reaction volume containing 5 μl of cDNA, 10 mM Tris HCl (pH 8.3), 50 mM KCl, 2 mM MgCl₂, 0.2 mM dNTPs, and 1.25 U of Taq DNA polymerase (PerkinElmer Life Sciences, Boston, Mass.). After an initial denaturation at 94° C. for 10 min, amplification was performed for 30 cycles under the following conditions: 30-sec denaturation at 94° C., 30-sec annealing at 55° C., and 30-sec extension at 72° C. The last cycle was followed by a final 7-min extension at 72° C.

Amplification of glyceraldehyde phosphate dehydrogenase (GAPDH) mRNA was used to control for template concentration loading. The primer pairs selected specifically for SALL4 isoforms were the following:

| | |
|---|---|
| SALL4A primers | |
| (sense primer: | |
| 5′-ATTGGCACCGGCAGTTACCACC; | (SEQ ID NO: 9) |
| antisense primer: | |
| 5′-AGTACTCGTGGGCATATTGTC-3′) | (SEQ ID NO: 10) |
| and | |
| 2) SALL4B primers | |
| (sense primer: | |
| 5′-ATGTCGAGGCGCAAGCAGGCGAAAC-3′; | (SEQ ID NO: 11) |
| antisense primer: | |
| 5′-TTAGCTGACCGCAATCTTGTTTTCT-3′). | (SEQ ID NO: 12) |

PCR products were electrophoretically separated on 1% agarose gel. DNA sequencing was also used to confirm amplification products.

### Antibody Generation

The peptide MSRRKQAKPQHIN (SEQ ID NO: 13) of human SALL4 was chosen for its potential antigenicity (amino acids 1-13) and used to prepare an antipeptide antibody. This region is also identical to that of mouse SALL4 so that the generated antibody could be expected to cross-react with mouse SALL4. SALL4 antipeptide antibody was produced in rabbits in collaboration with Lampire Biological Laboratories Inc. (Pipersville, Pa.).

### Gel Electrophoresis and Western Blot Analysis

Sodium dodecyl sulfate-polyacrylamide gel electrophoresis (SDS-PAGE) was carried out in SDS 10% w/v polyacrylamide slab gels according to Laemmli, and the proteins were then transferred to nitrocellulose membranes. Immunoblotting of rabbit immune serum with the SALL4 antipeptide antibody (1:100) was performed with an electrochemiluminescence detection system as described by the manufacturer (Amersham Biosciences, Piscataway, N.J.).

### Leukemia and Normal Tissues

Leukemia and normal samples, either in paraffin blocks or frozen in dimethylsulfoxide (DMSO), were collected from the files of The University of Texas M.D. Anderson Cancer Center, Houston, Tex., and the Dana-Farber Cancer Institute, Boston, Mass., between 1998 and 2004 under approved Institutional Review Board protocols. The diagnosis of all tumors was based on morphologic and immunophenotypic criteria according to the FAB Classification for Hematopoietic Neoplasms. CD34+ fresh cells were purchased from Cambrex.

### Real-Time Quantitative RT-PCR

TaqMan 5′ nuclease assay was used (Applied Biosystems, Foster City, Calif.) in these studies. Total RNA from purified CD34+ HSCs/HPCs from normal bone marrow and peripheral blood, 15 AML samples, and three leukemia cell lines was isolated with the RNeasy Mini Kit and digested with DNase I (Qiagen). RNA (1 μg) was reverse-transcribed in 20 μL with the use of Superscript II reverse transcriptase and a poly(dT)12-18 primer (Invitrogen). After the addition of 80 μL of water and mixing, 5-μL aliquots were used for each TaqMan reaction. TaqMan primers and probes were designed with the use of Primer Express software version 1.5 (Applied Biosystems). Real-time PCR for SALL4 and GAPDH was performed with the TaqMan PCR core reagent kit (Applied Biosystems) and an ABI Prism 7700 Sequence Detection System (PE Applied Biosystems). The PCR reaction mixture contained 3.5 mM MgCl₂; 0.2 mM each of deoxyadenosine triphosphate (dATP), deoxycytidine triphosphate (dCTP), and deoxyguanosine triphosphate (dGTP); 0.4 mM deoxyuridine triphosphate (dUTP); 0.5 μM forward primer; 0.5 μM reverse primer; 0.1 μM TaqMan probe; 0.25 U uracil DNA glycosylase; and 0.625 U AmpliTaq Gold polymerase in 1×TaqMan PCR buffer. cDNA (5 μL) was added to the PCR mix, and the final volume of the PCR reaction was 25 μL. All samples were run in duplicate. GAPDH was used as an endogenous control. Thermal cycler conditions were 50° C. for 2 min, 95° C. for 10 min, and 45 cycles of 95° C. for 30 sec and 60° C. for 1 min. Data were analyzed with the use of Sequence Detection System software version 1.6.3 (Applied Biosystems). Results were obtained as threshold cycle (Ct) values. The software determines a threshold line on the basis of the baseline fluorescent signal, and the data point that meets the threshold is given as the Ct value. The Ct value is inversely proportional to the starting number of template copies. All measurements were performed in duplicate. TaqMan sequences include the following:

| | |
|---|---|
| GAPDH forward primer: | |
| (5′-GAAGGTGAAGGTCGGAGTC-3′) | (SEQ ID NO: 14) |
| and | |
| reverse primer: | |
| (5′-GAAGATGGTGATGGGATTTC-3′), | (SEQ ID NO: 15) |
| TaqMan probe: | |
| (5′-CAAGCTTCCCGTTCTCAGCC-3′), | (SEQ ID NO: 16) |
| and | |
| SALL4 forward primer: | |
| (5′-CCTCCTAATGAGAGTATCTGGGTGAT-3′) | (SEQ ID NO: 17) |
| and | |
| reverse primer: | |
| (5′-TTAAAACATACAGCGCATGATTGG-3′). | (SEQ ID NO: 18) |

### Design and Construction of Tissue Arrays

Tissue arrays that included triplicate tumor cores from leukemia specimens were sectioned (5 μm thick). A manual tissue arrayer (Beecher Instruments, Silver Spring, Md.) was used to construct the tissue arrays.

### Immunohistochemistry

Immunohistochemical staining was performed according to standard techniques. Briefly, formalin-fixed, paraffin-embedded, 4-μm-thick tissue sections were deparaffinized and hydrated. Heat-induced epitopes were retrieved with a Tris buffer (pH 9.9; Dako Corp., Carpinteria, Calif.) and a rapid microwave histoprocessor. After incubation at 100° C. for 10 min, slides were washed in running tap water for 5 min and then with phosphate buffered saline (PBS; pH 7.2) for 5 min. Tissue sections were then incubated with anti-SALL4 antibody (1:200) for 5 h in a humidified chamber at room temperature. After three washes with PBS, tissue sections were incubated with antimouse immunoglobulin G and peroxidase for 30 min at room temperature.

After three washes with PBS, tissue sections were incubated with 3,3′-diaminobenzidine/H₂O₂ (Dako) for color development; hematoxylin was used to counterstain the sections. Neoplastic cells were considered to be positive for SALL4 when they showed definitive nuclear staining.

### Generation of Transgenic Mice

SALL4B cDNA, corresponding to the entire coding region, was subcloned into a pCEP4 vector (IntroGene; now Crucell, Leiden, The Netherlands) to create the CMV/SALL4B construct for the transgenic experiments. Subsequent digestion with SalI, which does not cut within the SALL4B cDNA, released a linear fragment containing only the CMV promoter, the SALL4 cDNA coding region, the SV40 intron, and polyadenylation signal without additional vector sequences.

Transgenic mice were generated via pronuclear injection performed in the transgenic mouse facility at Yale University. Identification of SALL4B founder mice and transmission of the transgene was determined by PCR analyses. The PCR primers used for the genotyping span the junction of the 5′ SALL4B cDNA to the CMV promoter (sense primer: 5′-CAGAGATGCTGAAGAACTCCGCAC-3′ (SEQ ID NO: 19); antisense primer: 5′-AGCAGAGCTCGTTTAGTGAACCG-3′ (SEQ ID NO: 20)).

### Hematologic Analysis

Complete blood cell counts with automated differentials were determined with a Mascot Hemavet cell counter (CDC Technologies, Oxford, Conn.). For progenitor assays, 1.5×10⁴ bone marrow cells were plated in duplicate 1.25-ml methylcellulose cultures supplemented with recombinant mouse interleukin-3 (IL-3) (10 ng/ml), IL-6 (10 ng/ml), stem cell factor (SCF) (50 ng/ml), and erythropoietin (3 U/ml) (M3434, StemCell Technologies, Vancouver, British Columbia, Canada). Colonies were recorded between days 7 and 14 (CFU-G, CFU-GM, CFU-M, CFU-GEMM, and BFU-E). Peripheral blood, bone marrow smears, and cytospin from pooled CFU cells were stained with Wright-Giemsa stain.

### Flow Cytometric Analysis

Cells were stained with directly conjugated antibodies to Gr-1, Mac-1, B220, Ter119, c-kit, CD34, CD45, CD41, CD19, CD5, CD3, CD4, CD8, propidium iodide (PI) or Annexin V (BD Biosciences Pharmingen, San Diego, Calif.). Ten thousand scatter-gated red cells were acquired on a FACScan and analyzed with CellQuest software (BD Biosciences Clontech).

Proliferating cells were first treated with and without IS3 295 for up to 48 hours. A portion of the cells were harvested to incorporate bromodeoxyuridine (BrdU) (Pharmingen) following the manufacturer's instructions and analyzed by flow cytometry. Harvested cells also were analyzed for apoptosis via detection by TUNEL assay using a Roche Applied Science apoptosis detection system (Fluorescein) according to manufacturer's instructions.

### Statistical Analysis

Student's t-Test was used for all the statistical analysis, assuming normal two-tailed distribution and unequal variance. Further, treatment with 5AC or a bortezomib combination that will affect SALL4B HSCs and HPCs will be determined over various doses. In addition, identifying an optimal dose will be carried out. The primary endpoint of such a study is post-treatment percentage of SALL4B HSCs/HPCs and apoptotic cells as compared to normal HSCs/HPCs within these populations after the animals are sacrificed. Other endpoints include determining the long-term self-renewal ability of LSCs in vitro and the expression of Bmi-1 after exposure to 5AC and a combination of 5AC with bortezomib.

### Cell Culture and Transfection

All cell cultures were maintained at 37° C. with 5% CO₂. HEK-293 (ATCC: CRL-11268) cells were cultured in Dulbecco modified Eagle medium (DMEM) supplemented with 10% heat-inactivated FBS (fetal bovine serum) and penicillin/streptomycin (P/S). The HL60 cell line was cultured in RPMI 1640 medium supplemented with 10% FBS and P/S. A murine hemopoietic multipotential cell line, 32D (ATCC: CRL-1821), was maintained in RPMI 1640 supplemented with 10% FBS, P/S, and mouse leukemia inhibitory factor (mLIF; 1×103 U/ml, Chemicon, Pittsburgh, Pa.). Transfection of plasmids into HEK-293, mouse 32D cells, and HL60 cells was performed using Lipofectamine 2000 (Invitrogen, Carlsbad, Calif.) according to manufacturer's recommendations. Cells were plated in 24-well plates at a density of ˜1×10⁵ cells/well. Cells were harvested 24 h after transfection. Plasmid DNA for transient transfection was prepared with the Qiagen Plasmid Midi Kit (Valencia, Calif.).

### β-Galactosidase and Luciferase Assays.

The cells were extracted with 100 μl of luciferase cell culture lysis reagent (Promega Corp., Madison Wis.) 24 h after transfection. The β-galactosidase assay, performed with 10 μl of cell extract, used the P-Galactosidase Enzyme Assay System (Promega) and the standard assay protocol provided by the manufacturer (except that 1 M Tris base was used as stopping buffer, instead of sodium carbonate). For the luciferase assay (Promega), 5 μl of extract were used in accordance with the manufacturer's instructions. After subtraction of the background, luciferase activity (arbitrary units) was normalized to β-galactosidase activity (arbitrary units) for each sample.

### Promoter Reporter Assays

In general, 0.25-0.3 μg of an OCT4-Luc construct (PMOct4) comprising an OCT4 promoter (SEQ ID NO:26) or SALL-Luc construct containing a SALL family protein (i.e., SALL1, SALL3, SALL4A, or SALL4B) promoter (i.e., SEQ ID NO:27, SEQ ID NO:28, and SEQ ID NO:29, respectively, where SALL4A and SALL4B share the same promoter) was cotransfected with between 0.1 μg and 0.12 μg of renilla plasmid and/or various amounts (0-1.0 μg) of plasmid expressing SALL family proteins or OCT4 protein in HEK-293 or COS-7 cells. Typically, pcDNA3 vector was used as the control. Transfected cells were then monitored for luciferase activity 24 hour s post-transfection.

### Human Samples

Classic seminomas, embryonal carcinomas, yolk sac tumors, mature teratomas, immature teratomas, and choriocarcinomas were obtained from as paraffin-embedded sections and used in immunohistochemistry staining. A tissue microarray of non-GCTs was purchased from the National Institutes of Health (NIH).

### Cell Culture

Human EC cell line NTERA2.c1.D1 (ATCC#CRL-1973) was maintained in Dulbecco's modified Eagle's medium (DMEM) supplemented with 10% FBS (fetal bovine serum). Cells were induced to differentiate by treatment with different amounts of retinoic acid (Sigma). Phoenix packaging cells (ATCC: #SD-3443) are cultured by means well known in the art.

### Virus Production and SALL4 Knockdown

Two siRNA oligonucleotides (#7410, #7412; Origen, Rockville, Md.) that targeted different regions of the SALL4 gene were transfected into Phoenix packaging cells using Lipofectamin 2000. Shed virus was harvested. NTERA2 cells were infected with the virus collected 48 hours post-transfection. Stable SALL4 knockdown NTERA2 clones were obtained under puromycin (1.2 ug/ml) selection after 7 days. The pcDNA construct expressing Bmi-1 was used for transfection into the NTERA2 cell line.

### Bmi-1 Promoter Constructs and Site-Directed Mutagenesis

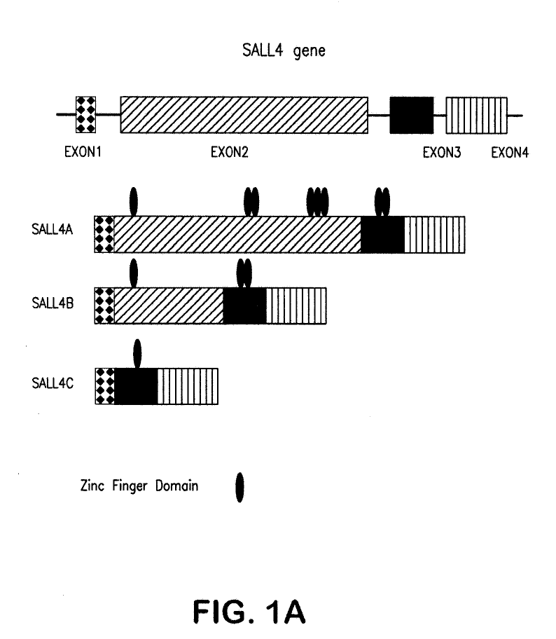

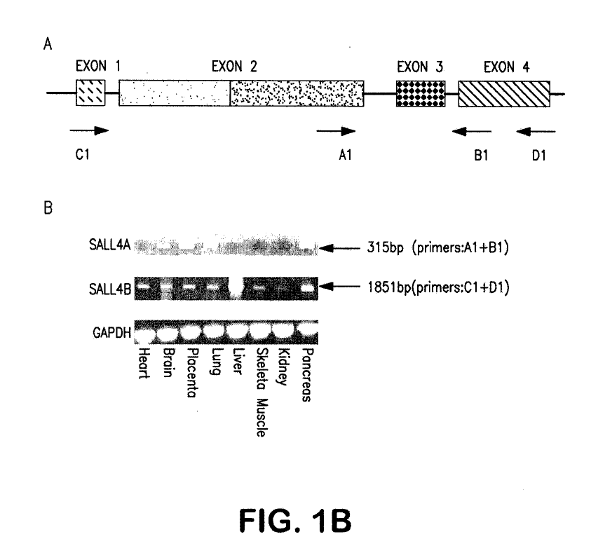

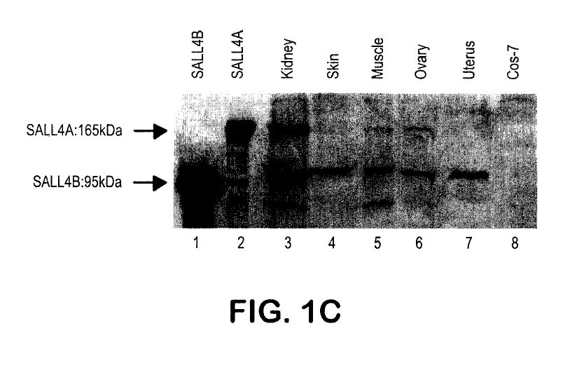

The 5′-flanking region of Bmi-1 was amplified with primers (5′ primer: 5′-CAT CCT CGA GGG CTG TTG ACA TCT GCA GAG ACT G-3′; 3′ primer: TCG TAG ATC TCA TTT CTG CTT GAT AAA AGA TCC TGG-3′) to generate a fragment from nucleotide (Nt)-1 to Nt-2102 upstream of the starting codon ATG with XhoI and BglII sites at each end respectively. Mouse genomic DNA isolated from ESCs was used as a template. The amplified PCR (polymerase chain reaction) fragment was cloned into the promoterless pGL3-basic luciferase reporter plasmid (Promega, Madison, Wis.) to generate plasmid Bmi-1 (P2102) (i.e. Nt-1 to -2102, see FIG. 1). Promoter fusion reporter fragments from Nt-1 to -1254, -683, -270 and -168 (P1254, P683, P270, and P168) were created in the same manner as Bmi-1. The deletion mutant of the Bmi-1-Luc promoter constructs P683 and P1254, which lack the −168-270 sequence, was generated using a QuikChange II mutagenesis kit (Stratagene, La Jolla, Calif.) according to the manufacturer's protocol.

siRNA Constructs

For down regulation of SALL4, 3 different sets of 60-bp oligonucleotides targeting different regions of the human SALL4 sequence were synthesized. These fragments were cloned into the HindIII and BglII sites of pSuper-retro-puro (OligoEngine, Seattle, Wash.) to generate pSuper-retro/SALL4-1 siRNA constructs, designated:

SEQ ID NO:30

(5′-gatcccccaacatcccttctgccaccttcaagagaggtggcagaag
ggatgttgtttttc-3′),

SEQ ID NO:31

(5′-gatcccccaccactgatcccaacgaattcaagagattcgttgggat
cagtggtgtttttc-3′),
and

SEQ ID NO:32

(5′-gatcccctcatttgccaccgagtcttttcaagagaaagactcggtg
gcaaatgatttttc-3′).

### Generation of Retrovirus

The Phoenix packaging cells (ATCC: SD-3443) were grown in DMEM with 10% FBS in 5% CO₂ at 37° C. Recombinant retroviruses were produced using the Phoenix packaging cell line that was transfected with the pSuper construct containing the control RNAi sequence or sequence directed against SALL4. The viral supernatant was collected 48 hours after transfection and filtered through a 0.45-μm filter.

### Bmi-1 Promoter Assays

Bmi-1 promoter luciferase assays were performed with the Dual-Luciferase Reporter Assay System (Promega, Madison Wis.). Twenty-four hours after transfection, HEK-293 cells were extracted with the use of a passive lysis buffer; a 20-μl aliquot was used for luminescence measurements with a luminometer. The data are represented as the ratio of firefly to Renilla luciferase activity (Fluc/Rluc). These experiments were performed in duplicate.

### ChIP Assay

HEK-293 32D cells (1×10⁶ cells/well in 6-well plates), with or without transient transfection, were processed using a ChIP Assay Kit (Upstate, Charlottesville, Va.) following the manufacture's protocol. Briefly, cells were cross-linked by adding formaldehyde (27 μl of 37% formaldehyde/ml) and incubated for 10 min. Then, chromatin was sonicated to an average size of approximately 500 bp and immunoprecipitated with SALL4 antibodies, preimmune serum, or anti-HA (hemagglutination) antibody. Antibodies for histone modifications, histone H3 trimethy K4 and histone H3 dimethy K79, were purchased from Abcam (Cambridge, Mass.). Histone-DNA crosslinks were reversed by heating at 65° C. followed by digestion with proteinase K (Invitrogen, Carlsbad, Calif.). DNA was recovered by using a PCR purification kit (Qiagen, Valencia, Calif.) and then used for PCR or QRT-PCR (quantitative real time polymerase chain reaction).

### Human Leukemia Samples and SALL4 Knockout

Leukemia and normal samples frozen in Dimethylsulfoxide (DMSO) were collected from the files of The University of Texas M.D. Anderson Cancer Center, Houston, Tex., under approved Institutional Review Board protocols. The diagnosis of all tumors was based on morphologic and immunophenotypic criteria according to the FAB Classification for Hematopoietic Neoplasms. The generation of SALL4 knockout mice was described (8).

### Cell Culture

W4 mouse ESCs (kindly provided by the Gene Targeting Core Facility, University of Iowa) either on feeders or in feeder-free conditions were cultured as described previously. For Sall4+/− deficient ESCs, G418 was added in the media at a concentration of 125 ug/ml.

### ChIP-Chip Assays

A complete protocol was provided by NimbleGen Systems Inc (Madison, Wis.). In brief, cells were grown, cross-linked with formaldehyde and sheared by sonication. The anti-SALL4 antibody and rabbit serum (ref.) were used for chromatin immunoprecipitation (CHIP). CHIP-purified DNA was blunt-ended, ligated to linkers and subjected to low-cycle PCR amplification. Promoter tiling arrays (RefSeq array) were produced by NimbleGen. The RefSeq mouse promoter array design is a single array containing 2.7 kb of each promoter region (from build MM5). The promoter region is covered by 50-75 mer probes at roughly 100 bp spacing dependent on the sequence composition of the region. The arrays were hybridized, and the data were extracted according to NimbleGen standard procedures.

Confirmation of the predicted binding sites was performed using Quantitative real-time PCR analysis of the amplicons that were applied to the arrays.

### Microarray Design and Analysis

A custom microarray was manufactured by NimbleGgen (Madison, Wis.) using maskless array synthesis. The mouse genes on this design (n=42558) were selected from the Mus musculus entries in the RefSeq collection. Each gene was compared with all others using the BLAST program to remove redundancies. Ten probe pairs for each target were selected from the 3′ 1 kb of each target. Probes were spaced evenly over the length of the target region (≦1 kb), so that the exact spacing depended on the length of the target sequence. Each probe was 24 nucleotides in length. For each perfect match probe there was also a mismatch probe, which differed by a single nucleotide.

Labeled cDNA was hybridized to the oligonucleotide probes on the microarray. After washing, arrays were stained with streptavidin-Cy3 conjugate (Amersham Biosciences, Piscataway, N.J.), followed by washing and a blow dry step. Slides were scanned using a GenePix 4000B microarray scanner (Axon Instruments, Union City, Calif., USA), and the feature intensities extracted from the TIF files were calculated by the scanner software using a proprietary application developed at NimbleGen (Madison, Wis., USA). This application calculates mean signal intensities for the pixels that define each feature (3×3 grid of pixels). The intensities for each gene are calculated by taking the mean of the intensities for the perfect match probes specific to each target minus the mean of the intensity of the mismatch probes. Probes that differed from the mean for the set by more than 3 SD were removed from the set and the mean recalculated. Average differences (recalculated mean) were used for subsequent analysis. Data analysis was performed using PANTHER and Ingenuity Pathway Analysis.

### Immunocoprecipitation and Western Blotting

For Oct4/SALL4 and Nanog/SALL4 interactions, plasmid pcDNA3/SALL4-HA was transfected into W4 ES cells to express SALL4-HA fusion protein. Lipofectamine 2000 reagent (Invitrogen) was used based on provided instructions. After 36 hours, cells were collected and treated with CelLytic M Cell Lysis Reagent (Sigma). The immunocoprecipitation were performed following the Catch and Release v2.0 Kit (Upstate) recommendations. Initially, W4 lysates were incubated with the anti-HA antibody (Bethyl Laboratories Inc.) or IgG at 25° C. for 40 minutes, protein bound to the beads was then washed and eluted with denaturing elution buffer containing 0.5% P-mercaptoethanol. Western blot was performed as described (ref.). The membrane was incubated with Oct-3/4 (H-134), Nanog (M−149) (both from Santa Cruz) or SALL4 antibodies at a 1:300 dilution at 4° C. overnight. Detection was done by using the SuperSignal West Pico solutions (Pierce).

### Generation of a SALL4 Floxed Allele and SALL4+/− Deficient ES Cells

The SALL4-flox vector was constructed by incorporating the 5′ NotI-SalI 2 kb fragment, the 3′ BamH/loxp-PacI-KpnI 3.2 kb fragment and the PacI/KpnI 3.4 kb fragment into a vector that contained pGK-Neo flanked by FRT and loxP sequences. LoxP sequences were placed so that exon 2 was excised upon Cre treatment, resulting in disruption of 6 zinc-finger motifs. These ES cells were infected with Ad-CMV-Cre or Ad-CMV-GFP (#1045 and #1060 Vector BioLabs) following the manufacturer's procedures. Conventional SALL4 deficient ES cells were established by methods known in the art.

### Example 1

### Molecular Analysis of SALL4

Molecular cloning of two alternatively splicing isoforms of human SALL4

Two full-length transcripts of SALL4 were isolated by 5′ and 3′ RACE-PCR (rapid amplification of the 5′ and 3′ cDNA ends-polymerase chain reaction) with the use of fetal human kidney Marathon-Ready cDNAs (BD Biosciences Clontech) as templates.

Sequence analysis of the larger cDNA fragment isolated revealed a single, large open reading frame, designated as SALL4A that started from a strong consensus initiation sequence and was expected to encode 1,053 amino acids. The other splicing variant of SALL4, designated SALL4B, lacked the region corresponding to amino acids 385-820 of the full-length SALL4A (FIG. 1a). The putative protein encoded by SALL4B cDNA was expected to consist of 617 amino acids.

To rule out the possibility that these two apparent splicing variants might result from artifacts, both variant mRNA sequences with corresponding sequences of the human genome were compared. SALL4A contained all exons (1-4) (FIG. 1a), whereas SALL4B lacked the 3′ large portion of exon 2. Both exon-intron splice sites satisfied the G-T-A-G rule. Both splicing variants had the same translational reading frame, but SALL4B mRNA encoded a protein with internal deletions. SALL4A contained eight zinc finger domains, while SALL4B had three zinc finger domains.

### Expression Pattern of the SALL4 Isoforms in Human Tissues

The alternative splicing patterns of SALL4 were delineated by reverse transcription (RT)-PCR in a variety of human tissues. A fragment of the ubiquitous GAPDH gene cDNA was amplified as a control (FIG. 1b). A 315-bp fragment representing the longer splice variant, SALL4A, was amplified in some tissues, achieving various expression levels. The SALL4B variant was present in every tissue at varying levels of expression. Detailed studies on SALL4 expression in hematopoietic tissues are described in the following results.

### Generation of SALL4 Antibody and Identification of SALL4 Protein Products

To identify SALL4 gene products and confirm the presence of SALL4 variants, a polyclonal antibody against a synthetic peptide (amino acids 1-13) of SALL4 was developed. This region was chosen because it is common to both SALL4 variants. The affinity-purified SALL4 peptide antibody recognized specifically two endogenous proteins in a human kidney total lysate. The two proteins were approximately 165 kDa and 95 kDa, which were identical to the molecular weights of overexpressed SALL4A and SALL4B in Cos-7 cells, respectively (FIG. 1c). Western blotting with this antibody confirmed that the SALL4 isoforms had different tissue distributions that were similar to those observed at the mRNA level (FIG. 1b-B).

### Failure of SALL4 to Turn Off in Human Primary AML and Myeloid Leukemia Cell Lines

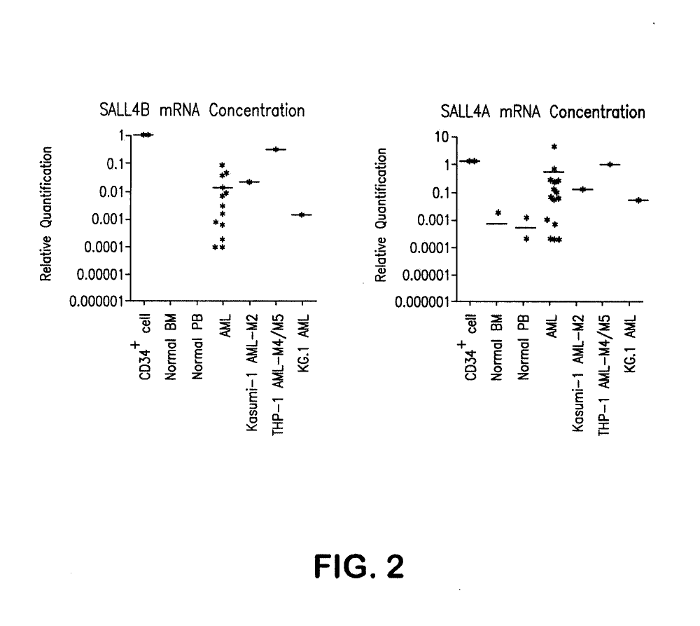

Because the chromosome region 20q13, where SALL4 is located, is frequently involved in tumors, SALL4 mRNA expression in AML was examined. Expression of SALL4 was quantitatively investigated by real-time RT-PCR in bone marrow cells derived from AML samples (N==15), myeloid leukemia cell lines (N=3) and compared with that of non-neoplastic hematopoietic cells from a purified CD34+ stem/progenitor pool (HSCs/HPCs purchased from Cambrex), normal bone marrow (N=3), and normal peripheral blood (N=3). With the use of isoform-specific primers (see FIG. 2a), either or both SALL4B and/or SALL4A, failed to be turned off (SALL4B) or down-regulated (SALL4A) in all AML samples and myeloid leukemia cell lines. The data were normalized to the endogenous expression of GAPDH and calibrated against the level of SALL4A or SALL4B expression in purified CD34+ cells. In contrast to the total absence of SALL4B in normal bone marrow, its expression in primary AML failed to be turned off in 13 of 15 AML samples and in all three myeloid leukemia cell lines. The median normalized level of SALL4A in primary AML samples was 40-fold higher than that in normal bone marrow. SALL4A expression levels in the myeloid leukemia cell lines KG.1, Kasumi-1, and THP-1 were, respectively, 8-, 25-, and 240-fold higher than those in normal bone marrow. Interestingly, both SALL4A and SALL4B expression levels were increased in 60% of AML samples and in all three cell lines, compared with those in normal bone marrow. In the remaining 40% of AML samples, either SALL4A or SALL4B failed to be down-regulated.

### Constitutive Expression of SALL4 Protein in Human Primary AML

To investigate whether the observed aberrant SALL4 expression was also present at the protein level, 81 AML samples were examined, ranging from AML classes M1 to M5 (FAB classification): M1 (N=20), M2 (N=27), M3 (N=8), M4 (N=16), M5 (N=3), and AML nonspecified (N=7); several samples of normal bone marrow, thymus and spleen, as well as normal CD34+ HSCs/HPCs.

Normal bone marrow, spleen and thymus showed no detectable SALL4 protein expression, and normal CD34+ HSCs/HPCs exhibited positive but weaker SALL4 protein staining; however, much stronger SALL4 expression was detected in the nuclei of leukemic cells (FIG. 2b-F). All 81 AML samples showed aberrant SALL4 expression, with the strongest staining seen in AML-M1 and -M2. These findings were consistent with SALL4 mRNA expression levels demonstrated by real-time RT-PCR (FIG. 2a). The data suggested that SALL4 was present in CD34+ HSCs/HPCs and down-regulated in mature granulocytes and lymphocytes. As a result, the constitutive expression of SALL4 in leukemia may have prevented the leukemic blasts from differentiating and/or gaining properties that were normally seen in HSCs.

### Generation of Transgenic Mice Constitutively Expressing Full-Length Human SALL4B

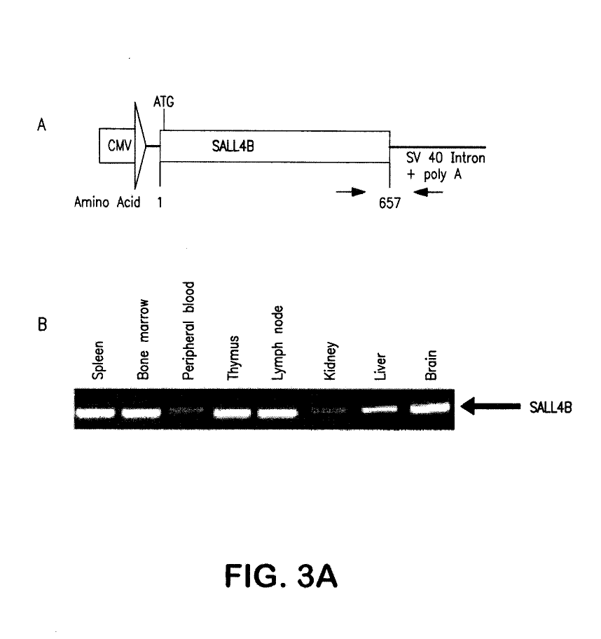

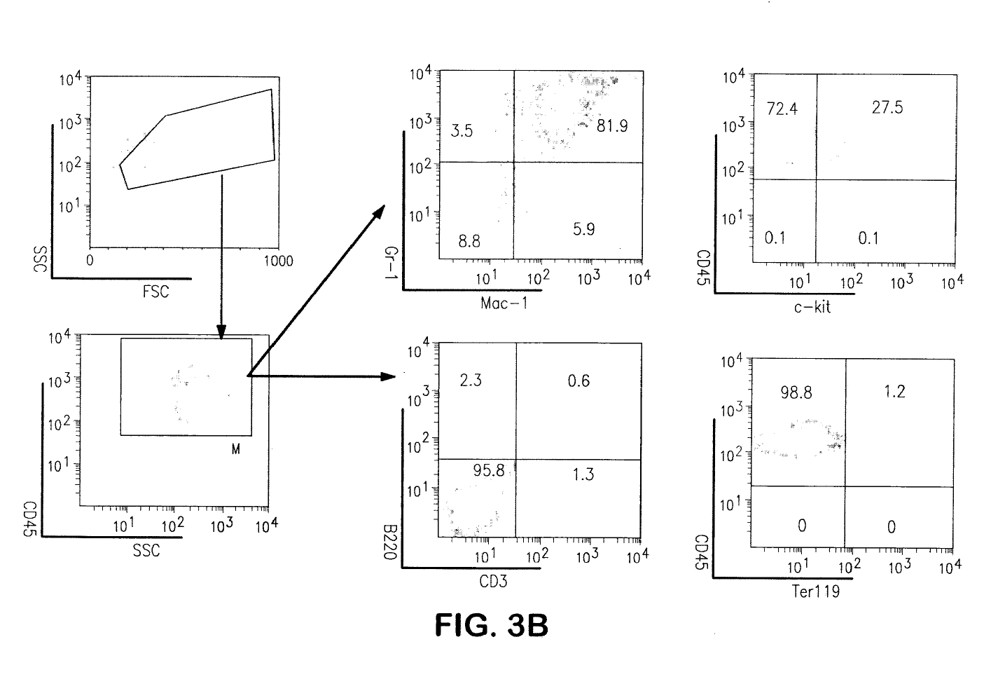

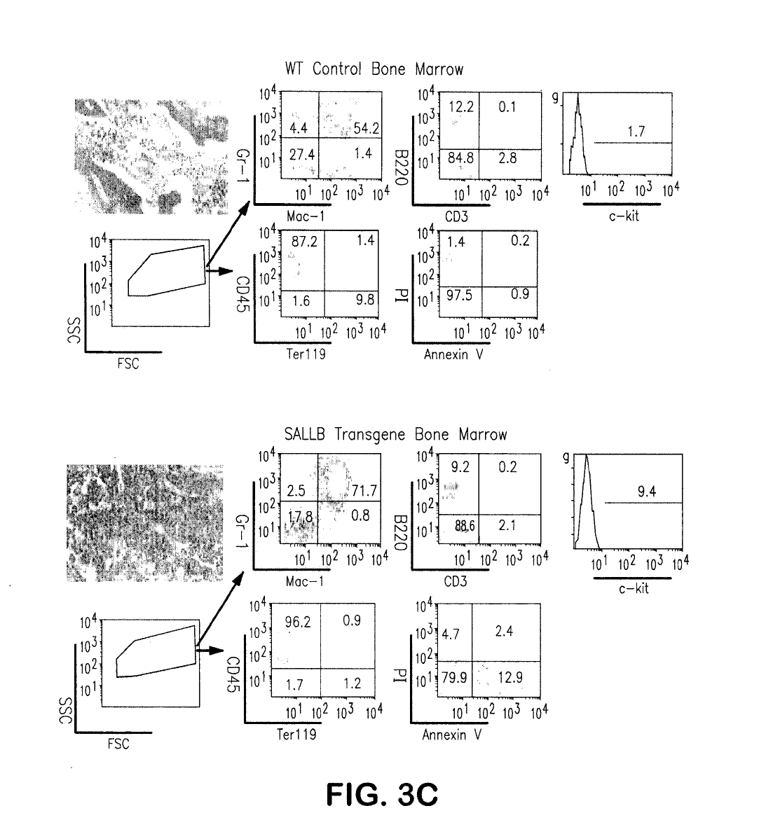

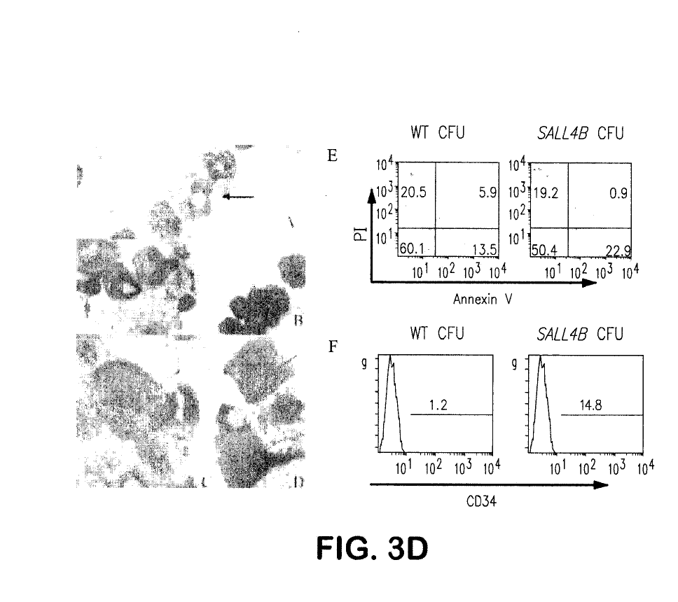

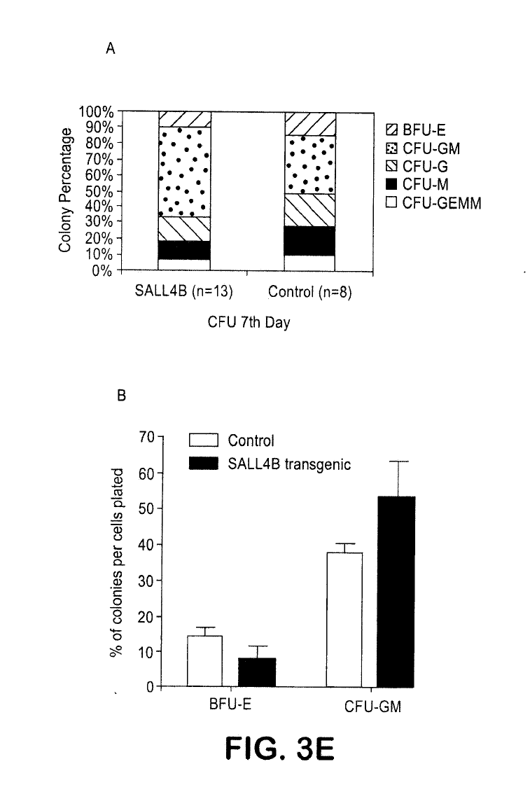

To directly test whether constitutive expression of SALL4 is sufficient to induce AML, a SALL4 transgenic mouse model was generated. The CMV promoter was fused to cDNA that encoded the 617 amino acids of human SALL4B (FIG. 3a-A), which was chosen because it was expressed in every tissue previously examined (FIG. 1b-B). The CMV promoter was previously used to ectopically express human genes in most murine organs. RT-PCR amplification was performed to examine the overexpression of wildtype (WT), full-length SALL4B in the transgenic mice.

A SALL4B transcript was detected in a variety of tissues from the transgenic mice, including brain, kidney, liver, spleen, peripheral blood, lymph nodes, and bone marrow (FIG. 3a-B). Abnormal gaits and associated hydrocephalus 3 weeks after birth were observed in 20% of the transgenic mice from multiple lines; 60% had polycystic kidneys. These findings suggest that SALL4B plays an important role in neural and renal development.

### MDS-Like Symptoms and AML in SALL4B Transgenic Mice

Monitoring of hematological abnormalities in a cohort of 14 transgenic mice from all six lines revealed that all mice had apparent MDS-like features at ages 68 months. Increased number of immature blasts and many atypical and dysplastic white cells, including hypersegmented neutrophils and pseudo-Pelger-Huet-like cells, were seen on peripheral blood smears (FIG. 3b). Nucleate red blood cells and giant platelets were also present, as well as erythroid and megakaryocyte dysplastic features, such as binucleate erythroid precursors and hypolobulated megakaryocytes.

Six (43%) of these 14 mice eventually progressed to acute leukemia (Table 1).

TABLE 1
Summary of MDS-Like/AML in SALL4B Transgenic Mice

| Mouse | | | | | |
|---|---|---|---|---|---|
| ID | Sex | Founder | Age | Phenotype | Outcome and Organs Involved by AML |
| 25 | M | 507 | 8 M | AML | Sacrificed, AML in BM, PB, Liver, Spleen, LNs |
| 509 | F | 509 | 8 M | AML | Sacrificed, AML in BM, PB, Liver, Spleen, LNs, Lungs |
| 87 | F | 504 | 8 M | AML | Sacrificed, AML in BM, PB, Liver, Spleen, LNs |
| 504 | M | 504 | 19 M | MDS-like | Sacrificed due to MDS |
| 506 | M | 506 | 19 M | MDS-like | Sacrificed due to MDS |
| 507 | F | 507 | 24 M | AML | Died, AML in BM, PB, Liver, Spleen, LNs |
| 510 | F | 510 | 24 M | MDS-like | Sacrificed due to MDS |
| 464 | M | 464 | 19 M | MDS-like | Died of MDS |
| 23 | M | 507 | 22 M | MDS-like | Sacrificed due to MDS |
| 27 | M | 507 | 22 M | MDS-like | Alive |
| 86 | F | 504 | 18 M | AML | Sacrificed, AML in BM, PB, Liver, Spleen, LNs |
| 4 | M | 464 | 15 M | MDS-like | Alive |
| 3058 | F | 25 | 12 M | AML | Died, AML in BM, PB, Liver, Spleen, LNs |
| 26 | M | 507 | 14 M | MDS | Sacrificed due to MDS |

Leukemic infiltration of many organs, including lung, kidney, liver, spleen, and lymph nodes, emphasized the aggressiveness of the disease (FIG. 3c). Leukemia blast cells were considered to be myeloid in origin because they were positive for CD34, c-kit, Gr-1, Mac-1, MPO, and nonspecific esterase; they were negative for B-cell (B220 and CD19), T-cell (CD4, CD8, CD3, and CD5), megakaryocytic (CD41), and erythroid (Ter119) markers (FIG. 3d).

### SALL4B-Induced AML was Transplantable.

Aggressive fatal AML with onset at approximately 6 weeks developed in immunodeficient NOD/SCID mice after serial transplantation of SALL4B-induced AML cells by subcutaneous injection. The transplanted disease was characterized by dissemination to multiple organs, with marked splenomegaly and hepatomegaly (FIG. 3e).

### Ineffective Hematopoiesis and Excessive Apoptosis in SALL4B Transgenic Mice.

Investigation of hematological abnormalities in younger SALL4B transgenic mice (2-6 months old) revealed that their peripheral blood showed minimal myelodysplastic features but statistically significant leukopenia and neutropenia, as well as mild anemia (Table 2).

TABLE 2
CBC from SALL4B Transgenic Mice and Wild Type Control

| | WBC | Neutrophil | Lymphocyte | RBC | Hb | HCT | MCV | PLT |
|---|---|---|---|---|---|---|---|---|
| | (×10³/μL) | (×10³/μL) | (×10³/μL) | (×10⁶/μL) | (g/dL) | (%) | (fL) | (×10³/μL) |
| Transgenic | 8.38 ± 3.52 | 0.93 ± 1.06 | 6.34 ± 4.62 | 8.85 ± 2.08 | 14.26 ± 3.04 | 50.52 ± 11.82 | 57.15 ± 6.42 | 1616 ± 662 |
| (n = 20) | | | | | | | | |
| Control | 11.59 ± 5.14 | 1.51 ± 0.86 | 9.04 ± 4.06 | 10.02 ± 1.84 | 15.66 ± 2.44 | 55.75 ± 9.62 | 55.78 ± 7.54 | 1384 ± 806 |
| (n = 18) | | | | | | | | |
| P value | 0.27 | 0.048 | 0.029 | 0.015 | 0.030 | 0.038 | 0.398 | 0.196 |

To determine whether the cause of cytopenia in these transgenic mice was related to production problems, their bone marrow was studied. Bone marrow samples showed increased cellularity and an increased myeloid population (FIG. 3f), compared with those of WT controls (Gr-1/Mac-1 double-positive population in SALL4B transgenic mice: 67±16%, N=10 vs. WT: 55.3±4%, N=11; P=0.048).

As excessive apoptosis plays a central role in ineffective hematopoiesis in human MDS, apoptosis in SALL4 transgenic mice in vivo and in vitro was examined next. Increased apoptosis was observed in SALL4B transgenic mice on both primary bone marrow (Annexin V-positive, PI-negative population in transgenic mice: 4.4±2.4%, N=10 vs. WT: 1.86±1.55%, N=7; P=0.03) and day-7 CFUs (Annexin V-positive, PI-negative population in transgenic mice: 20.1±6%, N=10 vs. WT: 10.9±4%, N=7; P=0.002) (FIGS. 3f and g). These findings may account for the fact that despite an increased myeloid population in bone marrow, these transgenic mice had statistically significant low neutrophil counts in the peripheral blood, secondary to an ongoing ineffective myelopoiesis in their bone marrow. An increased population of immature cells was also noted in SALL4B transgenic mice on both primary bone marrow (c-kit-positive population in SALL4B transgenic mice: 10.2±1.3%, N=14 vs. WT: 6.5±2.5%, N=10; P=0.008) (FIG. 3f) and day-7 CFUs (CD34-positive population in SALL4B transgenic mice: 11±2.2%, N=8 vs. WT: 6.3±2.4%, N=7; P=0.002) (FIG. 3g). Similar numbers of total colonies were observed in SALL4B transgenic mice (mean=51, N=10) and WT controls (mean=40, N=6). Increased myeloid and decreased erythroid colony populations (FIG. 3h), however, were found in SALL4B transgenic mouse CFUs compared with those of WT controls, as has been reported in human MDS patients and other MDS mouse models. These observations suggest that the defect in SALL4B transgenic mice lies at the stem cell/progenitor level affecting hematopoietic differentiation.

### Binding of SALL4A and SALL4B to β-Catenin In Vitro.

The potential signaling pathway that SALL4 may affect in leukemogenesis was explored next. In Drosophila, spalt (sal) is a downstream target of Wnt signaling. ALL1, another member of the SALL gene family, can interact with β-catenin. The high affinity site for this interaction is located at the C-terminal double zinc finger domain. This region of SALL1 was found to be almost exactly identical to that of SALL4. This finding prompted the investigation of whether SALL4 was also able to bind β-12 catenin. Expression constructs of SALL4A and SALL4B tagged with hemagglutinin (HA) were generated. As shown in FIG. 4a, endogenous β-catenin was pulled down by HA-SALL4A and HA-SALL4B, but not by HA alone.

Activation of the Wnt/β-Catenin Signaling Pathway by Both SALL4A and SALL4B.

To investigate the functional effect of the interaction of the SALL4 isoforms with β-catenin, a luciferase reporter (TOPflash; Upstate USA) containing multiple copies of Wnt-responsive elements to determine the potential of SALL4A and SALL4B to activate the canonical Wnt signaling pathway was used. This reporter construct has been shown to be efficiently stimulated by Wnt1 in a variety of cell lines. TOPflash reporter plasmid was transiently transfected in the HEK-293 cell line, in which both Wnt and its Wnt/β-catenin signal pathways were present. TOPflash reporter plasmid was also cotransfected with SALL4A or SALL4B. Significant activation of the Wnt/β-catenin signaling pathway by both SALL4A and SALL4B was indicated by increased luciferase activity (FIG. 4b).

### Similar Expression Patterns of β-Catenin and Sall4 at Different Phases of CML.

Dysregulated Wnt/β-catenin signaling is known to be involved in the development of LSCs. The best evidence for β-catenin's involvement in LSC self-renewal comes from the study of CML blast transformation. It has been demonstrated that Wnt signaling was activated in the blast phase of CML but not the chronic phase, where it was concluded that dysregulated Wnt signaling, such as activation of β-catenin, could confer the property of self-renewal on the GMPs of CML and lead to their blastic transformation.

Given the potential interaction between SALL4 and β-catenin and spalt's position as a downstream target of Wnt signaling in Drosophila, SALL4 protein expression in CMLs in different phases was examined. SALL4 expression was present in blast-phase CML (N=12, 75%) but not the chronic phase (N=11,100%) (FIG. 4c). In the accelerated phase (N=6, 10%), in which blast counts are increased, immature blasts expressing SALL4 were observed upon a background of nonstaining mature myeloid cells, such as neutrophils.

### Effect of SALL4 on OCT4 Promoter.

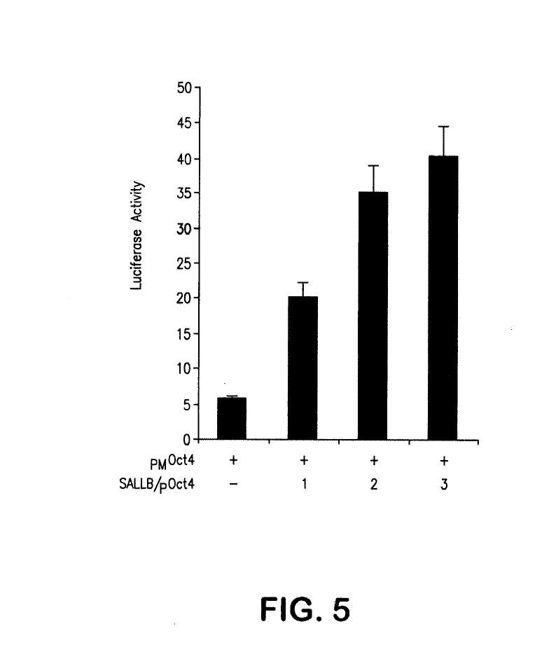

To identify the effect of SALL4 on OCT4, cells, OCT4-Luc constructs were co-transfected with renilla plasmids and increasing concentrations of SALL4B (FIG. 5). As the figure shows increasing SALL4B increased OCT4 promoter activity by more than 8 fold.

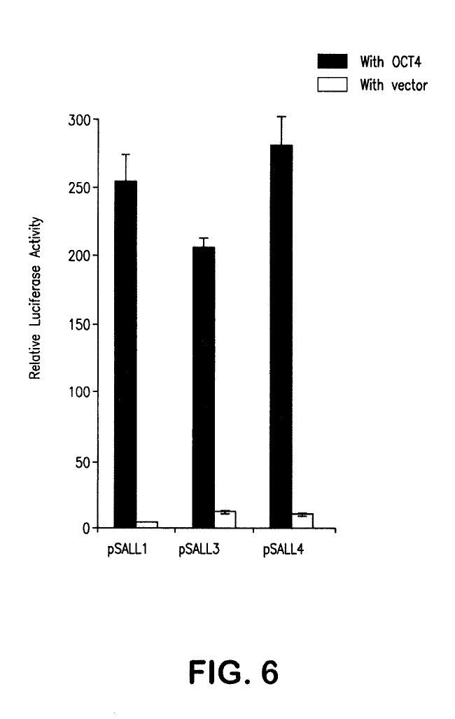

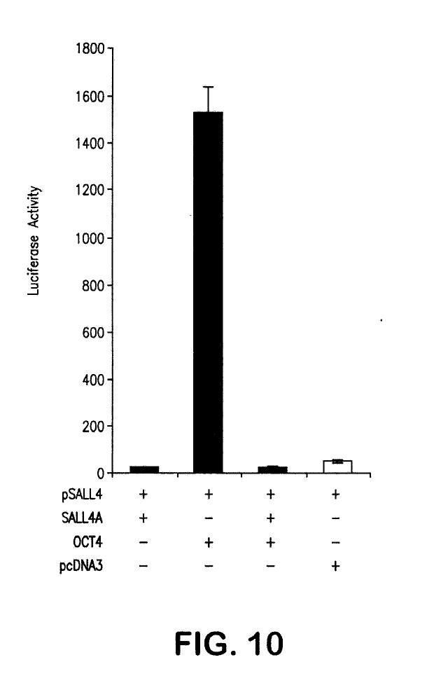

To determine if OCT4 stimulates the activity of SALL gene member promoters, promoter constructs (pSALL1, pSALL3, and pSALL4) were co-transfected with OCT4 in HEK-293 cells. As can be seen from the data (FIG. 6), after 24 hr post-transfection, the overexpression of OCT4 strikingly stimulated the promoter activities of SALL gene members SALL1, SALL3, and SALL4 when compared with that of the pcDNA3 vector control. Also, this activation was totally blocked by the presence of a small amount of excess SALL4 (FIG. 10).

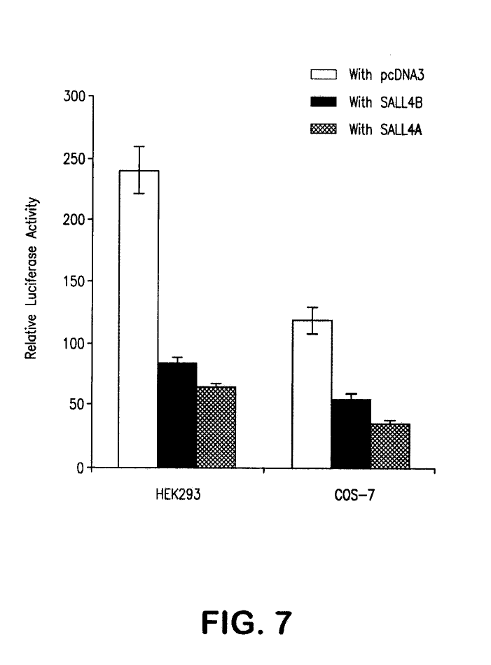

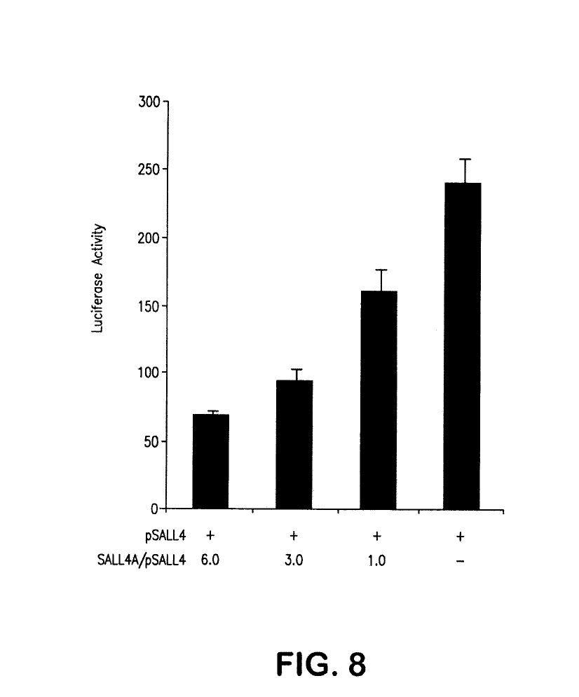

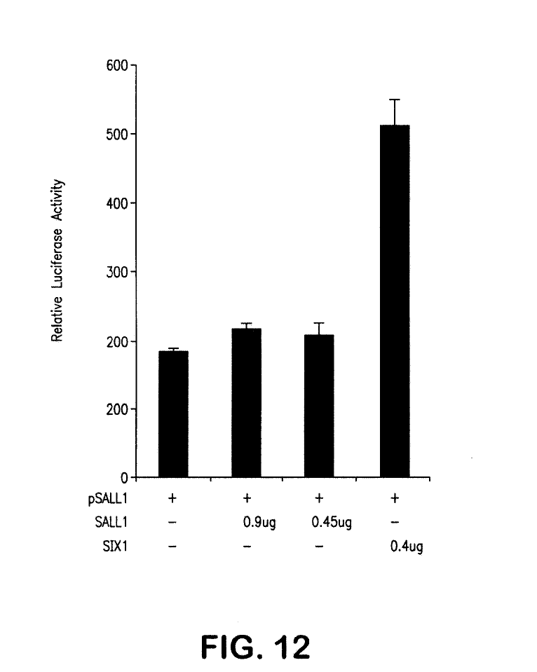

To determine whether there was any self regulation of SALL promoters by SALL family member proteins, SALL4-Luc was co-transfected with renilla reporter and either SALL4A or SALL4B expression plasmids is HEK-293 and COS-7 cells (FIG. 7). As shown in the figure, SALL4 (both A and β isoforms) suppresses its own promoter activity in different cell lines. Further, this self-suppression is dose dependent (see, FIG. 8). When the ratio of SALL4A with SALL4 promoter reached 6:1, the promoter activity dropped approximately 3.5 fold compared with the basal level. This data indicates that SALL4 bears a self-suppression function. This is not true for all SALL members, for example, SALL1 fails to demonstrate self-suppression of its promoter (FIG. 12).

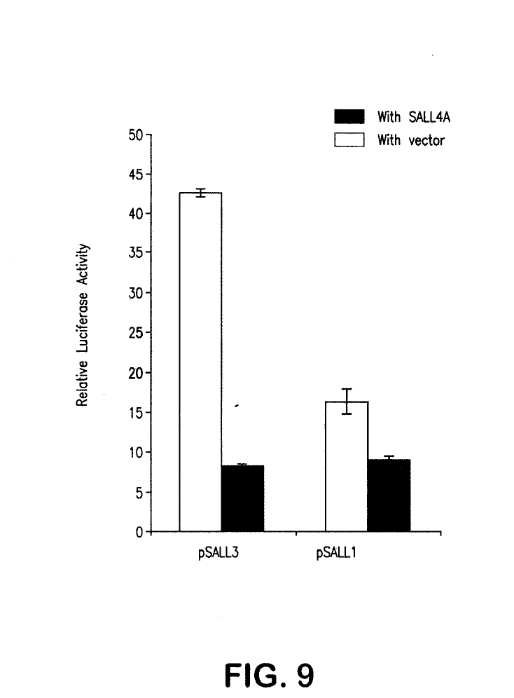

Data also indicates that SALL1 and SALL3 promoters were strikingly activated by exogenously added SALL4 (See, FIG. 9), indicating that SALL4 is able to regulate other members of the SALL gene family involving embryonic stem cell function.

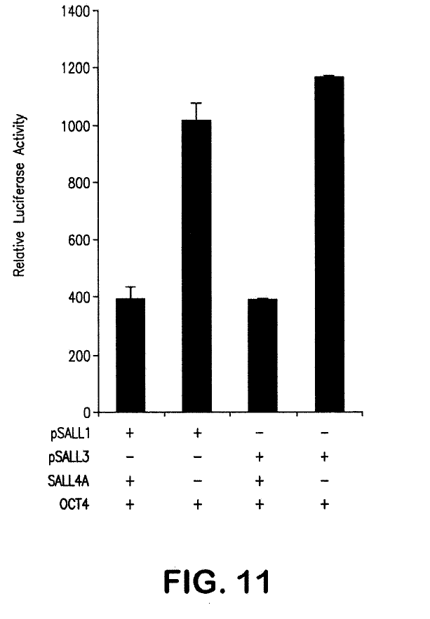

Since the stimulation of OCT4 on SALL4 promoter can be totally blocked by SALL4 (FIG. 10), SALL4 was examined to determine if it represses the activation of OCT4 on other SALL member promoters. As can be seen in FIG. 11, SALL4 also blocked OCT4 activation of other SALL member promoters.

### SALL4 in Adult Stem Cells and Embryonic Carcinoma.

The characterization of tissue stem cell populations remains difficult because of the lack of markers that can distinguish between stem cells and their differentiating progeny. For many tissues, panels of molecular markers have been developed to define the stem cell compartment.

The present data shows that SALL4 is a key regulator of embryonic stem cells in pluripotency and self-renewal. For example, embryonic carcinomas display the phenotype of early embryonic stem cells and possess pluripotent potential. Therefore, the expression of SALL4 protein in this type of tumors by immunohistochemistry was examined. Immunohistochemical data conclusively indicated that all tumor cells of embryonic carcinomas showed a nuclear staining, whereas all non-tumor cells were negative. These observations suggest that SALL4 can be used as a specific marker for normal and malignant embryonic germ cells and embryonic stem cells.

Given that SALL4 was expressed in very early embryonic stem cells, and embryonic carcinoma is reported to arise from transformation of these cells, immunohistochemistry also shows that a) SALL4 positive cells in normal breast lobules, accounted for less than 2% of the epithelium and b) in breast carcinoma samples, SALL4 protein expression in clusters of cells or scattered cells was observed. Further, SALL4 protein was expressed in the nucleus of normal breast epithelial cells and breast carcinoma cells. Moreover, this pluripotent gene expression was observed in other normal adult tissues such as prostate and lung, and carcinoma arising from these tissues with SALL4 antibody. The presence of a small number of SALL4-expressing cells in the broncho-epithelium and prostatic acini, and their stromal cells was observed, as well as the finding that SALL4 was expressed at a similar frequency in normal prostate and lung to that in lobular epithelial cells of breast. In addition, scattered tumor cells in the prostate carcinoma expressed SALL4 protein by immunohistochemistry studies with a SALL4 antibody.

The above examples reveal that (1) immunostaining with anti-SALL4 antibodies are useful diagnostic tools in the identification of embryonic carcinomas, (2) expression of SALL4 is found in several human stem cells and cancer cells; (3) identification of SALL4-expressing cells in human tissues can be used to identify the stem cells, their pre-malignant clones, and malignant cells, and (4) SALL4 represents an ideal marker for embryonic stem cells, adult stem cells and cancer stem cells.

### Example 2

### SALL4 is a Major Master Regulator in ES Cells

Growing evidence has shown that Sall4 plays a vital role in governing ES cell fate decisions. SALL4 is expressed early in embryonic development and exhibits a similar expression pattern to that of Oct4. SALL4-null ES cells exhibited significantly reduced proliferation and microinjection of SALL4 small interfering RNA into mouse zygotes resulted in reduction of SALL4 and Oct4 mRNAs prior to implantation. These findings prompt the investigation into global downstream targets of SALL4 in embryonic cells. Using a ChIP-chip assay, a genome scale mapping of SALL4 binding genes was carried out in the murine embryonic stem cell line W4. Using the RefSeq promoter tiling array provided by NimbleGen Systems Inc, a 2.7 kb region (2 kb upstream and 500 bp downstream from the transcription start site) of each promoter region was probed. Hybridizations to these arrays with SALL4 chromatin-immunoprecipitated DNA from W4 cells revealed a massive gene binding, with a total binding of 5,256 genes. Analysis of these Sall4 binding genes based on the PANTHER classification system showed that about 73% of the classified genes are involved in either proliferation and self-renewal or differentiation and development.

Based on recently published data, the stem cell gene binding pattern by SALL4 was compared with that of the gatekeeper genes Oct4 and Nanog.

Data derived from a similar Chip-PET assay shows that Oct4 binds only 1083 genes and Nanog binds 3006 genes. These binding numbers are strikingly less than that of Sall4, even though CHIP-PET method has a higher probe resolution.

During development, both SALL4 and Oct4 are expressed in the very early stage of the embryonic development. SALL4 expression is already seen in the 2-cell stage with Oct4, while Nanog is expressed once development reaches the blastocyst stage. The earlier expression and extensive gene binding may suggest that SALL4 exert an even larger and more massive role in regulating ES cell features.

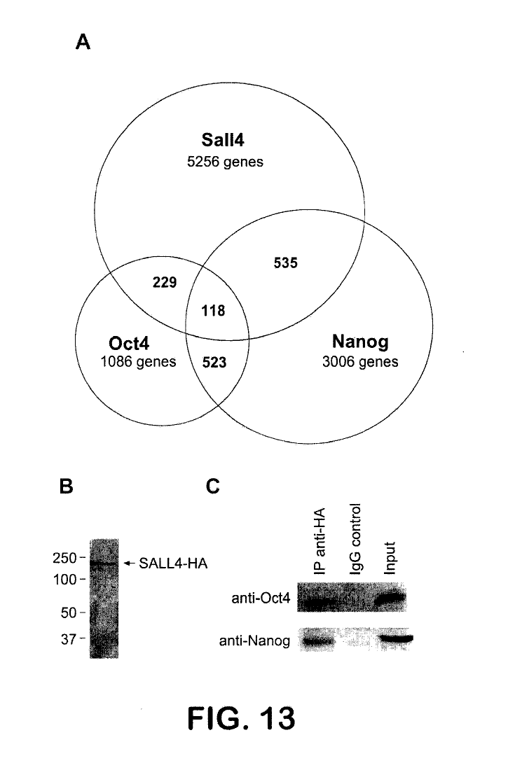

Next, determination of the distribution of the Oct4, Nanog, and SALL4 binding genes in ES cells was sought. Comparison of the three gene groups show that SALL4 binds a total of 229 genes which are also targets of Oct4, we will refer to these genes as co-bound or co-occupied. This represents 21% of all Oct4 bound genes. Similarly, SALL4 co-binds to 535 Nanog target genes, representing 18% of Nanog's total binding sites (FIG. 13). There are a total of 118 genes that are co-occupied by all of Oct4, SALL4 and Nanog. PANTHER classification shows that 79% of these co-occupied genes belong to either self-renewal/proliferation or developmental/differentiation processes. These findings raise a possibility that many pluripotency maintenance genes may be coordinated by a complex network consisting at least of Oct4, Sall4 and Nanog.

Interaction of SALL4 with Oct4 and Nanog in ES Cells

Given the similar gene promoter co-occupancies and gene expression patterns between Sall4-Oct4 and SALL4-Nanog, it was thought that an Oct4-SALL4-Nanog complex exists. For this purpose, an immunocoprecipitation experiment was performed on Sall4 and Oct4 using a transiently SALL4-HA transfected ES cell extract. As seen in the western blot result (FIGS. 13b and 13c), over-expression of SALL4-HA fusion protein was detected by both anti-HA and anti-SALL4 antibodies (the latter not shown). In the HA antibody treated cell lysate, a unique ˜45 kd band was successfully detected; its size matches the endogenous Oct4 control. By contrast, an IgG negative control failed to generate Oct4 band in the same extract, indicating a direct Sall4-Oct4 interaction (FIGS. 13b and 13c). Using the same method, the Sall4/Nanog interaction was also confirmed in the same anti HA-pulldown cell lysate (FIGS. 13b and 13c). Based on these results, it is not surprising that Oct4, SALL4, Nanog, and possibly others, form a complex which contributes to regulation of ESC features through internal interactions. This is strengthened due to the significant co-occupancies among Sall4, Oct4 and Nanog target genes. Further studies are still required to extend the knowledge of the Oct4-SALL4-Nanog complex.

### Genes Related to Differentiation and Pluripotency

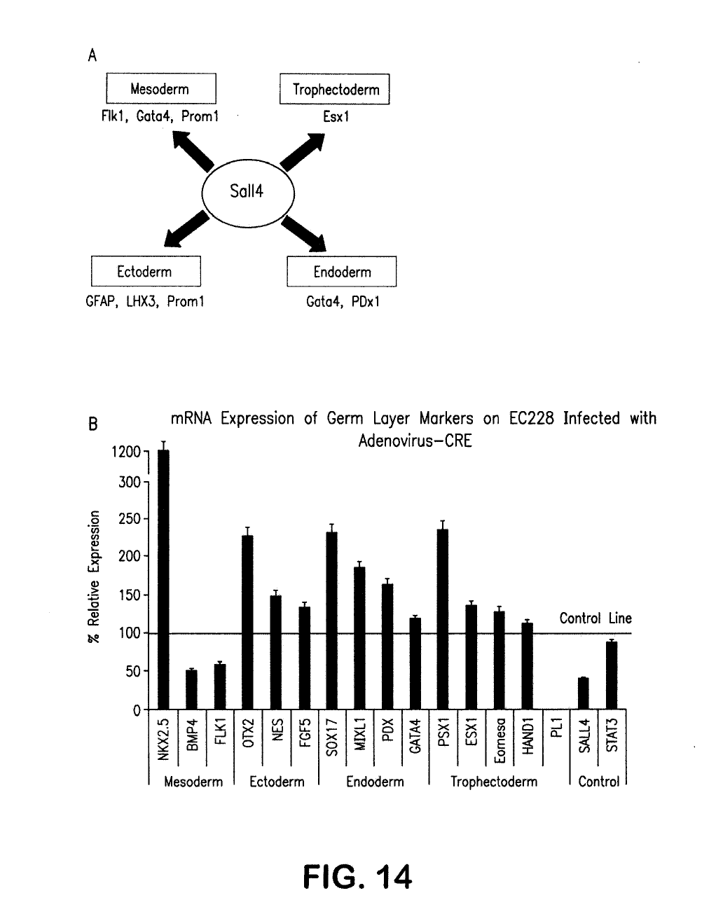

Based on this data, it seemed that SALL4 represses genes leading to differentiation and activates genes that are necessary for pluripotency. For this, 217 of the SALL4 bound genes identified as necessary for cell differentiation were analyzed, some of which are specifically expressed in different developmental lineages. As seen in FIG. 14, SALL4 binds with multiple markers from all of the lineages including ectoderm, endoderm, mesoderm and trophectoderm, suggesting a direct involvement in regulating cell differentiation and pluripotency. Using our conditional Sall4 knockout ES cell lines, we were able to verify changes of these marker expression levels after endogenous SALL4 knockdown. The W4 clone EC 228, in which one copy SALL4 allele was floxed, was treated with Cre expressing adenovirus for 9 hours and gene expression was evaluated by qPCR. For differentiation analysis, we chose 4 candidate markers for each cell lineage.

Data from three separate experiments show that Sall4 expression levels were consistently shutdown up to about 50%, confirming that EC228 is a successful and stable gene targeting system. Interestingly, the tested markers for ectoderm, endoderm, and trophectoderm were all suppressed by SALL4, while two of the three mesoderm markers are activated. In other words, it indicates that SALL4 has a role in suppressing ectoderm, endoderm, and trophectoderm differentiation, while activating differentiation into mesoderm lineages (FIG. 14b).

We also evaluated SALL4's binding to genes known to maintain pluripotency. We identified only 15 pluripotency genes (Assou et al, Stem Cells) that are common to SALL4 target genes suggesting that SALL4 has little role in maintaining pluripotency but rather, functions to inhibit differentiation.

### ES Cell Pluripotency and Proliferation are Dependent on SALL4 Expression

As described previously, embryonic endoderm ES cells can not be established from SALL4 deficient blastocyts. The W4-EC228 clone was cultured in feeder free T25 flasks and treated with Ade-Cre. Morphology changes were observed within 9 hours of treatment. Alkaline Phosphatase staining of ESCs was demonstrated. Analysis of layer markers was done by qPCR.

### Sall4 Binds to Target Genes of PRC1 and PRC2

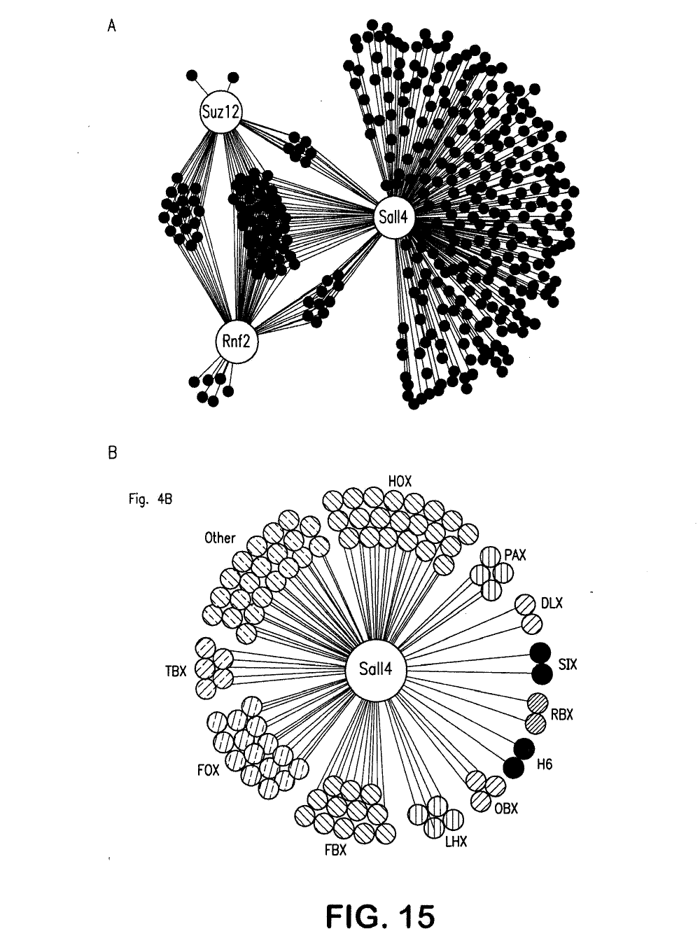

The term Polycomb-Repressive Complexes (PRCs) has been recently reported and consists of two distinct groups. PRC1 consists of >10 subunits including Bmi1, Rnf2, PhcI and the HPC proteins while the PRC2 contains Ezh2, Eed, Suz12 and RbAp48. PRCs maintain ES cell pluripotency through epigenic events such as methylation of lysine 27 on histone 3 (H3K27), thus suppressing differentiation related activators. To better understand how SALL binding genes are related to PRCs, the genome binding patterns by SALL4 were compared with those of polycomb genes which have been published previously. It is known in the art that 4 genes, Rnf2, Phc1 (from PRC1), Suz12, and Eed (from PRC2), co-occupied 512 common genes in murine ES cells, many of which encode transcription factors with important roles in development. Direct comparisons with these data show that 28.3% (360/1271) of Suz12 target genes and 27.8% (339/1219) of Rnf2 targets were co-bound by SALL4. Analysis of these two groups of common genes shows that over 75% of them are involved in proliferation/self-renewal or differentiation/development. This indicates PRC1, PRC2 and Sall4 are co-binding a large block of ESC feature governing genes (FIG. 15a).

The transcription factors bound by two PRC genes (Suz12, Rnf2) were selected and compared with those bound by SALL4. Suz12 binds to unique transcription factors Lrch4 and Lhmx2, however, it shares many overlapping sites with either SALL4 or Rnf2. The same can be said for Rnf2 (FIG. 15b). Genes bound by Rnf2, Suz12, and Sall4 include multiple homeobox genes, Zic1, Gata4, and Lef1. SALL4 is exceptional because it binds to 339 transcription factors many of which are involved in development. In fact, we found SALL4 binds to a large group of homeobox genes and other developmentally important genes, including HOX, FOX, F-Box, and T-box family members independently of polycomb binding (FIG. 15b and Table 4).

TABLE 4
Key developmental genes bound by Sall4
Hox Genes

| | |
|---|---|
| homeo box A1 | Hoxa1 |
| homeo box A11 | Hoxa11 |
| homeo box A3 | Hoxa3 |
| homeo box A4 | Hoxa4 |
| homeo box A5 | Hoxa5 |
| homeo box A7 | Hoxa7 |
| homeo box A9 | Hoxa9 |
| homeo box B2 | Hoxb2 |
| homeo box B5 | Hoxb5 |
| homeo box B6 | Hoxb6 |
| homeo box B7 | Hoxb7 |
| homeo box B8 | Hoxb8 |
| homeo box C10 | Hoxc10 |
| homeo box C11 | Hoxc11 |
| homeo box C4 | Hoxc4 |
| homeo box C6 | Hoxc6 |
| homeo box C9 | Hoxc9 |
| homeo box D10 | Hoxd10 |
| homeo box D12 | Hoxd12 |
| homeo box D3 | Hoxd3 |
| homeo box D4 | Hoxd4 |

Paired Domain

| | |
|---|---|
| paired box gene 3 | Pax3 |
| paired box gene 2 | Pax2 |
| paired box gene 9 | Pax9 |
| paired box gene 1 | Pax1 |

Lim Domain

| | |
|---|---|
| LIM homeobox protein 2 | Lhx2 |
| LIM homeobox protein 3 | Lhx3 |
| LIM homeobox protein 8 | Lhx8 |
| LIM homeobox protein 9 | Lhx9 |

Six/sine homeobox

| | |
|---|---|
| sine oculis-related homeobox 2 homolog | Six2 |
| (Drosophila) | |
| sine oculis-related homeobox 3 homolog | Six3 |
| (Drosophila) | |

Dlx family

| | |
|---|---|
| distal-less homeobox 1 | Dlx1 |
| distal-less homeobox 5 | Dlx5 |

Fork head box

| | |
|---|---|
| forkhead box A2 | Foxa2 |
| forkhead box B1 | Foxb1 |
| forkhead box C1 | Foxc1 |
| forkhead box D3 | Foxd3 |
| forkhead box D4 | Foxd4 |
| forkhead box F2 | Foxf2 |
| forkhead box G1 | Foxg1 |
| forkhead box H1 | Foxh1 |
| forkhead box I1 | Foxi1 |
| forkhead box J2 | Foxj2 |
| forkhead box N4 | Foxn4 |
| forkhead box O1 | Foxo1 |
| forkhead box P2 | Foxp2 |
| forkhead box P3 | Foxp3 |
| similar to forkhead box R2 | LOC436240 |

T-box family

| | |
|---|---|
| T-box 19 | Tbx19 |
| T-box 18 | Tbx18 |
| T-box 15 | Tbx15 |
| T-box 21 | Tbx21 |
| T-box 22 | Tbx22 |

Oocyte Homeobox Family

| | |
|---|---|
| oocyte specific homeobox 1 | Obox1 |
| oocyte specific homeobox 3 | Obox3 |
| oocyte specific homeobox 6 | Obox6 |

F-Box family

| | |
|---|---|
| F-box and leucine-rich repeat protein 10 | Fbxl10 |
| F-box and leucine-rich repeat protein 13 | Fbxl13 |
| F-box and leucine-rich repeat protein 18 | Fbxl18 |
| F-box and leucine-rich repeat protein 21 | Fbxl21 |
| F-box and WD-40 domain protein 10 | Fbxw10 |
| F-box and WD-40 domain protein 12 | Fbxw12 |
| F-box and WD-40 domain protein 14 | Fbxw14 |
| F-box and WD-40 domain protein 9 | Fbxw9 |
| F-box only protein 36 | Fbxo36 |
| f-box only protein 9 | Fbxo9 |
| F-box protein 28 | Fbxo28 |
| F-box protein 42 | Fbxo42 |

Paired-like domain

| | |
|---|---|
| paired-like homeobox 2b | Phox2b |
| paired related homeobox 2 | Prrx2 |

Other homeobox genes

| | |
|---|---|
| homeo box, msh-like 2 | Msx2 |
| even skipped homeotic gene 2 homolog | Evx2 |
| aristaless related homeobox gene (Drosophila) | Arx |
| brain specific homeobox | Bsx |
| caudal type homeo box 4 | Cdx4 |
| developing brain homeobox 1 | Dbx1 |
| diencephalon/mesencephalon homeobox 1 | Dmbx1 |
| extraembryonic, spermatogenesis, homeobox 1 | Esx1 |
| genomic screened homeo box 2 | Gsh2 |
| H2.0-like homeo box 1 (Drosophila) | Hlx1 |
| homeobox containing 1 | Hmbox1 |
| H6 homeo box 1 | Hmx1 |
| H6 homeo box 2 | Hmx2 |
| homeobox only domain | Hod |
| Iroquois related homeobox 6 (Drosophila) | Irx6 |
| ladybird homeobox homolog 2 (Drosophila) | Lbx2 |
| mesenchyme homeobox 2 | Meox2 |
| Unc4.1 homeobox (C. elegans) | Uncx4.1 |
| ventral anterior homeobox containing gene 2 | Vax2 |
| zinc finger homeobox 1b | Zfhx1b |
| reproductive homeobox 4B | Rhox4b |
| reproductive homeobox 7 | Rhox7 |
| Pbx/knotted 1 homeobox 2 | Pknox2 |
| prospero-related homeobox 1 | Prox1 |

### K4 K27 Bivalent Domains are Bound by SALL4

Recently it has been reported that the existence of bivalent domains regulate pluripotency through a balance of H3K4 gene activation and H3K27 gene repression. By comparing our data with previously published bivalent domains we show that Sall4 binds to over 40% (54/122) of non-duplicate bivalent domains identified in the study. Interestingly, SALL4 only binds to three K27 bound genes, and 27 K4 genes aside from the genes covered by bivalent domains. This indicates that Sall4 may control a select region of developmentally important genes through a balance of activation and repression methylations. However, it appears as though SALL4 plays a larger role in the activation of certain genes.

When genes are associated with bivalent domains, they have been shown to have low expression levels due to the methylation at K27 having a more pronounced effect on expression than the activating K4 methylation. Thus, we would expect the 54 genes identified in this study to have low expression levels in SALL4 expressing cells, but cannot predict the effects of SALL4 shutdown.

SALL4 Targets Important Signals that Control ES Differentiation and Lineage Specification

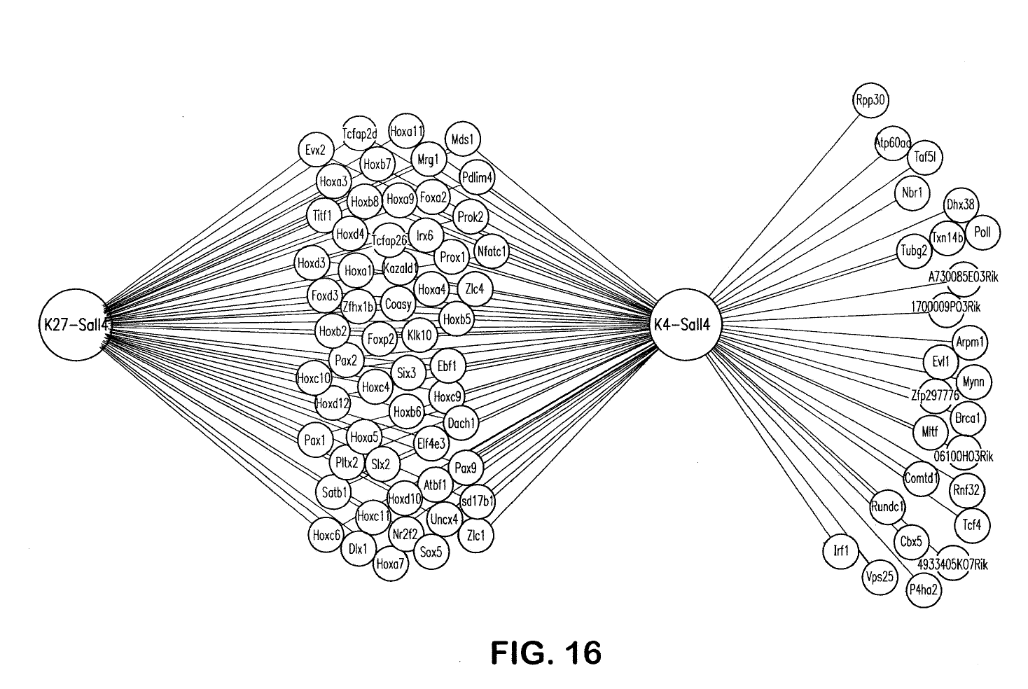

Key signaling pathways that play important roles in maintaining pluripotency during embryogenesis include the STAT3, Notch, Nodal, TGF beta and Wnt signaling pathways. In fact, SALL4 is binding to genes that are involved in each of these pathways (FIG. 16). The Wnt signaling pathway has important roles in embryogenesis and cancer, while the STAT3 pathway is the key signal required for murine ESC self-renewal following LIF binding to the LIF/gp130 receptor complex. Bone morphogenetic protein (BMP, TGF beta) signaling plays important roles in diverse embryonic events including induction of mesoderm, hematopoiesis and epidermis formation. The Nodal pathway belongs to the TGF-β superfamily, is largely restricted to stem cells and sustains pluripotent cells in the mouse epiblast before axial pattering. Notch signaling pathway affects a diverse range of development processes controlling cell differentiation, proliferation, morphogenesis and organ formation. Since more than 85 SALL4 binding genes are involved in Wnt pathway or as downstream targets, we will use this pathway as an example for further analysis (see below).

### Comparison of ChIP-Chip and Gene Expression

Based on a genome-wide expression profile, Kim et al (Nature 2005) classified the enriched binding genes into four categories to elucidate the expression of the target genes. A similar strategy combined with endogenous Sall4 knockdown was used here to confirm which SALL4 binding genes are indeed regulated by SALL4 levels. For this purpose, our conditional knock out W4-EC228 clone was used for expression microarray. Quantitative PCR validation indicates that Cre-induced SALL4 knockdown is much more efficient and consistent when compared to RNAi or other conventional knock outs that we tested. Comparison of expression profile after Sall4 knockdown shows that 46% of the binding genes have a dramatic change in the expression level.

Expression profile showed little change of expression for the pluripotency genes, only two bound genes were upregulated when SALL4 is knockdown. suggesting that SALL4 may have little role in maintaining pluripotency but rather, functions to inhibit differentiation. This supports the case for SALL4 has a differentiation repressor but indicates that Sall4 has little effect on pluripotency.

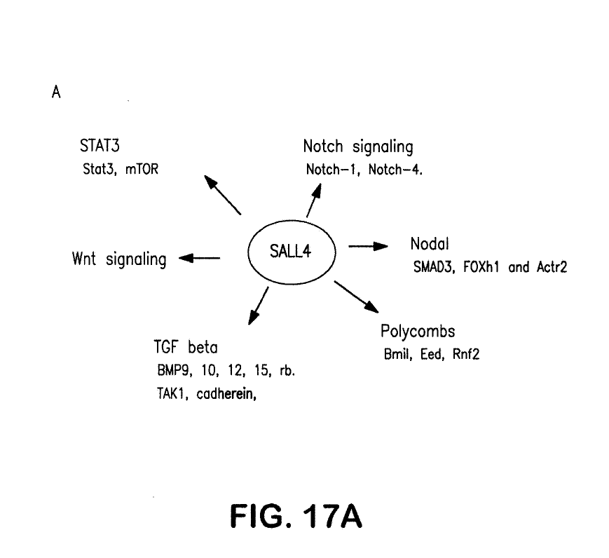

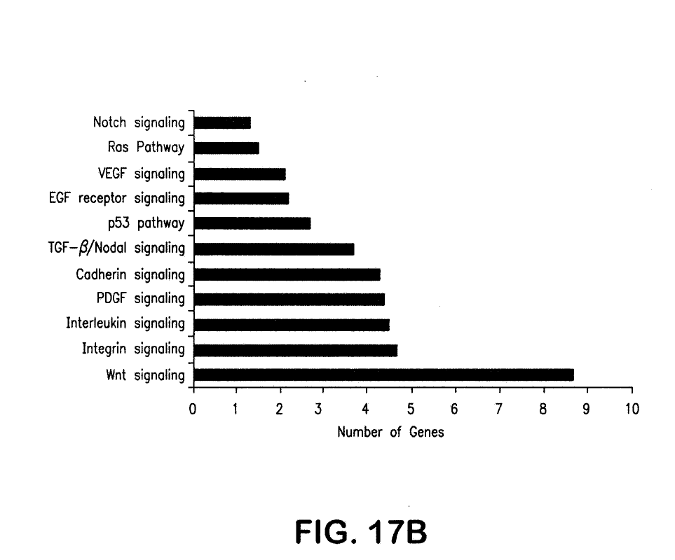

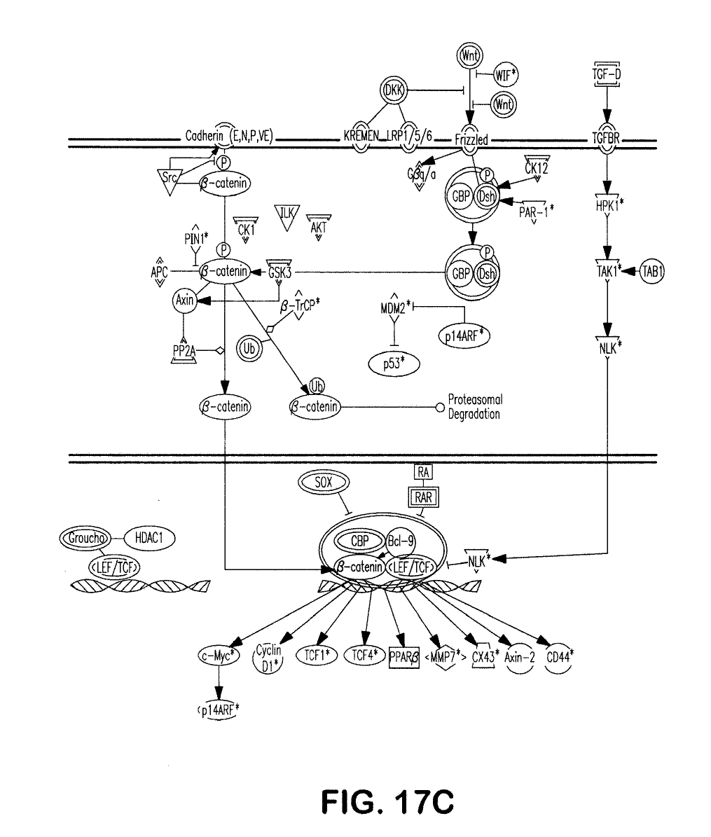

To elicit the effects that SALL4 may have on the canonical Wnt signaling pathway, we compared gene expression values in EC228 cells after Cre treatment. Interestingly, expression profiling shows that SALL4 shutdown has an effect on nearly all of the members of the pathway (FIG. 17). Down-regulation of SALL4 results in higher levels of β-catenin and in combination with other proteins changes transcriptional regulation. Of note, we show that SALL4 does not directly bind all the down-regulated genes in the pathways. In each pathway, SALL4 binds to select genes and regulates others through intermediate mechanisms.

Our data, when presented together with others data, outline a system in which SALL4: (1) has a similar expression pattern to Oct4 and is expressed earlier in development than Nanog, (2) binds to the promoter regions of more genes than either Oct4 or Nanog, (3) causes differentiation when deficiencies exist, (4) binds more bivalent domains than Oct4 and Nanog, and (5) is a lethal knockout, like Oct4 and Nanog. This suggests that SALL4 plays a central role in maintaining the pluripotency of ESCs.

We have shown that SALL4 binds over 5,000 promoter regions within the murine ESCs. An analogous ChIP-PET assay was done on murine ESCs to test promoter regions that Oct4 and Nanog bind to and results from this assay show that Oct4 binds to about 1,000 gene promoters and Nanog binds about 3,000. It is interesting that SALL4 binds nearly 2,000 more genes than Nanog and is expressed earlier in development. Because promoter binding does not indicate expression of a gene, this may or may not be significant. For our data, we can say that SALL4 binds to 5256 promoter regions and causes significant transcript level changes in X % of these genes.

Sall4 knockdown cells spontaneously differentiate. Previous studies have stated that this differentiation is into trophectoderm lineages. Here, it appears as though knockdown results in differentiation into endoderm, ectoderm, and trophectoderm lineages based on real-time PCR. These findings may differ due to different methods of transfection. In our experiment expression levels were measured right after endogenous SALL4 shutdown. This is in contrast to previously published data that use stable transfection and allow other genes to compensate for Sall4 shutdown.

Bivalent domains have recently been reported to play an integral role in cell differentiation and pluripotency through epigenic regulation. These domains consist of large regions of H3K27 methylation sites harboring smaller H3K4 methylation sites, which are often centered over developmentally important genes. Interestingly, SALL4 binds to about 40% of the bivalent domains reported. In contrast, Oct4 binds 10% and Nanog binds 20%. The roles of these proteins in regulation of bivalent domains is unknown, but it can be hypothesized that Sall4, or another regulatory gene, plays a role in the balance of activation and repression through epigenic events at these bivalent domains. Bernstein et al originally reported that Oct4, Nanog, and Sox2 bind to nearly 50% of the bivalent domains that they reported, however, this information was based on humans ESCs. Thus, our comparison in murine ESCs has varied slightly.

Polycomb group proteins occupy genes that are repressed in ESCs. They have been shown to co-occupy a significant portion of these genes with Oct4 and Nanog. Here we show that they also co-occupy a large portion of them with SALL4. Interestingly, SALL4 binding does not show preference over PRC1 or PRC2 as it binds about 30% of total genes from each group. Intuitively this makes sense however, because Sall4 is largely binding to developmental/self-renewal processes. By comparing the transcription factor bound by each Suz12, Rnf2, and SALL4 we are able to identify genes that may be regulated by SALL4. These included a large group of homeobox genes, as well as developmental genes Zic1, Gata4, and Lef1.

These findings have brought many interesting questions to the forefront. The binding of Sall4 to the recently reported bivalent domains is extremely interesting and will be the subject of further study. Similarly, many questions remain to be answered regarding evidence for an Oct4-SALL4-Nanog complex regulating gene expression and ESC pluripotency. This provides one mechanism by which the expression levels of the complex target genes are stably maintained.

### Example 3

### SALL4 in ES Cells and LSCs

SALL4 may be one of few genes that creates a connection between LSCs and the self-renewal properties of normal HSCs and ES cells. Interestingly, SALL4 protein expression is always correlated with the presence of stem and progenitor cell populations in various organ systems including bone marrow.

Constitutive Expression of SALL4 Protein in Primary Human AML and SALL4 Expression in MDS is Associated with High-Grade Morphology

Amplification of the SALL4 gene, as demonstrated by digital karyotyping or analysis through quantitative polymerase chain reaction (Q-PCR), is seen in approximately 75 percent of human AML cases. To determine if the observed aberrant SALL4 expression is also present at the protein level, 81 AML samples ranging from AML subtypes M1 to M5 (FAB classification) were examined. All 81 AML samples have shown aberrant SALL4 expression, which was consistent with the SALL4 mRNA expression levels as demonstrated by real-time polymerase chain reaction (RT-PCR) amplification. In normal hematopoiesis, SALL4 was present in the CD34⁺ HSCs/HPCs and down-regulated in mature granulocytes and lymphocytes. As a result, constitutive expression of SALL4 in leukemia may have prevented the leukemic blasts from differentiating and/or gaining self-renewal properties.

The expression of the SALL4 protein in human samples containing differing grades of MDS was also examined using immunohistochemistry with an affinity-purified SALL4 antibody. Using a cut-off of >5 percent SALL4 positive cells, all low-grade MDS groups (RA, refractory anemia, and RARS, refractory anemia with ringed sideroblasts) were negative for SALL4. SALL4 positivity-defined as more than 5 percent of immunolabeled cells—was detected in 10 of 11 high-grade MDS groups. The high-grade MDS groups were further contrasted with respect to the percentage of SALL4 positive cells. RAEB-2 (refractory anemia with excess blasts-2) and AML transformation showed a relatively high percentage (>10 percent). The highest percentage of SALL4 positive cells was seen in AML transformation (>20 percent). This indicates that the high percentage of SALL4-expressing cells correlates with a high-grade morphology in MDS.

### SALL4B Transgenic Mice are an Ideal Mouse Model for Human MDS

Monitoring hematological abnormalities in a cohort of 17 transgenic mice from all 6 founders revealed that all mice exhibited apparent MDS-like features at age 2 months. Increased numbers of immature blasts and many atypical and dysplastic white cells, including hypersegmented neutrophils and pseudo-Pelger-Huet-like cells, were seen on peripheral blood smears. Nucleated red blood cells and giant platelets were also present, as well as erythroid and megakaryocyte dysplastic features, such as binucleated erythroid precursors and hypolobulated megakaryocytes. Nine (53 percent) of the 17 mice eventually progressed to AML after age 7-15 months. Leukemic infiltration of many organs, including lungs, kidneys, liver, spleen, and lymph nodes, emphasized the aggressiveness of the disease. The SALL4B-induced AML was also transplantable to immunodeficient mice. The results cannot be explained as a consequence of insertional effects by the following evidence. First, all six founders for SALL4B transgenic mice were analyzed, and they all exhibited a similar phenotype. Second, mice expressing the truncated N-terminal 356 amino acids of SALL4 were generated. No MDS or AML were seen in all six founders.

MDS Progression is Driven by the Expansion of a Subset of Primitive Self-Renewing Stem Cells in Our Mouse Mode

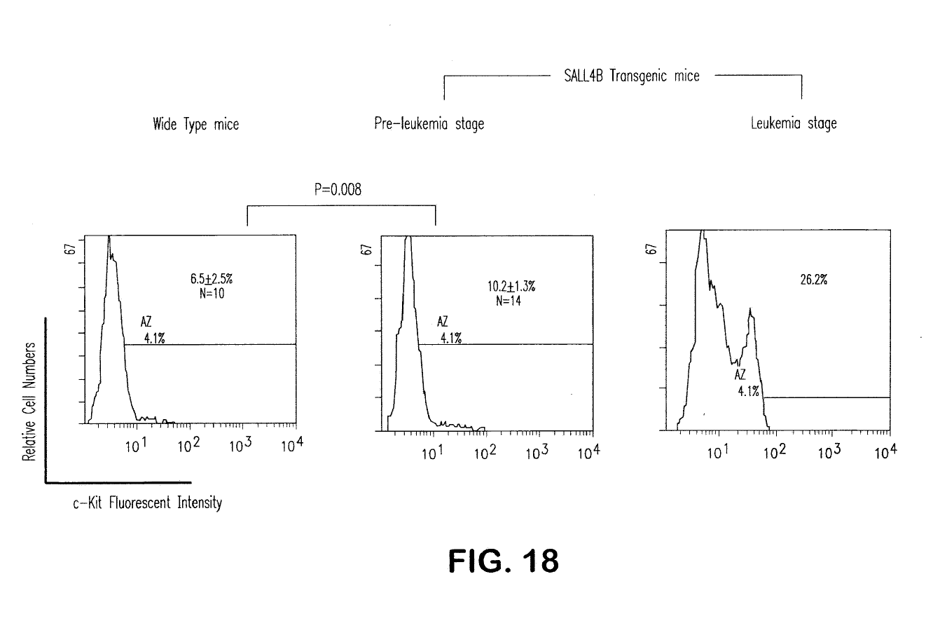

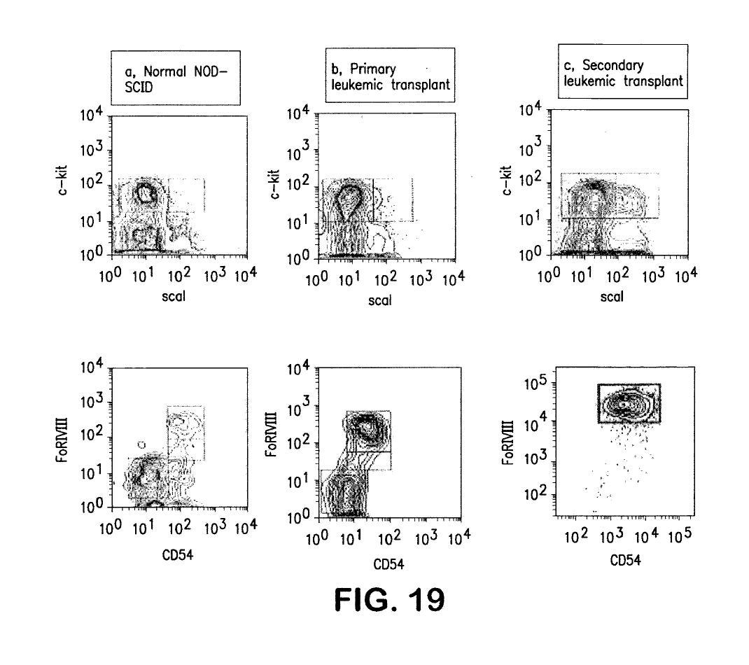

To determine if the cellular defect contributing to the leukemic phenotype was at the stem-cell or progenitor-cell level, the HSC and HPC sub-populations were analyzed with correlation to disease progression in SALL4B transgenic mice. The total number of bone marrow cells was similar among the wild type (WT), pre-leukemic, and leukemic SALL4B transgenic groups. The percentages of both HSC and the HPC populations were elevated significantly for pre-leukemic or leukemic stages in SALL4B transgenic mice as compared to the WT control littermates (FIG. 18). To identify the source of LSCs, serial leukemic transplantations were performed using a NOD-SCID. First, the HSC and HPC sub-populations were sorted from primary leukemic SALL4B transgenic donor mice. The sorting was followed with transplantations into NOD-SCID mice. The leukemic phenotype was noticed in the recipients. We observed that the granulocyte/macrophage progenitors (GMP) cells continued to expand in the transplanted leukemia (FIG. 19), becoming the only HPC population after the second transplantation. Similarly, the HSC population was elevated variably in the leukemic donor and its serial recipient mice. Both HSCs and GMP cells can give rise to the leukemic phenotype in the recipients thus indicating that both populations were LSCs. Moreover, Bmi-1, a gene that plays important roles in self-renewal of LSCs, has been associated with SALL4B-induced LSCs.

In summary, SALL4B transgenic mice exhibited excess blasts, ineffective hematopoiesis, and dysplasia in HSCs, which are all hallmarks of human MDS. Our model presents a novel theory: MDS progression is driven by the expansion of a subset of primitive, self-renewing stem cells.

### SALL4 and Bmi-1 Biochemical Pathways in Regulating LSC Self-Renewal Properties

To date, the polycomb gene Bmi-1 is the most studied gene in regulating LSC self-renewal properties. Knockout of Bmi-1 in mice results in a progressive loss of all hematopoietic lineages. This loss results from the inability of the Bmi-1^{-/} stem cells to self renew. Bmi-1^{-/-} cells display altered expression of the cell-cycle inhibitor genes p16^{INK4a} and p19^{ARF} resulting in the promotion of cell-cycle arrest and apoptosis mainly through the activation of the pRb and p53 pathways. Introducing genes known to produce AML into Bmi-1^{-/-} HSCs induces AML with normal kinetics. Importantly, the Bmi-1^{-/-} LSCs from primary recipients are unable to produce AML in secondary recipients due to exhaustion of the Bmi-1^{-/-} LSCs. Similar to Bmi-1, SALL4B is highly expressed in HSCs and is down-regulated as differentiation proceeds. The expansion of stem compartments is accompanied with MDS and progression of MDS to AML associated with up-regulated expression of Bmi-1 in the SALL4B mouse model. In addition, our data have shown that the SALL4B gene is able to transactivate Bmi-1. By chromatin immunoprecipitation (ChIP), we have demonstrated that SALL4 can bind directly to the Bmi-1 promoter in a region involving SALL4 stimulation, further indicating that Bmi-1 is a SALL4B downstream target that mediates LSC self-renewal.

### Massive Apoptosis and Significant Growth Arrest are Induced by Reducing SALL4 Expression in Cancer-Specific Cells

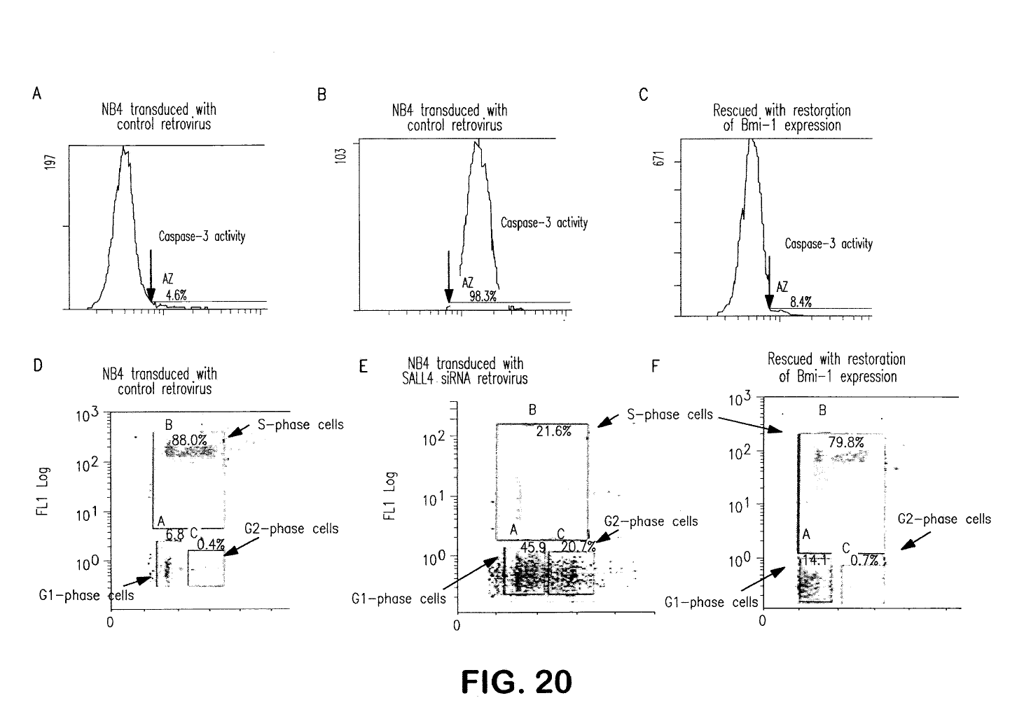

To understand the function of SALL4 in leukemic cells, we have investigated the effect of SALL4 knockdown in an AML cell line, NB4. We applied siRNA to suppress SALL4 expression in the NB4 cell line. Two siRNA retroviral constructs that target different regions of the SALL4 mRNA were made, and their ability to reduce SALL4 mRNA in NB4 cells was confirmed by Q-RT-PCR. In both SALL4 siRNA constructs, down-regulation of SALL4 also significantly reduced Bmi-1 levels. As shown in FIG. 20, a 21-fold increase in caspase-3 activity—from 4.6 percent to 98.3 percent—was seen in WT cells for NB4 cells that reduced approximately 50 percent mRNA of the WT levels of SALL4 (FIGS. 20A and B). Caspase-3 is one of the key protein markers for the apoptosis pathway. Similar results were observed in other cancer cell lines, such as an embryonic carcinoma (EC) cell line and a chronic myeloid Leukemic cell line, KBM5 (data not shown). In addition, the SALL4-induced caspase-3 activity was restored to a near normal level by overexpression of Bmi-1 (FIG. 20C). To further study the role of the SALL4 stem cell gene in cell growth, cell-cycle changes and cellular DNA synthesis were monitored in SALL4-suppressed NB4 cells and NB4 cells through BrdU, incorporation assay and FACS (fluorescence-activated cell sorting). NB4 cells that reduced SALL4 expression up to 50 percent showed about a four-fold decrease in S phase cells and a significant increase in the G1 and G2 phases (6 and 50-folds, respectively), which paralleled the drop in DNA synthesis as judged from the level of BrdU incorporation (FIGS. 20D and 20E). Similar results were observed in other cancer cell lines, such as NTERA2, an embryonic cancer cell line. In contrast, no significant change in the cell-cycle profile was observed when the NB4 cells were transduced with control viruses. To determine if restoration of Bmi-1 alone is sufficient to override decreased cell proliferation and cell-cycle arrest induced by SALL4 knockdown, Bmi-1 in SALL4-suppressed NB4 cells was restored by ectopically expressing Bmi-1. Restoration of Bmi-1 was sufficient to rescue decreased cell proliferation and cell-cycle arrest induced by a reduction of SALL4 (FIG. 20F). These results suggest that cell-cycle arrest and decreased cell proliferation in SALL4-knockdown NB4 cells could be accounted for by decreased expression of Bmi-1. This result is also consistent with Bmi-1 as a target gene of SALL4.

To determine if suppression of SALL4 affects only the survival of cancer stem cells but not normal ES cells, the effect of SALL4 reduction on EC cells, NTERA2, which are malignant pluripotent stem cells, was compared with the effect on normal (ES) cells. Approximately 50 percent reduction of SALL4 led to significant EC cell apoptosis (10 fold increase) as determined by measuring caspase-3 activity and cell deaths by morphology, whereas no significant cell death or increased caspase-3 activity was observed in SALL4^{-/+} ES cells.

To study the effect of reduced SALL4 on bone marrow stem cells, a mouse SALL4^{+/-} was generated through homologous recombination. Approximately 50 percent heterozygous, SALL4 knock-out mice (SALL4^{+/-}) survived despite the defect at the ES cell level. However, homozygous SALL4 mutant embryos died in very early gestation. Hematological analysis was performed on the surviving SALL4^{+/-} and WT control mice. Results showed that these heterozygous mice exhibited mild leukopenia in the peripheral blood. SALL4^{+/-} bone marrows were similar to those found in the WT controls. The immature HSCs/HPCs in SALL4^{+/-} mice were mildly decreased when compared with those in the WT controls (c-kit-positive population in WT mice: 17±1.8 percent, N=5 vs. SALL4^{+/-}: 13.9±0.9 percent, N=3). To determine the effect on HSC/HPC homozygous SALL4 mice, mice containing the conditional SALL4 allele(s) (floxed) were generated through homologous recombination.

In summary, since the reduction of SALL4 has a dramatic effect on the survival of EC cells but not normal ES cells, while not being bound by theory, it seems that SALL4 may serve as a survival factor to maintain growth and survival of cancer stem cells. These findings provide a foundation for developing a LSC-specific therapy targeting SALL4.

LSCs are quite different from leukemic blast cells, and LSCs are not effectively killed by standard chemotherapy drugs. Consequently, even for patients who attain a remission, the LSCs generally are not destroyed and are considered to be responsible for subsequent relapses with the disease. SALL4 is an ESC gene and over expression of this gene in mice transforms HSCs/HPCs into LSCs associated with up-regulation of Bmi-1. Reduction of SALL4 triggers massive apoptosis and cell-cycle arrest in AML cells associated with reduction in Bmi-1. These phenomenal responses can be rescued by restoring Bmi-1 to a relatively normal level (see above).

Using a conditional SALL4 knockout, whether a loss or reduction in SALL4 triggers LSCs to undergo apoptosis can be determined and whether the elimination of the SALL4 LSC compartment within the leukemia clone is sufficient to cure the disease.

To achieve this, SALL4^{flox/flox} and SALL4^{flox/+} mice are crossed to poly I:C (interferon)-inducible Mx1Cre mice. The Mx1Cre mouse has been shown to induce high levels of Cre recombinase in almost all cell types in the marrow, including stem cells or very early progenitor cells. In this Cre system, the Cre recombinase transgene is under the control of the interferon-regulated promoter in such a manner that induction of Cre expression-achieved by injecting poly I:C—causes an excision of a critical exon from the target gene. Bone marrow cells from 5-FU (fluorouracil)-treated SALL4^{flox/flox}/Cre and SALL4^{flox/+}/Cre mice will be retrovirally transduced with the Hoxa9-Meis1 fusion gene and transplanted into a lethally irradiated recipient to generate the AML mouse model. Since LSCs in AML are similar to LSCs in MDS progression with increased leukemic blasts and because there is no mouse model available for MDS progression, we will focus on an AML mouse model.

AML is demonstrated by a peripheral blood smear, and AML-bearing mice will be injected intraperitoneally with the interferon inducer polyinosinic-polycytidylic (pIpC) to excise the SALL4 gene. The deletion of SALL4 will be monitored to slow the leukemia progression and change the phenotype or clinical presentation. Leukemic blasts will be counted by a peripheral blood smear. The lower leukemic blast number in the peripheral blood or bone marrow could indicate an exhaustion of SALL4^{-/-} or SALL4^{-/+} LSCs. FACS will be used to analyze leukemic blasts. The main reason for analyzing both SALL4^{-/-} and SALL4^{-/+} LSCs, is because we anticipate a dose-response effect with SALL4 deletion on LSCs. To further evaluate a possible exhaustion of SALL4^{-/-} or SALL4^{-/+} LSCs, transplantation assays are performed. The AML cells derived from the bone marrow of SALL4^{-/-} or SALL4^{-/+} mice will be transplanted into synergistic mice. Recipient mice will then be monitored over time for the development of AML. AML cells from SALL4^{-/-} or SALL4^{-/+} mice will be analyzed for apoptosis and cell-cycle progression. Furthermore, the survival and growth characteristics of AML cells from SALL4^{-/-} or SALL4^{-/+} will be monitored through long-term in vitro cultures.

To correlate our preliminary studies on AML cells in vivo and in vitro, and whether the AML-inducing capacity of SALL4^{-/-} or SALL4^{-/+} LSC can be rescued in vivo by the overexpression of Bmi-1 and restored to a normal function similar to WT will be determined.

Lentiviruses that express Bmi-1 are prepared. Retroviral supernatants will be used to transduce SALL4^{-/-} and SALL4^{-/+} AML HSCs/HPCs cells sorted from AML SALL4^{-/-} or SALL4^{-/+} mouse marrows. GFP⁺ (green fluorescent protein) and GFP⁻ cells will be FACS-purified. Bmi-1 expression will be assessed by RT-PCR assay. GFP⁺ and GFP⁻ cells of SALL4^{-/-} AML HSCs/HPCs will be assayed for bone marrow transplantation and colony formation as previously described. If increased Bmi-1 restores the self renewal ability of SALL4^{-/-} AML HSCs/HPCs, then the GFP⁺ cells will be transplantable and demonstrate increased replating in long-term culture.

To address whether specifically targeting the SALL4 gene, it will be possible to preferentially induce apoptosis in the LSC population of whole organisms, an RCAS virus that facilitates delivery of siRNA into LSCs that express TVA is used. Mice are created that express the receptor for the subgroup A avian leukosis virus (ATV), specifically for HSCs and HPCs in SALL4B mice. This will be achieved by placing the gene which encodes this virus receptor (TVA) under the control of a promoter, scl, that is active only in HSCs and HPCs. SALL4B mice will be crossed to scl-TVA mice to generate SALL4B/scl-TVA mice. Therefore, all HSCs and HPCs of SALL4B/scl-TVA mice will express this receptor and be susceptible to infection by ATV, while other tissues cannot be infected because they lack the TVA receptor. LSCs of SALL4/scl MDS mice will express the ATV receptor since LSCs are transformed from HSCs and HPCs. TVA-based retroviral vectors have been successfully used in the development of cancer models with mice.

MDS progression will be characterized after intravenous and intra-marrow injection of variable titers of RCANBP viruses carrying the SALL4 siRNA sequence (which silences the expression of SALL4).

Oligonucleotides sequences will be inserted into the RCANBP(A)H1 vector, and the viruses will be produced in DF-1 cells. As a negative control, a vector containing a scrambled siRNA sequence will be used. The virus will be tested to reduce SALL4 expression in leukemic cell lines. The extent of the reduction will be assessed at the RNA level using Q-PCR and at the protein level by western analysis. The effect on cell death will be determined by cell count. The efficacy and duration of SALL4 reduction will be determined, as well as the extent of induced cell death, following delivery into blood and marrow of SALL4/scl-TVA MDS mice. When SALL4B/scl-TVA mice progress to AML or in early disease, as demonstrated by a peripheral blood smear, RCANBP H1 viruses carrying SALL4 siRNA will be administrated to mice to suppress SALL4 expression. The latency, penetrance, immunophenotype, and transformation of AML will be compared between three groups of mice: (a) SALL4B/scl-TVA mice with a control retrovirus, (b) SALL4B/scl-TVA mice with RCANBP H1 viruses carrying SALL4 siRNA, and (c) scl-TVA normal mice. In addition, the reduction of SALL4 as related to its functions in LSC vs. normal HSCs/HPCs through apoptosis, cell-cycle progression, long-term culture and bone marrow repopulation assays will be compared.

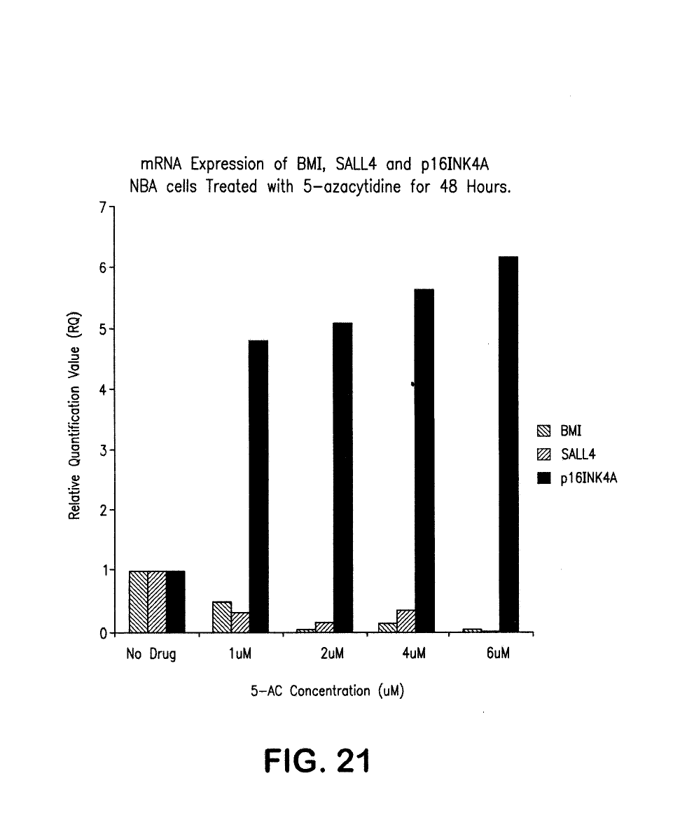

Recent progress in MDS treatment has been reported for 5-azacytidine (5AC), the only drug approved by the FDA for retarding progression in all types of MDS disease. However, the median duration of response to 5AC is less than 18 months. Treatment of a leukemic cell line, NB4, with 5AC significantly suppressed SALL4 and its downstream target, Bmi-1, (FIG. 21). Therefore, while not being bound by theory, it seems that 5AC influences self renewal and proliferation of LSCs through inhibition of SALL4B expression thus retarding MDS progression. Recent studies have also demonstrated that proteasome inhibitors can effectively destroy stem cells in AML, a disease that is closely related to MDS progression. However, proteasome inhibitors produce extreme toxicity, which is unbearable for many patients. There may be an advantage to using both 5AC and proteasome inhibitors.

### Example 4

### Dose-Dependent Activation of the Bmi-1 Promoter by SALL4 Isoforms

Transgenic mice that constitutively over-express human SALL4B, one of the SALL4 isoforms, progress from normal through preleukemic stages (MDS) to acute myeloid leukemias (AML). To search for specific gene targets of SALL4 in leukemogenesis, Affymetrix microarray hybridization (using U133 chips) of SALL4B preleukemic bone marrow mRNA was performed and compared the data with that of control bone marrow. Bmi-1 was identified as one of genes whose expression was significantly increased.

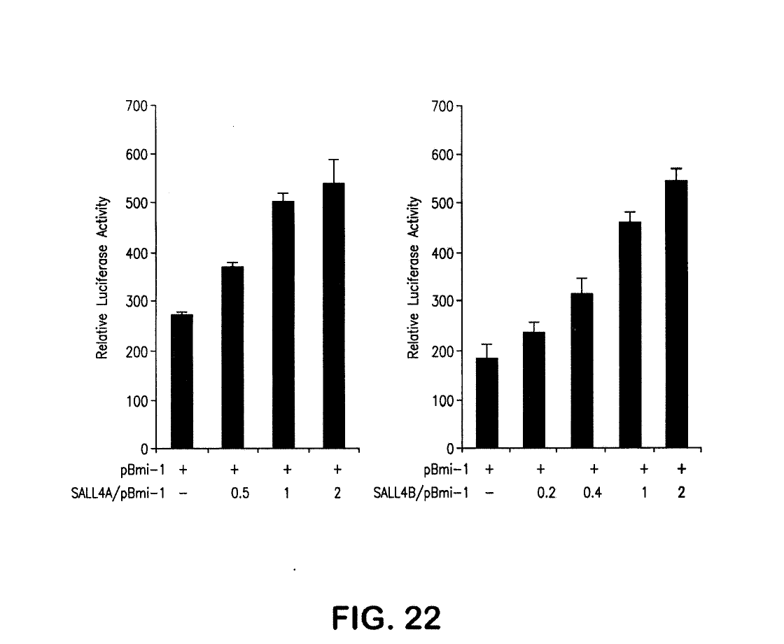

To examine the correlation between Bmi-1 expression and SALL4 expression, analysis of mouse Bmi-1 promoter activity was performed. A ˜2.1 kb sequence upstream of the translation start site was subcloned into the 5′-end of the promoterless pGL3-basic luciferase reporter plasmid. The SALL4 responsiveness of the Bmi-1 promoter then was evaluated through co-transfection of 0.25 μg of the Bmi-1 promoter construct and 0.04 μg of Renilla Luciferase plasmid together with increasing ratios of the SALL4A or SALL4B expression constructs relative to the Bmi-1 promoter construct (0 to 2 ratios). As one increased the molar excess of the SALL4A or SALL4B construct, the Bmi-1 promoter was activated in a dose-dependent manner (FIG. 22).

Mapping of the SALL4 Functional Site within the Bmi-1 Promoter Region by a Luciferase Reporter Gene Assay

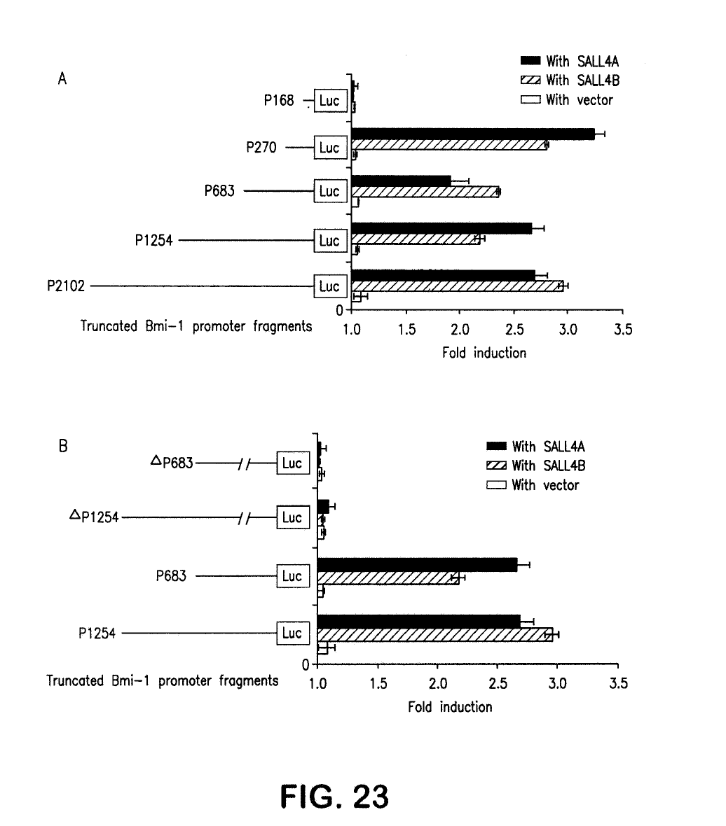

To define the minimal promoter sequence required to activate Bmi-1 by SALL4, transient co-transfection of SALL4 was performed with a series of deleted DNA fragments encompassing the Bmi-1 promoter fused to the luciferase reporter gene. The series of deleted promoter fragments used in the transfection is depicted in FIG. 23A. Each promoter reporter construct of Bmi-1 was transiently co-transfected with the SALL4 isoforms into HEK-293 cells. High levels of activation by both SALL4 isoforms were seen with constructs containing promoter sequences from 0 to −2102, 0 to −1254, 0 to −683 and 0 to −270. Removal of the upstream region between −270 and −168 lead to the inability of SALL4 isoforms to activate the Bmi-1 promoter, indicating the presence of a strong SALL4 activation site in this region. The SALL4 binding region (−270 to −168) then was deleted from the 0 to −1254 and 0 to −683 promoter fragments and two new Bmi-1 promoter constructs created. The luciferase activity of the resulting constructs (P1254 and P683) was compared with activity in the WT promoter constructs with or without co-transfection of SALL4A or SALL4B in HEK-293 cells. There was no significant difference in luciferase activity between the Bmi-1 promoter mutants P1254 and P683 and the WT promoter constructs in HEK-293 cells in the absence of SALL4. However, deletion of the −270 to −168 region abolished the activation of Bmi-1 by SALL4 when compared with that of the WT promoter constructs (FIG. 23B). These results indicate that the −270 to −168 region contains a functional site within the Bmi-1 promoter that is activated by the SALL4 oncogene.

### Binding of SALL4 Proteins to the Bmi-1 Promoter In Vivo

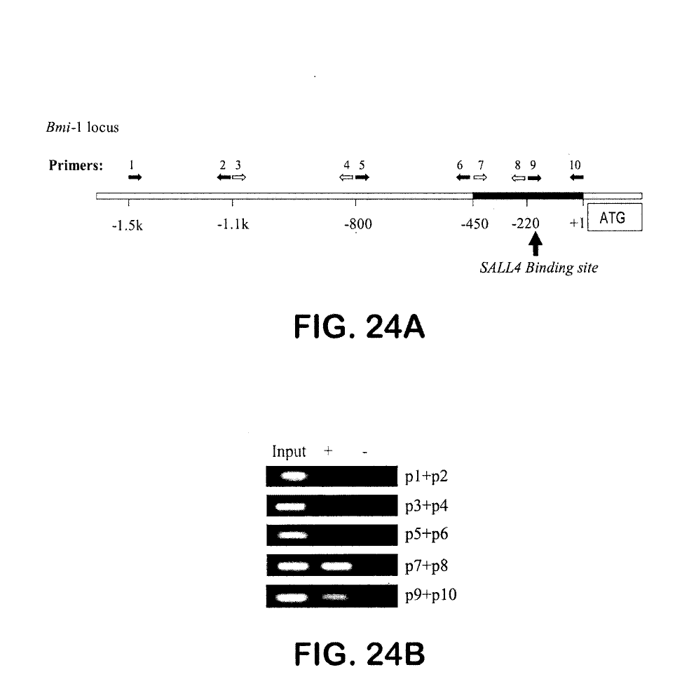

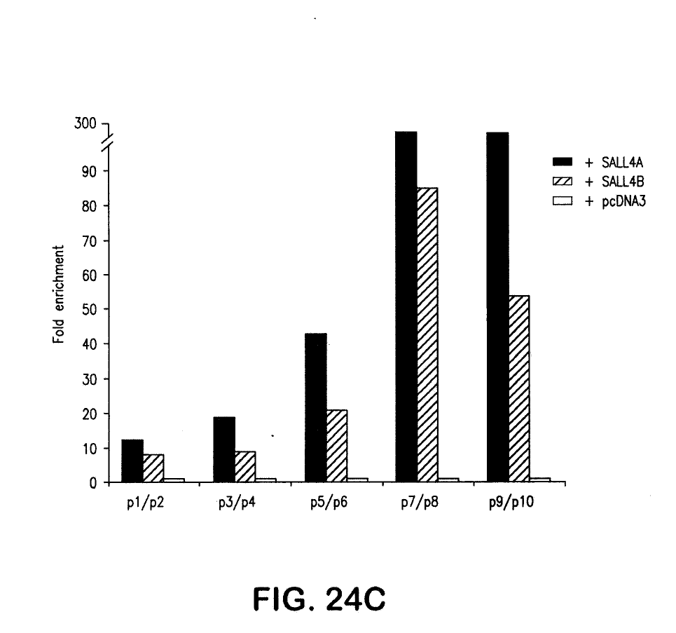

The myeloid stem cell line 32D expresses Bmi-1 but has very low levels of endogenous SALL4. Binding of SALL4 proteins to the Bmi-1 promoter in 32D cells was analyzed using ChiP assays. 32D cells were transfected with SALL4A and SALL4B cDNA constructs tagged with haemagluttin (HA). Chromatin was then extracted, sonicated and immunoprecipitated using rabbit polyclonal antibodies against an HA antibody. The forward and reverse primer sets (7+8 and 9+10) amplified strong 225 bp amplicons from the input sample (FIG. 24B, input lane) and immunoprecipitates (FIG. 24B, +lane). Immunoprecipitation reactions, using preimmune serum show very little amplification of the Bmi-1 promoter construct in the immunoprecipitated DNA (FIG. 24B, −lane). All ChIP samples were tested for false positive PCR amplification by sequencing amplicon DNAs to ascertain the specificity of the SALL4 that bound to the cis-regulatory elements. The intensity of each PCR amplicon was also normalized against the ChIP input band to show the relative abundance of SALL4A that bound to the Bmi-1 promoter construct (FIG. 24C) by Quantitative real time PCR (QRT-PCR). The observed binding was specific, as essentially no signal was observed in parallel ChIP experiments using cells transfected by an empty vector (pcDNA3). This study indicated that a region between −450 to −1 of the Bmi-1 promoter could be a binding site for SALL4A, consistent with the previous luciferase promoter deletion experiments. As expected, SALL4B also demonstrated a similar binding distribution on the Bmi-1 promoter. These studies indicate that the −450 to −1 region of the Bmi-1 promoter has a functional site for activation by both SALL4 isoforms (FIG. 24C). That SALL4 was able to bind the cis-regulatory elements of Bmi-1 in embryonic stem cells, HEK 293 cells, an acute leukemic cell line (NB4), and two AML human samples including M0 (FAB classification) and AML transformed from CML (chronic myeloid leukemia) using ChIP-on-ChIP assays was also demonstrated.

### SALL4 is Able to Affect the Levels of Endogenous Bmi-1 Expression

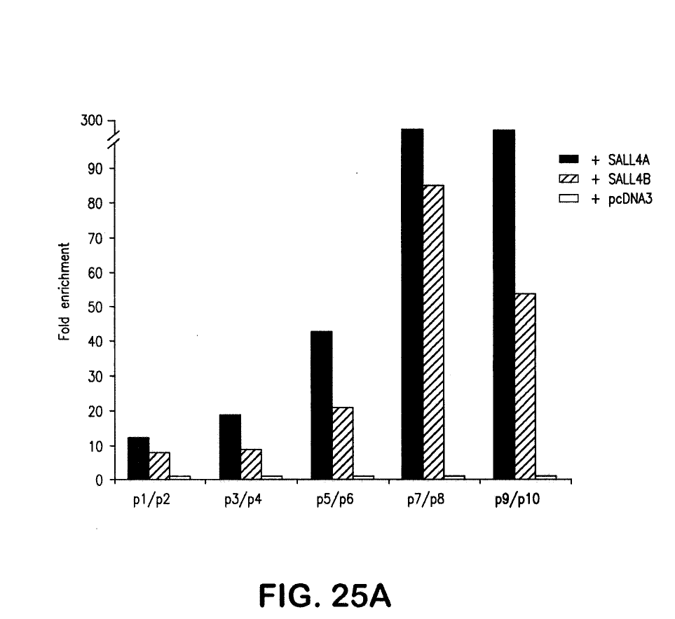

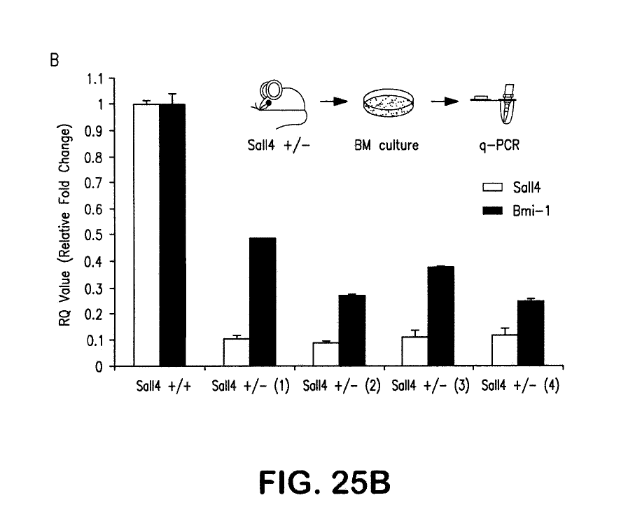

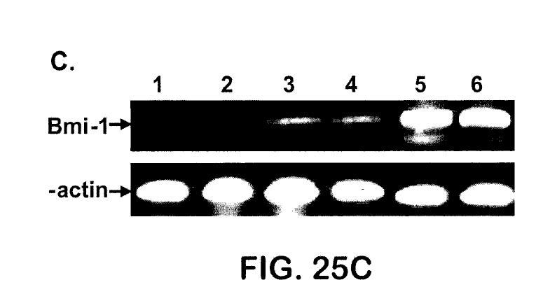

To verify regulation of Bmi-1 by SALL4, SALL4 expression was attenuated in a leukemic cell line, HL60, using siRNA-mediated knockdown. Three siRNA retroviral constructs that target different regions of the SALL4 mRNA were made, and their ability to knockdown SALL4 mRNA in HL60 cells was confirmed by QRT-PCR. Cells from the HL-60 leukemia cell line were infected with the virus collected after 48 hr of transduction. Stable infected cells were identified under G418 selection. In all three SALL4 siRNA constructs, down regulation of SALL4 significantly reduced Bmi-1 levels (FIG. 25A). SALL4 mRNA levels were knocked down by more than 90%, and Bmi-1 expression was reduced by 75-85%.

To gain further supporting evidence of Bmi-1 regulation by SALL4, we analyzed Sall4^{+/-} mice. Homozygous Sall4 mutant embryos die at very early gestation. Approximately 50% of heterozygous Sall4 knock out mice (Sall4^{+/-}) survive despite the defect at the embryonic stem cell level. Bone marrow cells from mutant Sall4+/− and wild type Sall4^{+/+} mice were isolated. Quantitative real-time PCR (QRT-PCR) was performed to compare expression levels of Sall4 and Bmi-1. The heterozygous Sall4^{+/-} bone marrow cells had reduced SALL4 expression as expected. In addition, these heterozygous cells also had significantly reduced expression levels of Bmi-1 as compared to normal mouse bone marrow cells (FIG. 25B).

Increased Expression of Bmi-1 in SALL4B Transgenic Mice Associated with Disease Progression

Transgenic mice that overexpress one of the SALL4 isoforms, SALL4B, exhibited MDS-like features and, subsequently, also exhibited AML transformation. In contrast to WT control mice, the mRNA expression for Bmi-1 was up regulated significantly in preleukemic bone marrows and leukemic blasts from SALL4B transgenic mice (FIG. 25C). Events associated with the progression of MDS and MDS transformation in SALL4B transgenic mice were associated with the up regulation of Bmi-1. Hemotopoetic stem cells (HSCs) and Granulocyte Macrophage Progenitor cells (GMPs) were isolated from three leukemic SALL4 transgenic mice and three non-leukemic SALL4 transgenic mice. Both leukemic HSCs and GMPs had much higher levels of Bmi-1 expression than observed in normal HSCs and GMPs by QRT-PCR. These values range from a two to a twenty fold increase. Variable SALL4B expression levels were observed in different founder mice but in each case the expression levels of Bmi-1 were correlated with the SALL4B expression levels in the HSC and GMP cell populations. In addition, SALL4 expression levels consistently increased as leukemia progresses due to expansion of HSCs and HPCs.

Expression of High Levels of SALL4 Expression in Human AML is Associated with the Expression of High Levels of Bmi-1

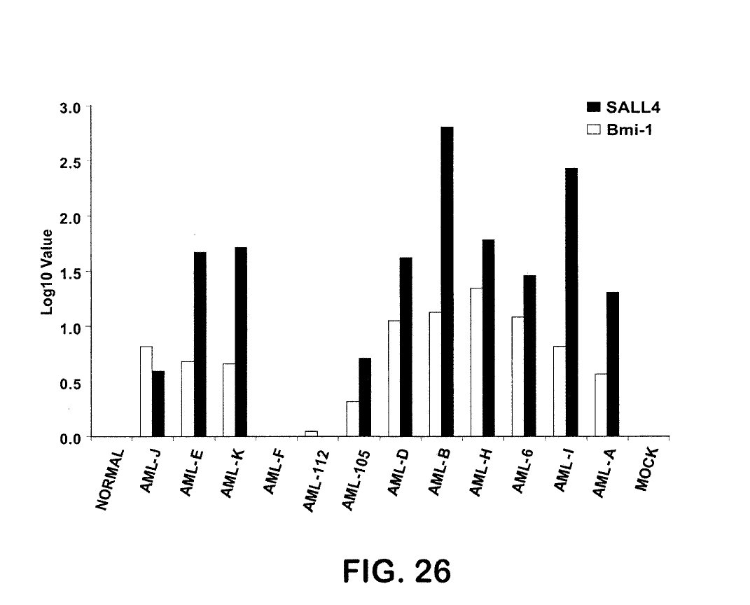

12 random clinical AML samples from bone marrows were analyzed using QRT-PCR to quantify relative mRNA expression of SALL4 and Bmi-1 (FIG. 26). Ten out of 12 AML samples showed significant SALL4 expression ranging from a 3.93- to 653-fold increase relative to the averaged normal controls. These results were consistent with SALL4 protein expression as demonstrated by immunostaining with a SALL4 antibody. Interestingly, the same 10 out of 12 AML samples showed high levels of Bmi-1 expression ranging from a 1.10- to 22-fold increase. There was a strong correlation between the SALL4 and Bmi-1 expression in the AML samples that were examined.

### Epigenetic Alterations at Bmi-1 Gene Promoter Induced by SALL4 Protein

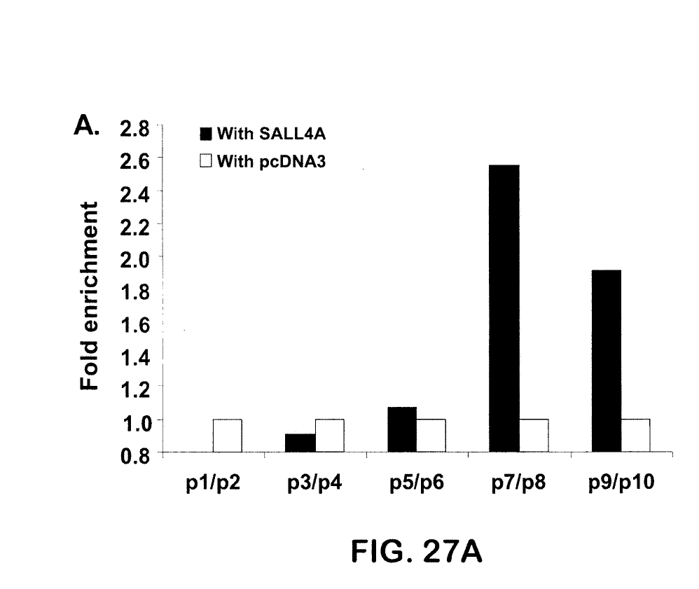

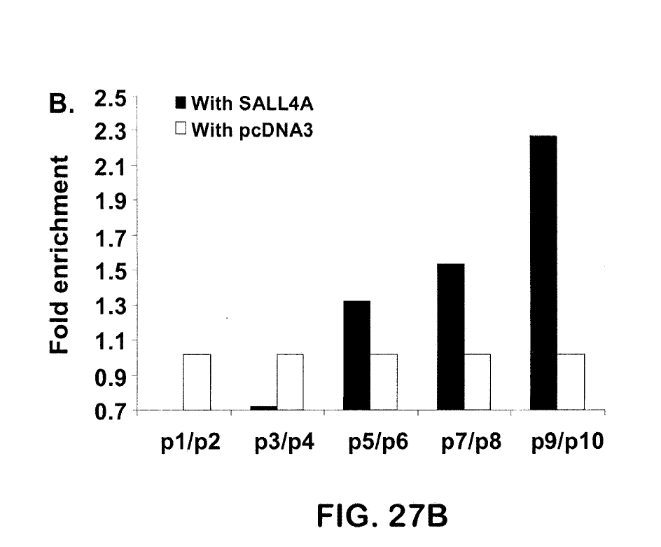

As shown above, SALL4 binds to the Bmi-1 promoter and the regulation of Bmi-1 by SALL4 has been noted in both in vitro and in vivo models of SALL4. H3-K4 trimethylation and H3-K79 methylation have been reported to couple directly to the transcriptional activation. Abnormal H3-K4 trimethylation and H3-K79 are associated also with leukemogenesis. ChIP analysis was performed on the 32D cells, which express no detectable endogenous SALL4, to analyze histone marks present on chromatin before SALL4 binds to the Bmi-1 promoter. ChIP analysis was then performed on 32D cells that had been transfected with SALL4A constructs tagged with HA, or a control vector, and then immunoprecipitated through ChIP using antibodies specific for histone H3-K4 trimethylation and H3-K79 dimethylation. DNAs recovered from these ChIP experiments were amplified by Q-PCR using primers that covered 10.5 kb of the Bmi-1 promoter. Consistent with binding of SALL4 to Bmi-1 promoter sites in the 32D cells transfected with SALL4A or SALL4B constructs, H3-K4 trimethylation was detected and increased roughly 2-3 folds as compared to a vector control (FIG. 27). Similar analysis with H3-K79 methylation revealed robust methylation at SALL4 binding sites and closely paralleled the pattern of H3-K4 trimethylation in the presence of SALL4.

### Example 5

### SALL4 is Expressed Only in Spermatogonia of the Testis

SALL4 is a stem cell gene acting as a gatekeeper in control of early embryonic development. Expression of SALL4 is down-regulated when ESCs are triggered to differentiate and is completely suppressed in normal somatic cells of differentiated tissues. The presence of SALL4 was tested by immunohistochemistry in the testis using an antibody against SALL4. A strong nuclear staining was found in the primordial germ cells of the testis, spermatogonia, whereas the later developmental stages of spermatozoa in seminiferous tubules were negative. In addition, Sertoli cells, leydig cells, and other supporting cells were SALL4 negative.

### SALL4 is a Biomarker for GCTs

Immunohistochemistry staining of various GCTs was done using an anti-SALL4 antibody. The results are summarized in Table 5.

TABLE 4
Results of immunohistochemistry staining from various tissue samples.
Greater than 90% of nuclei in all malignant GCTs stained positive
for SALL4. Negative staining samples had scattered positive staining
cells, but they amounted to less than 1% of the total cells.

| Tumor | Numb | Positi | Nuclei Staining |
|---|---|---|---|
| Classic | 5 | 5 | >90 |
| Spermatoc Semino | 2 | 2 | >90 |
| Embryo Carcino | 5 | 5 | >90 |
| Yolk | 5 | 5 | >90 |
| Immature | 5 | 5 | >90 |
| Mature | 5 | 0 | <2 |
| Non Germ Cell | 4 | 0 | <2 |

Both classic and spermatocytic seminomas (n=5) stained positive for SALL4. Many non-seminomas also stained positive for SALL4 including embryonal carcinomas (n=5), yolk sac tumors (n=5), and immature teratomas (n=5). All positive samples showed strong staining with the SALL4 antibody that was localized specifically to the nucleus of the cells. Negative staining, defined as tissues which had less than 2% of the cells staining positive for SALL4, only occurred in the mature teratoma (n=5). In each case greater than 90% of the tumor cell nuclei were positive with little to no background staining.

SALL4 expression was further investigated in spermatocytic seminomas. The intensity of staining in spermatocytic seminomas appeared to be similar to the staining of spermatogonia in normal testicular tissue. The analysis showed SALL4 to be one of most informative immunohistochemistry markers in identifying GCTs. The data also indicate that testis stem cells, the spermatogonia, are the testicular GCTs of origin.

### Analysis of SALL4 Immunohistochemistry on Multi-Tumor Tissue Array

SALL4 is expressed in very early ESCs, and GCTs are reported to arise from the transformation of these cells. To determine if SALL4 protein can be detected in tumors other than GCTs, immunohistochemistry for SALL4 was performed using a tissue array bearing a variety of epithelial tumor tissues. For comparison samples of normal tissue were placed on the array. All samples of lung (n=10), colon (n=10), breast (n=10), and ovarian (n=10) cancers were classified as staining negatively for SALL4. However, each of these tissues showed intermittent cells with a positive SALL4 nuclear signal in less than 2% of the cells. The normal adult control tissue samples (lung, heart, breast) all stained negative for SALL4, again with about 2% showing a positive nuclear staining. In samples of breast carcinoma, expression of SALL4 protein was observed both in small clusters of cells and scattered individual cells. The observed presence of a small number of SALL4-expressing cells in the non-hematopoietic tissues is consistent with our previous finding that SALL4 is expressed in normal hematopoietic stem cells of the bone marrow at a similar low frequency.

### Decreased SALL4 Expression During NTERA2 Cell Differentiation

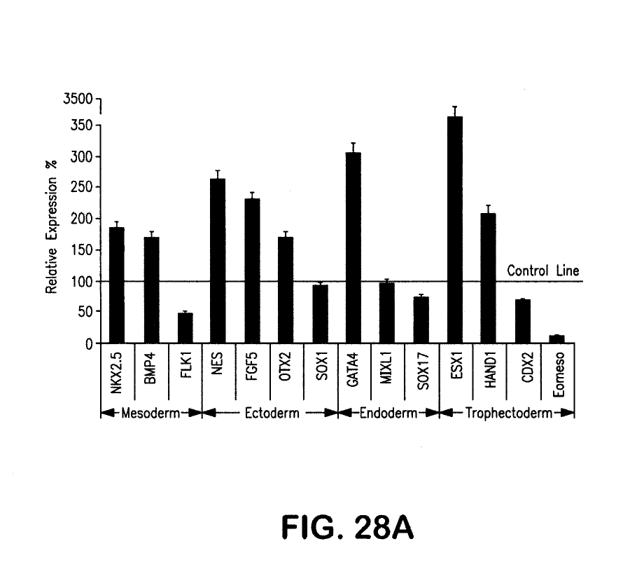

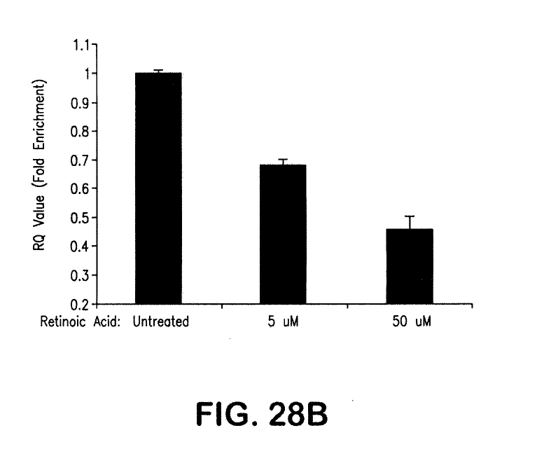

Since SALL4 is a key regulator of self-renewal in ESCs, the expression of SALL4 in NTERA2 cells, an embryonic carcinoma cell line, was analyzed before and after treatment with retinoic acid, a known inducer of differentiation in embryonic carcinoma cells. Retinoic acid treatment resulted in a significant reduction in SALL4 expression (FIG. 28) as well as its downstream target, Bmi-1. To determine the differentiation status of these cells, we assayed by Q-RT-PCR (quantitative real-time polymerase chain reaction) for expression of markers that represent lineage-specific cell differentiation. When NTERA2 cells were treated with 5 um retinoic acid for 24-48 hrs predominately an up-regulation of a panel of ectoderm markers was observed (FIG. 28A). In addition, some endodermal, mesodermal, and trophectodermal genes were also up-regulated. After 48 hours of retinoic acid treatment, SALL4 expression and its downstream target, Bmi-1, were significantly reduced when compared with untreated NTREA2 cells (FIG. 28B).

### Induction of Caspase-3 Activity by Reduction of SALL4 Expression in NTERA2 Cells

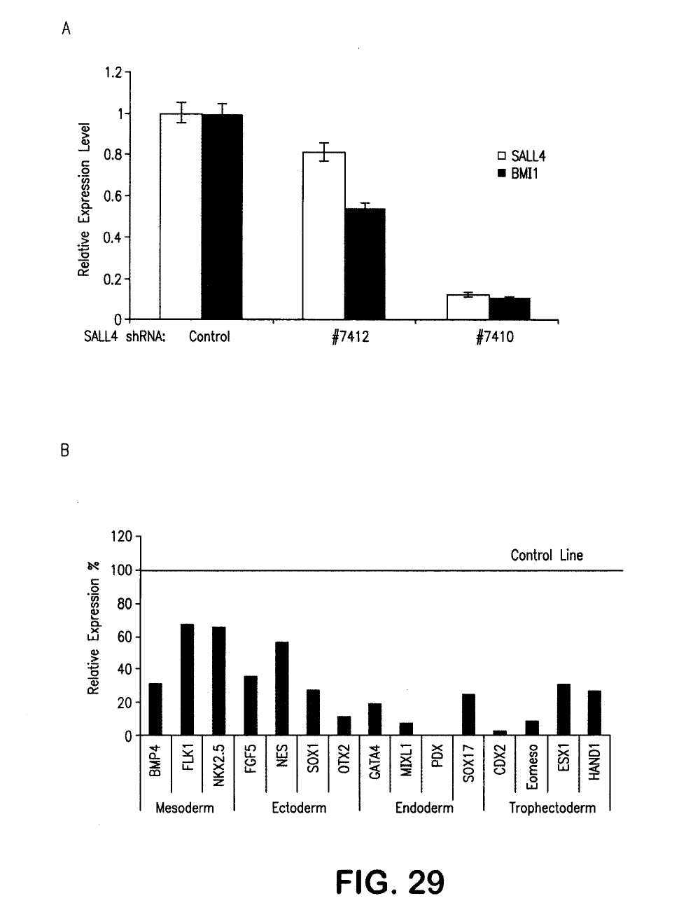

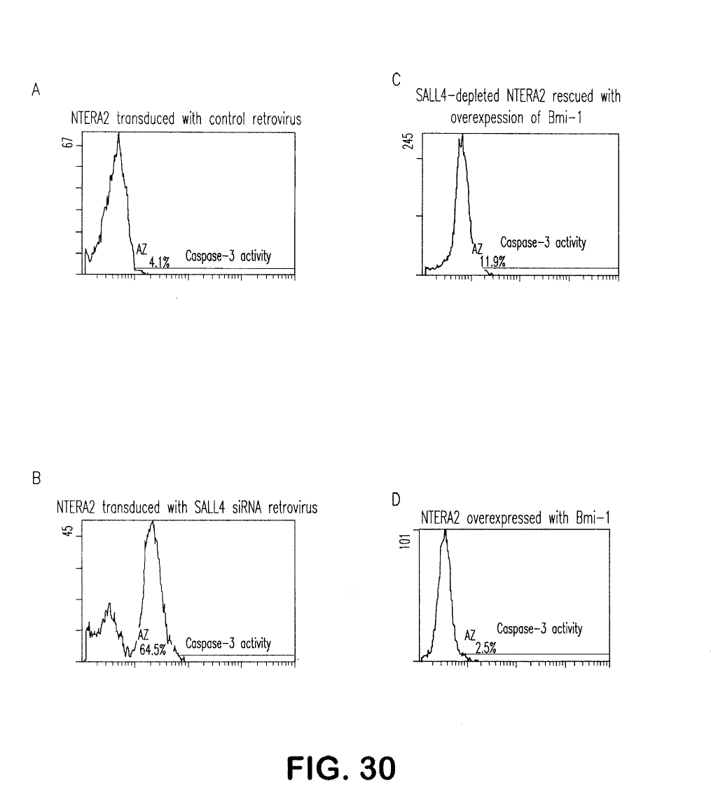

Aberrant expression of SALL4 in hematopoietic stem cells or hematopoietic progenitor cells results in expansion of these cells leading to AML transformation. To understand the function of SALL4 in GCTs, the effect of SALL4 knockdown in NTERA2 cells transduced was investigated with SALL4 siRNA (small interfering ribonucleic acid) retroviruses. Two siRNA retroviral constructs that target different regions of the SALL4 mRNA were made, and their ability to reduce SALL4 mRNA in NTERA2 cells was confirmed by Q-RT-PCR. In both SALL4 siRNA constructs, down-regulation of SALL4 also significantly reduced Bmi-1 levels (FIG. 29A). SALL4 mRNA and Bmi-1 mRNA levels were reduced by more than 90%. In addition, these SALL4 siRNA treated NTERA2 cells appeared to grow slowly and they were unable to differentiate further (FIG. 29B). To determine if reduction of SALL4 expression in NTERA2 cells lead to apoptosis, we measured the level of caspase-3, one the key protein markers for the apoptosis pathway. The level of caspase-3 induced by SALL4 knockdown, was measured by flow cytometry. In NTERA2 cells that retained 10% of the wild-type (WT) levels of SALL4, there was a 12-fold increase of caspase-3 activity to 64.5% from 4.1% in WT cells (FIGS. 30A and 30B). Similar results were observed in other cancer cell lines, such as NB4, an AML cell line.

### Increased Caspase-3 Activity Caused by Decreased SALL4 is Fully Rescued by Overexpression of Bmi-1

To determine if overexpression of Bmi-1 could rescue SALL4-induced caspase-3 activity, SALL4 siRNA treated NTERA2 cells were transfected with an expression vector containing BMI-1. The levels of caspase-3 activity were then measured by flow cytometry. As shown in FIG. 3c, SALL4-induced caspase-3 activity was restored to a near normal level by overexpression of BMI-1. However, overexpression of Bmi-1 has little effect on caspase-3 activity in WT NTERA2 cells (FIG. 30D).

### SALL4 Knockdown Leads to Significantly Decreased Cell Proliferation and Cell-Cycle Arrest

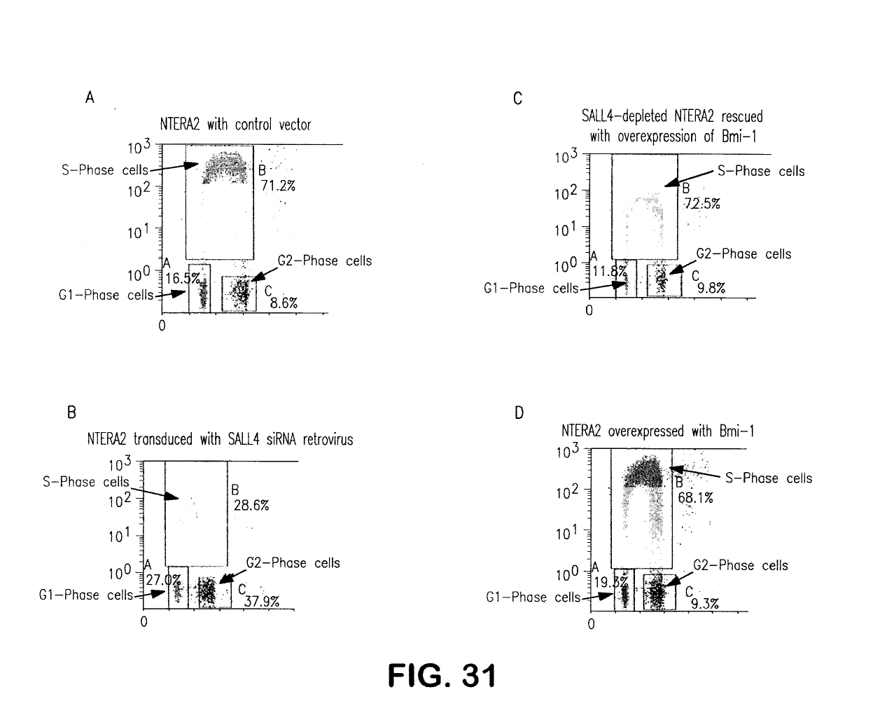

To further study the role of the SALL4 stem gene in cell growth, cell-cycle changes and cellular DNA synthesis were monitored in SALL4-reduced NTERA2 cells and NTERA2 cells through (BrdU) incorporation assay and fluorescence-activated cell sorting (FACS). SALL4 knockdown in NTERA2 cells resulted in G₀/G₁ phase (27%) and G2 phase (37.9%) arrest (FIG. 31B). About a two-fold decrease in S phase cells was also observed, which paralleled the drop in DNA synthesis as judged from the level of BrdU incorporation. Similar results were observed in other cancer cell lines, such as NB4, an AML cell line. In contrast, no significant change in the cell-cycle profile was observed when the WT-NTERA2 cells (which express significant amounts of SALL4) were transduced with control viruses (FIG. 31A).

### Restoration of Bmi-1 is Sufficient to Rescue Decreased Cell Proliferation and Cell-Cycle Arrest Induced by a Reduction of SALL4

To determine if restoration of Bmi-1 alone is sufficient to override decreased cell proliferation and cell-cycle arrest induced by SALL4 knockdown, Bmi-1 was restored in SALL4-deleted NTERA2 cells by ectopically expressing Bmi-1. The re-expression of Bmi-1 in SALL4-deleted NTERA2 cells resulted in an increase in the S phase population and a decrease in the G1 and G2 phases as determined through FACS analysis (FIG. 31C). In addition, as shown in the BrdU labeling assay, SALL4-depleted cells that restored Bmi-1 to a normal level incorporated BrdU significantly in a similar manner as the WT NTERA2 cells (FIG. 31C). These results suggest that cell-cycle arrest and decreased cell proliferation in SALL4-depleted NTERA2 cells could be accounted for by decreased expression of Bmi-1. However, overexpression of Bmi-1 has little effect on cell cycle and proliferation in WT NTERA2 cells (FIG. 31D) and this might be due to the fact that WT NTERA2 cells already bear high levels of Bmi-1.

### REFERENCES

1. Mufti, G., List, A. F., Gore, S. D. & Ho, A. Y. Myelodysplastic syndrome. Hematology (Am Soc Hematol Educ Program), 176-99 (2003).
2. Gilliland, D. G., Jordan, C. T. & Felix, C. A. The molecular basis of leukemia. Hematology (Am Soc Hematol Educ Program), 80-97 (2004).
3. Tenen, D. G. Disruption of differentiation in human cancer: AML shows the way. Nat Rev Cancer 3, 89-101 (2003).
4. Rosmarin, A. G., Yang, Z. & Resendes, K. K. Transcriptional regulation in myelopoiesis: Hematopoietic fate choice, myeloid differentiation, and leukemogenesis. Exp Hematol 33, 131-43 (2005).
5. Friedman, A. D. Transcriptional regulation of myelopoiesis. Int J Hematol 75, 466-72 (2002).
6. Gilliland, D. G. Molecular genetics of human leukemias: new insights into therapy. Semin Hematol 39, 6-11 (2002).
7. Bonnet, D. & Dick, J. E. Human acute myeloid leukemia is organized as a hierarchy that originates from a primitive hematopoietic cell. Nat Med 3, 730-7 (1997).
8. Huntly, B. J. & Gilliland, D. G. Blasts from the past: new lessons in stem cell biology from chronic myelogenous leukemia. Cancer Cell 6, 199-201 (2004).
9. Huntly, B. J. & Gilliland, D. G. Leukaemia stem cells and the evolution of cancer stem cell research. Nat Rev Cancer 5, 311-21 (2005).
10. Simon, M., Grandage, V. L., Linch, D. C. & Khwaja, A. Constitutive activation of the Wnt/beta-catenin signalling pathway in acute myeloid leukaemia. Oncogene 24, 2410-20 (2005).
11. Jamieson, C. H. et al. Granulocyte-macrophage progenitors as candidate leukemic stem cells in blast-crisis CML. N Engl J Med 351, 657-67 (2004).
12. Reya, T. et al. A role for Wnt signalling in self-renewal of haematopoietic stem cells. Nature 423, 409-14 (2003).
13. Reya, T. & Clevers, H. Wnt signalling in stem cells and cancer. Nature 434, 843-50 (2005).
14. Reya, T. Regulation of hematopoietic stem cell self-renewal. Recent Prog Horm Res 58, 283-95 (2003).
15. Staal, F. J. & Clevers, H. C. WNT signalling and haematopoiesis: a WNT-WNT situation. Nat Rev Immunol 5, 21-30 (2005).
16. Kohlhase, J. et al. Isolation, characterization, and organ-specific expression of two novel human zinc finger genes related to the Drosophila gene spalt. Genomics 38, 291-8 (1996).
17. Kohlhase, J. et al. SALL3, a new member of the human spalt-like gene family, maps to 18q23. Genomics 62, 216-22 (1999).
18. Kohlhase, J., Altmann, M., Archangelo, L., Dixkens, C. & Engel, W. Genomic cloning, chromosomal mapping, and expression analysis of msal-2. Mamm Genome 11, 64-8 (2000).
19. Al-Baradie, R. et al. Duane radial ray syndrome (Okihiro syndrome) maps to 20q13 and results from mutations in SALL4, a new member of the SAL family. Am J Hum Genet. 71, 1195-9 (2002).
20. Boube, M., Llimargas, M. & Casanova, J. Cross-regulatory interactions among tracheal genes support a co-operative model for the induction of tracheal fates in the Drosophila embryo. Mech Dev 91, 271-8 (2000).
21. Mollereau, B. et al. Two-step process for photoreceptor formation in Drosophila. Nature 412, 911-3 (2001).
22. Ma, Y. et al. Cloning and characterization of two promoters for the human HSAL2 gene and their transcriptional repression by the Wilms tumor suppressor gene product. J Biol Chem 276, 48223-30 (2001).
23. Ma, Y. et al. SALL1 expression in the human pituitary-adrenal/gonadal axis. J Endocrinol 173, 437-48 (2002).
24. Ma, Y. et al. Hsal 1 is related to kidney and gonad development and is expressed in Wilms tumor. Pediatr Nephrol 16, 701-9 (2001).
25. Marlin, S. et al. Townes-Brocks syndrome: detection of a SALL1 mutation hot spot and evidence for a position effect in one patient. Hum Mutat 14, 377-86 (1999).
26. Nishinakamura, R. et al. Murine homolog of SALL1 is essential for ureteric bud invasion in kidney development. Development 128, 3105-15 (2001).
27. Kohlhase, J. et al. Mutations at the SALL4 locus on chromosome 20 result in a range of clinically overlapping phenotypes, including Okihiro syndrome, Holt-Oram syndrome, acro-renal-ocular syndrome, and patients previously reported to represent thalidomide embryopathy. J Med Genet. 40, 473-8 (2003).
28. Kuhnlein, R. P. et al. spalt encodes an evolutionarily conserved zinc finger protein of novel structure which provides homeotic gene function in the head and tail region of the Drosophila embryo. Embo J 13, 168-79 (1994).
29. Llimargas, M. Wingless and its signalling pathway have common and separable functions during tracheal development. Development 127, 4407-17 (2000).
30. Sato, A. et al. Sall1, a causative gene for Townes-Brocks syndrome, enhances the canonical Wnt signaling by localizing to heterochromatin. Biochem Biophys Res Commun 319, 103-13 (2004).
31. Arroyo, J. L. et al. Impact of immunophenotype on prognosis of patients with myelodysplastic syndromes. Its value in patients without karyotypic abnormalities. Hematol J 5, 227-33 (2004).
32. Buonamici, S. et al. EVI1 induces myelodysplastic syndrome in mice. J Clin Invest 114, 713-9 (2004).
33. Cuenco, G. M., Nucifora, G. & Ren, R. Human AML1/MDS1/EVI1 fusion protein induces an acute myelogenous leukemia (AML) in mice: a model for human AML. Proc Natl Acad Sci USA 97, 1760-5 (2000).
34. Lin, Y. W., Slape, C., Zhang, Z. & Aplan, P. D. NUP98-HOXD13 transgenic mice develop a highly penetrant, severe myelodysplastic syndrome that progresses to acute leukemia. Blood 106, 287-95 (2005).
35. Marisavljevic, D. et al. Biological and clinical significance of clonogenic assays in patients with myelodysplastic syndromes. Med Oncol 19, 249-59 (2002).
36. Cullen, D. A., Killick, R., Leigh, P. N. & Gallo, J. M. The effect of polyglutamine expansion in the human androgen receptor on its ability to suppress beta-catenin-Tcf/Lef dependent transcription. Neurosci Lett 354, 54-8 (2004).
37. Dong, Y. et al. Wnt-mediated regulation of chondrocyte maturation: modulation by TGF-beta. J Cell Biochem 95, 1057-68 (2005).
38. Esufali, S. & Bapat, B. Cross-talk between RacI GTPase and dysregulated Wnt signaling pathway leads to cellular redistribution of beta-catenin and TCF/LEF mediated transcriptional activation. Oncogene 23, 8260-71 (2004).
39. Holmen, S. L., Salic, A., Zylstra, C. R., Kirschner, M. W. & Williams, B. O. A novel set of Wnt-Frizzled fusion proteins identifies receptor components that activate beta-catenin-dependent signaling. J Biol Chem 277, 34727-35 (2002).
40. Merdek, K. D., Nguyen, N. T. & Toksoz, D. Distinct activities of the alpha-catenin family, alpha-catulin and alpha-catenin, on beta-catenin-mediated signaling. Mol Cell Biol 24, 2410-22 (2004).
41. You, L. et al. Inhibition of Wnt-2-mediated signaling induces programmed cell death in non-small-cell lung cancer cells. Oncogene 23, 6170-4 (2004).
42. Warner, D. R., Greene, R. M. & Pisano, M. M. Cross-talk between the TGFbeta and Wnt signaling pathways in murine embryonic maxillary mesenchymal cells. FEBS Lett 579, 3539-46 (2005).
43. Ribeiro, C., Neumann, M. & Affolter, M. Genetic control of cell intercalation during tracheal morphogenesis in Drosophila. Curr Biol 14, 2197-207 (2004).

Although the invention has been described with reference to the above examples, it will be understood that modifications and variations are encompassed within the spirit and scope of the invention. Accordingly, the invention is limited only by the following claims.

What is claimed is:

1. A method of diagnosing disorders of primordial cell origin in a subject comprising determining the expression of SALL4 in a tissue sample from the subject.

2. The method of claim 1, wherein the disorder is associated with a germ cell tumor (GCT).

3. The method of claim 2, wherein the GCT is a classic seminoma, spermatocytic seminoma, embryonal carcinoma, yolk sac tumor, or immature teratoma.

4. The method of claim 1, wherein the tissue sample comprises cells of testicular origin.

5. The method of claim 4, wherein substantially all mature testicular cell types present in the sample do not express SALL4.

6. The method of claim 1, wherein the tissue sample is obtained from a site which comprises cells that have metastasized from a GCT.

7. A method of monitoring engraftment of transplanted stem cells in a subject comprising:
a) determining the level of expression of SALL4 in stem cells prior to transplantation into a subject;
b) grafting the cells of step (a) into the subject;
c) determining the level of expression of SALL4 in the grafted stem cells at time intervals post-transplantation,
wherein a decrease in SALL4 expression over the time intervals correlates with differentiation of the stem cells, and wherein such differentiation is indicative of positive engraftment of cells in the subject.

8. The method of claim 7, wherein an increase in SALL4 expression over the time intervals correlates with repression of differentiation, and wherein such repression is indicative of negative engraftment of cells in the subject.

9. The method of claim 7, wherein the cell is transformed by a vector encoding an exogenous or endogenous gene product.

10. A method for isolating stem cells from cord blood comprising:
a) obtaining umbilical cord cells (UBC) from a subject;
b) sorting cells that express SALL4 from cells that do not express SALL4; and optionally;
c) selecting by one or more markers, cells from the sorted cells that express SALL4,
wherein UBCs expressing SALL4 are indicative of isolated stem cells.

11. The method of claim 10, wherein the one or more markers are selected from the group consisting of SSEA-1, SSEA-2, SSEA-4, TRA-1-60, TRA-1-81, CD34⁺, CD59⁺, Thy1/CD90⁺, CD38^{lo/-}, C-kit^{-/lo}, lin⁻, SH2, vimentin, periodic acid Schiff activity (PAS), FLK1, BAP, and acid phosphatase.

12. The method of claim 10, wherein the step of sorting comprises sorting by fluorescence activated cell sorting (FACS).

13. The method of claim 12, wherein the step of sorting comprises sorting by magnetic bead sorting (MACS).

14. A method of treating a cancer of stem cell or progenitor cell origin comprising administrating to a subject in need thereof a composition comprising an agent which reduces the expression level of SALL4.

15. The method of claim 14, wherein the agent is an oligonucleotide sequence selected from SEQ ID NO:30, SEQ ID NO:31; SEQ ID NO:32; SEQ ID NO:33; or SEQ ID NO:34.

16. The method of claim 14, wherein the composition comprises a methylation inhibitor.

17. The method of claim 16, wherein the methylase inhibitor is selected from 5′ azacytidine, 5′ aza-2-deoxycytidine, 1-B-D-arabinofuranosyl-5-azacytosine, or dihydroxy-5-azacytidine.

18. The method of claim 17, wherein the composition further comprises a proteasome inhibitor.

19. The method of claim 18, wherein the proteasome inhibitor is selected from MG 132, PSI, lactacystin, epoxomicin, or bortezomib.

20. The method of claim 14, wherein the stem cell or progenitor cell is selected from a leukemic stem cell, seminoma, spermatocytic seminoma, embryonal carcinoma, yolk sac tumor, or immature teratoma.

21. An isolated oligonucleotide selected from SEQ ID NO:30, SEQ ID NO:31; SEQ ID NO:32; SEQ ID NO:33; or SEQ ID NO:34.


SEQUENCE LISTING (36 sequences)

SEQ ID NO: 1 - DNA, 3162 bp, Homo sapiens
atgtcgaggcgcaagcaggcgaaaccccagcacatcaactcggaggaggaccagggcgagcagcagccgcagcagcagaccccggagtttgcagatgcggccccagcggcgcccgcggcgggggagctgggtgctccagtgaaccacccagggaatgacgaggtggcgagtgaggatgaagccacagtaaagcggcttcgtcgggaggagacgcacgtctgtgagaaatgctgtgcggagttcttcagcatctctgagttcctggaacataagaaaaattgcactaaaaatccacctgtcctcatcatgaatgacagcgaggggcctgtgccttcagaagacttctccggagctgtactgagccaccagcccaccagtcccggcagtaaggactgtcacagggagaatggcggcagctcagaggacatgaaggagaagccggatgcggagtctgtggtgtacctaaagacagagacagccctgccacccaccccccaggacataagctatttagccaaaggcaaagtggccaacactaatgtgaccttgcaggcactacggggcaccaaggtggcggtgaatcagcggagcgcggatgcactccctgcccccgtgcctggtgccaacagcatcccgtgggtcctcgagcagatcttgtgtctgcagcagcagcagctacagcagatccagctcaccgagcagatccgcatccaggtgaacatgtgggcctcccacgccctccactcaagcggggcaggggccgacactctgaagaccttgggcagccacatgtctcagcaggtttctgcagctgtggctttgctcagccagaaagctggaagccaaggtctgtctctggatgccttgaaacaagccaagctacctcacgccaacatcccttctgccaccagctccctgtccccagggctggcacccttcactctgaagccggatgggacccgggtgctcccgaacgtcatgtcccgcctcccgagcgctttgcttcctcaggccccgggctcggtgctcttccagagccctttctccactgtggcgctagacacatccaagaaagggaaggggaagccaccgaacatctccgcggtggatgtcaaacccaaagacgaggcggccctctacaagcacaagtgtaagtactgtagcaaggtttttgggactgatagctccttgcagatccacctccgctcccacactggagagagacccttcgtgtgctctgtctgtggtcatcgcttcaccaccaagggcaacctcaaggtgcactttcaccgacatccccaggtgaaggcaaacccccagctgtttgccgagttccaggacaaagtggcggccggcaatggcatcccctatgcactctctgtacctgaccccatagatgaaccgagtctttctttagacagcaaacctgtccttgtaaccacctctgtagggctacctcagaatctttcttcggggactaatcccaaggacctcacgggtggctccttgcccggtgacctgcagcctgggccttctccagaaagtgagggtggacccacactccctggggtgggaccaaactataattccccaagggctggtggcttccaagggagtgggacccctgagccagggtcagagaccctgaaattgcagcagttggtggagaacattgacaaggccaccactgatcccaacgaatgtctcatttgccaccgagtcttaagctgtcagagctccctcaagatgcattatcgcacccacaccggggagagaccgttccagtgtaagatctgtggccgagccttttctaccaaaggtaacctgaagacacaccttggggttcaccgaaccaacacatccattaagacgcagcattcgtgccccatctgccagaagaagttcactaatgccgtgatgctgcagcaacatattcggatgcacatgggcggtcagattcccaacacgcccctgccagagaatccctgtgactttacgggttctgagccaatgaccgtgggtgagaacggcagcaccggcgctatctgccatgatgatgtcatcgaaagcatcgatgtagaggaagtcagctcccaggaggctcccagcagctcctccaaggtccccacgcctcttcccagcatccactcggcatcacccacgctagggtttgccatgatggcttccttagatgccccagggaaagtgggtcctgccccttttaacctgcagcgccagggcagcagagaaaacggttccgtggagagcgatggcttgaccaacgactcatcctcgctgatgggagaccaggagtatcagagccgaagcccagatatcctggaaaccacatccttccaggcactctccccggccaatagtcaagccgaaagcatcaagtcaaagtctcccgatgctgggagcaaagcagagagctccgagaacagccgcactgagatggaaggtcggagcagtctcccttccacgtttatccgagccccgccgacctatgtcaaggttgaagttcctggcacatttgtgggaccctcgacattgtccccagggatgacccctttgttagcagcccagccacgccgacaggccaagcaacatggctgcacacggtgtgggaagaacttctcgtctgctagcgctcttcagatccacgagcggactcacactggagagaagccttttgtgtgcaacatttgtgggcgagcttttaccaccaaaggcaacttaaaggttcactacatgacacacggggcgaacaataactcagcccgccgtggaaggaagttggccatcgagaacaccatggctctgttaggtacggacggaaaaagagtctcagaaatctttcccaaggaaatcctggccccttcagtgaatgtggaccctgttgtgtggaaccagtacaccagcatgctcaatggcggtctggccgtgaagaccaatgagatctctgtgatccagagtgggggggttcctaccctcccggtttccttgggggccacctccgttgtgaataacgccactgtctccaagatggatggctcccagtcgggtatcagtgcagatgtggaaaaaccaagtgctactgacggcgttcccaaacaccagtttcctcacttcctggaagaaaacaagattgcggtcagctaa

SEQ ID NO: 2 - protein, 1053 aa, Homo sapiens
MSRRKQAKPQHINSEEDQGEQQPQQQTPEFADAAPAAPAAGELGAPVNHPGNDEVASEDEATVKRLRREETHVCEKCCAEFFSISEFLEHKKNCTKNPPVLIMNDSEGPVPSEDFSGAVLSHQPTSPGSKDCHRENGGSSEDMKEKPDAESVVYLKTETALPPTPQDISYLAKGKVANTNVTLQALRGTKVAVNQRSADALPAPVPGANSIPWVLEQILCLQQQQLQQIQLTEQIRIQVNMWASHALHSSGAGADTLKTLGSHMSQQVSAAVALLSQKAGSQGLSLDALKQAKLPHANIPSATSSLSPGLAPFTLKPDGTRVLPNVMSRLPSALLPQAPGSVLFQSPFSTVALDTSKKGKGKPPNISAVDVKPKDEAALYKHKCKYCSKVFGTDSSLQIHLRSHTGERPFVCSVCGHRFTTKGNLKVHFHRHPQVKANPQLFAEFQDKVAAGNGIPYALSVPDPIDEPSLSLDSKPVLVTTSVGLPQNLSSGTNPKDLTGGSLPGDLQPGPSPESEGGPTLPGVGPNYNSPRAGGFQGSGTPEPGSETLKLQQLVENIDKATTDPNECLICHRVLSCQSSLKMHYRTHTGERPFQCKICGRAFSTKGNLKTHLGVHRTNTSIKTQHSCPICQKKFTNAVMLQQHIRMHMGGQIPNTPLPENPCDFTGSEPMTVGENGSTGAICHDDVIESIDVEEVSSQEAPSSSSKVPTPLPSIHSASPTLGFAMMASLDAPGKVGPAPFNLQRQGSRENGSVESDGLTNDSSSLMGDQEYQSRSPDILETTSFQALSPANSQAESIKSKSPDAGSKAESSENSRTEMEGRSSLPSTFIRAPPTYVKVEVPGTFVGPSTLSPGMTPLLAAQPRRQAKQHGCTRCGKNFSSASALQIHERTHTGEKPFVCNICGRAFTTKGNLKVHYMTHGANNNSARRGRKLAIENTMALLGTDGKRVSEIFPKEILAPSVNVDPVVWNQYTSMLNGGLAVKTNEISVIQSGGVPTLPVSLGATSVVNNATVSKMDGSQSGISADVEKPSATDGVPKHQFPHFLEENKIAVS

SEQ ID NO: 3 - DNA, 1851 bp, Homo sapiens
  misc_feature (337)..(337): n is a, c, g, or t
atgtcgaggcgcaagcaggcgaaaccccagcacatcaactcggaggaggaccagggcgagcagcagccgcagcagcagaccccggagtttgcagatgcggccccagcggcgcccgcggcgggggagctgggtgctccagtgaaccacccagggaatgacgaggtggcgagtgaggatgaagccacagtaaagcggcttcgtcgggaggagacgcacgtctgtgagaaatgctgtgcggagttcttcagcatctctgagttcctggaacataagaaaaattgcactaaaaatccacctgtcctcatcatgaatgacagcgaggggcctgtgccttcanaagacttctccggagctgtactgagccaccagcccaccagtcccggcagtgaggactgtcacagggagaatggcggcagctcanaggacataaaggagaagccggatgcggagtctgtggtgtacctaaagacagagacagccctgccacccaccccccaggacataagctatttagccaaaggcaaagtggccaacactaacgtgaccttgcaggcactacggggcaccaaggtggcggtgaatcagcggagcgcggatgcactccctgcccccgtgcctggtgccaacagcatcccgtgggtcctcgagcagatcttgtgtctgcagcagcagcagctacagcagatccagctcaccgagcagatccgcatccaggtgaacatgtgggcctcccacgccctccactcaagcggggcaggggccgacactctgaagaccttgggcagccacatgtctcagcaggtttctgcagctgtggctttgctcagccagaaagctggaagccaaggtctgtctctggatgccttgaaacaagccaagctacctcacgccaacatcccttctgccaccagctccctgtccccagggctggcacccttcactctgaagccggatgggacccgggtgctcccgaacgtcatgtcccgcctcccgagcgctttgcttcctcaggccccgggctcggtgctcttccagagccctttctccactgtggcgctagacacatccaagaaagggaaggggaagccaccgaacatctccgcggtggatgtcaaacccaaagacgaggcggccctctacaagcacaagtgtcggagcagtctcccttccacgtttatccgagccccgccgacctatgtcaaggttgaagttcctggcacatttgtgggaccctcgacattgtccccagggatgacccctttgttagcagcccagccacgcggacaggccaagcaacatggctgcacacggtgtggnaagaacttntcgtntgntagcgctcttcagatccacgagcggactcacantggagagaagccttttgtgtgcaacatttgtgggcgagcttttaccaccaaaggcaacttaaaggttcactacatgacacacggggcgaacaataactcagcccgccgtggaaggaagttggccatcgagaacaccatggctctgttaggtacggacggaaaaagagtctcagaaatctttcccaaggaaatcctggccccttcagtgaatgtggaccctgttgtgtggaaccagtacaccagcatgctcaatggcggtctggccgtgaagaccaatgagatctctgtgatccagagtgggggggttcctaccctcccggtttccttgggggccacctccgttgtgaataacgccactgtctccaagatggatggctcccagtcgggtatcagtgcagatgtggaaaaaccaagtgctactgacggcgttcccaaacnccagtttcctcacttcctggaagaaaacaagantgcggtcagctaa

SEQ ID NO: 4 - protein, 616 aa, Homo sapiens
  misc_feature (113)..(113): Xaa can be any naturally occurring amino acid
MSRRKQAKPQHINSEEDQGEQQPQQQTPEFADAAPAAPAAGELGAPVNHPGNDEVASEDEATVKRLRREETHVCEKCCAEFFSISEFLEHKKNCTKNPPVLIMNDSEGPVPSXDFSGAVLSHQPTSPGSEDCHRENGGSSXDIKEKPDAESVVYLKTETALPPTPQDISYLAKGKVANTNVTLQALRGTKVAVNQRSADALPAPVPGANSIPWVLEQILCLQQQQLQQIQLTEQIRIQVNMWASHALHSSGAGADTLKTLGSHMSQQVSAAVALLSQKAGSQGLSLDALKQAKLPHANIPSATSSLSPGLAPFTLKPDGTRVLPNVMSRLPSALLPQAPGSVLFQSPFSTVALDTSKKGKGKPPNISAVDVKPKDEAALYKHKCRSSLPSTFIRAPPTYVKVEVPGTFVGPSTLSPGMTPLLAAQPRGQAKQHGCTRCGKNXSXXSALQIHERTHXGEKPFVCNICGRAFTTKGNLKVHYMTHGANNNSARRGRKLAIENTMALLGTDGKRVSEIFPKEILAPSVNVDPVVWNQYTSMLNGGLAVKTNEISVIQSGGVPTLPVSLGATSVVNNATVSKMDGSQSGISADVEKPSATDGVPKXQFPHFLEENKXAVS

SEQ ID NO: 5 - DNA, 831 bp, Homo sapiens
atgtcgaggcgcaagcaggcgaaaccccagcacatcaactcggaggaggaccagggcgagcagcagccgcagcagcagaccccggagtttgcagatgcggccccagcggcgcccgcggcgggggagctgggtcggagcagtctcccttccacgtttatccgagccccgccgacctatgtcaaggttgaagttcctggcacatttgtgggaccctcgacattgtccccagggatgacccctttgttagcagcccagccacgccgacaggccaagcaacatggctgcacacggtgtgggaagaacttctcgtctgctagcgctcttcagatccacgagcggactcacactggagagaagccttttgtgtgcaacatttgtgggcgagcttttaccaccaaaggcaacttaaaggttcactacatgacacacggggcgaacaataactcagcccgccgtggaaggaagttggccatcgagaacaccatggctctgttaggtacggacggaaaaagagtctcagaaatctttcccaaggaaatcctggccccttcagtgaatgtggaccctgttgtgtggaaccagtacaccagcatgctcaatggcggtctggccgtgaagaccaatgagatctctgtgatccagagtgggggggttcctaccctcccggtttccttgggggccacctccgttgtgaataacgccactgtctccaagatggatggctcccagtcgggtatcagtgcagatgtggaaaaaccaagtgctactgacggcgttcccaaacaccagtttcctcacttcctggaagaaaacaagattgcggtcagctaa

SEQ ID NO: 6 - protein, 276 aa, Homo sapiens
MSRRKQAKPQHINSEEDQGEQQPQQQTPEFADAAPAAPAAGELGRSSLPSTFIRAPPTYVKVEVPGTFVGPSTLSPGMTPLLAAQPRRQAKQHGCTRCGKNFSSASALQIHERTHTGEKPFVCNICGRAFTTKGNLKVHYMTHGANNNSARRGRKLAIENTMALLGTDGKRVSEIFPKEILAPSVNVDPVVWNQYTSMLNGGLAVKTNEISVIQSGGVPTLPVSLGATSVVNNATVSKMDGSQSGISADVEKPSATDGVPKHQFPHFLEENKIAVS

SEQ ID NO: 7 - DNA, 39 bp, Artificial sequence
  Primer
ttatcaggatcctggtcgaggcgcaagcaggcgaaaccc

SEQ ID NO: 8 - DNA, 33 bp, Artificial sequence
  Primer
ccaggatccttagctgaccgccaatcttgtttc

SEQ ID NO: 9 - DNA, 22 bp, Artificial sequence
  Primer
attggcaccggcagttaccacc

SEQ ID NO: 10 - DNA, 21 bp, Artificial sequence
  Primer
agtactcgtgggcatattgtc

SEQ ID NO: 11 - DNA, 25 bp, Artificial sequence
  Primer
atgtcgaggcgcaagcaggcgaaac

SEQ ID NO: 12 - DNA, 25 bp, Artificial sequence
  Primer
ttagctgaccgcaatcttgttttct

SEQ ID NO: 13 - protein, 13 aa, Homo sapiens
MSRRKQAKPQHIN

SEQ ID NO: 14 - DNA, 19 bp, Artificial sequence
  Primer
gaaggtgaaggtcggagtc

SEQ ID NO: 15 - DNA, 20 bp, Artificial sequence
  Primer
gaagatggtgatgggatttc

SEQ ID NO: 16 - DNA, 20 bp, Artificial sequence
  TaqMan probe
caagcttcccgttctcagcc

SEQ ID NO: 17 - DNA, 26 bp, Artificial sequence
  Primer
cctcctaatgagagtatctgggtgat

SEQ ID NO: 18 - DNA, 24 bp, Artificial sequence
  Primer
ttaaaacatacagcgcatgattgg

SEQ ID NO: 19 - DNA, 24 bp, Artificial sequence
  Primer
cagagatgctgaagaactccgcac

SEQ ID NO: 20 - DNA, 23 bp, Artificial sequence
  Primer
agcagagctcgtttagtgaaccg

SEQ ID NO: 21 - DNA, 4668 bp, Homo sapiens
atgtcgcggaggaagcaagcgaagcctcaacatttccaatccgaccccgaagtggcctcgctcccccggcgagatggagacacagaaaagggtcaaccgagtcgccctactaagagcaaggatgcccacgtctgtggccggtgctgtgccgagttctttgaattatcagatcttctgctccacaagaagaactgtactaaaaatcaattagttttaatcgtaaatgaaaatccaggctccccacccgaaaccttctcccccagcccccctcctgataatcctgatgaacaaatgaatgacacagttaacaaaacagatcaagtggactgcagcgacctttcagaacacaacggacttgacagggaagagtccatggaggtggaggccccggttgctaacaaaagcggcagcggcacttccagcggcagccacagcagtaccgccccaagcagcagcagcagcagcagcagcagcagcggcggcggcggcagctcctccacaggtacctcagcgatcacaacctctctacctcaactcggggacctgacaacactgggcaacttctccgtaatcaacagcaacgtcatcatcgagaacctccagagcaccaaggtggcggtggcccagttctcccaggaagcgaggtgcggcggggcctctgggggcaagctggccgtcccagccctcatggaacaactcctagctctgcagcagcagcagatccaccagctgcaattgatcgaacagattcgtcaccaaatattgctgttggcttctcagaatgcagacttgccaacatcttctagtccttctcaaggtactttacgaacatctgccaaccccttgtccacgctaagttcccatttatctcagcagctggcagcagcagctggattggcacagagcctcgccagccaatctgccagcattagtggtgtgaaacagctacccccaatccagctacctcagagcagttctggcaacaccatcattccatccaacagcggctcttctcccaatatgaacatattggcagcggcagttaccaccccgtcctctgaaaaagtggcttcaagtgctggggcctcccatgtcagcaacccagcggtctcatcatcgtcctcaccagcttttgcaataagcagtttattaagtcctgcgtctaatccacttctacctcagcaagcctccgctaactcggttttccccagccctttgcccaacatcggaacaactgcagaggatttaaactccttgtctgccttggcccagcaaagaaaaagcaagccaccaaatgtcactgcctttgaagcgaaaagtacttccgatgaggcattcttcaaacacaagtgcaggttctgcgcgaaggtctttgggagtgacagtgccttgcagatccacttgcgttcccataccggagagaggccattcaagtgcaacatctgcgggaacaggttctccaccaaggggaatctgaaagtccactttcagcgccacaaagagaaataccctcatatccagatgaacccctatcctgtgcctgagcatttggacaatatccccacgagtactggcatcccatatggcatgtccatccctccagagaagccagtcaccagctggctagacaccaaaccagtcctgcctactctgaccacttcagtcggcctgccgttgcccccaagcctcccaagcctcatacccttcatcaagacggaagagccagcccccatccccatcagccattctgccaccagccccccaggctcagtcaaaagtgactccgggggccctgagtcagccacaagaaatctaggtgggctcccagaggaagccgaagggtccactctgccaccctctggtggcaaaagcgaagagagtggcatggtcaccaactcagtcccgacggcgagcagtagcgtcctgagctccccagcggcagactgcggccccgcgggcagtgccaccaccttcaccaaccctttgttgccgctcatgtccgagcagttcaaggccaagtttccttttgggggactcctggactcagctcaggcatcagagacgtccaagcttcagcaactggtagaaaacattgacaagaaggccactgaccccaatgagtgcatcatctgtcaccgggttctcagctgccagagcgccttgaaaatgcactacaggacacacactggggagaggccctttaagtgtaagatctgtggccgggctttcaccacgaaagggaatcttaaaacccactacagtgtccatcgtgctatgcccccgctcagagtccagcattcctgccccatctgccagaagaagttcacgaacgctgtggtcctgcagcagcacatccgaatgcatatgggaggccagatccccaacaccccagtccccgacagctactctgagtccatggagtctgacacaggttcctttgatgagaaaaattttgatgacctagacaacttctctgatgaaaacatggaagactgtcctgagggcagcatccctgatacacctaagtctgcagacgcctcccaagacagcttatcctcttcgcctttgccccttgagatgtcgagcatcgctgctttggaaaatcagatgaagatgatcaatgctggcctggcagagcagctacaggccagcctgaagtcagtggagaatgggtccatcgagggggatgtcctgaccaatgattcatcctcagtgggtggtgacatggaaagccaaagtgctggcagcccagccatctcagagtctacctcttccatgcaggctctgtccccgtccaacagcacgcaggagttccacaagtcacccagcattgaggagaaaccacagagagcggtcccaagcgagtttgccaatggtttgtctcccaccccagtgaatggtggggctttggatttgacatctagtcacgcagagaaaatcatcaaagaagattctttggggatcctcttcccttttagagaccggggtaaatttaaaaacactgcttgtgacatttgtggcaaaacatttgcttgtcagagtgccttggacattcactatagaagtcataccaaagagagaccatttatttgcacagtttgcaatcgtggcttttccacaaagggtaatttgaagcagcacatgttgacacatcagatgcgagatctgccatcccagctctttgagcccagttccaaccttggccccaatcagaactcagcggtgattcccgccaactcgttgtcatctctcatcaagacagaggtcaacggcttcgtgcatgtttctcctcaggacagtaaggacacccccaccagtcacgtcccgtctgggcctctgtcttcctctgccacatccccagttctgctccctgctctgcccaggagaactcccaagcagcactactgcaacacatgtggcaaaaccttctcctcatcgagtgccctgcagattcacgagagaactcacactggagagaaaccctttgcttgcactatttgtggaagagctttcacgactaaaggcaatcttaaggtacacatgggcactcacatgtggaatagcacccctgcacgacggggtcggcggctctctgtggatggccccatgacatttctaggaggcaatcccgtcaagttcccagaaatgttccagaaggatttggcggcaagatcaggaagtggggatccttccagcttctggaatcagtatgcagcagcgctctccaacgggctggcgatgaaggccaacgagatctccgtcattcagaacggtggcatccctccaattcctggaagcctcggcagtgggaacagctcacctattagtgggctgacgggaaacctggagaggctccagaactcagagcccaatgctcccctggccggcctggagaaaatggcaagcagtgagaacggaaccaacttccgcttcacccgcttcgtggaggacagcaaggagatcgtcacgagttaaagcagctcgggctggagacatagcattcattcctgttcagaatgcgacctatggtggcctcctactccttgccccccaccccgccccgccccttccttctgttccccagatctatgaactacaacattatgaagacattcttttgtaccttgttcaactttagagttctaagaaagcttatttattagcgatataaccttgctttgcaaacagaatgcaagcgttaactttggtcttctgtattttggactaaatactaattgactagagtgctgtaaacttgctgtaacatttatggcaattgcaagttgccctgctaggcagttgtaatctggcattaacttattttctatatccagtttaatatgaatctggtgttgatgcaatgcctcagtgatgcattagatctctaataaagtctgtatatacatgtacactttgatcctgctggaaaattttatcagcaaacacattgtctaatctttcaaaacagatttaaggaaaggactgaaagtacagactgaacagtgtggttctttgaaaggtttggttttttaatttttattctaaaattcaaccttttttttgtcgatttaaccatttccattttgaactgctatttgtattgtgctttttacttgagtcgtcttcaatgttaataagtttctgtacagtaataagcacgcagaattc

SEQ ID NO: 22 - protein, 1324 aa, Homo sapiens
MSRRKQAKPQHFQSDPEVASLPRRDGDTEKGQPSRPTKSKDAHVCGRCCAEFFELSDLLLHKKNCTKNQLVLIVNENPGSPPETFSPSPPPDNPDEQMNDTVNKTDQVDCSDLSEHNGLDREESMEVEAPVANKSGSGTSSGSHSSTAPSSSSSSSSSSGGGGSSSTGTSAITTSLPQLGDLTTLGNFSVINSNVIIENLQSTKVAVAQFSQEARCGGASGGKLAVPALMEQLLALQQQQIHQLQLIEQIRHQILLLASQNADLPTSSSPSQGTLRTSANPLSTLSSHLSQQLAAAAGLAQSLASQSASISGVKQLPPIQLPQSSSGNTIIPSNSGSSPNMNILAAAVTTPSSEKVASSAGASHVSNPAVSSSSSPAFAISSLLSPASNPLLPQQASANSVFPSPLPNIGTTAEDLNSLSALAQQRKSKPPNVTAFEAKSTSDEAFFKHKCRFCAKVFGSDSALQIHLRSHTGERPFKCNICGNRFSTKGNLKVHFQRHKEKYPHIQMNPYPVPEHLDNIPTSTGIPYGMSIPPEKPVTSWLDTKPVLPTLTTSVGLPLPPSLPSLIPFIKTEEPAPIPISHSATSPPGSVKSDSGGPESATRNLGGLPEEAEGSTLPPSGGKSEESGMVTNSVPTASSSVLSSPAADCGPAGSATTFTNPLLPLMSEQFKAKFPFGGLLDSAQASETSKLQQLVENIDKKATDPNECIICHRVLSCQSALKMHYRTHTGERPFKCKICGRAFTTKGNLKTHYSVHRAMPPLRVQHSCPICQKKFTNAVVLQQHIRMHMGGQIPNTPVPDSYSESMESDTGSFDEKNFDDLDNFSDENMEDCPEGSIPDTPKSADASQDSLSSSPLPLEMSSIAALENQMKMINAGLAEQLQASLKSVENGSIEGDVLTNDSSSVGGDMESQSAGSPAISESTSSMQALSPSNSTQEFHKSPSIEEKPQRAVPSEFANGLSPTPVNGGALDLTSSHAEKIIKEDSLGILFPFRDRGKFKNTACDICGKTFACQSALDIHYRSHTKERPFICTVCNRGFSTKGNLKQHMLTHQMRDLPSQLFEPSSNLGPNQNSAVIPANSLSSLIKTEVNGFVHVSPQDSKDTPTSHVPSGPLSSSATSPVLLPALPRRTPKQHYCNTCGKTFSSSSALQIHERTHTGEKPFACTICGRAFTTKGNLKVHMGTHMWNSTPARRGRRLSVDGPMTFLGGNPVKFPEMFQKDLAARSGSGDPSSFWNQYAAALSNGLAMKANEISVIQNGGIPPIPGSLGSGNSSPISGLTGNLERLQNSEPNAPLAGLEKMASSENGTNFRFTRFVEDSKEIVTS

SEQ ID NO: 23 - DNA, 4772 bp, Homo sapiens
atgtctcggcgcaagcaggccaagccccagcacctcaagtcggacgaggagctgctgccgcctgacggggctcccgagcacgccgccccgggggaaggtgcggaggacgcagacagcgggcccgagagccgcagcgggggcgaggagaccagcgtgtgcgagaaatgctgcgccgagttcttcaagtgggcggacttcctggagcaccagcggagctgcaccaagctcccgcccgtgctgatcgtgcacgaggacgcgcccgcgccgccccacgaggacttccccgagccttcgcccgccagctcccccagcgagcgcgccgaaagcgaggcggccgaggaggcgggtgcggagggcgcggagggcgaggccaggccggtggagaaggaggccgagcccatggacgcggaacccgcgggggacacgcgcgcgccccggcccccgcctgcggcccctgcacccccaacgcccgcctacggcgcgcccagcaccaacgtgaccctggaggcgctgctgagcaccaaggtggcggtggcgcagttctcgcagggcgcgcgcgcggcaggcggctcgggagcaggtggaggcgtggcagctgcagccgtgcccctgatcctggaacagctcatggccctgcagcagcagcagatccaccagctgcagctcatcgagcagatccgcagccaggtggccctcatgcagcgcccgccgccgcggccctcactcagccccgcggccgccccgagcgcaccgggcccggcccccagccagctgcccgggctggccgcgctcccgctgtcggccggggcccctgccgccgccatcgcgggctcgggccccgccgccccggccgccttcgagggcgcgcagccgctgtcccggcccgagtctggcgccagcacccccggcggccctgcggagcccagcgcgcccgccgcccccagcgccgcccctgcccccgctgcccccgccccggcgccagcgccgcagagcgcagcctcgtcgcagccgcagagcgcatccacgccgcctgccctggccccggggtccctgctgggtgcggcgcccggcctgccaagtccgcttctacctcagacttccgccagcggcgtcatcttccccaacccgctggtcagcatcgcggccacggccaacgctctggacccgctgtccgcgctcatgaagcaccgcaagggcaagccgcccaatgtgtcggtgttcgagcccaaagccagcgccgaggacccgttcttcaagcacaaatgccgcttctgcgccaaggtcttcggcagcgacagcgcgctccagatccacctgcgctcgcacacaggcgagcggcccttcaagtgcaacatctgcgggaaccgcttctccaccaaaggcaacctgaaggtgcacttccagaggcacaaggagaagtacccccacatccagatgaacccttacccggtccccgagtacctggacaacgtgcccacctgctcgggcatcccctacggcatgtcgctgccccccgagaagcccgtgaccacctggctggacagcaagcccgtgctgcccaccgtgcccacgtccgtggggctgcaactgccgcccactgtccctggcgcgcacggctacgccgactctcccagcgccaccccagccagccgctccccgcagaggccctcgcccgcctccagcgagtgcgcctccttgtccccaggcctcaaccacgtggagtccggcgtgtcggccaccgccgagtccccacagtcgctcctcggcgggccgcccgtcactaaagccgagcccgtcagcctgccctgcaccaacgccagggccggggacgctcccgtgggcgcgcaggctagcgctgcacccacatcggtggacggcgcacccacgagcctcggcagccccgggctgcccgccgtctccgagcagttcaaggcccagtttccgttcggggggctgctagactcgatgcaaacgtcggaaacctcgaagctgcagcagctggtggagaacatcgacaagaagatgacggacccgaaccagtgcgtcatctgccaccgggtgctgagctgccagagcgcgctgaagatgcactaccggacgcacacgggggagcggccgttcaagtgcaagatctgcggccgcgccttcaccaccaagggcaacctcaagacgcacttcggcgtgcaccgtgcaaagccgcccctgcgcgtgcagcactcctgccccatctgccagaagaagttcaccaacgccgtggtcctgcagcagcacatccgcatgcacatgggcggccagatccccaacacgccgctgccggagggcttccaggatgccatggactccgagctggcctacgacgacaagaacgcggagaccctgagcagctacgatgacgacatggacgagaactccatggaggacgacgctgagctgaaggacgcggccaccgacccggccaagccactcctgtcctatgcggggtcctgcccgccctccccgccctcggtcatctccagcattgccgccctggagaaccagatgaagatgatcgactcggtcatgagctgccagcagctgaccggcctcaagtccgtggagaacgggtccggggagagtgaccgcctgagcaacgactcctcgtcggccgtgggcgacctggagagccgcagcgcgggcagccccgccctgtccgagtcctcgtcctcgcaggccctgtcgccggcccccagcaatggtgagagcttccgctccaagtccccgggcctgggcgccccggaggagccccaggaaatcccgctcaagaccgagaggccggacagcccagccgccgccccgggcagcggaggcgcccctggccgcgcgggcatcaaggaggaggcgcccttcagcctgctgttcctgagcagggagcggggtaagtgtcccagcactgtgtgtggtgtctgtggcaagccttttgcttgcaagagcgcgttggaaatccactaccgcagccatactaaggagcggccattcgtctgcgcgctctgcaggcgagggtgctccactatgggtaatttaaaacagcacttactgacacacagattgaaagagctgccttctcagttatttgaccccaactttgctctaggtcccagccaaagcactcctagcctgatctccagcgccgcacccaccatgatcaaaatggaagtgaacggtcacggcaaggccatggcgctgggcgagggtcccccgctgcccgcgggcgtccaggtccccgccgggcctcagacagtgatgggcccgggcctggcgcccatgctggcccccccaccgcgccggacgcccaagcagcacaactgccagtcgtgcgggaagaccttctcctcggccagcgccctgcagatccatgagcgcacgcacaccggcgagaagccgttcggctgcaccatctgcggccgggccttcaccactaagggcaacctcaaggtgcacatggggacacacatgtggaataacgcccccgcgagacgcggccgccgcctgtctgtggagaaccccatggctctcctagggggtgatgccctgaagttctctgaaatgttccagaaggacctggcagctcgggcaatgaacgtcgaccccagtttttggaaccagtatgctgcagccatcactaacgggctcgccatgaagaacaacgagatctccgtcatccagaacggcggcatcccccagctccccgtgagtcttgggggcagcgccctcccccctctgggcagcatggccagtgggatggacaaagcacgcactggcagtagcccacccatcgtcagcttggacaaagcgagctcagaaacagcagccagccgcccattcacgcggtttatcgaggataacaaggagattggtatcaactagccagtgactcgctcatctgccctgcccaggcccacgttttgaagttggagcatcaggcctccgacctttcttgcctcggttctcattacactttcacccatagcagaaaacactttgtgcggctgccgagaggtggtcttgtaagcgctgcatggcgctcccttcaacagcaagcctgactgttctcgagaactctgcaatcttttaaataagcttccttcaaaaaaaaaagtgcttggaaaaccgccttaggaacagaaagagctcagaccatgtccacttcctttctcctgaaacctaataatctctccgagggagaaaggggttctctgcggtattccagtgaaactcatttgatggtttcttttgaattagttagacacttgaacggtgttttttagaactcttcatgttaaagacgtggtttagtactcccaatgctgtgtatcatgacactatcttcgtctgtagtatttatgatgttaagataatgcgggtaacagacaatataatagccccgaccttaaacgaagcttttgtactgcagaatacatctggctgtgtgattttttttttaagcaagatttgttttactataaataagtggattatttcaatgcaggcaaaattgtgaagttctgttgggaaagatagcatgcttttcgtgtgcaagtacctgtcagtaataagccttttttttttttttttaatttaaatgtttgtagctgctatgtggacagttgttttctagtgtggtctgtagcccaataactggggaacgagttacagacaaacatcaccgtaaatgactcacaacattataaacagttgtgagaaaatatttcacattatcaaagctgtacaataaa

SEQ ID NO: 24 - protein, 1300 aa, Homo sapiens
MSRRKQAKPQHLKSDEELLPPDGAPEHAAPGEGAEDADSGPESRSGGEETSVCEKCCAEFFKWADFLEHQRSCTKLPPVLIVHEDAPAPPHEDFPEPSPASSPSERAESEAAEEAGAEGAEGEARPVEKEAEPMDAEPAGDTRAPRPPPAAPAPPTPAYGAPSTNVTLEALLSTKVAVAQFSQGARAAGGSGAGGGVAAAAVPLILEQLMALQQQQIHQLQLIEQIRSQVALMQRPPPRPSLSPAAAPSAPGPAPSQLPGLAALPLSAGAPAAAIAGSGPAAPAAFEGAQPLSRPESGASTPGGPAEPSAPAAPSAAPAPAAPAPAPAPQSAASSQPQSASTPPALAPGSLLGAAPGLPSPLLPQTSASGVIFPNPLVSIAATANALDPLSALMKHRKGKPPNVSVFEPKASAEDPFFKHKCRFCAKVFGSDSALQIHLRSHTGERPFKCNICGNRFSTKGNLKVHFQRHKEKYPHIQMNPYPVPEYLDNVPTCSGIPYGMSLPPEKPVTTWLDSKPVLPTVPTSVGLQLPPTVPGAHGYADSPSATPASRSPQRPSPASSECASLSPGLNHVESGVSATAESPQSLLGGPPVTKAEPVSLPCTNARAGDAPVGAQASAAPTSVDGAPTSLGSPGLPAVSEQFKAQFPFGGLLDSMQTSETSKLQQLVENIDKKMTDPNQCVICHRVLSCQSALKMHYRTHTGERPFKCKICGRAFTTKGNLKTHFGVHRAKPPLRVQHSCPICQKKFTNAVVLQQHIRMHMGGQIPNTPLPEGFQDAMDSELAYDDKNAETLSSYDDDMDENSMEDDAELKDAATDPAKPLLSYAGSCPPSPPSVISSIAALENQMKMIDSVMSCQQLTGLKSVENGSGESDRLSNDSSSAVGDLESRSAGSPALSESSSSQALSPAPSNGESFRSKSPGLGAPEEPQEIPLKTERPDSPAAAPGSGGAPGRAGIKEEAPFSLLFLSRERGKCPSTVCGVCGKPFACKSALEIHYRSHTKERPFVCALCRRGCSTMGNLKQHLLTHRLKELPSQLFDPNFALGPSQSTPSLISSAAPTMIKMEVNGHGKAMALGEGPPLPAGVQVPAGPQTVMGPGLAPMLAPPPRRTPKQHNCQSCGKTFSSASALQIHERTHTGEKPFGCTICGRAFTTKGNLKVHMGTHMWNNAPARRGRRLSVENPMALLGGDALKFSEMFQKDLAARAMNVDPSFWNQYAAAITNGLAMKNNEISVIQNGGIPQLPVSLGGSALPPLGSMASGMDKARTGSSPPIVSLDKASSETAASRPFTRFIEDNKEIGIN

SEQ ID NO: 25 - DNA, 116368 bp, Homo sapiens
gatcttaccttgatgttgatggctgttcactgatcagggtggtggttgctgaaggttgggatggctatgccaatttctttaaaataagacaacaatgatgttggctatattgattctttatctgagatttctttgtaacttgtgatactgtctgaaaatattttacccacaacaggccgggcgcggtggctcatgcctgtaatcccagcactttgggaggccgaggcgggtggatcacaaggtcaggagatcgagaccatcctggctaacacggtgaaatcccatctctactaaaaatacaaaaaaaattacttgggcgtggtggcagacgcctgtagtcccagctacttgggaagctgaggcagaagaaccacttgaacatgggaggcagaggttgcagtgagctgagattgcgccactgcactccagcctgggtgacagagcgagactctgtctcaatctcaaaaaatactttctttgctcatctgtaagaagcagttctttattcattcaagttttctcctaagattgccaccattcagtcacatcttcaagcttcactgctatttccagctctcttgctatctccatcatatctgcagttacttcctccactgaattttgttgtttgtaaagacaggatttcactgtgttgactgggttggtcttgaactcctggctctcaagccatcctcctcggcctcccaaagtgctgggattacaggcataagccacggtgcctggctctttcactgaagtcttgaacccatcaaagtcatccatgagggttggaagccacttcttccaaactcctgtttatgttgatattttgaccttctcccatgaattacgaatgtttttaacaacatctagaatggtgaatcctttccaggttttcactttactttgcccagacccatcagagaaataacaaactatggcagcttacaaaatgtagtatttctgaaataataagacctggaagttgaaactcttagaggcatttgaaccagagccactccatctcgaacaggagctggggaaaataaagctgagacctactgggctgcattcccaggaggttaaggcattcttagtcacaggatgagataggaggtcagcacaagatacaggtcataaagaccttgctgataaaacaagttgcagtaaagaagccagctaaaacccaccaaaaccaagatggcgacaagagtgacctctggttgtcctcactgctacactcccaccagcgccatgacagtttacaaatgccatggcaacatcaggaagttaccctgtatggtctaaaaagggaaggcatgaacaatccaccccttgtttagcatatcatcaagaaataaccataaaaatgggcaaccagcaactctcagggctgctctgtctatggactaaccattctttcattcctctgctttcctaataaacttgctttcactttatggactcatcctgaattctttcttacgtgagatccaagaatcctctcttagggtctggatccagactccttaccagtaacaaaaccactccttgatccagactacaaaatgaatgttgtgttaacaggcatgaaaaacagtattaatctcagtattaatctccttgtacatttccatcagagctcttgacagatgcattagatgcattgtttttttgttttttgagacgggagtcttcctctgtcgcccaggctggagtgcagtggcgtgatctcggctcactgcaacctccgcctcccaggttcaagcaattctcctgcctcagcctcttgagtagctaggattacaggcgcccatgaccacgcccagctaatttttgtgtttttagtagagacagggtttcaccatgttggtcaggctggtctcgaacccctgaccttgtgatccacccgcctcggcctcccaaagtgctgggattacagatgtgagccactgcgcccggcctagatgcattgttaatgagcagccataatttttgaaagaaatctttttttttttttttctgtgcagtaggtctcaacagtgggcttaaaatattcagtaaatcatgtgataaacagatatgctaaactgggcgcggtggctcacacttgtaatcccaaaactttggaaggccgaggcgggtggatcacttgaggtcaggagtttgagaccagcctgaccaatatggtgaaaccccatctctaccaaaaatacaaaaggcagcaggtgcctgtaatcccagctactcaggaggctgaggtaagagaatcacttgaacccaggagacagaggttgcagtgaactgagattgtgccactgcactccagcctgagtgacacagagactccatctcaaaacaaaaatgagatatgccatcatccaggctttgtcattccatttccagagcacatgcagagtagaaatagcatcattcttaaaggccctaagattttcagaatggtaaatgagcattgctttagcttaatgtcaccacctgcattagcccctaacaggagtcaccttgtcctttgaagcaaggtgtttttttcctttctagctatgaaagtcctagatggcatcttcttccaatagaagactgtcttgtctactttgaaaatatgttgtttagtgtagctactgtagttattgtagctagatcttctggataacttgctgtagcttctacatcagcatttactgccttaccttgcttttttttttttgttttctttgagacagagtttcactctttttgcccaggctggagtgcgatggcacaatcttggttcaccacaacctccacctcccatgttcaagtgattctcctgcctcagcctcccgagtagctgggattacaggcatgcacctccaagcccagctacttttgtatttttagtagaaatggatttttctccatgattgtcaggctggtctcgaattcccaacctcaggtgttctgcccccctcagcctcccaaagtgtgaggattacaggtgtgagccaccacgcccagctgcatttttatattaggaagatgtcttcttgggcctggtgcagtggctcatgcctgtaatctcagcactttgggaggctgaggcgggtggatcatctgaggttggaagttcgagaccatcctgaccaacatggtgaaactccgtctctactaaaaatacaaaaattaactgggtgtggtggtgcgcacctgtaatcccagctactcgggaggctgaggcaggagaatcacttcaatgcgggaggcagacgttaaaaaattaagcagtacttaattttttttttttgagacagagtcttgctctgtcgcctaggctggagtgcagtggcgtgatctcggttcactgcaatctctgcctcccaggttcaagtgattctcctgtctcagtctcctcagtagctgggattacaggcgcaatgccaccatggccagctacttttttgcattttagtagggacagggtttcactgtgttgcccaggctggtctcgaactcctgagctcaggtaatttgtccactttggtctcccaaagagctaggattacaggcgtgagccactgcgcctggcctataaacaaatttaaacagagaaaaggtacagtaaaaattagtattacaatcttatgggaccatggttgtatatgcggtctctaactgacaaaacaaagttatggccacactatgcgggcataatctacttgcataggtacaatcacaatctaccctttagaactgacttcaaagattaaggctgagaggaatgatgcacacatatacacactacacactgtattatggaaaacagcacacacacacaaagacaatgataatcttttccccagctatcccaagcaggaatttaccctggaagtgaactcaagtataagaaaagaaataccacaaaggcaatcaaaaacaaaataaaaaagttcacggggccaggcacagtggctcatgcctgtaatcccagcactctaggaggccaaggccagtggatcattcgaggccaacctggggaacatgatgagaccatctctgcaaaaaaaaaattagctaagaatgatggtgtgtgcctgctactccacaggctgaggtggaaggatcacttgagtccaggagctcaaggccagtcagctgtgatcatgccactacactgcagcctgagtgatagggacgtttatgggtaaacaccagcagtgttgtcaatagatgactagacatcattgttacgtaattgcagttattttaaaaggattagctggaattgggttttaccttggaaagatgtctgaaatatgtgagaggggagaaaaaaggctgggcaaggtggctcacgcctgtaatcccagcactttgggaggccgaggcagatggatcatttgaagtcaggagttcaagaccagcctggccaacatgctgaaacctcatctctactcaaaatacaaaaattagccaggtgtggtggtgggcacctgtaatcccaagtaaggaggctgaggcaggagaatcgcttgaacctgggaggcggaggatacagtaagccaagatcgagccactgcactcccttgagcctgggaggtcaaggctgcagtaagctgagatcataccattgcactccagcctggatgaaagagtgagaccctgtcttaaaaaaaagaaaaaaggccgggtacggtggctcactcctgtaatcccagaactttgggagaccgaggtgggcgggtcacctgaggtcgggagtttgagaccagcctgaccaacatagtgaaaccccatctctgttaaaaatacaaaattagccgagtgtggtggcgcatgcctgtaatcccagctactccggaggccgaggcaggagaattgcttgaacctgggaggcgaaggttatagtgagccgagaccatgccattgctctccagcctaggaaacaagagggaaactgtcaaaaaaaaaaaaaaagtgaggcaagtatggacatagccaggtgtgcatctttttgacgctaggaacaagagtgtcacaaggcagctgaaagtgattgtcaagtaaagattctcaggtatagagcagaaagtaccagaaaaatttatatacacctgggtgagaaaaaaaaacattcaaattttattttccaacagacagacagcatcagcaggtacaactacaggggtttctccgtagatcatacattcacaaggcattattagctcaacagtgagaaagccactggtgtgttttctgtaacaatatccacttcacagtgtaaacaggtactattatcgtgttcacttacaattccagaaggaaaggcacaacttggcaaaaaaaaaaaaaaaaaaaaaaaaggggggcggaatcctaaagtcaggtgcaacgatgaagagacaacactttggctaatcgtcttggatgcatatttccgtcaggaccttccacatagaggggaaagacttttctcccagaaattagagtttttttctttctttcttgttaaaccaagagcaatgtttcgtttgctcaatatcacatttacaaaggaaactacaaaaaaaaaaggcatcacaaaatcatcttgaacgttcacctcttcccaccaatacatcaactcttaggctttagacagggcctgggaatacttcagcggtcttaaaataggaaaatagacactatggctacaaaaaataaaaaataaatgaggtagataaattaaagctttcacacccaggacgtgcctgttctaacttcgtagccttcatgaaatattcagcaaggaaacaaaacaaaacaaaaaatcgtcataattacttggcataagtcgcaccaaggaaattcaacaaaagcaatcagtatgtgaacctgtgatgggaaacacgcccttccacgagtttcttctcgagcatgaaaacccagttcttcaatagagtaggcactggcaaactaagtcacctgagattccccgctcaggtgcccacctcttctgggatttgggaagaggaaggcatccaagaggtgtggggaaaacattggcactctcggggctccaactgaactgtatttaaataagcagcaacacacacattgactccctaaggactaacgtaagtccatttgagggcctcctacttaaggttctaaggttgaagcagttttataattctacaaccctagttttttgttgttccttttttaagtacattcaaaaaaaaacaataagatggggacagggttggggcatataaaacaggtccctaaagagatgacagaccttgtccttttacccgtgtccagttaccagtaagtatacagtacccatctcccctcaatgaggctgtgtagttgtcttttccactgttgttcagtaagttccaacgattctaccttgaaataggtaacagtctttggaaaaagtcactctaggttgtacaaaggttctatgtatacgtctgttacaagtaacaaagctactttgcaagacccgcttctttccaaaattagaaaaaaaaaaaaaaagtgccaggcgtggtagctcatgcctgtaatcccagcactttgggaggccaaggcgggtggatcacgaggtcagcagttcgagaccagcctgaccaacatggtgaaacccagtctctactcaaaatacaaaattagccgggcgtggtggcacctgcctgtaatcccagctactctggaggctaaggcaggagaatcacttgaacccagaatggggaggttgcagtgagccgagatcatgccactgcactccagcctgggtgatagagcgagactccatctcaaaaaaaaagagtctgtatttgttttggtatgcattttttttttattttttcaactttttgcaaagcagcatagcaacaatcgtgattgtagcacttgcctgaggttgtggtcacaaccaacgtagtaaacatcatttgcatatcagtaagaaaaagaaaacaggaggagatgagttcttacaaaacaaagcagattctagagatttcactgtgtctgcattgctccttccacgcaagttctcccttagctgaccgcaatcttgttttcttccaggaagtgaggaaactggtgtttgggaacgccgtcagtagcacttggtttttccacatctgcactgatacccgactgggagccatccatcttggagacagtggcgttattcacaacggaggtggcccccaaggaaaccgggagggtaggaacccccccactctggatcacagagatctcattggtcttcacggccagaccgccattgagcatgctggtgtactggttccacacaacagggtccacattcactgaaggggccaggatttccttgggaaagatttctgagactctttttccgtccgtacctaacagagccatggtgttctcgatggccaacttccttccacggcgggctgagttattgttcgccccgtgtgtcatgtagtgaacctatgggaacaggacagaaaggtttttaccaaaatagagatttgaagctcactggcaagccaagaatcaatctgcatatggatttcattctacccagtgttttaacatatagatgatccttgacttacagcggggttatgcccagataaactgattgaaagtagaaaatattcttagtctaaaatgcatttaatacccctaactagccaaacatcatagctcagtttagcctacctcaaacacactcagaacatttgcattagcttacagttaggcaaaatcatctaacgcaaagcctattttattttatacatgagcactgaaacagctcatgtaggccaggcacggtggctcacgcctgtaatcccagcactttgggaagctgaggtgggcagatcacttgaggtcaggagttcaagaccagcccggccaacatgatgaaaccctgtctactaaaaatacaaaaagtagccaggcatggtggcacatgcctgtaatcccagctgctcgcgaggctgaggcacgagaatcgcttgaacctgggaggcggaggttgcagtgagccaagatcatgccactgcactccagcctgggtgacaaagcaagactccatctcaaaaaaaaataaaaaataaaaaattagctcatagctcatgtgattgagcatgtactgaaagtgagaaacagaatggttgtatgggtgctcaaagtctgatctctactgaatgcctatcacttttgtatcatcgtaaatcaaaccatcttaagtcagggactgtctgcactttatatttagaaaatcagatttcttatatcacctaacaaacattcctccccagagcaaccaagatggggcttatagtctccaactgtctctgctccaccaatcatgggtgtacctatgtgaaaacctactatatcctgggcattgatccaagtgccagggttaacatttttttttgagtccttttttttttgagacggagtctcgctctgtcgcccaggctggagtgcagtggcgcaatctcggctcgctgcaacctccgcctcccgggttcaagcgattctcctgcctcagcctcctgaatagctgggactacaggcacgcgccaccatgcccggctaatttttgtatttttagtagagacggggtttcaccatgttggccaggatggtcttgatctcgtgacctcgtggtccgcctgcctcggcctcccaaagtgctggaattacaggcgtgagccaccacgtccggccttttttttgagtcttaatctgtcacccaggctacagtgcagtggcacagctcactgcagcctcaacctccaaggctcaagcaacccacccacctcagcttcctgagtacctgggactacaggtgcacaccaccatgcccagctaattgtttttttttttttttgagacagagtctcgctctgtcgcccaggctggagtacggtggaatgatcttggctcaatgcaagctccacctcccgggttcacaccattctcctgcctcagcctcccgagtagctgggactacaggcgcccaccgccaccacgcccggctaattttttctattttgtttttagtagagacagggtttcatcgtgttagccaggatggtcaggatctcctgaccttgtgatccgcccacctcggcctcccaaagtgctgggattacaggcgtgagccactgcacctggccggctaatttatttttatttgtagagactggatctccttatgttgctcaagctggtctcgaactcctggcctcaagcaatcctcccaccttgtcctcccaaagtgctgggattacaggcatgagccactgcacccagcctagagttaacatttttaaaatgcacaaacctacgcaggtaataaaactgtagagtgcacaacatgcacacctgagtacaagtaaaatgggaaaatctgaatgttgattgtaacaatgtcaatatcttgattatgctacaatgttgatactaccgttttgcaaagtgctaccaatggggaaaactgggcaaagcatacaaggatgtctctgtattacttttcaaaagtccagtgaaaatctatacttatttaagtaaaaatttcagttaaaaacaatttgtgggcagggcacagtggctcacgcctataatcccattactttgggaggctgaggcaggcagatcacctgaggtcaggagtttgagacgagcctggccaacatggtgaaaccccatctctactaaaaatacaaacagtagccgggtatggtggcacacgcctgaagtcccagctactcaggaggctgaggcaggagaatcgcttgaacccaggaggcggaggttgcagtgagtcaagatggcaccactgcactccagcctgggcaacaagagcaaaactgtctcagaaacaaacaaacaaacaaaacaaaccaaaacaaaacaaattttggggcaggacgcagtggctcacacctacaatccaggactttgggaggtcagagcaggtggatcacctgaggtctggagttcgagatcagcgtggccaacatcgtgaaacctcgactctactaaaaatatacaaaaattagccgggcatgatggcgggcgcctgtaattccagccactcaagaggctgaggcaggagaattgcttgaacccgggaggcagaggttgcaatgagccgagattgcaccgttgcactccagcctgggtgacagagcaaaacgaccattccctgatgcagctgacatctaaatatagagaacatcaatgtctctaataattgctacagagaaaaatgtatctggctgggaaagctacagaaggccatggtgggaataggatgctatttaatctaaggtggtaggcaaggcttctgctgaagaagggaccttactagcacctggctgctttctttaattatttttcctgtctggctttcaagttttgaaatccttttcagcttcttttttttttatttatatgaatttaatttttgtagagatggcatcttgctatgttgcccaggctggtcttcaactcctggggtcaagtgatcctcgcaccaccacctcccaaagtgctgagagtacaggcatgagccactgcgtccggcccttttcagcttcttaagagtttaaaactcttaagagacagggtcttactctcttgcccaggctggagtgcagtggtgtgatcacagctcacagaagcctcgacctcccaggttcaagtgatcctcccacttcagcctcccaagtagctgggcctacaggcgcatgccaccacgcccatctaatttttatttttattttgtagtgacagggtctccttatgttacccaggctggtctcaaactcctgggctcaagcaatccacctgccttggcctctcaaagtactcagattatgggtgctgagccactgcacccaggtagtatttctaatttttaaaattaaaatgtaaacattttaatttttttaagagacaaggtctcactttgttttctaagctggagtgcagtggcatgatcacagatcactgcagccttgatctctcaggctcaagtggtcctcccacttcagcttcccaattggcggggacccaagcatgcaacaccatgcccaactaatttgtgtgtgtgtgtgtgtgtgtgtgtgtgtgtgtgtaaagacgttgtctccctatgttgccaggctggtctcaaactcctggactcagctgggtgcagtggctcatgcctgtaatcccagcactttgggaggccgaggcaggcggatcacgagatcaggagatcgagaccatcctggctaacgcggtgaaaccccacctctactaaaaatacaaaaaaattagccggccatggtggcggacgcctgtagtcccagctactcaggaggctgaggcaggagaatggcatgaacccgggaggaggagcttgcagtgagcttgagcttgagatggcgccactgcactccagtctgggcgacagagtgagactccgtctcaaaaataaataaataaataaacaaactcctggactcaagcaatcctcccacctcggcctcccaaaggaggaatggaaggatggaagaactcatcacggcttgtgccaataagaagacacctggtgcctagcccccatcctgctgaaagcccacacaaacccacctttaagttgcctttggtggtaaaagctcgcccacaaatgttgcacacaaaaggcttctctccagtgtgagtccgctcgtggatctgaagagcgctagcagacgagaagttcttcccacaccgtgtgcagccatgttgcttggcctgtcggcgtggctgggctgctaacaaaggggtcatccctggggacaatgtcgagggtcccacaaatgtgccaggaacttcaaccttgacataggtcggcggggctcggataaacgtggaagggagactgctccgacctagtacacagaggggaaaaaagccagacctttatcatccaaccttcattctttctcttcaaagcaaaagagatctttaaaaataactgcctgctagtttgagagtctggagctggctttgtaagtaattaacaccttcccctttgaattgtttatctgcacaaagaataaagcttcaaatgcttgggaaaaggtatcactttgggttttttttttttaaaggcatgggcaacatataaacacatatgtaatttaagttcaaatttctgcataagaaactacctttaaagcatatgcccagacaccatctttctttgattctttgatacagcttttcccttattttaccattaatgacatcaacaaacctctcaatagatatgtgcatttaaaatgggttcatctttccctgcttcaagagtggctaatttgtgaacattacatcaggcagtggggcctcagagtcagggaaccacggcttatttcagtggtcaaaaagcagtggccacttcctcaaacatactcttaagacacccatatacagaatcatagtgtggagaaacaaaaagaaaaaaactataacaaaatagaacgactacccacccctatgtcccaaggggctcaaagacaaaaataaaaaccaacaaaaaaggtatgctgtccatacccattttgcattaaccatcataaattaaaagcatgttcgttactaggatagaggcagttactattgccctctacccagaagtaaagaaaagctaattgccctatagaaggggctgcttcaagtcatactctccgtttgtaaagttcaacccaggctcctttttgatgaccttgagcccaaaatggacataagttaaatccaaagctctatcaccttccatctcagtgcggctgttctcggagctctctgctttgctcccagcatcgggagactttgacttgatgctttcggcttgactattggccggggagagtgcctggaaggatgtggtttccaggatatctgggcttcggctctgatactcctggtctcccatcagcgaggatgagtcgttggtcaagccatcgctctccacggaaccgttttctctgctgccctggcgctgcaggttaaaaggggcaggacccactttccctggggcatctaaggaagccatcatggcaaaccctagcgtgggtgatgccgagtggatgctgggaagaggcgtggggaccttggaggagctgctgggagcctcctgggagctgacttcctctacatcgatgctttcgatgacatcatcatggcagatagcgccggtgctgccgttctcacccacggtcattggctcagaacccgtaaagtcacagggattctctggcaggggcgtgttgggaatctgaccgcccatgtgcatccgaatatgttgctgcagcatcacggcattagtgaacttcttctggcagatggggcacgaatgctgcgtcttaatggatgtgttggttcggtgaaccccaaggtgtgtcttcaggttacctttggtagaaaaggctcggccacagatcttacactggaacggtctctccccggtgtgggtgcgataatgcatcttgagggagctctgacagcttaagactcggtggcaaatgagacattcgttgggatcagtggtggccttgtcaatgttctccaccaactgctgcaatttcagggtctctgaccctggctcaggggtcccactcccttggaagccaccagcccttggggaattatagtttggtcccaccccagggagtgtgggtccaccctcactttctggagaaggcccaggctgcaggtcaccgggcaaggagccacccgtgaggtccttgggattagtccccgaagaaagattctgaggtagccctacagaggtggttacaaggacaggtttgctgtctaaagaaagactcggttcatctatggggtcaggtacagagagtgcataggggatgccattgccggccgccactttgtcctggaactcggcaaacagctgggggtttgccttcacctggggatgtcggtgaaagtgcaccttgaggttgcccttggtggtgaagcgatgaccacagacagagcacacgaagggtctctctccagtgtgggagcggaggtggatctgcaaggagctatcagtcccaaaaaccttgctacagtacttacacttgtgcttgtagagggccgcctcgtctttgggtttgacatccaccgcggagatgttcggtggcttccccttccctttcttggatgtgtctagcgccacagtggagaaagggctctggaagagcaccgagcccggggcctgaggaagcaaagcgctcgggaggcgggacatgacgttcgggagcacccgggtcccatccggcttcagagtgaagggtgccagccctggggacagggagctggtggcagaagggatgttggcgtgaggtagcttggcttgtttcaaggcatccagagacagaccttggcttccagctttctggctgagcaaagccacagctgcagaaacctgctgagacatgtggctgcccaaggtcttcagagtgtcggcccctgccccgcttgagtggagggcgtgggaggcccacatgttcacctggatgcggatctgctcggtgagctggatctgctgtagctgctgctgctgcagacacaagatctgctcgaggacccacgggatgctgttggcaccaggcacgggggcagggagtgcatccgcgctccgctgattcaccgccaccttggtgccccgtagtgcctgcaaggtcacattagtgttggccactttgcctttggctaaatagcttatgtcctggggggtgggtggcagggctgtctctgtctttaggtacaccacagactccgcatccggcttctccttcatgtcctctgagctgccgccattctccctgtgacagtccttactgccgggactggtgggctggtggctcagtacagctccggagaagtcttctgaaggcacaggcccctcgctgtcattcatgatgaggacaggtggatttttagtgcaatttttcttatgttccaggaactcagagatgctgaagaactccgcacagcatttctcacagacgtgcgtctcctcccgacgaagccgctttactgtggcttcatcctcactcgccacctcgtcattccctgggtggttcactggagcacctgtaacaagacagaaaaagctaaggactctgcccaagtaaaagatgtggggggagccgggcaccgtcgctcacatctataatcctagcactttgggaggctgaggtgggcagatcacctgaggttgggaattcaagaccagtctggccaacatggtgagaccctgtcactactaaaaacacaaaaattagctgggcatggtggtgggctcctgtggtcccagctactcgggaggctgaggctcaagaattgcttgaacccaggaggcagaggttgcagtgagctgagatcccgccattgcactccagcctgagcgacagagcgagactccatctcaaaaaaaaaaaaaaaaaaaaaggcaaaaaggctgatccctgaatttcttttgtaactggggcttacttactttagggttagtgccagcctacacattgcaactccgaacttgggactgtgttttagccagatgctggggtccttcacatgatgcctggaagtctctcgaagctacttcattgttctcacctccctgctgtcctctcaggtataccccatcatcaagatatcaatggagcttggggccacaggacaaagacaggcatatctcagacccagcagctaagcctataaatctaaaccacacaagacattcacacgagcatacaatgatctaggttttcttgttttttcagaaatgttcacagcagcaaagaagtaaattttggcccacccacccatagtgtgagaaatatgcagaatttctgtatgccacagaaattaagtgaaagaaacgggttgcgcaacaatatgtacaacatgaccattttgcttaaaacaaaacctgtgtttgaaatgtacaaatgtggctgggtgcagtggctcgcaccagtcatcccagcactttgaggccacttttgagaggccgggagtttgaaaccagcaacacagtgacaccccgtctctataaaaaaaataagaagataaaaatagctgagcatggcagcgtccatgtatagttcaagcaactccagaggctgaggcaggagaaccgcttgagcctgggaagtcaagccactccagcctgggtgacagagtgagaccctgtctggaaaaaaaaagtacaaatgcacaaggaaaaggccaggaagggctggagtgcagtggcccaatcttggctcactgtaacctccgcctcctggattcaagctattctcatgcctcagtctcccgagtagctggctaatttttgtatttttagtagagatggggtttcaccatgttggccaggctggtctcaaactcctgggtgcaagtgatccgtctgcctcagcctcccaaagtgctcggattacaggcatgagccaccgtgcccagccaaagccacggacttttgacagcacagtccacccacgggtttccaggaggggctgatcttgaagcatggccttaacaaactgatttaatctttacttatttcagactgggtcacaatatgaagtcttgcaaggtatgtgtaggctgggggtgggtcaggggaagagacaacagccatgggcaggtctggcctgccagagttaagaaagctaggcagggccaacctagccttgcctatcaataacaggtagttattgataggcaagtcggcacacccctaagaaagcctactgggcacgcgtaatcgcgagatgacaacctcgtgattagatgattaggtagatgcctggatttactgattcaaccaggactgctgagaacaatgccttacaataaaacaatgtaatgcctctcgttccacaatactaacctcctactgcgtgcaaagcaggggttggggacccaatggtgaaaagatgagccccattatcttcctataattcagattttgtaccagcagatgcccatactgtaatcattcaatcctattaaatatcagctccattaaaggctgggcgtgatggctcacacctgtgatcccagcactttgggaggctgagagttcgaaaccagcctgaccaatatggtgaaactccatctctactaaaactataaacttagcccagcgtggtggcgcatgcctaatcccagctactcaggaggctgaggcaggagaatcacttgaacccaggaggcggaggttgcagtgaactgagattgtgccattgtactccagcctgggcaacaaaagcgaaactccatctcaaaaaataaataaataaataatgaaaaaataaagatgtgaaaaacagagactctgagaaataatgtcactcgtgcaacataacaaaagttattgacagataacttttgagcacttactttgtatgtgccaggtatgattctaaacaatttgttatattaacaactctgaaatagaaactattaccctatttgacagaaggggaaactgaggcacagaggacatgaacttgcccaagctaacatgttaagtaaactgcagagccagtattaaaccccagacatgcagttcccacaccctctattggaacccttcatttgtacccctctgatcatgtttcttctgtaaagattgcaactttctttcctctcacagagatgttaagatgggacaggaaagcctgggaggttgccttggttaggaaggggtgggagtcgaccacccattcctgtctcatttctaggacttccagaagttgctctgaaagtgttaagatgcagtgcgatgctgctacttccaggtagagttctagtagcaggtaccagcacaaactctcctttcttaaggccccaatactgctgcgaatctgcctcacttcagtgatccagataaaaatgttcggccgggcacggtggctcacaaggtcaggagatcaagaccatcctggctaacacggtgaaactccatctctactaacaatacaaaaaattagccgagcacggtggtgggcgcctgtagtcccagctactggggaggctgaggcaggagaatggcgtgaacccgggaggcagaggttgcagtgagccgagacagtgccactgcactccagcctgggcgacagagcgagactccgtgtcaaaaaaacaaaaacaaaaacagataaaaatgttcgtcaattcccagaaccctggcaactatttatctgggaaccgccaactctcagataaatagtatctatctgtatgattttccaagactggaaaccagcactagtgggagtgctaggtggcacaatctttctagaaaagcaattttgacaattatgtgtcaagaatgttaagaagtgaaactttttaaaacagtttctcttgttagggaaggaaatacacgctgctggtacgagtgtatagacagatacagctctttcagggagtcatctggcaacatcttcattttagtacaattttcctttgacccagcaattcctgctttaggaatttcctcaatgtacttgcagcatttctcatgttaacaaacagcaacaaaatggatgcaccttggtccatcagtaagattttggttaaataagttagggtgcagaaaaggcgaacgctttatagcctccagaaataaagaagaccaaggctgggcacagtggctcacacctgtaatcccagcactttgggaggccgaggtgggtggatcacctgggttcaggagtttgagaccagcctggccaacatggtgaaactctgtctctactaaaaatagaaaaattagccaggtgtgatggtgtgcgcatcgctggaatcccagctactcgggaggctgaggtgggagaattgcttgaacccgggaggtggaggttgcagtgagccgagattgcgccactgcactccagcctgggcaacagaacaagactgtctcaaaaaaaaaaaaaaaaagaaagaaagaaagaaaagaaaagaaaaaaaaaaccctcaataaagcccatgcaaaaacattaaccactccatactactttttagaggccaaagctaaccaccaccaacctctccaagcaagaacaaaaacatacttcatactcttacaccttaaggcaagcttgtccaaaccaggcccaggccagctttgaattcagcccaacacaaattcataaactttcttaaaacgtgagatagttttacaatttgttttaagctcatcagctatcattactgtccgtgtcttttatgtgtggcccaagataatccttcttccaatgtggaccagggaagccaaaagattagacacccttgccttaggtcgcttatcttcatagccacataatcaaacaagctcctttgtttccattttgcagatgtggaaaacaggctcagaactaaatggctcaagatgacatactgcttgctagtcacaatacagaggtgacttaaatactatttaacagcactaaaattagtggcccaattcataatcgtttcttctagagcggagtttctcccctctgtgggagaaacttttttattttgcccctctatggaggcttttaagttatttttccctaatagctccccacttataaagttctaatatcacaaacatattatatacatttatctcctttggaaggccacaaaccactgtactatcaataaaacgttttcatcccattggtgaaaatatcacttgattgaaaatgcatgttctatttaacacaggcatttttaaacagggggtctatgaaattagataagaaaaaaatactaattttcactaagctgaactaaaattcaacctttctttcaatgatgaatataagcaacataccacagtggtattaatagaacttgtgactttgtcatcagtggagaccacagatattttcatatagtgttacaagttgttatgacttggaaattcttgttatctgacaggctactagatcttgttatttattgtattaataaagtagctgatattactaagttaaaaatgttttcatttttgatgactgtatttcaatgcaatttatttccttggtcatctatgtattttatacactatgttgagaagggatctatggcacaaaaaaggttaagctccctcccctagaaggatccagcagttcccagagtgatttacagcctgaataccagcttgcctttgctgtatacacaagcttgcctcttctgtagcctccaccagcaacatcccacttgactcttaaaacagcagcagctctgtttgcttgtgcagtaaaaagtgcttctacatccaggccaccactggtaaataccgcaactgtatttgccaaggagtaagaagattctgttatgaacaattgctgggcatgcttaagttaaccaaaaaggaaaaaaaaaaagcaaagttcagagggattacacagcaactagttcttccatttctccaccccacccccaccccgaattttaggaatgttatcttgcagcatctagttatccaataagttggaagaatgcagtaaacccagcaggggtcttagattcaatcactgccaaatggatagaaaatactcttactattgtataggaagaaagttcatctactagtggccaatcaaattctggagacactgaaatgcttaaaaagtaaaacctgtatatcttgataaaagcattatgactcaagcttatctttttttcattcctacatttaaagaacgacaatgagtagattatctaaagcacacaacatcttctaccctcccccaaccccaggataattgcttaccaaaaaacaaaaggaaattatgtgccttcttaagtatttaaccagttgaaagaaaactgcctctttagcagcttaattttagcagtcaaaaagccttcaactcaattcatagaaggcagataagactttaagtttccacctaattaagatctaaaaaaggcaaagtttcaaacagccttgaaccaaaattcggggatgctgctggagaggaaaaaaagagtaaatggctcaaagaaagtttcacaggcgctaaggcgtgtgacttctgtttgcttccaaggttagggaagtgtcccactcgtccccaactagagatcaggcaagtttgcaaactccatgagggcaggggtttttgtcgcctgtctccagagcccccctgaacaaatcctgacacagacatacaatttgttggggcgggggtggggggggattgtggtgactggcgatgcagttcttatctggatttagtggcttttaaaaatgtttagacattttaaaaaatagtcaaaagtactgtaggctaaaggtgatgaggaagggacaacttctggaactcttttcgccatcaatttttccagggtagtacaacccagagaagccgaccaaactccaacattgttttatttctgggacctcttcatctaaaccgtgtgaaggcacatcccaacatttaagatggcagaggagaggccaccaaacccaaccagccaagcaccaatcacggctcccttgtcgaagatgccaaaaagaacatctcatcacatcaccaactgacctaaggttatttttaaaaaaccaataaaaacaaaacaaaacaaaacaaaacaaaacaaaaaaaacagttccttgggctctggggcgaggcaggacaagacaccatgggccatcaagcagggcaaggcaccacgggcaaagccatttcaaacaggtgctgcaatttaaaatgagtaaaaacatgactatcacttttggtctttaatatcttgaattaaagatagcgcactcgagtccccagcctggtgtctggcaggcagcacactgccatttctgctagattgcctcaactggcagctcaacaccatcttgacacaataaacacaaataaaaacattttggtattttgaacaaaattaagactgatcacattcccaattcatagtagcccttctatagttgaaattatctaaacagggcattttatcactgctatatagaggaggaacccaaaaactcagaaaaaacaaaaaaacagaaaacgccaccaagctggcttcaaggttacacagctgaaagtcacacagccacagtggaacgcggatcccattctaaaacctgacttcccaggcatcaacctaacacttcacattttaaaaaaggaaatgctaactgccttctgcctcccaagatggttccccaacccacagcccaggaggaagctcactaacagcagcacgaatgacaaagtatcatttgcttctgcactgcacatttttattcaacaaaaggagctgtttggcaaattgtagcttctctccctatgggaaacccacatgaagtctaacgtagaaagttgacctgaaacgttaactaacgtcaaaatccttctaagccagcgtaggtcattttggtgagtaattggaaataggaagttattcgaaacttgggaatcctaatcgttgatgtttgtaatccagtttagcacaaagggcatttgaaactcttttgagaaaatgctaatttttcctgtatgtagtgttagccctgcgaccacctgcagattttttgatctgctaattgatcgccagacgtcattttcctccctcaaaaactgcattctcgaatggctttgcaaaataagaccagctcaatctgcattctaaaaaattatgtgagacatatacatacatctaagttagatatgtatgtctacattatatacctaacacatataattttaggcagctcaaatgcaaacaacccaaacgcagccattaaaaacgcactgcttgggaaaacaaccttctctcccaaggaggcaattggaggctctgcaaacacattctagccgccaggagttattaccaagaattccacacttggcaaagaacagcgggcacagacgacgatcaagcaggcgaccggcacgaatcccctagggtacccatccttggcccagcccagcactctccattcacccccacaacccgtcccacccaccaggtgtgagccagtacctgcgaagggaccccgcctcccagcaaagggctaggcttcaagctgggcctccagggggcgcccgcgcgcaagccgggtgggctgtccgcgacaaccctggtgatcaatagccaactcccctccctcccaaatcgcagtggcagcgcacggccaggcccgaggttttagctcagctcaggttgactctgattccaaactccaaccatcgttaatttcagaagccctctcgtaacaagcttccctcccgaaaggtgccaaacaccaccaaaagccccacgtgtgtgcgcccacccaaaatgaagggggaaggggaggtcagcgtaggaccaggggaggggtcagaaccaggagctcgcccctcctctccggaggaagcaggcgggctgagggttaacccttttgttccaggtgggccaggccacgaagccttcacgctcccctcaacccccaccccaccccgcccccaaccaaggcacaaagagagtccctccaggcgcctgggacctcgggtgcgcgctgcgctgaccacccggcgccccgaaaacgcccagccatcccctccgtcaagccgatccttttaatttgccctggaaaaatgttaaacggggaattctattgacgtattataaacttgggaaatgtattacattcagtctccgacaaaaaggcggggatacggaagccaaaaagtggcgaccgtcttctcgggaaaccccgggtatcagagatggtctctgagagtcctccggggttgccgcgcacacccgtctgctgcagggaaattagaaagcaggcggggagcggagcgacgaacaaggccggagaggaggctacaaatcccccctccccccacgcgcacgctaaaaacttatcaggcgacaatttggcgggccgggatggagacgagatagtggaaaaccgcctcgctcctaaaacctccgcggtgaagtgatgaagtgggaacagagttggccccgccgttcctggcgcggctggggtcccgaactcccctcgatctgggaaacgctggccgcggaagcgtgggccaggtcagccgcgggagttcgctctggccccccacctctcccctttcacacccaagcgcaaatgtggaaagccgagcgctccagaaaagccacgcaaaacgggaggccggtgcaccccagtggggctgccgcgcgcccgccaccccggccactgggggctcgcaccctcgggcttgccgcggttatttttaggaagtcggaaatcaaagatggctggaggtcaggccccgaaaggttagggcgaagatcggcaggcggcgcagcccgggcagaaaaggcgaaatccagaaaagaaaaaaattttaaagggaaacattgagccctcctcccccacctctctgggccctccaatcccctgcgcagcgcgggtggcccgcaccccagctcctgagggtggggagaggtcgcgccccctccccccacttggcccgggaggggcgcgtgcaacagatcgtcctcctccggggggtcttccaggggtgcgcccctcctccagcctccaagaaaagccccaaggggctggtggagagggtggggatcccccggggttctttcttttgagagcgacgcgagggagggggacctaagttacaaggggggggggtaacaccagtgaggggagtggaaccaattaagtgaggggcagaggacaaggagaagggaacctttcgggagcgagaacagaggagcgagtgggggtcaattttacaattaggggagaaagctgggggtgaaacttacacaagaggggagaagaggaaaaaagtgtgaaatgtgcaaaaggcggggtggtggggagaggaggagacctctcctcccgggcactgagcccccaaatctcggctcctgaatttgcgctggacgccgcccgctccccgaagcctgcgccctcgaggatcccgcgtacgtccgggaagctcccctccccgggcgggcgccccagccccactcacccagctcccccgccgcgggcgccgctggggccgcatctgcaaactccggggtctgctgctgcggctgctgctcgccctggtcctcctccgagttgatgtgctggggtttcgcctgcttgcgcctcgacatggtgcgagcatcggggcgccgggagagccgcagttatttgccctctccgccacaaattcctggagttgggaaatttaccccccttcggccggaacgcgcatgtcccagtaattattattatcaataatgcattgcgatttatcatgagccctgacagctgattggcctgggtccaagacctcagagatcatttaaataagccctcattgatcagggaggggtgaggcattctgggctttgtagtccgggcctacttgtgacaaaggcgtgttcatcaggggagcgcagctaattagcatccgggctcagattggctggggcggggacgaaatcagagggggacccatccttgctccagctatctcagtccttggagaggaaaagaaaatggggagaaatgcgtactcattaaaaagtaggagcttacgtagaagttttggaacggctttatttggtagttacttgctccaaagattacttggtaaggttgatgtccaccagccgagaagggagatctcttcacagtggaatattttggctcagttttcacatctacacgcacacacattcccccaaacctgagatgtgacccgtgtcccctcccatctttttgattaaaaatgtttaagttgagtctcgagataactgaagaatgatgaatggtggtgttttcagccctcccacccatctcaactcttaaagtgagctcggaacttccaaaatttcagataccccaccaccaccatgacttacctcctttaaaagctgattaatagcctccaggattcctgttcacaaagactggtttgtggttacaatgcaaaccttgatttttttttaacttcaaaatgactggctactaaatatagaaatggcaaatgataagcaagcatagtcagagataatatcaaaacagcaagcaattgcatttttttccccaggagccataaatgcctgataacatcatattttattactgagacaatgctcttgcatctcccatggaatggcaatggggcactgtcacagtatctggttgttactaatcactattggaatttttttgtgcaatacacacattattacagaaaaggtgtatcgttcaaagtgattgtatcattgacacatgatgcctggaattttttaaagtggggcttgtgtgtgtgtatatgatatattacatgtatacacatttataccgtaaattcatgtatacttaatttggctaactgctttacacaatgtacatgctcaataaatgttaaatgaatgatgaaatcatcaacctgctaagtctttgattggtctttccataactgctctgggcatgcgtctttgtttggcatttacttcattgtaagggactgtgcagatataaaccctccctccacatttgtgcattgcattctcttgattagttattcatcacgttcccagcttgtaaactcatcaggaatacaagcaaaatggaatgctgggctcatcaatgtgttcacctgggtatttggacaacccaaggcagaaaataaatcagcttgatcatcttttttatattcgtggccatcagtaaaaacttgttgaaggaagattaaccttcaatttttgagtctctgaaaaagtcttgccttattttatgtataatatttaaagcaagtaagacagaattatagccaacagccagattgaaaaggtaagaatttggtttaataatacatagaagcagtttagaattctgatactgagtaaaaaggccaattagcacctgccttgctgactaatctactaatattctcaacaccttttctgtgtaagatagtgtggcaggagatctaacttacaaatctaaacgaatgtgcagtgggtttatggctcaaaaaatactattgggttaaggaaaatttcatttatatagttgtttatatttattttagtagatgatagttgtaacatgttagccttactacgagacaaaattgtgccgcttgactctccaagtgatagtaataataataattgattgagccccttgtacatgccacagactgcattaaatgaatgcacaccgccttgttttatcttcccatggaggttggccctattgtcctctccattttgaaactgaggctcaaagacatgaagtgacttaattagaaactggctgagctggtggtcagtttctaagccggtggtcagtttctaagtctgactccaaaattctatcacatcctaatacctgtcttccaactatagctgaatatagagaagatagctaaagaagaaaaatcggagtacagtaaactaaatttaaagtgtcctagaaaaatcttgggtcttcagaaatcactccaacagatgaagtcaagaagtttccctcaaatggaaaccttccacaaatgtcatatcaagaaaagcaggacacagtacatgttttgaatctacccccagtttaacagctgcagccaggagccagttggtggaaaataaagtaagtcctagagcaaagcaggggatgaacggagggcatagcaacaggcaagtccgtaatcaaaagttgtttcaggctgagcacagtgtctcacgcctgtaatcccagcactctgggaggccaagactggcgggtcaccagaggtcaggagttcgagactagcctggccaacacggtaaaactccatctctactaaaaatacaaaaattagctggttgtggtggcgcatgcctgtaatcccagctactcaggagcctgacggaggagaatcacttgaatccaggaggcggtggttgcagtgagccaagatctagctactgcactccaacctgggcgacagagtgagactctgtctcatgaaaaaacaacaacaaaaaagttatttattaaaccaaaggacctggttgggacccaagcttgaaaagaaaatgttcagagtaaaggatgatattaaactttttcatgttaacgtcctttgttaggtgactgggtttatataatccagtcattgtcaatgtttatgtaatccagtcatgttcaatgtttgtgccaggcaggaatattcaaaaaatacacttcttggtatatttttcagcagtcttattgtttataaggagatttgggatgcataatcccattttatcctcactaatatccttttttaattcctttttttttttttttttttttttgagacagaccactcttgtcacccaggctggaatgcagtggcacgatctctgctcattgcaacctctgcctgggttcaagcgattctcctgcctcagcctcctgagtacctgggactataagcacatgccaccacactcggctaatttttgtatttttagtagagatggggtttcaccatgttcaccagttgtctccaactcctgacctcaagtgaggcccacctcagcctcccaaagtgctgggattacagacgtgagccaccgtgtccggcttatttctacttcatttatggaaaaacagaagctcattgctcccatcagctcctagcgagtactaacgaatgcatgccaaattatggattcagtaactttgcacaggtctcagtggaaggcagatctttccctgaaaaccccacctctgctcttttctacaaggaatcgtcttcctttccactgcttgcctctcttcacctggcccactcctaaaaagcctataatctcttcaaatttcccttccttaaaccagccttccctcttccaaactaggtctcatttattagttctctcaggcttcaagaaaccatatctttctttttatagcgcacttcaaaatatataatgacagactttctcatgtatctgtttattgcttatttctcacactgcaggctccgtgagggtagacaccgtcatcacggtgtccttagcacttagccacagtaatggaacacagaagtcacaacaaataaatagcgatggactcaatccatgctgagtaactttagaggcctgaatatgaaatgaagtgttaaaggcaatatagtaggtgggaatgtaaaatgctgcagccactctagatggcagttcctcaacaaatgtaaaaaaggattaccatatgatccagcaattccacatgtgggtatatatccaaaagaattgaaagcagggtctcgaagcagtatttatatacccatgttcacagcagaattattcacaatagccaaaaagtggaaacaactcaagtgtccatggacagatgaatgaataaagtaaatgcagtctatccatacaatggaatattactcagccttaaaaaggaaggaaattctgacacatggtaccacatggatgaatctttgttttcttttgggtttttttgttgttgttttttgagacagagtctcgctctgtcacccaggagtgcagtggtgcgatcttggctcactgcaagctctgcctcccgggttcacgccattctcctgcctcagcctcccgagtagctgggactacagaagcccaccaccacgcccggctaattttttgtttttttagtagagatggggtttcaccatgttagccaggatggtcttcatctcctcaccttgtgatccgcccgcctcggcctcccaaagttctgggattacaggcgtgagccaccatgcccagccaacatggatgaaacttgaagacattatgccaagtaaaatgagccagtcacagaaggacaaatagagtagtcaaattcatagagacaaaaagtagaatggtggttgccaggggctggagggagggagagtggggggtggaggggccgtttctgctcaataggcatgaagttccagttttgcaagatgaaaagggatctagataaatgtttagattttagctgctttgccacaaaaacaaacaaaatcaggtaacaatgcaagacggataggttcatttgcttcactggtaatctttttactatctatataaatcccataacatcatgtagtatgccttaaatatacacaataatttttttttttaaaagatctggactgggcacggtggctcacgcctgtaatcccagcactttgggaggccaaggcaggcggatcatgagttcgggagttcgagaccaccctgaccaacatggtgaaaccccatctctactaaaagtacaaaaattaaatgggcgtggtggtgggcacctgcaatcccagctacttgggaggctgaggcaggagaatcacttgaacctgggaggcggaggttgcccagatggtgccactgcactacagcttgggagacaaagcgagactccaactcgaaagaaaaaaaaaaaaagaaaaaagaaagagagagatctggaaattggttgctcaacaatgtcaatgtactcaacatttcaaaacagcacacttaaagatgggtcagacagtaaatcttatgttaagtgtattttaacacagttgcagagaaaaaaaggcaattacaaactagattatcacgatggctgcatactgtatggatttactaaaaataattcaactgtacactcacagtgagtgagtttcatgatatgtaaagcatatgtacatgaaatggtttttaggaagacaattacatacttggctatccctgtcattgaaaggaccacagttatgtgtgtgtttttttctgtgtgggattcccagcatcttaagctcgaaatgactaaagtagcgaggttgagtcccataaatcatgatctgccactgcaaaagtattgcccctttggacctctgtttattcatgccaggaagcaaagtcgactgacttggtaaccacagctgcttcaatttgggaaatgtaagggttggatttgggaagtatcttaacatggaccaacgtcttagagtctatggacttttatgtatcatagtacatttgatatctgcctaccccaaatggaaggcttgatgtctggaaggcactcttcaaattctagagatttagaagttaagctaaaattgcctccaaaatcatgcttgtaattcaatgggactgttttattatggggccaaatgtccacttttctgagtgtcctggcccagagcccttctgctgggacgatttatgttcgcaaagttggctaattgcaataatcaccacttaactgatgatatttcccccattttaatatctctctgcttgttgccatttcatcaattccaaattggtattgaacaatttctagctccaattagtcttcttagttgcagctgatgtattgaagcctacaactgtgtattcaaaaaaaaaaaaaaaaggtaaaaatattccaggccagacaccgtggctcatacctgtattctagcactttaggaggctgagaccagaggactgcttgagcccagcccaggagtttgagagcagcctggacaacatagggagtccccatctctacagaaaataaaccacaaaaaaattagccaggcatggtggcacgcacctatagtcccagctactctggaggctgaggtgggagatcacctgagcctggagaagtcgaggctgcggtgagctgttatcgtgccactgcattccagcctgggtgacggagcgagaccctgtttcagaaaaacaaaacaaaacaaacaaataaattaatcaggcatggtggtgtgtacctgtagttccagctactcaggagactgaggtggaaggattgattgagcccaggagtttgaggctgcagcgagccttgttcatgccactgcactccagcctgggcaacagagtgagagtcagtcttaaaaaacaaacaaacaaaaaagggcctggcacaatggctcacgcctgtaatcccaacactttgggaggtcgaggccagcagatcacgaggtcagaagttcgagacagcctggccaacatggtaaaaccctgtctctactaaaaatacaaaaattagccagacgtggtggtttgcgcctgtaatcccagctactcagaaggctgaggcaggagaattgcttgaacccaggaggcagaggttgcaatgagccgagatcgcgcaacagcactccagcctgggacagagtaagactctgtctcaaagaaacaaaaaaattctgtctgggcgtggtggctcacacctgtaatcccaatatttgggaggccgaggtgggtggggcatctgaggtcaggagttcgagaccagcctggccaacatggtgaaaccctgtctctacttaacaaaaaaaaaattagcaggcatggtagcgggcgcctgtaatcccagctacttgggaggctgaggcaggagaatcacttgaacccgggaggcggcggttgcagtaagccaagatcatgccattgcactccagcctggacgacaagagcgaaactccgtctccaaaaaaaaaaaaaaaaaaaaaaaaaatttccaacaagcttattaaagcaagagtgaattgtcagaaatgatactttcttctgtaagtttgctggttaaagactaaatggattaagaataaaagatttggccgggcatggtggctcactcctgtaatcccagcactttgggaggccgaggtgggtggatcacgaggtcaggagatcgagaccatcctggctaacacggtgaaaccctgtctctactaaaaatacaaaaattagccgggcttggtggcgggcccctgtagtcccagctactcgggaggctgaggcaggagaatcgtttgaacctgggaggcggagcttgcagtgagccgagatcgtgccactgcactccagcctgggtgacagagcgagactccgtctcaaaaaaaataaataaataaaatagtttgtgtgactagctttttttactcctcagtcgcttgaaactcatgctagtccaagcatgcagtctttcggtcaatctcaggcacacacatacagtcgagaatgttggttctctaagtccacagcagcagttcttaatgggaacaatgtgccttttcccccaccaggggacatttggcaatgtctagagacattttcggttgtcactactgtctagtaggtagaggcccggggtcatgcttaagatcctacaatgcccaggataacctccacaacaaaggaatagctgacccaaaaggtcaacggtgccagagatgagaaacgaggcttacaggtaatatgatttcaaggctatattagcacctaactaataccattaaaaaaaaaaatagtaaagtcctttctaaaaagtattaagtcacaatcaaatcctcttatcttctccaggggaggcttcttaaccttcccaactaaatcaaacttcagcatttcgaccctttccctcaaagattgcacaattaccgtcttatattcactcatgtgatgatttgagtaatttatctacttctccgcctgcagattagagaaagataacattttggaattctgtttatttcactgaatgggctatcataagattctcctgccgcattgaaagaaatctttgtaaatgtgttttgttattttttcactctgtggatgaacctttaattcacttaaccaacccctgttgagtatttgagatctttgtctttactttagtaacatttcagggaacgtctttgctcatagacggctctgtccaaattttggatttcttgccttaaggcctaccatcttgagtctttctgaagtgaatgttgaaatgaatttcacagattcacagccctttgagagcttttttcaaatactatcttactccttttgtaggctcttgtgcaagaccagggcctcagtaacatgagtgatcaagctaaatgggtcatctcataaacatgcttatcgggacactcacatattgcgtaactggacagcagaaatgtatagattttcaaaaacaagtcaaaggttcaattgattgaacatttttttttctgctagaaatgctttccttttcatttcctttttacataagtctgattcactctttacttaaatatttctgtactggcgtccaaaacccttcttaaaagttttctcttgccttaaaagaaaatttttttttgttatctaccaggcacaaatccaaccccttagtcccctaaaaagtgcctattgtaaactctatgtttaaaaactattactgagaaggcgcttcttcaaattcgctgcaaactttattataacaaagcttaaagagctaaaagcgcggcacaatggaaagttgactgttataaatatctatccggctttgcaatataaattataattaaaaataaacttgtcagagaactctaggcaacacacaaaaagtgagacgtttagaacgaggcaattaagaatgttaattggaacacagagaaaagcaaaggacagtcattgttaagataactgctctataaacacctaaatggattctgtgaaggggaagaaaggatcatcttttgaccatgaaagggaatataaaatgttcttttctttgttttaatgtgtggagagattttaaacttcttttctcgctcagacgctcaagtaaacaaatgacttggccctagtctggtgtgggtaactaaagagagaaatggagggaaagagagaaaagccaactgggtaggcgagaagagaagagagcaaatgctgaagaagaagaaaaacaaaggaggatgataaatgtagaggaataaagggaaaaaccaaaacagaaggcataaacccctaggaaacagaacaagagttaaacacagagaataagaaaggagtcatctccctttcttcactctccaaatgatctgcaagttaatttcttaaagacagcagagtcgacaagcacagtcagacagacccagatgtagatcttgcttcttggcagtttccatctgggctggataatttcctccaggttctcaacctctctatgccttgatttcttccctagtcaatggggagggtaatcgctgcttgtggcttcgctgcaaaaatcagaaatgatttgtgtgaagcccctggcatataattagtgtttaggaaacatctattaccacatgaattatttcagggtacagcaggaatgtttaagctttctcataatattaagcagacgcacaacatacatgatagttttggagtcggagttatttaatttacatggtcatttatttgatcattcattcattcaaggagtaataattttgtctgctatgcacagggccttgagctcacccagtgctgggaatttagacatgaaaaaccttaagtagttcagcctaaaatgtctaaaagtttaatgtcaaaattgttggcttttgcaaaaatgttagcctgtgggccaatggtggtctatggtacattgactctgcctagaatatggctaggatgggatggaaacacgaaagacatgatttagaacatattcttttccagaataagcattccgtgacagaaggggccatattcatctcattcaccgttgcataaacaaattgcattgcataagcaaatatctttaatgaacagcatctaggagcacgtgctttgaagacaaagcaaggtaaaaggacagaggccatcagtggatgcttttcttgtagggtgatcaggaaaggcttcactgaaaaggagacaagtggccgagacatgaaggaggtgagggagaaagtcatatagatctgtgggaattgcattccagacagaggcaacagacaatgcaaaggccttgaggcaggaacgtgcttggcgtgtttgaggaaccagaaaaaggccagtggggctggggtggagtgagtgaggagtaataggggcagaggacatatgaccttagagatcatggctctagatactttgttttcctattttttttattgtggttaaatatccataatatgaaatttatttatgtatttatttttctgagatgcagttttgctctgtcgcccaggctggagtgcagtggcatgatctcagctcactgcaacctccgcctcccaggttcaagcgattttcctgtctcagattccccagtagctgggattataggcgtgtgccaccacactcagccaatttttatagttttagtagagacggggtttcaccatgttggctggtctggtcttgaactcccgatctcaggtgatccgcctgcctcggcctcccaaagtgctgggattacaggcgtgagccaccgcatttccacttattttaaatgtcatctcgtagtggcagcttaccaaatagagatccatataactgctcgaaaattttaaggcttaagaaatactcaaaaggagtgattagtagtagaatgcttaataaattcttaattgtcaggctgggcatggtggctcacgcctgtaatcccagcactttgggaggccgaggcaggtggatcacctgaggtcaggagtttgagaccagcctggccaacatggcgaaaccctgtctctattaaaaatacaaaaattagccgggcatggtggcacacacctgtaagcccagcttactcaggaggctgagacatgagaattgcttgagcctgggagatggaggttgcagcgagctgagataatgccattgcactccagcctcggtgacagagcgagactctgtctcaaaaataataatgataataaaaaataaattcttgtttgtttgtttgttttgttttgttttgtttgagacggagtctcactctgtcacccagactggagtacaatggcgtcttctcagctcactgcagcctccacctcccaggttcaagcgattctcccgcctccacctcccaagtagccgggactatacgcgcgtgccaacacacccggctaatttttgtatttttagtagagatggagtttcgctatgttggccaggctgatctcgaactcctgaccttgtgatccgcccacctcggcctcccaaagtgctgggattacaggcatgagccaccgcgcccggccaataaataaattcttaattgtcctgcagcaaaaatgtctaagggaaccaagcatggtggcacatgcctgtagtaccagctactggggagggtgaggtaggaggatcacttgagcccaggagttgaaagatgtataccccacaatcgtgcctgtgaatagccacagtgacccagcctgggcaacatagtgagacctggatctctaaaatcaaaaaacgtaagagttgaaatatttagataggtttcatcttaatatgtctgccaagtcaattagcgataggacagccctccccaccccggtttttttgttttgttttgtttttaagaattttgctagggctgggcacagtggctcacacctgtaatcccagcactttgggaggctgaggcaggtgtatcacgaggtcaggagattgaggccatcctggctaacacggtgaaaccccttctctactaaaaacacaaaaaattaactgggtgtggtggcacgcgcctgtagtcccagctactcaggaggttgaggcaggataaccgcttgaacctgggaggcagaggttgcagtgagccaagactgcgccactgtacttcagcctgggcgacagagggagactctgtctcagaaaaaaaaaaaaaaaagaactttgcttttttttttttttttttttttttggtgacatgaaaggctgtcagagtgttttgagcaggggaatgacaggattttcctaggttttaggagaactctgagggaggttatagacccaagtaggtgggggataggatggatgtggggagagcagtgaggacgctcttgcagtagtccaggaatgagaaaatggagcttggaccaaggtggaagtggcaggggtgggagagataaaacagagatgtggggaattcattgtgatcatgtggctgatagaacttgcaaactgatcggatgcaggtataagacacaggggtcaaggatgacgcctagattttcagcctgagcatccaaaaggttgcaggtaccaggtagagaaatagggaaggctgtgggagacgtagatttggcagagggggtgcttcagaactcgtttggaacatgttaggtttgatttgccaattagacattcaagtggaaaagttagatcaacagttggagacaccagtctaaagttcagaggcaaggaccagcaggctgttgacatcagtatgagatggtttttgaagccgagaaactggctgagatcatctaggcagtgaatggagatagggaagagaaaagagcaaggatgaggcctggggaactcctgggtttacaggagggaagttgaggagaaaaatacataggtgcagagaaagagcagccagtgaatcaggatgacagagagagcagtccaggaagccaagagaggaacgtgcttcacagggaggcagggactcactgtccaaacatgacactgatgtgtcaaggcagacaagcattggaactgagtgttgggtttggcaacacagagacaactagtcattaagaagatgcctggggatacctaggggcaacagctgattggagagggttggggaaggatgagaggagatggcgaactctttagggtcaattcttttaaaactgtctcatggtgagtggggagtggggagcatcagggtgaatatgagtgtgagggagataacatagcaagtgctggatgacgttggaaggatccgagggaagaggaaatgttgatgatgcaggagagaaagggaacaattacagagtacagatgctccttgacttctgatgggtttacgtcccgataaacccactgtaagttgaaaatataataaactgaaaatgcatttagaggcctaggcgggtggatcacttgaggccaggagtttgagaccagcctggccaacatggcaaaactccgtctctactaaaaatacaaaaattagtcaggcttggccaggctcaatggctcacgcctgtaatcccagcactttgggaggcccaggcaggtggatcacgaggtcaggagttcaagaccagcctggataacatggtgaaaccccgtctctactaaaaatacaaaaattagctggacgtggtggtggtcacctgtaatcccagctacttgggaggctgaggcaggagaattgtttgaacccaggaggcagagcttgcagtgagccgagattgtgccactgtactccagcctgggcaacaagaaagagcgagactccgtctcaaaaaaaaaaaaatagtcaggcttggtagcgcacacctgtgatcccagctactttggaggctgagggacgagaattgcttgaacctgggagacagaggttggtgtgagctgagatcacgccactgcactccagcctgagtgacagagagagactgtcttaaaaaaaatgcattgaatacacctaacctaccaaacattacagtttagcgtaccttcaacatgctcaaaacacttacattagtccacagttgggcaaagtcatctaacacaaagcctattttataataaaatgttaaatagctcatgtaatttattgaatactgtactaaactgaaaagcaaaatggttgtgagtacttgaagtacagtttctactgaatgtactaattgcccttgtaccactgtaaaattgaaaaatcttaaattgaaccattgtaagttggttcatctgtagtgatctcaggaaagtaggaaagagctgggcatggtggctgctcatgcctgtagtcacagcaactcggggggccgaggcagggagatcccttgagcccaggaattcgagaccagcctgggcaacataaggtgactgtgtctcaatttattttattattatttttttgagacagagtctcactctgtcacctaggctcgagtgcaatggcgcaatctcggctcactgcaacctctgccccccgggttcaagcaattcttctgcctcagcctcgccagtagccaggattataggcgtgcaccaccacacccagctaattttttatttatttattttgttttttgagacagagtctcactctgtcacctaggctcgagtgcaatggcgcaatctcggctcactgcaacctctgcctcccaggttcacgccattctcctgcctcagcctcccgagtaactggaactacaggcgcccgccaccatgcccggctaatttttttgtatttttagtagagacggggtttcaccatgttagccaggatggtcttgatctcctgacctcatgattcacccacctcagcctcccaaattgctgggattacaggcatgagccaccgcgcctggccttttttttttttttttttttttttttttttgagacggagtctcactctgctgctcaggctggagtacagtggcgggatctcggctcactgcaacctccaccttcccgggttcatgccattctcctgcctcagcctcccgagtagcttggactacaggcacccgccaccacgcctggctaatttttttgtatttttagtagagacgaggtttcactgtgttagccaggacggtcttggtctcctgacctcatgatctgcccgcctcagcctcccaaagtgctgagattacaagcgtgagccaccacgcccggcccacacccagctaatttttatattttttgcagagatgaggtttcaccatgttggccaggctggtctcgaactcctgaccttaagtgatttgttcaccttggccttccaaagtgctagaattacaggcacgagtcaccgcacctagcctattttattttactttattttagttttcagacagagtcttactctattgcccaggctggactgcagtggcgcatatcagctcactgaaacctctgcctcccgggtgcaagtgattctcctgcctcagcctcctgagtagctgggattacaggcacgtgccaccaagcctggctactttttttgtagtttttggtacagacgcggtttgtccaggctggtctttaactcttggcctcaagtgatccgcccaccttggcctcccaaagtgctgggattataggcatgagccttttatattttatttaaaaatttttaaaaattctttaaaaaaggaagagatggagtgctggcctatgacatggtaacatagttcatccgctgcaatagcagggaaggcagaatagttgggtgcagttcaaggcaggtaggttaatttagcttgggaatgtgtgggagttcactactgattgcctctattgtgtctattacattagtgaaattagaaacaagagtaaggaagggaagaggatgctgagtgttagaagagagaaagaaaggggtaaaagtcttccaggagagaaagagaaagaatgaaggagggaaatgtagtggggtggctgggtccatgtgggcgctcatgcatttctttctttctttcttttcttttcttctttctttcttcctttcttctttttctttcttctttcttttctttctttcttctttcttttctttcttctttcttttctttttcttttctcttccttccttccttctttccttccttccttccctccttcttctctctccttccctccttctttcctttgttccttctttccttccttctctccctccctcttttcttttcttttcttcttttcttttctttcttttctttttcttgagatggagtttcactctcattgcccagactggagtgcaatggcatgatctcggctcactgcaacctctgcctgccgggttcaagcgattctcctgcctcagcctcccgagtagctgggattacaagcctgtgccaccacgcccagctaattttgtttttttgttttttttttttgagacagagtctctctctctgttgcccaggttgcagtgcagtggtgccatctcggctcactgcaagctccgcctcctgggttcacaccattctcctgcctcagctaattttgtattttcagtagagacggggtttcaccatgttggtcaggctggtcttgaagtcctgtccttgggtaatctgcccaccttgacctcccaaagtgctggaattacaggcgtgagtggacgcccggcctatttcttcatttttcttaaaattatatatatatatatatatatatatatatatatatatatatatatttaaatagatatttatatattttttttttcagagggatttgctctgttgctcaggttggagtggtgcgatcatggctcacggcagcctccacctcccaggctcaagcaatcctccatccttagcctcctgagtagctgggactacaggtgcacaacaccacacctggctaatttttttattttttgtagagacagggtctcgctatgttgctcaggctggtcttgaactcctggcctcaagtggtcctcttgccaaagtcctgggatgacaggcatgaggttcggcacctggctatggtcatgcatttgaagggaaaccagttagctgtggtagtgcttttttttttccctcagacattttcagtggtacctgtccaagtgcagaatggactaagagttggattgaaaaggaatggggtttagccaagcaagtacaacagagaataaaagaacaggggcactgaggctgaatgtgggggattacaatgatggaccctagactcaaggcctggtaagagaggcagagcaggaagaagtgaagaggagtaaaacaggtttgtgagcatggaggttgggggaaaatcaagcgattgttggacttggggtcccaaagggacaaagccagaaagattttaggggatagtcggaggaaaagatgcttgaaattgagatggtggagccgatgtcgtggctggaaaggatgagggtatggagcacctgctcaggtggggtggatgacaggagcctcatggagaggaggacaaggagctgagcagctgaggtatcaaagggtcatcttaccaaatactgaaaatgtcaagaagtattgtaggtgagcaagacagtcgggtgacagtgtctttaggaactgaggagtaggctctcgggtctgcagaagaggcaatgaggagggggcatagacaagaacaggtgagtctgagcttctgagtctcagctgaatgctttggggaggaagaatggataattgaggcctcaagaggcaagaaactcacttagcccaactccagtcagtgcaccagggtgacagagggaaacagagcccctggagagagctgggggagaagcagtgtccccaggggagaaccaagggccaattagggcaagaacattccaaaaaaaaagtgaggataaaggagactttgccatgacgaaccagaagacactgaggaagtgttgggggtcggtgacagggctgaggattgggtcagataagaaacaggtttatgggccgggcgcggtggctcacgccggtaatcccagcactttgggaggccgaggtgggtggatcacaaggtcaggagttcaagaccagcctggccaacatggtgaaaccccgtctctactaaaaatacaaaaattagctgggcgtggtggtgcgctcctgtaatcccagctactcgggaggctgtggcaggagaatcacttgaacccgagaggcagaggtagcagtgagccaagatcacgccactgtactccagcctggatccagcccaggctacagagcgagactctgtctccaaaaaaaaaaaaaaaaaaagaagggctttatagaattggtcacgtaggaccagacgctgtccgaaccactttgcttgtatctaatcgtttaccccatgggataattactattattaacagatgcattttccagatgagaactttgaggtatagaaagtaatgaccaagattcagctatagaactaggccaccgcctcaggaccatactgggtgtcagcccctgagccaaatccagcccaccacttgtctttgtatgaccttcgagctaacagtgggctttagagatgaacatttcaatctatttgatgatgaacatttactttgaatcccagttaaccaacatgtcatcccctctaaaagaattccattcttctcattagtagacctatattaggagaaaattaaattgttattatattttgaatttcatcaattgcaaatttgtgaaaatttgttttctcacttgtgtacctacatcatattcttgattgtgcctcttggtctgtaaaacctaaaatcttccctttccttccttccttccttctttccttcctctccttctttccttttctcctttctctctttctctctttctttctttctttcttactttcttttttgagatagggtgtcactccatcactcaggctggagcgcagtggcacaaacatggttctgcagtctcaacctcctgggcttaagcggttctcctacctcagcctaccatgtagtgagactataggcacactaccgtgcgtggctaatttttttttgttgtttttttagacggagtctcgctctgttgcccaggatgcagtgtagtggcacgatcttggctcactgcaacctctgcctcccaggttcaaacgattctcctgcctcagcttcctgagtagctgggactacaggcacccacctctacgcctggctaatttttgtatttttagtagagacggggtttcacgatattggccaggctggttgcaaactcctgacctcaggtgatctgcccgcctctgcctcccaaagtgctgggattacaggtgtgagccactgtgcctggcctaattttttatttttatttttttgaggcagagtctcactctgttgcccaggctggaatgcagtggtgccatcttggctcactagaacctcctcctcctcccaggtttaagcctgattctcctgcctcagccacccaagtagctgggattacaggtgcacaccaccatgccaggctaatttttgtagttttaatagagacggggtctcaccatgttgaccaggctgttctcaaactcctgacctcaagtgatctgtccaccttggcctcccaaagtgctgggattacaggtgtgagtcactgagcctggcctaatttttcttttctcttttttttttttttttttttgagacggagtctcgctctgtcgcccaggctggagtgcagtggcacaatctcggctcactgcaagctccgcctcccgggttcacgccattctcctgtctcagcctcccaagtagctgggactacaggcacccgccaccatgcctggctaattttttgtattttttagtagagacggggtttcaccgtgttagcgaggatgttctcaatctcctgacctcgtgatctgcccacctcagcctcccaaagtactgggattacaggagtgagccaccgcgcccagcctggcctaatttttctgtagagacaatgtcttatcatgttgccaaagctgaactcctgggctcaagccatcctcccgccctggcctgccaaagtgttggtattacaggcatgagccaccatgcccggcccctaaaatatttctgtctgttcctttacagaaagagtgtgccaacctccgtattatattcacatatctctcggtggaggaaatgctcaataaatgtccgatattattgttgatattattatcatcattgacacctaattcacaggacttcaaaacaagctagctatgattccagccctgcgctgtcacgcaaaagatattaaacaaatgttggctaggcacagtggctcacacctataatcctagcagtctgggaggctgaagagggtggatcacctgaggtcaggagttcgagaccagcctggccaacatgacgaaaccacgtctctactaaaaatacacaaattagccaggcgtggtggcgcatgcctgtaatcccagctactcgggaggctgaggcaggagaatcgcttgaacccaggaggcagaggttgcagtgagctgagatcgtgccactgcactccaccctcggcgaaagagtgaaactccatctcaaaataaataaataaataacaaaaaacaaaggttagctattgaatgaatgagtgagtgagtgaatgaatgagtgggtaggtgaagtggaggaggatgtgaagttggtgggagtgagggtgctagaaatctccacatctttcaccaaggcagagagcttatcgggggaggaggagcagaagcagtgagttcatttccagaaatgttgctggaggtgagttgactagctccacataaaccctgggaatgatcactgaagctgggtaatttcaccattgtcctcagtatctgacccccgatggaaaattccacagcttcactaaaagacctgtatacctctggaatatttttatagggaacataaaagaaatgaatgtgggatttgggggactggggagaagattctgaggagctggtcctctgagaagagttaatcaggcaagtccagtaattatctggttccaactacatttgaaacactgtgctgggtcccttatgtaattgtccttccaactctgagaagtaattttatcattcccttttcacatatgcagaaattgggattgaaagaagctgtgctccatcccagggcattgcctcctccagcactgggtctaggctttgcatcaacctgtctactgccggttggccatttgtgtcttcatgctacccacagggccctcgaggctctacctctgagattcagcaaaggaggtcctgcctcatggagctcacagtccagggagaaagacagacaataaacaagcaaacaatacacaaataaaataggccaggcatggtggcccacacctgtaatcccagcactttggaaggctgagacgggtggatcacttgaggtcaggagttcgagaccaaccatggccaacgtggcaaaaccctatctctactaataatacaaaaaattagatgggactggtagtgagtgtctgtaatcccagctactgggaggcaggagaatagcttgaatccaggaggcagaagttgcagtgagccaaaattgtgccattgcagtccagcatggatgacagagcaagactatgtctctaaaaataaaataaaataataaaaaaattggctgggcacagtggctcacacctgtagcactttgggtggccgaagcaggcagatcacctgatgtcaggagtttgagaccagcctggccagcttggtaaaaccccaactctgctaaaaatacaaaaattagccgggcatggtgacacgcacctgtaatctcagctactggggaggctgaggcatgagaatcgcttgaacctgggaggcagaagttgcagtgagccgcaatcacaccattgcatgatcaactgtctagcactgagcaacagggctagactccatctcagaaaaaaaataaaataaaataagaaatgcaaaattgttacaggagttgtaagggaataacaagataatagaattagggagagactgcaggtggaggccagatgagatgttcatgggtgcccccttggagcagagacctgtagagctacaggaaggatgttctagaaaacaccaagggccctgaggtaggaaggagctttgaggcatcaaaaatgaagctactgtccttggtgtacagccaagaaaaaaaaggtgggagaagttgaggttaggggtgggtgggaccagatcacaggggccttgtaggccctgcctgtgatttgaatttcattctaagtgatgggggctttaagctggggagggaggtgacctgattcaccctggaaaagagtgccctagatgcttgtggagaagaaaatgaagagggcaagatggaaggcaacctcgagacagacctcaaggatgccattccctgcctcacccccatcgctccccagaagcttgggtgaaaccaccttccatctggtccttccagttcctacctctctaattgagagggagcctagcaagttggggtagggcctgctatataattagttaggcccagtgcaaaatgaaaataaggactccaagccgggcgtggtggctcgcacctgtaatcccagcacttcgggaggccgaggagggtggatcacaaggtcaggagatcgagaccatcctggctaacacggtgaaaccccgtctctactaaaaatacaaaatattagccaggcgtggtggcgggcgcctgtagtcccagctacttgggaggctgaggcaggaggatggcgggaacctgggaggcggagcttgcagtgagccaagatcgcgccactgcactccagcctaggcgaaagagcaagactcagtctcaaaaaaaaaaaaaaaaaaaaaaaagaaatgcaagatgctaacagcagagtgttcagccaagcatgatgcccttctgagcacagaccccatgaagttgacagtggggagaagactttggacccaatggttagggaggctgctctgagaaggtgacacctgacctccttcagagatcatggccttcttgggcctggtgcggtggcttacacctgtaagcactttgggaggcccaggcaggaggatcaccggaggccaggagttcaagacaagtctgggcaatatagtgaaacccttgtctctatcaccgtgcccagcctacaaaaaaatttttaaaaatattaactgggtatggtggcgcatgcttgtagtcccagcttctcgggaatctgcggtgggaggatccattgacttcaggagttcaaggctgtgatctcactgctgcgctccagcctgggtgacagagccagccccttgtctttaaaaaaaaaaaaaaaacaggctggttgcggtggctcacgcctgtaatcccagcactttgggaggccaaggtgggtggatcaactgaggtcaggagttcgagatcagcgtggccaacatagtgaaaccccatctctactaacaatacaaaaaattatctgagcgtggtggtgggtgcctataatcctagctacttgggaggctgaggcaggagaaactctggaacctgggaggcggaggttgcagtgatccgagatcgcaccactgcactccagcctgggagacagagtgagactccatctcaaacaaacaaaaacacagagagagagagagagagagagattatgactgtcttggtaccagcagatgttgctttgtaatcaccaagtggggagagggggtggcagcctgtcttacacacctggaccacattgcccaacaacactgctccacccagtccgtaaaacctccctcggtggctatcttcacctaacatagacacctattcaaagcatctgatgaggtcatgtgcgtgctaccacttgggaaaacgaaactaagagcactataggaacaatgatgaatatcatcatcatcatcatatttacaaaatgcttgcatatcacacctgtaatcccagcactttgggaggctgaggcggcgaatcacctgaggtcgggagttcgagaccagcctgaccaacatggagaaaccccttgtctattaaaaatacaaaattagccaggcatggtggcaggcacctgtaatcctagctacttgggaggctgaggcaggagaatcgcttaaacccaggaggtggaggttgtggtgagctgagatcacgccattttgcactccagcctgggcaacaagagcaaaactctgtctcaaaaaaaaaaaaaaaatgcttgtgtaagtatttattatgttaatacaaaatatttatcatcaaaaaataaacaatacatttgattgaaccatcttgtatcatccttcttcttagtggaagacattgcatagacactgtatctcttatatttctcctgataatactacaagattgatccttgtccaacagatatttcctgaacacctactatgtgctgcaagtactgagatccacagtgcaatccggcagccagggagcacccccgatcacagacactgtggccccgcaatggatgggcgcttccattgctggagctcacttttcctgctctgtaagtactgagatccacagtgcaatccggcagccagggagcacccccgatcacggacactgtggccccgcagtggacgggcgcttccgtcgctggagctcacttttcctgctctattgttgtcatagaagaaagttccatacccaacatcacattctctacttatttgtttgtctctcttttcctctagaactgcaggagctgggaatttgaactgtttctctcacttctggatcccagcatttagaacagggctccactcacagcagccactattgctgaagaagcaaatcccgcgggattgcttgaggtcattggacttcacaagagatgtctggggtggagacaggacttgggaagatgctggttcatggatggtgtgggggctgcaggaggagatgcctggggagagagtatctagtaagaagtgacaatgttggttgggcacgatggctcatgcttgcaatcctagcactctgggaggccaaggtgagaggatcacctgaggttaggagtttgagaccagcctggccaacatggtataaccccgtctctactaaaagtacaaaaattagccaggcgtggtggtgggcgcctgtaatcccagctacttgggaggctgaggcaggagaattgcttgaacctgggaggcggaggttgcagtgagccgagatcatgccactgcactccagcctgggcgacaaagtgaaactccatcacaaaaaaaaaaaaaaaaaaaaaagaagtgacaatgtccttgggtctgccttggtgccgcaggggagtgtggtcataggtcgaagacagagaaagagccattaagaaatgggagtaggctgggcgcagtggctcatacctgtaatcccagcactttgggaggctgaggtgggtggatcacctgaggtcaggagtttgagatcagcctgaccaatatggtgaaactctgtctctactaaacatacacaaaattagctgggcatggtggcgcatgcctataatcccagctactcaggaggctgaggcaggagaatcacttgaacccaggaggcagaggttgcagtgagctgagatggcgccattgcactccagcctgggggacagagcaagactccgtctcaaaaaaaaaaagaaatgggagtagcccaaaggggtgagtgatggttacctgagggctctctctggaaagcaggacttatccacactccttagtatcttgatgctgctgagagatcaacgtcagggctgggaaaagggctgttggatttgagagaagggatgagagtcatgaagcacactggggggccgcttccagggacaggagacatcaatggaggggaaatgagaaataagtggacctgcaccttttgctaagggaggctgttaagggaggagagagaccgggcagttgctagacggatggtggccaagggagggttttgtttttgttttaaagatgggagagattataagtgctgaagggaaggatgtaattcaaaaggaaagcttgcatattcatgggggatcaggagtaaggaaggttctgaggtggcaggcagggctaggaggcggcgggattggtttttggagggacatccgttaacaggaggagagaagctggggaccaggagcaaagcccaaggccttcccaggccctggaaaccctccgtgctgggccggtcccctgctgcttctctggtttcacttcctttctcagccctgttcccacttctgctctggccccctggctccctggcttttccctgagtcctcccaccaggctcctgcctttgcctcactttcctctctccctaggatatttttgctgcagagatccacgtggctcagtccctcgctttatctgggtctctgctctcctgtccatttttcagagctcttccctgactaccacataggtaacggtatcctcctattaccttccattcagaccctctcctttaccatagggcctttgcacctgctattctccctgcctgaaatactgtgccttgcctgattattattattattattattattttgagacagagtctcactctgtcacccaggctggagtgcaatagcacgatctcagctcactgcaacctccacctgctgggttcaagtgagcaagcatggctaatttttgtatttttagtagagatggggtttcaccatgttggccaggctggtctcgaactcctgacctcaagtaatcaacctgcttcagcctcccaaagtgctgggattacagacatctgccaccacacccccggcccctgattttttgtttgtttttttgagacagactctccttctgctgtctaggctggagtacagtggagcgatcttagctcactgcaacctctgcctcccgggttcaagtgattctcctgcctcagtctcctaagtagttgagattacaggtgtccgccaccatgcctggctaatttttgtatttttagtagagacagggtttcaccatgttggacatggctggtcttgaactcctgaccacaggtgagctgcctgccttggcctccgaaagttctgggattacaggcataagccactgcgcctggcctgatttttttttttttttttttttttttttttttgtagagatggggtcttgctttgttgcgtaggctggtctcgaactcctgggctgaagctatcctcccgcctcagcctcccaaaatgctgggattacgggaatgagtcaccaagactggccccttccctgatcttgttaatgactcaatcttcacccctagctcaggcatctgtcttcagtcttgtctcttctttctccccaactaggtcaggttccctcacacaggacccttggagagctgggcacttctccaatagcatttgttgaagttactattttttatctgtgagattggctaagacctctttcttgctccagactacaggtaccatccaagcagaggccacatccatcttacccaaatgtgtattttcagtgcctgctggagtgccaggcacccagcgggtacccagaacttagggtgttgggtgtactcatggatcagaacttcatttcagcccgagctgtagacctgaagactgaggctcaagatattccatcatcagatatcgagggtcggagggtaaaaaaaagaaaaagagccaggcgcagtggctcccgcctgtagtcccggcactttgggaggccgaggcaggcagatcacttgaggtcaggagttccagaccagcctggacaacactgtgaaaccccgtctttcccgaaaatacagaaaagtcccagctactcaagtggctgaggcaggataattgcttgaacctgggaggtggaggttgcagtgagccgagattgtgccactgcactccagtctgggtgacagagtgagactctgtcacgaaagaaagagagaaagaaaaagagagagagagagagagcaagagagagagcaagagagagagagcaagagagcaagagagagagagcaagagagagagagagagatcatcgatactcatttatcaaatactcaaacccgtatctgtctacacatgctattgacctaaactatacttttcaagaatattcttcgctgccaaggctggagtgtagaggcatgatctcatctcactgtaacctctgcctcctgagttcaagcgattctcctgcctcacctccctagtagctgggactacaggcctgcactgccacacccagataatttttgtatttttggtagagatggagttttgtcatgttggtcaggctggtctcaaactcctgaccttaggtgatctgcccgccttggcctcccaaagtgctgggattacaggcatgagccactgcgcctggcttcaagaatattgttcatttacagaggcatagaggttaagagtaccctctgaaattacacagcctggaattgactgctagctctcccctctgcaatctttctggcaccagtcatgttggttcagctccctctgggcctcacttacccatctgtagaatggggataataacacctgcattgtacatctattaggaggatgaatgtgattatcacatgtaaaaggctagagccatgcttgacacacgatacatattcaaattccagtcaagcttctctctttttttttgagacggagtttcgctctgtcgcccaggctggagtgcagtggcacgatctcggctcactgcaagctctgcctcccgggttcaggccattctcctgcctcagccttccgagtagctgggactacaggcgcctgcaaccaagcccggctaatttttttttgtatttttagtagagacggggtttcaccgtgttagccaggatggtctcaatctcctgacctcctgatccgcccgcctcggcctcccaaagtgcagggattacaggcgggagctaccgcgcccggcccagcttctctttttgaatgccaggctttgtgctaaaactcttactggtttttacactgagcccccaaagattatcaacccaaatttcaggacgcatgtgtttttattaatgtctcagaaaagtcagattggaagttgcatgcacaattcctgccggaacaaagtacagccatctccctgcactaatgcatttccagctggtggagttcagtttttaaagagcagtaaatacttgcaagagacgaacggcaggaagtgaatgaaggaatagatacatttttaagatccactgacagcttgtacattctggaagccatgtgggtcagggggatgaatttattggctttaacgtcagacgctctgcaggtcgtgagatgtgccttttgtcctgacgtcagctgagagcagatctttggaaggatttgcccattgagcacatttgctgaaacatcttggcttcattacatttatgagttttaaaagcatgtaaaatattttatgtggttcttgcaaaagggtaacaacataaataacctgcagggataggatgattttcaagaaatggggtcagccggcaagccagtgttcactctgtggacattctgagtgtacagaatattaatacttcatttgcccctccagatccactctccattttctcttccctcctctgtgcccaagaggccgggcagtggggaccacatctcctggttctgtgacatctggtttcttgctgggttcagccaataaggaggagtgggagatgggagggccaaggtggggctgggctattaactccctggctggccatggtctggcagtggctgtgtccctctactgaaacccacagctcccatggggtggcctttttcctatggatcttgccattttctgataaccactccctcctgctgcctttggaggccttggagagggaagggcttttggttgttggtagccccggggtgcttcatcaaccctgctgatttctctataccttgcttatatcttggtaaataccccttttattaaacattcctcagccggacatggtggcatgtgcctgtagtcccagcactttgagaggccgaggcaggaggatctcttgaacctgggaggcggaggttgcagtgagccaagattgcgcctctgcactctagactgggcgacagagtgagactgtctctaaaaaaaaaaaacaaagaaccccaaaagactctcctcagttactctgctcaagtgagccttttaattatcttggggaccttgtataggtacagttattattttgtttgtttgtctgttttgagacagagtctcactctgttgccgaggctggagcgcagtggtacagtcttggctcactgcaacctccacctcctgggttcaagcaattctcctgccttcgcctcccaagtagctgggattacaggcatgcacccccaagcccggctaatttttgtatttttggtagagacggggtttcaccatgttggccaggctggtctcaaactcctgacctcaagtgatcctccgccttggcctcccaaagtgctgggattgtaggtgtgagccatcacgcccaacgggcacagttgttaatatcatgttagattaaaaatcaaaatcttacagaaacttcagcctttatatcacgatggcttcctgtccctatggcaaggtaatccttaagagagaggccaagctcaaagctgtagccaaataatttaatatctatgaggtgactttcacatacctcatactaaatatgcaagctacacatgacctctaccatccagaaatacctgttttagcagcgaagatagactatctgatgagaaatcgtactatgggaggaacatggcttggcaattaagagtctctatttgttttctggggctgctgtaacagagagccacagacggagcggcttacacagcacagatgtattgtctgaaggctctctgggtgctgtgagggaaggatctgtgccaggcctctctccttggctcgtagatgaccgtcttctccctgtgactctgccgtcatcttccctctatgtctgtctgtctctgtgtccacatttctcccacaggtcaccagtcctattggaatagggtcttggtaaagaccctatttccaagtagaaacacattctgaggtgctgggtctcagagctccaacatctcctttttgggaggatacaattcaacgcagagcagaggccgacatccatgagtttaaatccaggttgtgtcaccttgggcaaattatttatttatttatttattcatttttgagacggagtctcactctgtcacccaggctggagggcaatggcgtgatctcagctcactgcaacctctgcctcccgggttcaagtgattctcctgcctcagcctcctgagtagctgggattacaggcacccgccaccacacctggctaatttttgtattttttagtggagacggggtttcaccatgttgaccagactggtctcgaactcctgacctcaggtgatctgcccgcctcagcttcccaaagtgctgggattacaggcgtgaaccaccatgcccggccaccttgggcaaattatttaaactgctcagtgtttgtgtttcatcatctagaaaatggggatagcctgggcttggtggctcgcacatgtaatccagcaccttgggaggcagaggcaggagaatcacttgaacccaggaggcagaagttgcagtgagctgagattgggccactatactccagcctgggctacagagcgagactccatctcaaaaaaaaagccaggcatggtggtatgtgcctgtagttccagctacttaggaggctgaggctggaggattgcttgagcctgtgtggtcaagctgcagtgagctgtgatggtgccactacactccagcctgggcaacagagcaagacctatctaaaatataaagtaaaataaaatagggataatatttttacaatcactttgcaggatactgtgaaataaggggtgggagactgcttaaaaagtgcctggcataggcagggcgcggtggctcatgcctgtggctcatgcctgtaatcccagcactttgggaggctgaggcgggtggatcatgaggtcaggagatagagaccatcctggctaacacagtgaaaccccgtctctactaaaaatacaaaaaattagctgggcgtggtggtgggcacctgtagtcccagctactcgggagactgaagcaggagaatggcatgaaccctggaggcggagcttgcagtgagccgagactgcgccactgcactccagactgggcgacagagtgtcaaacaaacacaaaaacgtgcctggcgtgtggcaaatgttgtcggtgtgggctttcgctattattaacctaaagtgttgtactttcacataacaactccattgagcgtcttcaggcatctcaaacttccccgtccagttggctcgttttctaatcctacccatcttggaaaatggcaccctcaccagcccagttgctcaagctgtccatgatccttctcatcattgccactttcactccaccagtgaacctgctggatacccctccaaggacacgctgtattctgaattcattgcttctcttcatctcattagttagatctgatgagacggccctgccgtcctctcttggacactctcttgaacaaagcagtaacctcttagcttgttttcccgctttcactgcaccccttctagagatcatcctctgtacaggagccaagggatctttaaaaacatggatcccagccgggcatggtggctcacgtctgcaatcccagcactttgggaggccgaggagggcagatctcttaaggtcaggagttcgagaccagcctggccaatatggtgaaaccctgtctctactaaaaatataaaaattagccaagtgtggtggcgggcgcctatagtctcagctacttgggaggctgaggcaggagaatcgcttgaacctgggaggctgaggttgcagcaagcagagatcacaccattgcactccagcctgggcaacagagcgagactccgtctcaaaaacaaacaaacaaacaaacaaaaacaaaacacggatcccatcatgtcttttcctgcgtaagactctggatggctttccattgaaactagaacaaaatctacacatctttgttctaaggccctcttccaaggccctaatgatctggtctctgcctgcctctccaatgtgacttcctaccattctcccctggatcactagattttagttacatgggtcttcctgcttttcctccaatataacaagcagatccagcttcagtgcctgtgcacttgctgttctccgggcttggaatacccttccaagaactttctccctcacttcattcaggtctttgctcaaatgttacctcttcaaggaggtcttccttgcccaccatttttttttgtttgtttgtttgttttcttttctttttttttttttttgagacagagtcttgctctgtcacctaggctggagtgcagtgatgcgatctcggcccactgcaacctctgcctcccaggttcaagcaattcctgtgcctcagcctcccgagcagctggaacgacaggagacacaagccaccatgcccagctaacttttgtgtttttagtagagacagggttttgccatgttgtccactctggtctcaaactcctgaactcaagcaatctgctcacctcagcctcctaaattgctgggattataggcatgagccaccatgcctggccgagattccctatttctttttttttttttttttttttgagatggactcttgctctgttgctcaggctggagtgcagtggtgcgatcttagctcactccaacctccacctcccgggttcaagcgattctcctgcctcagccccctgagtagctgggactataagcgcatgccaccacacccggctaatttttgtatttttagtagagatggggtttcaccatgttggccaggatggtctcggtctcctgaccttgtgatccacctgccttggcctcccaaagtgctgggatcacaggcgtgagccactgcacccggccagctcttaattgcattattttatattaccattctcacagcattaatcaccctcagcagttctatttacgcacttctttgttttgcttacggtccacctctaccaccagaatgcaattgatctgatagttgagaggtctcccatgctcactgctgaatccccatcacctagaacagtgcctggcacttaataggcatccagtaaatctttgcgggggtgggatgggaagcaaacacaaaatgatatgtggttctcttctctcttttctgccccctctccaaacgaaaccaccagcagaggaaagaaacaaaaagtctatgagttgtcaagatttctgctttggggcacagtggtcatgcctgtgatcccagctttttgggaggctgagttgggatgatcatctgagcctagttgtttgagaccagcctgggcaacatagcaagactccatctctaaacaaatttttttttttttttgagacaaggtcttgctctgccacctgcattggagtgcagtggcactatcatagttcactgcaccctcaacctccaaggctcaagtgatcctctcaccttagcctcccaagtagcttggactacaggcgtgcactaccacacctggctaatttttttgtagtgacgaggtttcgccatattaaccagactggtcttgaactcctggcctcaagtgatccttccttcttggcttcccaaagtgctgagattataggtgtgaaccaccatacctgattctacaaaaatttttttaaaaagttagccaggcatggtggcgcatgcctgtagttccagcgaggtgaggtgggagcattgcttgagcccaggagttggaaactgcagtgagctttgatcctgccactgtctgggcaacagcatgagactctgtttctagaaaaacaaaacaaaacaaaacaaaaaaagattcccgctttggaacaatggctaggggctgggaatctaggatggatccagagtgggcaagtcttggcttcttataactgcagaatcccacgatggatgtgtgggtgggctggggtaacagatctactcagggtgggggctgggggtatcgcgaggtgtttaagcctgtcaataaaaaaagtgaccgctgctttgtatctgcagctactcaagggaaataaagccgtttgcttataaagaatttccataaacatttctgaggatgtgtattaggggtaatcttgggatgtgcattaaactaaacagggtagtataaacacaggaacaggctgaaagggggagaataaatggcccaggtttgccctggcccaatggccttaggcaacttgtgcagcctgcagaaacccagagacttcagttcattatttaaagaaggcggagacaaagagggaaaaccaaacgcagccctcccatgggtagatagattcttgaacaacctctggaattaagctttttttttctctcctcctagtgttcaatgctttggattcctgaagttatctatgttgaaatactcgagagatggtaaggagaacaggaacaggtagctttgggaggcacgtgctctgggctaggcatagttcttttttttgggggggcggggggagggagtctcgctctgtcgcccaggctggagtgcagtggcgcgatctcggctcaccgcaacctccacctcccgggttcaagcgattctccctcctcagcctccagagtagctgggattacaggcacctgccaccacgcccagctaattttttgtatttttaatagagactgggtttcactgtgttagccaggatggtctcgatctcctgacctcgtgacccacctgcctcggcctcccaaagtgctgggattacaggcgtgagccactgtgcccggctggctaggcacagttctaaaagctgcgtgtgcacgatctcatttcatcctcccggtaccgctttggggaaggtaacactattgcctccactttgcagaagaggaaactgaggcagagagaagggtgatgacttgctgaaggatacagagctcattacagtggaggttcagggtctgagctctagaaaagatgctccaccctatgaaggaaaaggttcattggtattatctatctatctatctatctatctatctatctatctatctatatatttttttctgggatggagtctcactctgttgcccaggctggagtgcagtggtgcgatctcagctcactgcaaactctgcctcccgggtttaagctattctcctgcctcagcctcctgagtagctgggattacaggtgtgtgccaccacacctggctaatttttgtatttttagtagagacggggtttcaccatgttggccaggctggtctcaaactcctgacctcaggtgatctgcccgccttggcctcccaaagtgctgggattacaggcgtgagccaccacgcccggcctaaatcatttgaatttgctaactcattcatgtgttgataatataaataaataatttttaaagtgtccccttgtgcaaaccaatgatgaaagcaggccatgaatagaggcaggtgatcagctccactctgtgccccggagtaccctcaaaataaaccccaaggctcaggaccaaatatagatgatgataggctaggcggtggctcacacacaaaaaatcagaggtggataagaatctatatctgcacctgccaggagccaggtcgaggaggattaagagttgggggagactttccactgtgcccattctttctacattttgggtcatatgaatgtattatcttttccaaagtttgaattattatttttgttttgtttgttttgtttttttgaggcggagtttcactcttgttgcccaagctggagtgctatggcacgatctcggctcacctcaacctccgtctcccaggttcaagcaattctcctgcctcagcctcccaaagtgctgggattacaggtgtcagtcaccgcgctcggcctgaattattttttaaaaactttgataggatataaatgaaaagaatttttttttttgagacggagtcttactctgtcacccagcctgaagtgcagtggtgcgatcttggttcactgcaagctccgcctctcgggttcacgccattctcctgtctcagcctcccaagtagctgggactacaggcacccaccaccatgcctggctaattatttgtatttttagtagagatggggtttcaccatgttagccaggatgctcttgatctcctgacctcgtgatccgtctgcctcggcctcccaaagtgctgagattacaggcgtgagccaccgcgcccggccaaaattttttttaatatgatacaaaataattttttttttttttttttgagacagagtcttgctctgtcgcccaggctggagtgcagtggcacgatctcggctcactgcaagctccgcctcccgggttcatgccattctcctgcctcagcctcctgagtagctgggactacaggcgccctccaccccgcccagctaattttttgtatttttttagtagagacggggtttcaccatgttagccaggatggtctcaatctcctgacctcgtgatccgcccacctcggcctcccaaagtgctgggattacaggcgtgagccaccgcgcccggactaatatttttttttaaataaaaaatatgaaactggcctgggcacggtggctcatgcctgtaatcccagcactttgaggggccgaggtgggtggattacctgaggtcaggagttcaagaccagcctgaccaacatggtgaaaccctgtctcttctaaaaatacaaaattagttgggcgtggtggcgcatgcctgtaatcccagctatttgggaggctgaggaaggagaatcacttgaacccaggatctggaggttgcagtgagccgagatcacgccattgcactccagcctaagcaatgaagagtgaaactctgtctcaaaaaaaaaaaaaacaaaaaacaaacaaaacaaaacaaaaaactgaaaaggtttcaggaatgcaaaggcaacatgtcccaaagtattaaaatggtacaaccttgcttttgacgtgaaggtcagttacagaacgaaattagccagcaagcaggtgagagcagagattaaaacggtaatgaagatggccattccacagccttttctaactacatggaaattatcttccttgacagtagccaacctgtgcttacattctctctctctctctctctctctctctttctctctctctcctcccacctgcttgccatgacctatttcatagtcctggcacgtgtgaaatgcctgccaagaactgcagaagacagagacacagtgctccaaaaaggttgaatggcaactttatcatggacattttggtgattacaatatctacatttcctggggggtctcagaatcacagaaattatttcaagttagtccgaggctgctcaacgctgaggtcaaaacatctgagagaaaaggttaagtaaaaaatctggttgtttctataaaactggactcattcattcaatggctatgtactgaatacctaccatgtaccagaccacgctgaggaccagccagaggcaaggcaggcacctcaggcacaaaaggcaagaaggcctcactctctgtcactgctacacctgcaaaacccctagcgtaaggttccctttgcatttcctgcccgtggtgcctgcctggcctcatcctagtgcctattccataccagacgctgttctaagcactgacgctgcagcagtgaacaaaacaaaaagaagctggacaaatgctcaagtctccacatttcagaattggatattttggtgctgaattgactgcgttaagaaacagaggtttcccggatacagtggctcatgtgtgtaatcccagcactatgagaggctgaggcgagaggatggcttgagcccaggagttttgagaccaacctggacaacacggtgaaatcccagctttacaaaaaatatgaaaattagctgggcatggtggtacacgcctgtagtcccagctactcggaaggctgaggtgggaggatcacctgagtctgaggaggttgaggctgcagtgagttgagattgtgccactgcactccgcctgggcaacagagtgagactctgtctcaaaaaaaagaaacaggtcaggcgcggtggctcacgcctgtaatcccagcactttgggaggctgaggcgggtggatcacctgaggtcaggagttcgagaccagcctgaccaacatattgaaaccccgtctctactaaaaatacaaaaattagccaggcgtggtggcacacgcctgtaatcccagctacttgggaggctgaggaaggagaatcacttgaacctgggaagcggaggttgcagtgagctgggatcatgccactacactccagcctgggcaacaagagcgaaactccatctcggaaaaaaaaaaaaaaaaaaaagaaacagaggtctttgcccactcttcagcttggagttactaagtgatctcagtcttgcttcagttgacttcttactctgcatccccaggtagccctttgttgttcaggactagcttggacaatggaaagccccatcttctgagtgctccccaatgtctggggctatagatagattgaccaatgcaatctatgtcatgccccatgaggtgggaccagtgattctcaccactttccaaactgaggtcacagttggtctgaaagctgggagggttcacccatagagaaaactggccaagacaagattcagatctggcgtgggtccgagagactcctgcaagggtgtactctagcactccacacattacttaaggttggttgagctcagaggtacagatatatgttggcgacttactgaaggctgcaaagctcattaagatcaggggtccagggtctgagctcttcaaaagatgttccaccctaggaaggaaaacgctcgttgttatcatcattgacgtttggtcactgagccttggggtttatttttaggaaactcctaaaaataatgtgttttaggacacacagtgaaactgatcatgtgtctccattcatggcctgctttcatcattggtttgtgcatgaagacacttctaaaattatttattcatattatcgatatatagatgtattagccattgcttctccctgtcacgtataagacttgacaagactatcatccccgaggtgacccccgaagtgctatcattactcaccgtcctaccctctggcccagctgcaaagaatgggtctagcttagctttctgatcctttctgccatcccaacccaatctgctctgattcatcacttcctccctaagcatcctcaccaaagcccttcagcaccagtgtcagcaacaacagccaatatgccctgggtcttccttcctgtgctctttcaaataaccagattattactggactgcctattatatgccaggccctgtgattggcactggaatgcaacaataagcaaaaacagacatggtacctacccatcttcatttagcacacagtctagtggagggaggtggacatcagttaaataatcacccccaagtacgcagaaagatcttattgcacagtatgataatggatatggagaaaaagtgccaggcaccgccaccaggagttggaactggtccaggagggcttctctgaggaagggaccttggagctgaaacctgtaggatggggcaactgggtaaagtgggcagagatagcctctgggcagagggttgagcagtgacactggagaggaagccagaggctcacttgggaaagaccttgagatggatcagaggcaggtgatggcgggcatccatggggtcttactctgagcattcatgggattcagtttgggtgcaatgcagtcgcggccggctacagttgcagaactcactggagggaggtgagagtgcatgggaagaccagtgaggaagctcctgcccgtctacaggtgatggacgatcgggatagtggggtggtagcaaggtgcaaacaaatggactcatgatgtacttggagatttggtgatggagtgcatgagggataaggagcagagtgaggggtccctgacctcctgctcgagcaaatagagcaacagtagatcctcaggtgatcttcataaattcaaaccatccttccctcccttgcacaggcaagaaacaacattgaagtatctacattgaagacagaactttgtgttcctatttgtttttctctatgctaattaaaatgccctttgtaatggccgggcatggtggctcacacctgtaatcccagcactttgggaggccgaggcaggcagatcacctgaggtcaggagtttgagaccagcctgaccaatatgatgaaaccccgtctctactgaaaatacaaaaattggccgggcacggtggctcacgcctgtaatcccagcactttggggggctgaggcgggtggatcacctgaggtcaggagttcaggaccagcctggccaacatggtgaaaccccatctctactaaaaatacaaaaaaattagccagtcgtggtggcaggcgcctgaaatcccagctactgggtgggtggggggctgaggcaggagaattgcttgaacccggaaaggggaggttgcagtgagccgagattgcgccattgcactccagcttgggggacaaaagcaagaagctgtctcaaaaaaaaaaaaagtacaaaaattacccgggcatggtggcatgtgcctgtaatcccagctactcgggaggctgagacaggagaattgcttgaacccgggaggcagaagttgcagtgagcccagatcgtgccattgcactccagcctgggcgacaagaaagaaactctgtctcaaaaatatatacataaaaacaaaacgaaacaccctttgtaaatcaatccagaggaaacttttcctttttttgccaatgaaattaattatgtcgagaaacctgaatctcatctcttctgtcttcatgtctgttacattctcaaaagacacactattgctcagtgtcagggtaggataatgagctgatcagaaacaattttcatgtcatttgaagcttaatttcattagcaaataaaaactgtttcaaataccaccattggtcctctaaagttgatttagagattcaggttgaaaatcgattaaagtgatttttccttcataaaagtttatagctctgcaggcacacacagactccttagacttgcaaagccacatccatattattttccagaatgaccgttaccgaaaaaaagtgagttgcagagcaataagtagatatgatctcgtttatgtaaaaacaaaacaaagcagtgacaaaagctaaaactgaaaatcactccaaattcagcagcattggcatcacagtggagcttgctaaaaatggggaatctcgcccgggcgcagtggctcacgcctgtaaatcccagcactttgagaggccgaggcgcgtggatcatgaggtcaggagatcaagaccgtcctggctaacacggtgaaaccccgtctctactaaaaatacaaaaaattagccgggcgtggtggcgggtgcctgcagtcccagctactcgggaggctgaggcaggagaatggcgtgaacccgggaggcggagcttgcggtgagccgagatcgtgcccctgcactccagcctgggcgacagcgagactccatctcaaaaaaaaaaaaaaaaaaaagaaatggggaatctcagatcctacccaagacccactgaatcattttaacaaaatcaacagctgattcttcagcacattgaagtctgacaagcactgctgttaacagttctttttcttttcttttctttttaagacaggttctcactctgtcacccaggctggagatcacagtccaccacagcctcctgggctcacgcaatcctcccgtctcagcctcccaagcagctgggactgtaggtgcatgctatcatacccagctaatttttttctttcttttcttcctttttatttttatttttatttattttttattttttctagatacaaggtctcactatatggcccaggctggtctcaaactcctggactcaagcaatccttccacctcagcctcccaaagggctgggattataggcgtgagccaccacacccagtctatatttatatttctacacacacacaaacacttgtgtgtgttatatatgtagataaatatattatctatgtatctaatatatctattatcctagacatacatagatatgtgtatatgaatagaaatataaatgcatagaaaagtctggactaaactgctcacggtggttaattttgagaagtttggatttcaaggtgaatgtggtggtgaatagtaagtgggaaattcaaagggggctttcacattttaccagatataccagataacctagatttactgatctaggcgcccgccaccacgcctggctaattttttttgtatttttcagtagagacggggtttcaccgtgtttgccaggatggtctccatctcctaacctcatgatctgcccgcgtctgcctcccaaagtgctgggattacaggtgtgagccaccgcgcccggccagacagaactgcttaatgcattacttgggtaatttaaatttaaatattaagatagtataaagctgaggacctaaaaaactaatcttacaaataacacggacatctcaagtccctcctctctttgacctaccaattcctcctggggggaggtgccctcagaattactcacacatgtgagaaatgatgtgcaaccgagataattcacaggagcactgtttgtaatagcccaatattgaagacaacttaaaggcccatcatagcccgggcacggtggctcacgcctgtaatcccagcactttgggaggcccaggcaggcggatcacttgaggtcaggagtttgataccagcctggccaacatggtgaaaccccgtctctactaaagacacaaaaagtagctgggtgtggtagcgcgtgcctgtaatcccagctacttgggaggctgagagaggagaatcgcttgaacctgggaggtggaggttgcagtgatccaagatcgcatcacagcactccagcctgggtgacagagaaagactgtctcaaaaacaataaaggcccatcatttgggaattggttaaatagaataaaggacatttacagcgtaacactatacagtcatagaaaagagagatcaaagggctgggcgtggtggctcgtgcctgtaatcccagcactttgggacgctgaggtgggaggatcacctgaggtcaggagtttgagaccagcctggccaacatggtgaaaccctgtctctactaaaaacacaaaaattagccgggggtggtggcacacatctgtaatcccagctactcaggaggctgactcaggagaatcacttaaacctgggagatggaggttgcagtgagccgagatcgcgccactgcactccagcctgggtgacagagcgatactcttgtttcaaaaaaaaaaaaaaaaaaaaaacaagatcaagctctatatgtgttgagatggaacaatggccaagagcaatggttcatgcaaggacaaaggggctgagccatctggatgatatgttgacaattgtgtagaaagggagcggggagtggaatacatatttatatttggttgtatatgcacaatgtagctctgagtatcatattatacaacataaaggagtagtggttgactatgagaaggggaacaaatgggtggctgtgggacagtggttaagagacttttcaccatttgttaaacttggtgattatttgaagcacttggatgcattacctattcaaaatcttactaaaacaagaacaaaacaaacaaacagaagcttctaggattcttttatttctctgagaatccttactcctgggaactacatctggagaactcattgtgagcccaataacacaggggcttggtttcccatgaaaagaaggctgcctttcaaggaaggagagggacattctccatcctgaggggacaccccccacacaccccatgcttcctttattgccatttcgtcctttgctgctctcatttgatgtggctcggcttttttttttttcttaaagttcccaccagttctctttaaagacagcaccaaaactaaaactaaaaaccaatttaattttacttttaaagcactccccctttttttttaatttttacaataatgaagatcttattctaggatgaaaataatgaataggaaagagctgtttattcttccttcttcccacccctgaatattgaagtaaaactaaataacaatgaaagtgactaggaacagcagacaaggggaaatatgtcagaatgagaacatctcatgtcatctccagggctgggggagaaacatctttcctggtacaaggtcccggtaccgaagaaatacaatttttcaaaaagttctgtaagtgatacatgcttgttacaaaaaatctctaccattttcaaagcttagaaggcagaaactccttgttagttggtaatttctttatctctctcatgctttcctgatatctgaattacgcttgagaaaaagaggaaaataaaacaagacagaaaataatgcttccttaggtaaatacaatcggctaagaaaggcttccaagaagaagcataattcactgagtcccagcttattgagagaatccccaaacaaaggacttttgtgaagggatcagtctcagccaccataacttagcatacaataggatggcttaataaaagaggtttccaaaggccaagatggttatcaaagatctcccaaggagagagggagaagaagagacagagagaaaaagagagagagaaagagggaaggaataaccttctggaatgttcccaactatctttccagttagcattcatgaatacgtgaaaggtttcatatcacttggagtcccacccatttttcaaaataggaagcttccgctattttcttctggatccgttattgggtcaagagaagtctctgcctcttgtccaggacatttggatatttcttatttgttatttatttatgagatagggtctcactgtgtcgcccaggctggagtgtagtggcacaaccatggctcactgcagcctcgacctcccaggttcaagtgattctcccgtcccagcctcctgagtagctaggacgacaggcaccattgcacttggctaattttaaaatattttttagagatagggtcttgctatgttgcccagcctgctctcaaactcctggcctcaagtgatcctcccccaccttggcttcccaaagtgctcggattgcaggtgtaagccacccactgcacctggcctagatttggaagtttatataattacttttcaatgaatgttttaatgaatgttgcaatgaatgtctccttgtttaccttagatgactgggtgaaataaacaactcctcaaattttgttatgttactctgccatattatatcacgtatgacatatattgttatattatattacatatgtatgtctatacatatgtttctgtgtatgtagatgtttatacatccagcatattaaatccatcaagcttttcgacaaaggtgaaaggttgtagacttcacaaacttaaataatgtaggttgtagagtcagagcaacttctggttgtcccagtgaacttggataagtgacttaacctggctgggcctccatgtcctcctggcaagtaggggtggtaaccgcccatatggagggggttccacaagataaatttggaaagacataggcaagtggcacaaagcaacggtagctgtggttgtcccctccggacagtggttattaaaaagacttataggttgggcgcggtggctcctgcctataatcccagcactttgggaggccaaagcaggcagatcacttgaggtcaggagatccagatcagcccagccaacatggtgaaaccccgtctctactagaaatacaaaaattactgagtatggtggcgcaagcctgtagtcccaacctactcaggaggctgaggcatgagaatcacttgaacctgggaggcagaggttgcagtgggccaagatggcgccactgtactccaccctggcaacagagtgagaccttgtttcaaaaaaaaaaaaaaaaaaaaaaaagaagatgtatgttcccggctttaggagactaccttgggggctgtgcaaacacaggctactctattcaaattctggatttctttaaaaaacaaaaccacaaacaccaaaatactctgatagtgcttcaaaacctcaaactggccagtgtgtggctcatgcctgtaatctagcactcgtgtgtggctcatgcctgtaatctagcactctgggaggctgaggcaggtggattgcttgagcccagaagtttgagaccagcctgggccacatggtgaaaccccatctctacaaaaatgataaaagttagccaggcgtggtgactgtgggcctgtagtcccagctactcaggaggctgaggtggatcaccagagcccaggaggtcaaggctgcagtaagccgagatcatgagatcatgccactgcacaccagcttgggtgacagaatgagaccctgtctcaaaaaaaaaaaaaaaaaaaagccctgaaattcagtggtctcaaaatttagagtatattcagctattcagcaaaaaagagaaaagaataaaattcttacttgaggccgggcatggtggctcacgcctgtaatcccaacactttgggaggccgaggtaggtggatcacttgaggtcaggaattcgagactagcctgaccaacattgtaaaaccccatatctactaaaactacaaacttagccaggaatggtggcacatgcctgtaatcccagctacttgggaggctgaggcaggagaatcgcttgaacccaggaggcagaggttgcagtgagctgagatagcgccactgcactacagcctgggtgacagagtgtgtgaaactccatctcaaaaaaaaaaaaaaaaagattatggtgagcattgcaatgactgtgcgcacactaaagaccactgaattggtcactttcgataggtaaattgagtatgaagtatatctcattaaagctgttatgaaacacattttaaggccgggcgtggtggctcacgcctgtaatcccagcactttgggaggctgagggatcacctgaggtcgggagttccagaccagcctgaccaacatggagaaatacaaaattaccccggggtggtggcacatgcctgtaatcccagctactcgggaggctgaggcaggagaatcacttgaaaccgggaggcgaaggttaccgtgagccgagatcgtgccattgcactctagcctgggcaacaagagcgagactccgtctcaaaaaaaaaaaaaaaaaaaaaagtaagaaacacattttagtgcactttcatatgccctgggcacgcttgtttaaaatgtaggctggtacactttcttccagggattttgcttggagagaccattcttatgccaccaggggcctttcaaagctttgagaaacaccgcaggaggcctgacccaagtcttatccaccatctagacatccactgtgcctgatgttccatcctgaacaccttgttttgtccatcctttctgaaattttatgtccaggttgctatcattgctttattttacaggggaggaaatggaagtggtataaaacaaaacagcgaagctcagggctgtggcttcagcgggcctggaaatgggtagatggggactataggtcaagtgtgttctcctgcagcccacacctgatcactgcagttctttttttttttttttttgagacacagcctcgctttgtcacccatgctggagcgcagtggtgtgatctcggctcactgcaagctccgcctcccaggttcacatgccattctcctgcctcagcctcccgagtagctgggactacgggcacccaccaccatgccaggctaatttttttgtatttttagtagagacagggtttcaccgtgttagccaggatggtctcgatctcctgacctcgtgatccgcctgtctcaggctcccaaagtgctgggattacaggcgtgagccaccgcggccggcctggtcactgcagttctagcggcacctaggaatgtggttaacacaggcttttcttgctgtggtgggccttactatatacgacacctaatatagtaaaatacgaccatatttcacattcagttaaacaaatgtgtgtaatttttgaaatgttctgaagtgtgcaattactaattttgctcagatagaatattttgctcttgtgattttttttttttttgagatggagtctcgctctgtcgcccaggctggagtgcagtggcgcgttctcggctcactgcaacctccgcctcccagattcaagtgattctcctgcctcagcctcccaagtagctgggtttacaggtgtgtgccatcacacctggctaatttttgtatttttagtagggatagggtttcaccatattggccaggctggtctcgaactcctgacctcaggtgatccacccgcctcggcctcccaaagtgctgggattacaggagtgagccaccacacccagcttgctcttgtgatctaaagacctctaaaaaccaccagttgcagatgccagttagtatgatgtaacaatttgtagccatttgtgaattctctgtgtatattttatcacttattaaatttttatttaatttttagagaggaagggtctcactctgtcacacagactagagtgcagtggggtgatcatagttcattgcagtctcaaactcataggctcaggcagcttctcacctctgcctcccaagtaactaggactacaggcatgcaccaccacaccaagctaatttttcaattttttgtagagacagggttttgtcatgttgcgcaggctggcctccaactcctggcctcaagccatcctcctgccttggcctcccaacatgttgggattacaggcatgagccactgcatcagcctctgtctatttttaaatttgttttggtaagccaggatttgatatttttctttggttcttgcaaagctcttgcgtccttggatctagtactgcagcaccctgtaaaaatatccctgaaagtgtaggcaaagtcatgcttggttttgtggtggacgtgatcccacttgttgtctggtttcattcactatttatctcaaagcgcatttctgatttctctacttcattctgtccattgtagcctgaggaggtggaggtggggctctgaagatggctgagaggtggggactgtgtccaggtgtgtctagtaatctgaatatttcttaatcttccactgtatgcaaatactttactgagctacatgataggaagaaatataaaacaaaacccctttagaagattgcaaatgaatactgtagaggaagctagcaggcatgtataagaaaagattcttgacctcaggcattataagaaaaatgcaaatataattagtatatcttttttttccttatcagatcaacagatatccaaccttttgataatgccgtctttgcagggtgtggggaatcaggctctgacatacactgccaagagggtgggagttaaaaatgctttagtctccttgggagtatttggaaacataaactaaaatagaaactatacacgttcctttttttttttcttttgagacagggtctcgctctatcgcccaggctggagtgcagtggcacgatcagctcactgcaaccttcacctcctgggttcaagtgattctcatgcctcaggctccagagtagctggggctacaggcgcgcgccaccatgcccagctaattttttgtgtgtgtgtgtatttttatgagagacggggtttcgccatgttggccaggctggtcttgaactctcaagggatccacctgccttggcgtctcaaagtgctgggattacaagcatgaaccaccatgcctgactgagatgtacatactctttgacctaacaattattctactggaaatttatcctatagacccccactcaggaatatatgataagtgatgtacgtataaggaaatacattgcaacattgtatgtaaaagctaagtaatagaaacaacccaaatgtccattagaggaagctgtcaaaataagttatggtcatctatgatatggcattgtatttctgcagctggaggcagctgtgtttgaactgacattctgtgagagatagactgaaattaaaaacacaagctggggccaggtgcggtggctcacgcctgtaatcccagcatgggaggctgaggtggttggatcaccttgaggtcaggagtttgagaccagcctgaccaacgtggtgaaaccccgtctctactaaaaatataaaaataacccgggcgtggtggcacatgcctgtaatcccagctacttgggaggctgaggcaggagaatagcttgaatccggaaggcagaggttgcagtgagccaagatcatgccattgcactctagcctgggagacaagagtgaagagtgaaactttgtctcaaaaaaagaaagaaaaacacaagctgccagactgttttggatgctaccaactgagtaggaaaaaatattaggttgagccatgtgaaagtaccaatatgagaccatttttgacatataaacatgacagtttcatgtacttcaatgtcatatataggtatgtagggttttttttttttttttttttactttttattttgaagcgaagattcacatttttttttgtttttcttcttctttttttttttttttttttttgagacggagtctcgctctatcgcccaggctggagtgcagtggtgtgacctcagctcactgcaagctctgcctcccgggttcacgccattctcctgcctcagcctcccgagtagctgggactacaggcgcccgccaccacgcctggctaattttttgtacttttagtagagacggggtttcaccgtgttagccaggatggtcttgatctcctgacctcgtgatccacccgcctcagcctcacaaagtgctgggcttacaggtgtgagccacctcgccgtgcccggcctcatgtgtacttttaagaaataatagatactgtgtaaactttacccagttctcctaatggaaacctcttacaaaactttagtacagtatcataaccaggatgttgacattgatatattcaagatacagaacatttccattatcataaagattcctcatgttatacttttatagccacacccacttccctccaggcctaacccctccttcattcctggcaaccactaatctgttctacatttctatagtttcgtcatttcaagaatgttacataaatggagtcatatagtatataatcttttgagatcagctttttctctgtatttatttattttttgagatagggtctcgctctgtcactcagacggcagtacagtggcactatctgggctcactacagcctccgactcctgagctcaatcaatcctcccacctcagcctcctgagtagctggggctataggtgtgtgccaccatgcccggagattttttttttttttttttttttgagacagggtctcattctgtcacccaggctggagtgcagcggtgcgatctcggctcactgcaacctctgcctcccgggttcaagtgattctcctgcctcagcctcccgagtagttgggattacaggcacctgccactatgcctggctaatttttgtatttttagtagagacggggtttcaccatgttggccaggctggtctcaaattcctgacctcaggtgatccgcccatctcagcctcccaaagtgctgggattacaggcgtgagccactgctcccagctgaacacagtttttatttctctgtgataagtgccccggagcgcaactgctgggtcatgtggtactgcatgtttagtttttttttttttaacaaactgacagttttccagagtggctgtaacacgtgacattacctccagaaatgcatgggtgctatgtacagagtggtaaaaaaaaatctctgtcgccaaaccatctgaactctagtgaaagtggggtgggtgtgaacaaatgaacaaaatctacattgtattagacaaggataagtgctttggagagaatgaggcagccagtgggggtggggtctgcaagggtctggatggatcaactgcagtggtgaaaagggaaggctttggagggaaagtgacacctgaagatagaataaatgtgttattgtggaatgcaatgctgctccactgcccccccacacctttttttttcaatgccttgaaaagacttaatccagacgaactctcttctcgcctgctttctttttttttttttgagacagagtttcgctctgtcacccaagctggagtgcagtggcacaatctcagctcactgcaacctctgcctcctgggttcaagtgattatcctgcctcagcctcctgagtaactgggattacaggtgcatgccaccacgctcagctaattttttgtatttttagtagagacagggtttcaccatgttggtcgggctgatcttgaactcctgacctgaagtgatccacccgccttggcctcccaaagtgctgggattacagacatgagccactgtgcctggcttgattttagattcccatctgatgttctctataactttgatgactccccaaacaaaggctcccaaacacactctgaggcttatggtaatgaatttaaataagccagcgtgtgtactgggtgtagcacatgtccagaagcaggaaaccctcactgtcctctataagctgctgttctgtcaggaacattgtgacagcactgctgctgatattatcgttttcatatcccagaatgggaatggtttcttgccatcctcagacagagttgcctcatcttgatgacagtggtaggatgagtaataattatctaccattgtctaacttagatatcaagcactttgcaccaattcttattgggtgctcaaagaaacctcaggaggaatatgctatcagtataactccctttttatagctaaggccacaaaggctctgagtggcgaagtggtccatgagggtctcagagcagtaatggggaagaatgagattttgaatcctaaagcactagaggaaatgttcttcaactatgcctcagaggtgtcctagaaaatcagaccggaagagtattcaccaaatttagtatttgtgctgcaacgattggaaatccataagtaaaagaatgaaagtggacttttccctcacaccatatgcaaaaattaatttgaaatgaatcaataacctaaacagaagagctaaagccataacactcttaggagaaaacacaggaggaaatttccatgaacttggaattggcaatgggttcttagatatgaacacacaaaacatgagcaacaaaataaaaaataaagcacaaggaacaaaaggaaataaattggacttcattaatttcttttcttttttttttttgaggcagagtcttactgctgtctcggctcactacaacctctgttccccaggttcaagcaattctcctgcctcagcctcccaagtagctgagaatacagctgtgcaccaccacacccggtttcaccatgttggccagggtggtctcaaactcctgacctcaagtgattgtcccaccttggcctcccaaagtgctgggattacagaagtgagccaccacacccggccgggacttcattaaatttaaaaacttttgtacatcaaaggacattatcaaggaaggaaaaagacaactcacagaatgggagaaaatattcgcaaatcaatcataaatctgataagggtctgggatctaaattttttttgtagccaccacgcctggctaatttttgtgtttttagtagagatggtgtttcactatgttggccaggctggtcttgaactcctgatctcaggtgatccaaccacttcggcctcccaaagtgctgggattacaggtgtgagccaccactcccagcctgggatctaaaatttgtaaggcattcttgcaaagcaacaacaagacaaacaactcagtcaaaatatgggcaaaggacttgaatagatattttcttccaaagaagatgtacaaaaggccagaaagcacatgacaagatgctcagcatcattagtcactagagaatgcacttcaaaatcacaaggcatcatttcacatccactaggatggttattatcaacaaaaccaaacagaacacaaaaacccaggttgggtatggtggctcatgcctgtaatcccagcactttgggaggccaaggtggttggattacctgaggtcaggagttcgagaccagtctggccaacatggcaaaaccccatctctactaaaactacaaaaattagccaggcatgatggcacacgcctgtaatcccagctactcaggaggctgaggcaggagaatagattgaacccgggaggcggaggttgcagtgagccaagattgctccactgcactcttgcctgtgcgacagtgagactccatctcaaaaacaaacaaacaggccaggcgcggtggctcatgcctataatcccagcactttgggaggtcaaggcaggtggatcacgaggtcagaagattgagatcatcctggcgaacacggtgaaaccccgtctgtactaaaaatacaaaaattagcctggcttggtggtacgcacctctagtcccagctactcaggaggctgaggcaggagaatcacttgaacccaggaggcagtggttatagtgagccgagatcgagatcatgctactgcactccagcctgggtgatacagcaagactccatctcaaaaaaacaaaacaaaacaaaacaaaacaaaacaaaaaaacaaaaaataagaaaacaggcattcaaatacacgtacatacgtgttcagagtagcactatacacaatagccaaaaggtgtaaatatcccaaatgtccatcaactggtgagtgcagaaatgagatgaagtactgtataagttctgttgtaccacagacaattattcagccataaaatggaatgagatactgatacgtgctacaatgtggatgagccttgaaaacattacagtaagtaaaagaagccagacacaaaaagccacatttgattttgttgataagaaataggaatctgaatctgttcactttaaccagaataggtaaggccatggcgagatgccgattggcggttgccaggtcctgggggctagggtagaactggggagcgactgcctactggctacacggtttccacttggggtgatgaaaatgtttcggtgctggacagaggtggtgataacgcaatactgtgaatataccaactgctcctgaattggtcactttaaaatggttaattttatgttatgtgaatttcatgtcaattttttttcttttttcttttttttgagacagagtttcacacttgttgcccaggctggagtgcagtggtgcaatctcggctcaccacaacctccgcctcccgggttcaagcgattttcctgcctcagcctcccgagtagctgggattacaggcgtgcgccaccacgcccagctaattttgtatttttagtagagacagggtttctccatgttggtcaggctggtctcgaactcccgaccttaggtgatctgcccgcctcggcctcccaaagtgctgggattacaggcgtgagccaccgcgcccagcttcatgtcaatttttaaatttcacgtttgattccaggggttgggagtttgctgcccttcatcatggagtttgctgcagccagtgcaggggtgaaaagggaagagcaaaacaggcagatctcgtttaggagagatactgcaatttatctgatgctgatcttttaagtcacttctggtgccaaagagcactggacttggagcctgaagaactgagacttcctgacatttggccttaagcaagcctgagcttagacttcctgagcttttcccgatgttttgtgttgggcagccccatttccctggacagctgcagggatggagggaagtgatgcgtgtatgcatgtgggcttgttccttccttctctattccacaggctgtcagtgtacaggtatgtttgcacacaagaaagatgttggtcctggggtctcagaggcaccaaagaactggccagctttgtccaattcatccaaaatccagtcagtggcgtaaagaggaagccatcacaccaggcctttgccaacaaagcagacaggctgcccagcccaggagctgcctactcttactcgcctgttggtttgagcagatgcagccatgacagcagggttacaaaggccaccccagccaggccaccaccctccaccagggctctgccaggatggattgaaccctggccctcatgtgttacttgatgacagcaagatacctgataagaaactgtttggatgtcacagagaacacattttaacagcccagagaggtgggattatcacagagtggtgtaaaagaagcaaaattatttcggataaaccatgaaggaattttattacccagtgcagttataaatcaagttctctgtaaatgcaataaaacaattgtttagtctgcatgattcataccacaaacctcaggattcttggttttgatcttattgccgcaataaactgtctagacagcaattaacaaactgtttttccttcagatgtttggagtgctggtacagcgggaatcggcacttagaaattcaggggcttcttcctggctcctattaattatcaggttgggtgagtgggaaaagcccagaatcttctcagaattagccttctggaagtgtccttcccctgtgaatgtgccacttcaccacataatattcacttataactggcagaaaatccaaatatacagattttcttaaaccttggcctgtcactttagagagaagtgagcagaattaactttttttttttttttttttggagacagtcttgctctgttgcccaggctggagtacaatggcatgatcttggctcactgcaacctccacctcccgagttcaagtgattctcctgtcttagcctcccaagtagctgggattacaggaatgcaccaccacgccctgctaattttgtatttttagtagaaacagggtttcatcatgttggtcaggctggtctcgaactcctgacttcatgtgatccacctgccttggcctcccaaagtgctgggattacaggcatgagccaccatgccatgcctggcctagcttttgtatatccttggagcaaaatttatatacagataagtcatttcttcatgtattcattcatttattcagcattagggtataatagtttagaacactcactcaggagtctaatcgactttcgtgccatttcaggtacgtgatttcaccttcctgaagtttattttgcatatgttgaaagtcatggaggaaaattaaatgaaattatctgtgtaaattgcctggaacataataagagctcaaaactattcactattattgtctatcatccatgcattcatctgtccatccacgtattcaccatccctctatctatccatccatccacccacctatccatccattccttcacccttcctttcttccttccttctatcatttattcacctatctgtccattcttccttcttttctttccttccttccaacatttatccatccatccatccatccatctatgcatccacccacccacccacccatccttccatccatccctccatctatccctccatctgtccatttatctacacactcactcattcattccttctttgcctggtactctatgccagattgttgagattataaaaacaaataagatatatcccgacttcatggagatcccagtttagctggaaagacagcattaggtctctggctgtaggcagcatctgatcctgaaggaaatagtgagagaaaacctggggaagtgtttagtttctggtctagccctggtgatttaagatgatatgtgagacgcactcacctcccagggctcctgatggctcagagctcagtggatgaacatggcttcatccacacagtcagttacacggtgagactacacatttggcaaatgccccaaacaaacaaacaaacaaacaaaataatttgggttcaaggtaacagtaaatcacactcttcaaacctaaatattcttctctaggcttcttcccaccttttcttcccttctttctgccaactccttctcctccattttgcgtcagacaccatgtgcattgggaggatgttgctaggtcacagtggggacacttgctctatttacagagaaaacagggctcctcagattcacagggtttgcaaaggaaagccctcccagaaaggaattccagccttcccttctgaggctggtgcagcagggctgggggctgcaggaacagaccagagaaagtgactcagaattcccttcggctacagtacgattgcgagaaccattttaagtatgagctgtggctcccctctccctttttcctcagcctccagggagctgccatttaaaaaatcttggcctggccaggggcggtggctcatgcctgtaatcccagcactttgggaggctgaggtgggcggatcacctgaggtcgggagtttgagaccagcctgacccctgaccaacatggagaaaccccgtctctactaaaaatacaaaattagctggaggctgaggtaggagaatcgattgaactcgggaggcagaggttgtggtgagctgagatcacgccattgcactccagcctgggcaacaagagcaaaactccatctcaaaaaaaaaaaaaaaaaaaagaaaagaaagaaagtaatccatgtctttctcacgtaccaacatccagatagaagcagtccaggatgggtagagcagctcctttggtagaacccacctctattgcgctgccatgcctggcacgttgctcccatccccatggtccaagggtcctctcctccacatccccatccagccagcagaaaggggagacaatgaagggaaggcacgccccagaaggatgtgcctccgaggcctctcacatcactggaatgctctccccatagcctaggatttagtcacgagaccgcacaggggagctgagctctttgttctggttggccatgtgtccacatctattactacacaaaagtggggatggccatggagggagaaccagcaaattctttcatacgctcctatggccttgatccatccagtgggtacatttctgagcccctcgctttctcactgcctcttctgctgggagcatgactcccgggctgcagaaggacaggctggtgcattgagagtctttaggctttgcttgaattgcaaatgctctggttttctggcaagacggaagtaggtccctgggcagaagtgtcagaagacctggggttgcctctgaacgtcactgtttctgggtctgagaccttggactcagaaatgaaaaaaacctctgaacctcagtgaaatgggggaaaaaataaatgacttccatagagttattgggaggaccaaactgcattaccaaatacattctgaaaaggtgaaagttctccaaatgattgtgtgttttatcatctccaccaccaccatttttctttttcttttttttttgagacagagtcttgctctgttgcccaggctggagtgcagtagcgcgatctcggctcactgcaagctccacctcccgggttcaccccattctcctgcctcagcctcccgagtagctgggactacaggcacccgcctccacgcccggctaatttttgtatttttagtagagacggggtttcaccgtgttagccaggatggtctcgatctcctgacctcgtgatccacctgcctcggcctcccaaagtgctgggattacaggagtgagccaccgcgcacggcccatttttcttttttcttaatgcatgtatgtagctgaatttgtgtaagtacaatggatccatgatgagagtctatttattgaatttttttttttttttcagatggagtctcactctgtcacccagggtagagtgtagtggtgtgatcttggctcactgcaacttccgcctcctgggctcaagtggttctcctgactcccaagtagctgggattacaggcacccaccgccacgccaagctaattcttgtgtttttagtagagatggggtttcgccatgttggccaggccggtctcgaactcatgaccttaagtgatccgcccgcctcccaaagtgctgggattacaggcatgagccactgcgccttgcctaattatagaaattgtatttggagagaaaagtatactctttctctaagcatttctgacattacattctttcaaagcaatgcgaacaacttctgaaggtactggtctgggctccaagctcagatctgggtggcggaagattctatgaacaggtcctggccaggtttgggggaggtccatgagcaggtcttctcaccaggagcaatatccctaggaaaccaacccacagaatagaaattgaaagagcaaaaaggcttccttttctaagaaaggccatgatgtctccaggaatagcataaatatcccctgcagggcttatcccagaaaagaacattcagaggggcctctgagttgctctgcacccccagcagatgccctgctccaaatggtctcttgtgtctccagctctccacccaggctctacctggaaggctcttctctgacttgtcctcacgcagccccttcaactgagccacagtgacgtcccccataggcatcctggtgagtcctggtggggtatcgtagaggctcaatgacctttaaaatgagttcaggccaggcacggtggctcccacctataatcccagcactttgggaggcgaggcaggcggatcacctgaggtcaggagttcaagaccagcttgaccaacatggcatctctactgaaaatacagaagttagccgggtgtggtggtacatacctgtaatcccagctactcgggaggctgaggcaggagaatcacttgaacctgggaggcggagattgcagtgagccaagactggccactacactccagcctgggttacagagcgagactccgtctcaaagaaaaaaagtcagttcaaactttgcgattcaacaactgggtgactctccggagtcaatgaattcgccaatccatggatactgatgagcttgacctgagctgcttgggaagctgagctgcagtgaatacgatgcagcagatggagccaacgctgcaaagcagagccgcgggcaacacggagttcccgggactccctcaaaatcctcccctttatctacactggctcccgcagacccgcgggcaacacagagagtgcttgggactccctcaaacttctctcctttatctacactggctcccacaacaggatctcaaccctggttgtacttgagaatcagcagggaaacttctaacagttctgatgcctgagccatgcccacagatcaattatatcagaaaccccgtagctgggacctgggcataagtattttaaagctccccaggtgattccaaagtccagccaaggtagacatccactgctctggggaccttgtccatcccacaggtagaaggccccatctgtgaccacttttctgaggtccagacttgccaatttattttctcaacatcaccatttcagtgcctaagagacaactcatcttgccagagtaaagtggattgtcccctccacacctgcttctcttctcaccagggtcccccagctgtttcagggcaaaaactgaacaccttctgattcttttctttgtctcacagcccatagccaatttttttggaattctcaacagctataatgtcaaaatatatccccattctaatctctgctccccacctcccctgctagcctggagatcccctattcttcttttgaatccttctagtttggtctctgccaacagctcatgtaaaatctatgaaaaaatgaagctccgggttgggtgtggtggctcatgtctgtaatctcagaacttgttgttgtcgttgctgttgttttgagactgagtcttgttctgtcgcccaggctggagtgcagtggtgaaatctcggctcactacaacctccacctcccaggttcaagcaattttcctgcctcagcctcgtgagtagctgagactacaggcgcgtgccaccacgcccagctaattttttcttttttttgtatgtttagtagaagcgaggttttaccatgttggccaggctggtcttgaactactgacctcaagtgatctgcctgcctcggcctcccaaagtgctgggattacaggcgtgagccaccacacccggccaatcccagtactttggaaggctgaggtgggaggattgcttgagcccaggagatggaggctgcagtgagccatgattgtgccactgcactccagcctgggcaacagagcaaaaccctgtttccagaaaacaaacaagcccaaaaactcagcatgaaaagatgctcagctcattagccattggggaaattcaattaaaaccaaaagaagggccgagcacggtggctcatacctgtaatcccagcactttgggaggctgatgtgggtgagtcacctgaggccaggagttcaagactagcctggccaacttgatgaaaccccatctctactaaaaataaaaaaattagccgagtgtggtgatgcacgcctgtaatcccagctgctcgggaggctgaggcaggagaattgcttgaacctggaagacagagattgcagtgagccgaaattccaccactgcactccagcctgggtgaaagagcgagactccatctcaaaaaaaaaataaagatatgttgagatacacaaataccattgtgttacaattgtccacatcattcaaggcaataacatgtacgtgtttgtagcctaggagcaataggccataccatatagcctaggtgtgtagactcactccataacgtacccataataacgaaatgccataatgtaccaacaggagaacagatgaacaaattatagtatgtttatacaatgaaatactactcagcagccgggcacggtggctcacgcctgtaatcccagcactttggggggccgaggtgggcggatcacgaggtcaggagatcgagaccatcctggctaacacagtgaaaccctgtctctactaaaaatacaaaaattagccgggcatggtggcggggcgcttgtagtcccagctactcgggaggctgaggcaggagaatggcgtgaacccaggaggcggagcttgcagtgagccgagatcacgccactgcactccagcctgggcgacagagcaagactctgtctcaaaaagaaaaaaaaaccaaaaaactactattcagcatacaagaaatgaattactgacgcaagcagcaacgtcaataaatcttatagacattcctctgagcaaaagaagctgggtaaactttatgaatgcatttataagaggttctagaaaaaaatgattcaagggtgatatgttaaaatatggcttctagccagtgggatggtcaggggtttgactggattgattgaaaaggagcatgagggagccttctggagtgatggaaatgatttttatcttgttttgggtggtgattacaggtgtccaaagttactgaactcacttaagagctgtaagtcttggctgggcagtaatcccagcactttgagaggccgaggtaggtggatcacctgaggtcaggagttcaagagcagcctggccaacatgatgaaaccctgtctttactaaaaaaaaaaaaaaactacaaaaattagctgggcatggtggtacgtgcctgtagtcccagctacttgggaggctgaggcaggagaattgcttgaacctgggaggcggaggttgcacgctgcagcctgggccacagagcaagactccatctcaaaaaaaaagaaaaaaaaaaggaactgtaagtcttactgcatgtaaatcataccttaaaaagtaatttgggggccaggcgcggtggtgcatgactataattccagaacttttggaggccaaggcaggcagataacttgagaccaggaatttgagactgagactaggctgggcaacatggcaaaactctgtctctataaaaaatacaaaaattagctgggtttggtggtacacaccagtagtcccagctacttgggaggctgaggtgggaggattgcttgagcccaggaagttgaggctgcagtgagccatgagtgcaccactgcactccagcctggatgacaaaacaagaccttgtctcaaaaaaaaaaaaagaaaaaaaaacacagagtcccacaaacctctgctcaaactccccctaccctcacccccaacctttaggacaaaatctggagcacccactctggtcttcagagccttgcatgaactgccgtcttttccctccttggcagtgtctacaaccacccttccctttgctcttttttttcagacagagtctcactctaacacccaggctggagtgcagtggtgccatctcagctcactacaacctctgcctcccaggttcaagcgattctcctgccttagcctccatgtagctgggattacaggcatgtaccatcgctcctggctaattttttgtatttttagtagagacggggtttcaccatgttggtcatgctggccttgagctcctgacttcaaatgatctgcccgcctcagcctcccaaagtgctgggattataggcgtgagccaccgtgcccggctggcttttctttatagaatgcctgccggtgtatacatcactaagcaatcattcttttagttctcacacacttttttggaaacaccatgcattcttttgtacttaggggctttgctttaatattatgttcctgagattcatccaggaccactattaattttcatagttgtgttctaatctatgatgatcacataatttagtcacccattttcttgggaaggggcatttaggttgtatccagatttttgctgttactcacaatgctgccaagagcattgttgtacatggctcctgggacatgtatgccagaaatgtttaggatatttatctatgaatgaaattgcttggtcagagggtatgtgcgttttcagctgtatctgatatttcaaagctattttttaaggtggttgtaccaactacacttttcaccaactgtgtaaaagagtttcctttttgtcctatatccttatcaatactgggtgcccttaaatttattaatttttgccaatcttgcggttttaatttgcattgtctcaatcattaataaggttaatcatcactttatatattgggcatttatcaaaaaataaagctgtgtatttttagatgggaacaaatgcacagaaaaagatctaaaaccgtgattctaagaaggggagtggaaaaggaggggtgggatgtgaatactttgttctgtgtgatcttcacatttttacaacaaaaattatttaatatgcttataaaattcatataaaaatggccaggcatggtggctaatgcctgtaatctcagaactttaggaggccctgggtgggcgaaatcacttgaggccaggagttcgagaccagcctggccaacatggcaaaaccccatctctactaaaaacacgcacacaaaaaaatcagctgggggtggtggtgcacacctgcagtcccggctacttgggaggctgaggcaggagaattgctggaacctgggaagtcaaggttgcagtgaggcaagatcatgccactgcactccagcttggatgacagaatgagactctgtctcaagaaacagaaaaaactggctgggcgcggtgtctcatgcctgcaatcccagcactttgggaggctgaggtgggcagatcacaaggtcaggagatcgagaccatcctggtgaacacggtgaaaccccatctctactaaaaatacaaaaaattagccaggactggtgggcgcctgtaatcccagctacttgggaggctgaggcaggagaatcacctgaacccgggaggcggaatttgcagtgagctgagatcatgctactgcactccagcctgggagacggagtgagactccatcccccccgcaaaaaaaagaaaaaattgatctaaaaattaagcaagatatagatcagtacgcatcattaatttttttattttaaaaaattatatgtaaacacacacatacatgcattgctttatatgtgtgtgtgtatgtgtgtgtatagaagaaggcctgaaataattttctcagagtttttaagggtggttttctgtgtggaggtggggatgcaactgtgcagtggtggagttaaggagggtaggggtagtagggggcggtatataattgttccttttaaattttatgcaccttttaaattggatattttcttcagtctttaatacaaaattcactagttctgcgatttaacatttttatgttattttacaaaaatctttctgatatttccttcattctcaatatccaaattttcttttttttttttgagacagaatcttgctctgttgcccaggctggactgcagtggcacgatctcgactcactgcaagctgtgcgtcccggattcatgcctttctcctgcctcagcctcctgagtagctgggactataggcgcccgccaccacacctggctaattttttttgtatttttagtagagacggggtttcactgtgttagccaggatggtctcgatctcctgacctcgtgatccgcctgcctcggcctcccaaagtgctgggattacaggcgtgagccactgtgcccagcctaaatatccaaattttctgtggactgtcacaggtataaagtaaagtgccccacccccacacccaccatgtccaaaaagaaaagagtaaacaaggttccagtgacctggaccatacctaactccgcctcagttggtaactctgatgtgaacagcacacatcagaagaacatagggccaccactgtgatataggtggtaaaattccctttatcataaacatgtacaatgattacatatccataataattagaaataaaaacatttttaaaggtaaagctaagcagaaaaataaaaaataaaggttcctttatgttcttgagatctgaaaaggaaattatacgtacagcctttacaataccttttattataaatctgtttatgagtttaaaactatgctgtccaatatgtagccactagccatatgtggctatttaaatttaaattgactgaaattaaataacattaaaaattcagttccttagtcacatgggcccactatttcaagggtccaacagttactttgggctagttgctattgtattatataatgcagattaaaaatatttataccaatgtggaaagttctgatggacagtactggattaaaaccttatatagatattattcttgttcaccaccatgtgagaaggtttttaccatgggaaggaattcttgtttgttttttttgtttttttgtttttttgttttttaaattgaggtagggtcttgctctgttgcccaggttggagtacagtggcacaatcacagctcacttcagcctcgacctccaggtctcaagtgatcctcccacctcagtctcccaagtagctggaactacaggtacctgccaccacactcagctaattttttttattttttgtagaaatgggatcttactatgttgcccaggctggtatggaactcctgggctcaagtgatcctcctgcctccagcctcccaaagtgctgggattacaggtgtgagccaccattccttgggccattcttgtattttaagtactcagagacatccaggaaaggtaaccgagtgggtaacttcagcacttcagagagtgcctagtgtgtgattctaagctcatcactccagggacccagcacccaattccagctgtgtctttcctccatttaaatcaccctctgtggatcctattaagtattctcccattctggtcaaactggtttatgaaactactatttgagaagtccagtcttgcaggccgatgggaccctgcctttgtgtggccgtaataaggttatttatgagggctggagccgattctcttgattcttcgggaagatgaaaggtttggtggtaaaggaaagcagaagcttggggagcaggtttctccttatagcaatgactctccagataggataaatcacccaggaccattaaaccttcaagaagagtgctcgtaaacctccgctggaaactcacaaagcatcttctctcttggagcagcctatgtggagagggactcgcctctttttccaaaccaccagccagtcacagattaaccctgctgccatctctgcccaggacactgaagcagaaaatcactttctttttttttttgagacagagtctcactccgtcacccaggctggagtgcagtggcgcgatcttggctcactgcaacctccacctccctggttcaagcgattctcctgcctcagcctcccgagtagctgggattacaggcatccgccaccatgcccagctaattttttgtatttttagtagagatggggtttcaccatattggccaggatggtctcaatctcttgacctcacgatccgcccacattggcctctcaaagtgctgggattacaggcgtgagccactgcacccagccgaaaatcactttcaaagtcatcctttcaggtggccaactgtccctgtttgtctgagatgaaagggtttcctgggacaagagatcaggaaagtcccaagcaagtctggatgaactggtcactctatttcctccgtaccttgggggcaagcaatgcctgcgactcaacaggacaaaaacatttgttcatatttagctcccatttatgaggctctgcaatgtgtcaggtgctgggcaaagacagtgctttacctgacgtgtctcgtttcatcttaacaaccaaatgagcttggtgttattattatccccatttgacaggtgaagaaaccgaggctcagaaagctaaagtgacttgtccaaggtcacaccttaaatggtgtctgtgtctccatagctcatgctctgaactgctcctctaattcttccttatctttcttttttttttttttttttttgagatggagtctcgctctgttgcccaggctggagggcagtggtgcgatcttggctcactgcaacttccacttacggggtgcaagcgattcttgtgccttagcctccagagtagctgggattacaggtgtgcaccaccacattcggctaatttttggatttttatttgtaattttttttgagttggagtctcactctattggccaggagtacagtggcacaatcttggttcacggcaacctccatctcctgggttcaagcgatcctcctgccttagcctcctgagcttaccaggcgcccgccaccacacccggctaatttttgtatttttagtagagatagggtttcaccatgttggccaggctggtctcaaactcctgacctcaagtgatctgcccgcctcagcctcccaaagtgctgggattacaggcgtgagctaccgtgcccagcctgtatttttagtagagacggaatttcgccgtattgcccaggctggtctcaaactcctgaactcaagcgatccacccgccttggcctcccaaagtgctgggattacagtgtgagccactgcacctggccttcatctttcttaatctgccttggatttttggggttccagaagcttcccacaaaaagagtcattggagatacttatattgttaaccaacggggtaggagaatgactttcagctgaactttatttgcacgttaaaaagtcaagtctaattttctaaaactatctggactctttcactggcaaatgccgagttggaaggaaagtccataaatcactcacagacctacattggccgtggctatatgcaaacccagtactgctggtctggccacctgggagcaccaggcataggatggggtgagatttccccaccaaattcagggcatatcagagctcagagtacctgcacccaaagggaaataccccaaacactggcttcaggaaagttctcattagatagacacaagaaagttttgccctaatcctcagtattggctaacagatcacttttattgaggaattactatgtgccaggcaatgcctgttttcatccacttctcatgtccaattatctgcgaaatagataagattattatcccagcccagagatgaggaaactgaggtgtggagagctggcgagattgatctagaccagtggttctcaagcaggggagggtttgccctccatgcacatttgccactatctggacacatttttgatcatcacaacctgggatggggttgctgctggcatctagtgaatagaggccagggatgctgctccacattctacactgcacagaacggcctccctctaccccagtaaaagaaattacatggccccaaatgccaatagctctgaggttgagaaactcttgtctaaatcaagaaaagggtggcaactcctggggaacccttcactccagagtttgaggggctcacatgcctagcaaggggtagagcagtgattagatcccacttgcttttaccttcctatgccctggagaaggattactgaaaggtcagttcaagagggtatgaacttgttgtgtgatgctagaacagtgctgggcacaaataggtgcccagtaaatatttgttgaatgaatgaatgaatactaagtattaataccatttattgagggctttctgtcaatgaacacaccaagtatattgccagcatcatctcatttgattcttggagtagaccccagaggcaggtaccactccccacagtcttattttacagagtaagagacagagagggctgggggcggtggctcacgcctgtaatcccagcacttcgggaggccaagacgggtggatcacgaggtcaggagaccaagaccatcctggttaacatggtgaaatcccttctctactaaaaatacaaaaaagttagctgggcatggtggtgggcacctgtagtcccagctactccggaggctgaggcaggagaatggtgtgaatccgggaggcggagcttgcggtgagccgagatcgtgccactgcactccagcctgggtgatagagtaagactccgtctcaaaaaaaaaaaaaaaaaaaagagacagagagaagaggtctcatgccagagtcatacagcttggccagtgactgaggttttccattcagggtggtccaagtttaacttagaattacattgcctcataccagaatctctggaaacaagaataatttccagggcttacatcatgtagcaactttttttttttttttttagacagagtctcaactctgtctcccaggctggagtgcaatctcagctcactgcaacctcttcctcccaggttcatgccattctcctacctccgcctcccaagtagctgggactacaggtgcccaccaccatgtccagctaatttttttgtatttttttagtagagacagggtttcaccgtgttagccaggatggtctcagtctcctgacctcatgatctgcccgccttggcctcctaaagtgctgggattacaggcatgagccaccgtgcccggcccacatgtagtaacttttttcactgttccaggttttataaatatatcccctttttcagcacttgctctcgcttaccttttttcttttgtaggcagggtctccctttgtcactcaggctggagtgcagtggtgcgatcatggctcaacctcctgggttcaagtgatcctcccgcctcaggctcccaagtagctgggactatatacgtgtgacaccacacccagctaattttttaattttttgtacagacagggtctcactatgttgccctggctgggttcaaattcctgggcttaagctatctctgccacctcagcctcacaaagtgctgagattacaggcatgagccactgcacccaggcagcccctttctaaaaaaataaaatgggccgggcgtggtggctcacacctgtaatcccagcactttgggaggccgaggagggtggatcatgaggtcaagagaccgagaccatccaggccaacatggtgaaaccccatctctactaaaaatacaaaaattagctgggcgtggtggcgggcgcctgtagtccagctacttcggaggctgaggcaggagaatcgcttgaacctgggaggcggaggctgcagtgagctaagctcatgccactgcactccagcctggcgacagagtgagactctgtctcaaacaaacaaaaacgagttttacactattctataaataaaagggtttcagggttagccatgaaaggccccagtgacaaagttgtaatgtgatcagaaatgaaaagctcaaaaaaagatttcagttggtgatgaaacaaggcaaggattatgaatctcacaggcgtttcctttctccagaaactgaaggcacctttgccaggagaaaaaggcagtttccatggcagggacccagccttgacttccccaggatctgtccacatgaacgttgtaatgagtccacccgagatggggactctccattgtatttacattttatgacatctttttatatttcacaggagaggattctgaataaaaatgagcccggcttttagggctgttttttattatcagcatttggtaaaaaacatccttaatgaggtgttaagagtcatcctgcatcctattgtggaggaggcctgggagctcataaacccctgaaaagccacagggtaatttataatccttgatgccaccccttcctgacacaagcgcttcagagggagcatctttagttctcgggcacttcctgcaatttacacgcccaagctgcttcgaccacaatctgaattcaaccttattaagtaaagttcagcccaggattcttcttgccaagctgttcacctctgagaaactctggccagcctgcctgaaattaattagagtttccgttgagtttggactgaaggtgtggctctagaaagtgttcacatttctctccttactggtgaggaatttaacagttatggttctggggaaaaacaataacataacaacaacaacaacaaaaaaaaccacttttgcttctctgcaagaagaggagtttcttgatactgtgatgttttactcataagttcatatcctttctgaaatagacttcaattaagactgagggtgtcctaagccccagaatgatatactacttcattgagaaaaaaatactgccacttatataagggatctaaaagagccagattcatagaatcaaagtgtggaatggtggttgccagggggttgtgggagagggcaatagggaattaccaatcagccaataatcaatgggcataaggtttcagttaagcaacaggaataagttctagagatctgctgtatgacatttaacctagagtcaacgacagtggattatacactgaaaaatttgttaagagggccagccttgtatggctgggagcagtggctcatggctgtaatcccagcactttgggaggccaaggcgggcgcatcacctgaggtcaggagttcgagaccagcctaaccaacatggagaaaccccgtctctactaaaaatacaaaaaaattagccaggcgtggtggcgcatgcctgtaatcccagctactcgggaagctgaggcaggagaatcgcttgaacccaggaggtggaggttgtagtgagccgagatcgcgccactacactccagcctgggcaacaagagcgaaactccgtctcaaaaaaaaaaaaaaaaaaaaaaaaaagaagggccagccttggtgggtcacacctgtaatccgagcactttaagagcccaaggcggaaggattccttgagcccaggagttcgaggtcagcctcggcaatatagtgagaccctgcctctatttttcatttaaaaaaaacccaaaatttgttaaaagtgtatatctcatgataagtgttcctctcaatcaagtaaaataaaacgaccgccacaaaccagactttgtgatggcgacaaatatctcaggtttaatcttgaaatctgtaaattgaagaataaagaaaataatttaatcctttcctttatatccttttttgtttgtttgtttgtttttgtttgagatggagttttgatcttgttgcccaggctggagtgccatggtgcgatctcagctcactgcgacctccacctcccaggttcaagcaattctcctgcctcagcctccccagtagctgggattacaggcatgtatcaccacacccggctaattttgtatttttagtagagacggggtttcaccatgctggtcaggctggtctcgaactcctgacctgaagtgatccactcaccttggcctcccaaagtgcagggattatagacattagccacggtgactggccctttcctttatactttatatccttcttatttggggatctcttttttattttttatttttttaagaaaagtacatatgacatggtatatattgacccttcctgactttattcaattaaatattataattgactattagatggttatttctttattgctgttaatttttttttttttttgagacagtctctctctattgcccaggctggagtgcagtggcgcaatctcggctcactgcaagctccgcctccctggttcacaccattctcctgccttagcttcccgagtagttgggactacaggtgcctgccaccacgcccggctaattttggtatttttagtagagacggggtttcaccttgttagccaggatggtctcgatctcctgaccttgtgatctgcctgcttcagcctcccaaagtgctgggattacaggcgtgagccactgcacctggcctattgctattaaattttaacatcagcatttcacatagtccctatttggctttcatagttcctgttgactgaatatcatttatatttaacttgaaaaatagatatttatatttcacttgtgtggacatagttgaaaaagtgtaggaattatagattttatcatgcaacattttatgttgtttccagcctctgaacgaataaataatttatatgtgcaatgtatatgttttgttcctaggtcctctggggaggcattgtaggggaaaaacccacaaaacctggtgttggagggtcagggttcaagttctgttcttagcaataatgtgatcttgagcccatcaatcattgtttctgaccccacatgtccttattagtaaagtgaaactgataatacatatgccactgcattgttaaattactattataataataattattattatttgagacggagtctcactctgccacctaggctggagtacagtggcgcaatcttgcctcactgcaatctccacctcccgggttcaagcaattctcctgccttgagtagctgggattataggtgcatgcaaccacgcccaggtaatttttgtatttttagtagagacagggtttcagggtttcagggtttcaccatgttggtcaggctggtctcgaacttctgaccttgtgatccgcccgcctcggcctcccaaagtgctgggattacaggcatgagccatcccgcccggactgttaaattagtatttattttctttttcctttttttgagacggagtctcactctgtcgcccaggctggagtgcaatggcgcgatctcggttcactgcaacctccgcctcctgggttcaagcaattctcctgcctcagcctcccgagtagctgggactacaggtgcacactgccacacctggctaatttttttctattttagtagagacagggttcaccatgttgcccaggatggtctggaattcctgagctgaggcaatccacccttctcagcctcccaaagtgctgggattacaggtgtgagccaccgtgcctggcccttctttttttttttctgagacgaattcttgctctgtcacccaagctagagtgcagtggcggatcttggtttactgaaacccccgcctcccaggttcaagcaattctccagcctcagcctcctgagtaactgggattacaggcatgtgccatcatgcctagctaatttttgtatttttagtagagacggggttttaccatgttggccaggctggtcttgaactcctgacattgtgatttgcccacctcggcctcccaaagtgctgggattacaggcatgagccaccatgcctggcctatttattttcattataattgacagttatttaattaagtataaacttttagctacgtgttgtgaatgtgtgttatgtcacttgactaacctataaagtgtgaacagttattagcatcacaggtgaggttcttgagcctcagagaggttaagtgacttgcccatggtcacacagcctgaaagtggcaaacctgggatatgaacctaggaacatatgactgcaaaaacagtaccccaagtcattcgacgtgaagctgccttttgatgatgtaagtgagattgactgcgacttgtaaagctgtctgtaaatgcctagttaagagagatatggccaagacatcagaaattttgagcagaagaaggttctaggaatcaactaattaaataacgtgattttagaatgatgaggccgagagtctaccttgggtcagaaactaagatagagatgtcagatgagaagacagatgcctagctgcaccctgggctccctgtagtgctgtgtttaggttctcagacccgggagtcagagcctgggctcaatattgagctccggttcatactagctgtgtaccttggacaagatatgtaaattctctataccttagtttctgcatctctaaaatgggatagctaagagtaattacaggctgggtgcagtggctcatgcctgtaattccagcactttgagaggccaagatggatggatcacgcgaggccaggagtttgagactagcatggccaacatggagaaaccccatctctactaaaaatacaaaaattagccgggctggtggtgcatgtctgtgattccagctacttgggagcctgaggcagaagaattgcttgaacctaggaggcagaggttgcagtgagctgagattgcactgctgtacgcaatcctgggtgacagagcaagactctgtcttcgggaaaaaaaaaaaaagtgtaattatagaaagattaaacaagtttaaaatgtgtgacatgcttaaaatagtggtaatttataaatgtatcattattctggccaccactgatcgtctttaaaactgcaccattcctatttgatgagctgaacatacaaaaacatccatggataccattatcatctttatctcttgcgaaacagcaaatgatgaataaacccgtacaattcatttttctttttttcttttttttttgagatggagtctcactccgtcacccaggctgcagtgcagtggcgcgatctcagctcactgcaacctccgcctcccgggttcaagcaattctcctgcctcagcctcctgagtagctggtaccacaggtgtgcaccaccatgctcggctaatttttgtatttttagtagcgatggggtttcaccatgttggccaggatggtcttgatctcctgaccttctggtccgcccgccttggcctcccaaagtgcagggattccaagtgtgagccaccatgcccggcctgtacaattcatttttctaagccactaatctgatcaattatttcactgccttgattaacacttttccatgaagtatttacaagtcacatatctgataagtatccaaaatatgaaaacaactctcaccacttgacaattgtattcattgtttttaagacaaaaaatccaattaaaacacgggggaaaaaatggaatagacatttccccaaagtacatatacaaatggccaaaatgtacatgaaaagatgctgaggatcattagtcattagcgaaatgcaaatcataactgcaatgtgatgccacctgagaccctgtagggtggctataataaaaaatatggagagtaacaagtgttggcaaagatggagagaaattggaactctcatacattgttggtgggagtataagtggtacagctgcttttgaaaaatctggtagtttcttaaaatattaaacataattttcatttgatccagaagttctactcccaggtatatatttgagagaattaaaaacatatgtctacaaagaaactgtttttttttgtttgtttgtttgttttgtttttttttttagacagggtcttgctctgttgctcaggctgaaaaacagtggtgtgatcttggttcactgcagcctcttcctccagggttcacacaattctcgtgccccagcctcccaaatagctgggattacaggcatgtgccaccatacacggctaatttttttttttttttttttttttttttgcatttttagtggacatggggtttcaccatgttggcctggctggtctgagactcctggcctcaagaggtctacccaccttggcctcccaaagtgctgggagccactgtgcctggccctctacacagaaatgtgtatacaaatgtttatagcagcattattcctaaactccaaaaagtagaaataacgtaaatgtccattgactgatgaactgataaacaaaatatgctctacctatacaacggaatattattcagctgtaaaaagggaagaagtagatcactggcacagaatagagagcccagaaataaaccttcaggtatatgggccaatgatctttgacaagggtgccaaaactacacaaggggaaaaggattgtctcttcaacaaatggtgttgggaaaattggatatctacacaaaaagaactaaagacatacccttttcttcttgtgccatatacagaaattaattcaaaatggattaggaaccagggctttgtgtggtgtctcacgcctgtaatcccctcactgtgggagacccaggcaggaggattgcttgaggccaggagtttgaggccagtctgagcaacacagcaagaccacatgtctacaaaaataataattgtaaaaaagattaaaaacctaaacataagacctgagattataaaacttctagaaggaaatatactggggcggggcggagtggctcatggctataatcccagcactttgggaggccaaggaaggcggatcactggaggtcaggagttcaagaccagcctggccaacatggtgaaaccccgtctccactaaaaatacaaaaattagctgggcatggtggtgggtgcctgtaatcccaggtactcaggaggctgaagcaggtagaattgcttgagcctgggaggcagaggttgcagtgagccgagatcctgccactgcactccggcctgggcaacaagagcgaaactctgtctcaaaaaacaaaacaaaacaacaaaacaacacaaaaacagaaaacaaatattggcaagaaattggtacccttagatactgttggtgggagtgaaaaatggtgtagccaccatggaaaacagtatagtggttcctcaaaaaatttaaaataaatggctgggcgcagtggctcacacctgtaatctcaacactttgggacactgagacgggcagatcacctgaggtcaggagtttgagcctggacaacatggtgaaaccccgtatctactaaaaatacaaaaattagccagaaatggtggtgcgcgcctgtaatcccagctactctggagactgaggcaggagaattgtttgaacccgggagtttacagtgagctgagatcgcaccactgcaaccccagcttgggcgacagagcaaaacttcatctcaaaataattaattattaaattaattaatgaccataaaaaactatcacctgattcggcaatcccacttctgggtatatatccaaaagaattgaaaacaagatctcagagagagatttgcaacatgttcattgcggcactattgacaatagtcaagatgtagaagcaaccaaaatgcccactggattaatggataaagaaaatgtgggccaggtgcgggggctcgcacctgtaatcccagcactttgggaggctgaggcaggcagatcacttgaggtcaggagtttgagaccagcctggccaacatggtgaaatgccgcctccattaaaaatacaaaaattagcccggcatggtggcgtgcgcctgtggtcccagctactcaggaagctaaggcaggagaatacgatgaacccaggaggcggaagttgcagtgagctgagatcgcgccactgcactccagcctgggtgacacagcaagacttcgtctcaaaaaataaataaaaataaaataataaataaatacacccagaaaaggcacttgtttatagaatgagagctgcaattagaaggtagcgtgttttcttgtccatgttgggcactgctctgtgcacatctgcaaacgaccttaagagcaccaggagcactaatttgggggttacaaataaatttcagtgagtaggtgaatttgaaaatatgcaggcctcaattaatgaggatcactgtcttttgaagaccattattgcataagtgcatttggaaaagccagcctcattctgatgaaaaaccgtgcgagtattgctggagaaatgttagagtcgaaatttttcctgatttgcagctgagagtctcctaactgatccagaatcccagatttcaccaagaatcatgaatgaaagggagtgttttccatagcatttgggagacagagatttcacacccccaagatgagtcacaactgattattgagggattctctgtggctaggatatttcagccaagagttgaagaatgcagaagtccttcttcctccgtacaggagagcccatcttattatttccctctctcttgttcctaagacatggctgtttactcttttatttttgagacacagtttcactctgtcgcacaggctggagtgcagtggcatgatcttggctcactgcaatctccgcttcccgggttcaagtcattctgccattctcatgcctcagcctcctgagtgactaggaattacaggtgtacgccaccacgcctggctaagttttgtatttttagtagatacggagtttcaccatgttggccaggctggttttgaactcctgacctcaagtaatctgcctgctttggcctcccaaagtgctaggattacaggcgtgagccatcacacctggctggtttcgctgtttaaaaacccattctgggccaggcgcggtggctcacgcctgtaatccaagcactttgggaggccgaggcgggcggatcatctgaggtcaggagttcgagaccagcctgaccaacatggagaaaccctgtctctactaaaaatacaaaattagccgggcgtggtgaggcatgcctgtaatcccagctactcaggaaggctgaggcaggagaattgcttgaacccaggaggcggaggccaggaggcagaggttgcggtgagtcgagatcacgccaccgcactccagtctgggcaacaagagtgaaactcggtctcaagggaaaaaaaaaaaaaagaatctgctggatgcggtggctcacacctgtaatcccagcactttgggaggctgagttgggtggatcatgaggtcaggagtttgagaccagcctggccagcatggtgaaaccccgtctctactaaaaatacaaaaaattagctgggcatagtggcacacgcctgtagtcccagctactcgggaggctgaggcaggacaattgctggaacccagcaggtggaagttgcagtgagccgagatcgcgccactgcactccagcctgggtgacagactgagcatctgtctcaaaaaaaaaaaaaaaaaaaaaaaaccccaacaaaaaccaattctaacatgcttttggttccagagcgctctggaaaactcaaataagtcactatagttaatataatagtagatccagtttccatttggctgcctgggttctaatctgagctctattctagctgtgcaatgttgaaccagcttatgtgcctcagtttcccttgtctgtaacaatgtaaaatttttgcagtagatgtaaaaatcattgtgaagattggtgggctttttgaggaagatgcagttacaggtactgcgaagcggtggtctgcctgctcctgatcctgcctcaccgctgtgggctctcaaggaacaagtgggtcaggaagccacacgctgcagccatggcttttatctgtttattgatttttagagctgggggtctcactgtgttgcccaagatggtctgaaactcttgggctcaagcgatcctcctgccttggccgcctgaattgctgggactacaggggtgagacactgtgctcagctcaaaatgtattaagagcaggcattgtggacctcgtgaggcaattcacattgccattttaagctacatatttaaatctaagctgtccactaggctttggagaggcatttcctgaatgtctgttctaaaggaggaactcttccaaccctcttgttctgtggcagataaccctgatcatgacttgcaattactttatttcaatttatattttggtttgttctcatcactagaaatgaaagcctcataaaaacagggactttattgctcctgttcatctatatatccccaggacagtgccaggcacatagtagatgctcaataagtatgtgttgactgaataaacgaatgaataacagaatttggtgtctgactcccaagcccttgtacttaacacaaggctgtgcttcttctcacacccttctttgtgtggtagtgggtttggtcatgacttagagcagcattgtccaatatggtagccactagccacatgtggcaatttaaatttaactttaaagtagtgaaagttaaataaagttaaaaacccagctccccaggcatgctagtatatttccagtgctcagtacctacatgtgacaaatggctagtggagagttcccatatagaacatttcaattgtcacagaaagttccattaacggagctgattgagatcagtggttctcaactggagaagattttgcctcccaaggggcatttggcaatgtctggaaacctttctagttatcacagcttggaagatggacctgacatctagtgggtagaggccagggatgctgctatacatgctacaatacacaggacagcttcacgctagagaagtatttggcccaaaatgtcagcagtactaaggctgagaaacatggtttacaggaaggcactggggtgacagttttctctgtatgaccagaatgctaaattcctcaggatctggatgggttaatttgcaattaaaagcctgataaggactcttcagtgtatgtggcatggtgtctagtttcctggcttgaattcctggaattcctcctaatattttgaataggatattaaaatccagaaatcattgcccagctgggttttcaagtgcccacacttccctgtgagactcattcaacgggattcacaacactcagaccaaaaggcatttctaaggctagattgttctagatcaaggctctgtaagacacagtcccggccaagcctgcccgtgaacagaatggcttttatatatttctttaattggatgagaaacaatcaaaagaaggccagatgaggtggctcatgcctgtaatcccagaactttgggaggccaaggcaggcggatcacctgaggtcaggggttcgagcgagaccagcctggccaacatggcgaaaacccatctctactaaaaataacaaaaattagccgggcatggtggtgcacactgtagtcccaactactcaggaggctgaggcaggagaattgtttgaacccgggaggcggaggttgcagtgagctaagatcgcaccactgcactccagccagggtgatggggtgagactcggtctcaaaaaaaaaaaaaagaaacaattaaaagaagaatattttatgacacatgaaaattatataaaattcaaatttcagtgtccacaaagaaaacttctttggaacagtcaccaccataatttatataaaagtattgcccagggctgctttcgtgttgttgagtggtccagccacagaagccacagggtccgcaaagctgaaaatattcaccacctgcccttttacagaaagactttgctaattcctgctctagatactaaagaccttactggattttttctttccttccctccccctacccatttgttcttttattttcttaagatcaaaattatcctgataatttgcaggtgtagacacagagcaaatgaccaaatggactttcttcgggaagccgagggcggaggggattagcttccatgcaggggcgttcatgtgaaagattcctcttagatgaatttagggaacaaagaccccgtggtcaatgatttctgtgggtctgaaagtaaaagactggctgagttcttttgtcagttttgcagcacagatcatacagttactgaatactcccctccacggtagtattcttttaacgcttcactccatgacctcacaaaaataattgctgggaagcaaatttacttcgccctctgcctttatttgctgaccaaggggctcagccgatagccgtattgttgatggaaatgcaaaagggggtggagggaaggagaaagaatacaatttggccagggaagatggatatattgaaaggattcgaggatttgtccttgctttataattggagtcaggattttccttcaagttaccctgcacagactggaaggaaacctctccagcctcctccacccccatggcctgttaaactctttctcaacaaattgaaatctggcttttaaatccttgtcatttttatgattatatgattatagcgaacattacaacttgtccagtgaatggactttaaaaaaaattttttttttttttttttgtagtgagcatcaggctatgttgaccaggctggtcttgaactcctggtctcaagcaatcctcttgccttggtctcactttttcaaattctgagaatgtctttattcagaaagaaagtaaaaactgattaaaaatgatttgtatgcattgggaccagttgggatttgatttttttttctctctaagtctacaggagttttcaccgatggctttcaattctttggaatttctggaaatggaagcttgcatcttggaagcaaacctacaaacaaccttatattgtcccataccctcattttagaaaacagaccactctgattactgtggggattacctggagtgatctgggattcccttggcaggaacaaagtggaaagattttccggccctttaatttgtaaaacgaaaacctgtgttggctgctactggtagaaaaataaaaaattaatacacccaaagtaaaagttagcaaggataactgccaggccagacacagtttgttataagccccgcaatgtgcaatattgaaagtggtttccttcccctagtacttaggcatccaatcagtcaaaccatctgataactgcttatctcctctaatgcgactgagatgtgtttgatgactctttgcatttcaatcgtaaagctcaccttaggtaagcaccttgaggagaaaaaagaaattcacaagtctcagcttttgcattcatggaattttcttttcacaatggaaggaaagttaagctggtgcccgttagggggaggggtgcagaggtggttgaagatctttgaaagggaggggcgcagtgttcagtttgtgtgtagtgcacacacacatacacacacatacacaaaggtaaaacacttcatttgggttaaatggttgagaaacaatcaaaagaagaatattttatgatgcataaaaagatatgaaattcccatttcagtgtccattaagaaagctttattggaatgtagcttcttgtctcaggagccaaaatagtaaagcaaagcaaagcaatcccgagagagggggaagatgaaagatgaacgggccactctatgcttcatttttagctacctgaggacacaggaaaaatagaaaatgtgtataaggaaagatttccaggattttggtaatcataacttggcatgtctgactccctgcacccacagtgctttttataatcctctacccagcaatcttctgtgaaatggggaagaagcggggcacctctctcataggagtaaaaggttgatagataggcacgtgtttattctagcctggcacacagtacatgctcagaaaatcttagctgttattcttatcggggtggttattgttttgttttgttttgagatggcgtctcgctctgttgcccaggctggagtgcaaggcatgatctcagctcactgcaacctccgcctcccgggttcaagcgattctcccgcctcaaccacccgagtagctgggactacaggcgcccgccaccacgcccggctaatttttatacttttaatagagacagggtttcaccatgttggccaggatggtctcggtctcgtgaccttgtgatccaccagcctcggcctcccaaagtgctgggattacaggggtaagccaccgtgcccggccaggggggtggttattgttaacagcagttgctcttgtacttttccatctcctattgttggagaagtattagattggtgcaaaagtaattgtagtttctgccttactttatttttatttttatttttttattttttatttttgagatggagtcttgctctgtcacccaggctggagtgcggtggtgtgatttcggctcactacaacctccgcatcctgggttcaagagatcctcctgcctcagtctccccagtagctgggattacaggcgctgcaacttctgcctcctgggttcaagagatcctcctgcctcagtctccccagtagctgggattacaggcgctgcaacttctgcctcctgggttcaagagatcctcctgcctcagtctccccagtagctgggattacaggcactacaaccaaggctggctaatttttgtatttttagtagagacaggatttcaccacgttggccaggctggtctcgaattcctgacctcaggtgatccacccgcctcggcctcatggagtgctgggattacaggtgtgagccactgtccccgaccacttttttgccttacttttaatggcaatttatagttggttggtgcaaaagtaatttctgcaattacttagtataacttcaacttggttataacttggtataacttcatttgggagcagtttggcaatattcgccaaactttaaacatacactttcacccagcaactcagtttataggagttcttcctatggatcaattccctcttcatatgccaatcagaattcatatttttgttttgaattccaaaccacgtgcatgtattattgttttagttttgtgttatcaggatatacatatttaggattatgtcacttggtgaattgactccattatcattttgaaatgtcccattttttccctggaaatatttctcaaattgaaatctattttatctgaaattaatgcagccaatccactttattaggctttgtgtttgcatggagtatctttttcaatcccttacttctaatctacctggtctttatatttaaagtggggtttcaggctgggtgcgatggctcatgcctataattccacaactttgggaagctgaagcaggcagatcacgtgaggtcaggagttcgagaccagcctggccaacacggtgaaaccccttctctgctaaaaacacaaaaattagctgggcatgctggcgcacgcctgtaatcccagctacttggaaggctgaggcaggacaatcacttgaacccaagaggcagaggttgcagtgaactgagactgtgccactgcactccagcctaagtaacacacacctaaggtggtgtgagactccacctcaaaaaaataataaataagaaataaagcagagtttcttagagacagcatatggttgggtcttgctctcttacaaaaccaacaacttctgacttcaattggaatatttagatcatttacccttataggaattttttatatggttgaacttaaatttaccatcttgctctttattttttattgtcctatttgtagtctattctttttccttctttttgcctggctccctttggaatgagttattttttaggattccattttatttccatgactggtttattagctatatcttgtttttttttttttggttgctctaaggtataaagtatgcatcttcagcttatcacaatgtaccatcaaatgataacagttcacatataatataaaaatcttgtagcaatatacttccctctccccatgtcttttgcactgtatttgtcatagattttattactacttatgttataaactctgaaatatttgttttttttttcttgctttagagtcaattgtcttttttttttttttttttgagatggagtctcgctctgtctcccaggctggagagcagtggcgtgatctcggctcactgcatcctcagcctcctggttcaagcgattcttctgcctcagcctcctgagtagctgggactacaggcatgtgccaccacgcccagctaatttttgtatttttagtagagacggggtttcaccatgttggcctggctggtcttgaactcctgacctcgtgatccacctgcctcagcctcacaaagtgctgggattacaggcatgagccattgcgcccagccaattctcttcttcataaataaaattttaattttagaacagttttaggtttacagaaaaattgaaaagattgtactaagagttaccatataccctaaacacagttttccctgttattaacatcttacattagcatggcacatttgttttaatcagttaaactattagccaaacattattattttattattattattatttttgagacggagtcttgctctgtcacccaggctggagtgcagtggtgcaattctggctcactgcaacctccacctccaaggttcaattgattctcctgcttcagcctcctcattagctgggactacaggcgcccaccaccacacccggctaattttgtgtatttttagtagagacaaggtttcaccatgttggccaggctggtctcaaacttctgacctcaagtgattcactcgccttggccttgcaaagtactgggattacaggtgtgagccactgcgcccagccaatttttgtatttttagtagagatggggttttaccatgttggccaggctggtcttgaactcctgacctcaagtgattcacccagcttggccccccaaagtgctgggattacaggcatgagccacctcacccggccaatttttgtatttttagtagagatggggttttaccatgttggccatgagtcagtctcaaactcctgacttcaggtgatccacccaccttggcctctcaaagtgctgggattacaggtgtgagccactgcgcccggccagccatacattattgttaattcaattgcatactgtattcaggtttccttggtttttacctaatgtccttttactgcgttaggatc

SEQ ID NO: 26 - DNA, 220 bp, Homo sapiens
ctgagatcaagttttgggagcagacagacaaacatcatccctcacagacaggcattccgttggctattctcttgcaaacagaatcaagcactagaccagcagcatgagcctcaggatactgtgggactggggagggagagaggggttgagtagtcccttcgcaagccctcatttcaccaggcccccggcttggggcgccttccttccccatggcgggaca

SEQ ID NO: 27 - DNA, 269 bp, Homo sapiens
cagaaacattcatcggcacacacacacatttacaccttaaaagaaaagatttaagaagctaagagaaagggaagggtcctctaccggagcgcagtacaggtccggagtacacgaagtcggctcgggagaccggtgcccacttcgcgcgaaccgaccgtggcaccggccagactcaagggctttccgtaattttgagaagatttttttttgtaattttttattctgccccagctgatgtttgagccagcatgtcgcggaggaagcaagcg

SEQ ID NO: 28 - DNA, 320 bp, Homo sapiens
taaacgggataactagagatttcaaacaccttttatttgcctgtcttgaaaaaaaaatctaaatgaatacgcccgctaccaaaaggcaaaataaaaccaaccttaagggtttttgttgttttttttttttttcaaaagtggcgatagggactgtttggacattcaccagcctaattgctcagccccatgcgcggcccgcgcagccgccgccgccccgcgccccgcgccgcgcgcccgccaggccgccccgcgccgtccccgccggccgccccgctgatgccgctgccccgcgcggggcccgagcgccgctagcagcatgt

SEQ ID NO: 29 - DNA, 300 bp, Homo sapiens
tgcagtctgtggcatgtacaaggggctcaatcaattattattattactatcacttggagagtcaagcggcacaattttgtctcgtagtaaggctaacatgttacaactatcatctactaaaataaatataaacaactatactgtcagggctcatgataaatcgcaatgcattattgataataataattactgggacatgcgcgttccggccgaaggggggtaaatttcccaactccaggaatttgtggcggagagggcaaataactgcggctctcccggcgccccgatgctcgcaccatg

SEQ ID NO: 30 - DNA, 60 bp, Artificial sequence
  Synthetic construct
gatcccccaacatcccttctgccaccttcaagagaggtggcagaagggatgttgtttttc

SEQ ID NO: 31 - DNA, 60 bp, Artificial sequence
  Synthetic construct
gatcccccaccactgatcccaacgaattcaagagattcgttgggatcagtggtgtttttc

SEQ ID NO: 32 - DNA, 60 bp, Artificial sequence
  Synthetic construct
gatcccctcatttgccaccgagtcttttcaagagaaagactcggtggcaaatgatttttc

SEQ ID NO: 33 - DNA, 29 bp, Artificial sequence
  Synthetic construct
gccgacctatgtcaaggttgaagttcctg

SEQ ID NO: 34 - DNA, 29 bp, Artificial sequence
  Synthetic construct
gatgccttgaaacaagccaagctacctca

SEQ ID NO: 35 - DNA, 34 bp, Artificial sequence
  Primer
catcctcgagggctgttgacatctgcagagactg

SEQ ID NO: 36 - DNA, 36 bp, Artificial sequence
  Primer
tcgtagatctcatttctgcttgataaaagatcctgg